(19) United States
(12) Patent Application Publication
(10) Pub. No.: US 2025/0084385 A1
(43) Pub. Date: Mar. 13, 2025

(54) ADENO-ASSOCIATED VIRUS WITH ENGINEERED CAPSID

(71) Applicant: Tenaya Therapeutics, Inc., South San Francisco, CA (US)

(72) Inventors: Ze CHENG, South San Francisco, CA (US); Timothy C. HOEY, Hillsborough, CA (US); Christopher A. REID, Walnut Creek, CA (US)

(21) Appl. No.: 18/912,105

(22) Filed: Oct. 10, 2024

Related U.S. Application Data
(63) Continuation of application No. PCT/US2023/065598, filed on Apr. 10, 2023.
(60) Provisional application No. 63/378,983, filed on Oct. 10, 2022.
(60) Provisional application No. 63/329,778, filed on Apr. 11, 2022.

(51) Int. Cl. C12N 7/00; A61K 48/00; C07K 14/005; C12N 15/86

(52) U.S. Cl. CPC C12N 7/00; A61K 48/0008; A61K 48/0058; C07K 14/005; C12N 15/86; C12N 2750/14122; C12N 2750/14143; C12N 2750/14145

(57) ABSTRACT

The present disclosure provides engineered capsid proteins and recombinant adeno-associated virus (rAAV) virions with an engineered capsid protein. In particular, the disclosure provides AAV9 virions with engineered AAV9 capsid, AAV5/9 chimeric capsid, or combinatory capsid that achieves increased transduction efficiency in heart, increased heart-to-liver ratio, and/or other desirable properties.

### CROSS-REFERENCE TO RELATED APPLICATIONS

This application claims the benefit of U.S. Provisional Patent Application No. 63/329,778, filed Apr. 11, 2022, and U.S. Provisional Patent Application No. 63/378,983, filed Oct. 10, 2022, each of which is incorporated by reference herein in its entirety.

### REFERENCE TO SEQUENCE LISTING

The contents of the electronic sequence listing submitted herewith (TENA_037_01WO_SeqList_ST26.xml; Size: 1,210,478 bytes; and Date of Creation: Apr. 7, 2023) are incorporated by reference herein in their entireties.

### FIELD OF THE INVENTION

The present disclosure relates generally to adeno-associated virus vectors. In particular, the disclosure relates to engineered capsid proteins and recombinant adeno-associated virus virions having an engineered capsid protein and uses thereof.

### BACKGROUND

Adeno-associated virus (AAV) holds promise for gene therapy and other biomedical applications. In particular, AAV can be used to deliver gene products to various tissues and cells, both in vitro and in vivo. The capsid proteins of AAV largely determine the immunogenicity and tropism of AAV vectors.

For cardiac tissues, AAV subtype 9 (AAV9) is a preferred AAV vector due to its ability to transduce the heart following systemic delivery. While AAV9 can achieve moderate transduction of the heart, the majority of vector trafficks to the liver. Moreover, in order to achieve therapeutic levels of transduction in the heart, relatively high systemic doses are required, potentially leading to systemic inflammation and in turn, toxicity.

There is a need for developing an Adeno-associated virus with engineered capsid protein that achieves improved cardiac tropism, and optionally improved selectivity of cardiac tissues over liver. The present disclosure provides variants of the AAV9 capsid and/or chimeric AAV5/AAV9 capsid that form rAAV virions capable of transducing cardiac tissues and/or cell types for more efficiently and/or with more selectivity than rAAV virions comprising wild-type AAV9 capsid proteins, which can be used for safe and efficacious cardiac gene therapy.

### SUMMARY OF THE DISCLOSURE

In some aspects, the present disclosure provides recombinant adeno-associated virus (rAAV) capsid proteins, wherein the capsid protein shares at least 80%, at least 85%, at least 90%, at least 95% polypeptide sequence identity to an AAV9 VP3 reference sequence according to SEQ ID NO: 487, and wherein the capsid protein comprises, relative to reference sequence SEQ ID NO: 1, one or more of the modifications described herein.

In some embodiments, the capsid protein described herein comprises one, two, three, four or more substitutions in the VR-VIII site. In some embodiments, the capsid protein described herein comprises one, two, three, four or more insertions in the VR-VIII site. In some embodiments, the capsid protein described herein comprises, relative to reference SEQ ID NO:1, one, two, three, four or more substitutions at positions from 584 to 590 in the VR-VIII site, or one, two, three, four or more substitutions at positions from 585 to 590 in the VR-VIII site. In some embodiments, the capsid protein described herein comprises, relative to reference SEQ ID NO:1, one, two, three, four or more insertions at positions from 584 to 590 in the VR-VIII site, or one, two, three, four or more insertions at positions from 585 to 590 in the VR-VIII site.

In some embodiments, the capsid protein described herein comprises at least two, three, four, five or more substitutions in the VR-VIII site. In some embodiments, the capsid protein described herein comprises at least two, three, four or more insertions in the VR-VIII site. In some embodiments, the capsid protein described herein comprises, relative to reference SEQ ID NO: 1, at least two, three, four, five or more substitutions at positions from 584 to 590 in the VR-VIII site, or at least two, three, four, five or more substitutions at positions from 585 to 590 in the VR-VIII site. In some embodiments, the capsid protein described herein comprises, relative to reference SEQ ID NO:1, at least two, three, four or more insertions at positions from 584 to 590 in the VR-VIII site, or at least two, three, four or more insertions at positions from 585 to 590 in the VR-VIII site.

In some embodiments, the capsid protein described herein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence. These characteristics may be assessed in cells (e.g., iPSC-derived cardiac cells or cardiomyocytes) in vitro, or in mice or primates in vivo, by any methods known in the art or described herein.

The capsid protein may comprise an amino acid insertion at position 584 (relative to reference sequence SEQ ID NO: 1) comprising one or more of an asparagine (N), a threonine (T), a tyrosine (Y), phenylalanine (F), and an alanine (A).

The capsid protein may comprise an amino acid insertion at position 585 (relative to reference sequence SEQ ID NO:1) comprising one or more of a histidine (H) and a methionine (M).

The capsid protein may comprise an amino acid insertion at position 586 (relative to reference sequence SEQ ID NO: 1) comprising one or more of a histidine (H), a tyrosine (Y), a valine (V), a threonine (T), an alanine (A), an isoleucine (I), a tryptophan (W), a methionine (M), and a leucine.

The capsid protein may comprise an amino acid insertion at position 587 (relative to reference sequence SEQ ID NO:1) comprising one or more of an isoleucine (I) and a proline (P).

The capsid protein may comprise an amino acid insertion at position 588 (relative to reference sequence SEQ ID NO: 1) comprising one or more of an isoleucine (I), a threonine (T), and a proline (P).

The capsid protein may comprise one or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 1).

The capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D (relative to reference sequence SEQ ID NO:1).

In some aspects, the disclosure provides a recombinant adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion at position 584 comprising one or more of an asparagine (N), a threonine (T), a tyrosine (Y), phenylalanine (F), and an alanine (A);
an amino acid insertion at position 585 comprising one or more of a histidine (H) and a methionine (M);
an amino acid insertion at position 586 comprising one or more of a histidine (H), a tyrosine (Y), a valine (V), a threonine (T), an alanine (A), an isoleucine (I), a tryptophan (W), a methionine (M), and a leucine;
an amino acid insertion at position 587 comprising one or more of an isoleucine (I) and a proline (P);
an amino acid insertion at position 588 comprising one or more of an isoleucine (I), a threonine (T), and a proline (P);
an amino acid insertion at position 589 comprising one or more of a glycine (G) and a glutamine (Q);
one or more amino acid substitutions selected from the group consisting of N452K, N452A, N452V, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some aspects, a recombinant adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion between position 583 and 584 comprising one or more of an asparagine (N), a threonine (T), a tyrosine (Y), phenylalanine (F), and an alanine (A);
an amino acid insertion between position 584 and 585 comprising one or more of a histidine (H) and a methionine (M);
an amino acid insertion between position 585 and 586 comprising one or more of a histidine (H), a tyrosine (Y), a valine (V), a threonine (T), an alanine (A), an isoleucine (I), a tryptophan (W), a methionine (M), and a leucine (L);
an amino acid insertion between position 586 and 587 comprising one or more of an isoleucine (I) and a proline (P);
an amino acid insertion between position 587 and 588 comprising one or more of an isoleucine (I), a threonine (T), and a proline (P);
an amino acid insertion between position 588 and 589 comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

The capsid protein may comprise an amino acid insertion at position 584 (relative to reference sequence SEQ ID NO: 1) consisting of a TY, FN, or AT.

The capsid protein may comprise an amino acid insertion at position 585 (relative to reference sequence SEQ ID NO:1) consisting of MH.

The capsid protein may comprise an amino acid insertion at position 586 (relative to reference sequence SEQ ID NO:1) consisting of HY, VT, AI, WM, or ML.

The capsid protein may comprise an amino acid insertion at position 587 (relative to reference sequence SEQ ID NO:1) consisting of PI.

The capsid protein may comprise an amino acid insertion at position 588 (relative to reference sequence SEQ ID NO:1) consisting of IT or PT.

The capsid protein may comprise one or more amino acid substitutions selected from the group consisting of T582D, T582E, N583V, H584Q, S586K, A587P, A587S, Q588G, Q588M, A589S, A591I, G594Q, and G594D (relative to reference sequence SEQ ID NO:1).

The capsid protein may comprise one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R (relative to reference sequence SEQ ID NO:1).

The capsid protein may comprise one or more amino acid substitutions selected from the group consisting of Q585C, Q585S, and S586I (relative to reference sequence SEQ ID NO: 1).

The capsid protein may comprise one or more amino acid substitutions selected from the group consisting of Q585C, Q585S, S586I, A587V and A587G (relative to reference sequence SEQ ID NO:1).

The capsid protein may comprise one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L (relative to reference sequence SEQ ID NO:1).

The capsid protein may comprise one or more amino acid substitutions selected from the group consisting of A587V and A587G (relative to reference sequence SEQ ID NO:1).

The capsid protein may comprise an amino acid sequence selected from SEQ ID NOs: 599-692 and wherein the capsid protein shares at least 80%, at least 85%, at least 90%, at least 95%, at least 98%, or 100% identity to SEQ ID NOs: 496-589.

The capsid protein may comprise the amino acid sequence ANYG at positions 586-589 or at about positions 586-589 (relative to reference sequence SEQ ID NO:1).

The capsid protein may comprise two or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 1).

The capsid protein may comprise the amino acid substitution N452K, N452A, or N452V (relative to reference sequence SEQ ID NO:1).

The capsid protein may comprise the amino acid substitution N452K (relative to reference sequence SEQ ID NO: 1).

The capsid protein may comprise the amino acid substitution G453A or G453N (relative to reference sequence SEQ ID NO:1).

The capsid protein may comprise the amino acid substitution S454T or S454D (relative to reference sequence SEQ ID NO:1).

The capsid protein may comprise the amino acid substitution G455N (relative to reference sequence SEQ ID NO:1).

The capsid protein may comprise the amino acid substitution Q456L or Q456K (relative to reference sequence SEQ ID NO:1).

The capsid protein may comprise the amino acid substitution N457L or N457V (relative to reference sequence SEQ ID NO:1).

The capsid protein may comprise the amino acid substitution Q458I or Q458H (relative to reference sequence SEQ ID NO:1).

The capsid protein may comprise an amino acid sequence selected from KGSGQNQ (SEQ ID NO: 590), NASGQNQ (SEQ ID NO: 591), NGTGQNQ (SEQ ID NO: 592), NGSGLNQ (SEQ ID NO: 593), ANDNKLI (SEQ ID NO: 594), VNDNKVI (SEQ ID NO: 595), NGSGQNH (SEQ ID NO: 596), or ANDNKVI (SEQ ID NO: 597) at positions 452-458 or at about positions 452-458 (relative to reference sequence SEQ ID NO:1) and wherein the capsid protein shares at least 80%, at least 85%, at least 90%, at least 95%, at least 98%, or 100% identity to SEQ ID NOs: 488-495.

In some embodiments, the capsid protein described herein comprises relative to reference sequence SEQ ID NO: 1, at position 452 an amino acid selected from the group consisting of: K and N. In some embodiments, the capsid protein described herein comprises, relative to reference sequence SEQ ID NO: 1, an amino acid substitution N452K.

In some aspects, the disclosure provides a recombinant adeno-associated virus (rAAV) capsid protein which shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1, amino acid substitution N452K. In some embodiments, N452K is the only substitution in the capsid protein relative to the parental or wild-type AAV9. In some embodiments, N452K is not the only substitution in the capsid protein relative to the parental or wild-type AAV9.

In some embodiments, the capsid protein comprises, relative to reference sequence SEQ ID NO: 1:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and/or at position 590 an amino acid selected from: I, S, G, H, R and Q.

In some embodiments, the capsid protein comprises, relative to reference sequence SEQ ID NO: 1:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and
at position 590 an amino acid selected from: I, S, G, H, R and Q.

In some embodiments, the capsid protein comprises, relative to reference sequence SEQ ID NO: 1:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and/or at position 590 an amino acid selected from: I, S, G, H and R.

In some embodiments, the capsid protein comprises, relative to reference sequence SEQ ID NO: 1:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and
at position 590 an amino acid selected from: I, S, G, H and R.

In some embodiments, the capsid protein comprises, relative to reference sequence SEQ ID NO: 1:
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q; and
   optionally at position 452 an amino acid selected from the group consisting of: N and K.

In some embodiments, the capsid protein comprises, relative to reference sequence SEQ ID NO: 1:
at position 452 an amino acid selected from the group consisting of: K and N;
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

In some embodiments, the capsid protein comprises, relative to reference sequence SEQ ID NO: 1:
at position 584 amino acid R;
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G;
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M; and
   optionally at position 452 an amino acid selected from the group consisting of: N and K.

In some embodiments, the capsid protein comprises, relative to reference sequence SEQ ID NO: 1, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M.

In some embodiments, the capsid protein comprises, relative to reference sequence SEQ ID NO: 1:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q; and
optionally at position 452 an amino acid selected from the group consisting of: N and K.

In some embodiments, the capsid protein comprises, relative to reference sequence SEQ ID NO: 1:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

In some embodiments, the capsid protein comprises, relative to reference sequence SEQ ID NO: 1:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or
at position 590 an amino acid selected from the group consisting of: I, S, G, H and R; and
optionally at position 452 an amino acid selected from the group consisting of: N and K.

In some embodiments, the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, G, M, C, V and T;
(iii) at position 586 an amino acid selected from the group consisting of: N, T, M, G, and D;
(iv) at position 587 an amino acid selected from the group consisting of: T, L, I, K, S, N and V;
(v) at position 588 an amino acid selected from the group consisting of: V, F, Y, L, T, S, I and R;
(vi) at position 589 an amino acid selected from the group consisting of: S, N, L, T, I and R; and
(vii) at position 590 an amino acid selected from the group consisting of: I, S, G, H and R.

In some embodiments, the capsid protein comprises, relative to reference sequence SEQ ID NO:1:
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q; and
optionally at position 452 an amino acid selected from the group consisting of: N and K.

In some embodiments, the capsid protein comprises, relative to reference sequence SEQ ID NO:1:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

In some embodiments, the capsid protein comprises, relative to reference sequence SEQ ID NO: 1:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or
at position 590 an amino acid selected from the group consisting of: I, S, G, and R; and
optionally at position 452 an amino acid selected from the group consisting of: N and K.

In some embodiments, the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N;
(iv) at position 587 an amino acid selected from the group consisting of: T, N, and V;
(v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

In some embodiments, the capsid protein comprises, relative to reference sequence SEQ ID NO: 1:
at position 452 an amino acid selected from the group consisting of: K and N; and
at position 587 amino acid substitution A587T.

In some embodiments, the capsid protein comprises, relative to reference sequence SEQ ID NO: 1:
at position 452 an amino acid selected from the group consisting of: K and N; and
amino acid N or R at one, two or more positions selected from the group consisting of: 584, 585, 586, 588, 589, and 590.

In some embodiments, the capsid protein comprises, relative to reference sequence SEQ ID NO: 1:
at position 452 an amino acid selected from the group consisting of: K and N; and
amino acid S at two or more positions selected from the group consisting of: 585, 586, 587, 588, 589 and 590.

In some embodiments, the capsid protein comprises, relative to reference sequence SEQ ID NO: 1:
at position 452 an amino acid selected from the group consisting of: K and N; and
at three, four or more positions in the region 585-590 of the VR-VIII site, an amino acid selected from the group consisting of: N, S, T, R and I.

In some embodiments, the capsid protein comprises, relative to reference sequence SEQ ID NO: 1:
at three, four or more positions in the region 585-590 of the VR-VIII site, an amino acid selected from the group consisting of: N, S, T, and R.

In some embodiments, the capsid protein comprises, relative to reference sequence SEQ ID NO: 1:
at position 452 an amino acid selected from the group consisting of: K and N; and
at three, four or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 1:
at three, four or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 1:
at position 452 an amino acid selected from the group consisting of: K and N; and
at four, five or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 1:
at four, five or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 1, at least two, three, four or more of the amino acid substitutions Q585E, S586N, A587T, Q588V, A589S, Q590I, and/or N452K (or any combination of these substitutions). In some embodiments, the capsid protein comprises, relative to reference sequence SEQ ID NO: 1, amino acid substitutions Q585E, S586N, A587T, Q588V, A589S, Q590I, and N452K.

In some embodiments, the capsid protein comprises, relative to reference sequence SEQ ID NO: 1, at least two, three, four or more of the amino acid substitutions S586T, A587L, Q588F, A589N, Q590S, and/or N452K (or any combination of these substitutions). In some embodiments, the capsid protein comprises, relative to reference sequence SEQ ID NO: 1, amino acid substitutions S586T, A587L, Q588F, A589N, Q590S, and N452K.

In some embodiments, the capsid protein comprises, relative to reference sequence SEQ ID NO: 1, at least two, three, four or more of the amino acid substitutions Q585N, A587T, Q588Y, A589L, Q590G, and/or N452K (or any combination of these substitutions). In some embodiments, the capsid protein comprises, relative to reference sequence SEQ ID NO: 1, amino acid substitutions Q585N, A587T, Q588Y, A589L, Q590G, and N452K.

In some embodiments, the capsid protein comprises, relative to reference sequence SEQ ID NO: 1, at least two, three, four or more of the amino acid substitutions Q585G, A587I, Q588L, A589T, Q590H, and/or 452K (or any combination of these substitutions). In some embodiments, the capsid protein comprises, relative to reference sequence SEQ ID NO: 1, amino acid substitutions Q585G, A587I, Q588L, A589T, Q590H, and N452K.

In some embodiments, the capsid protein comprises, relative to reference sequence SEQ ID NO: 1, at least two, three, four or more of the amino acid substitutions Q585M, S586M, A587T, Q588T, and/or Q590R (or any combination of these substitutions). In some embodiments, the capsid protein comprises, relative to reference sequence SEQ ID NO: 1, amino acid substitutions Q585M, S586M, A587T, Q588T, and Q590R. In some embodiments, the capsid protein comprises, relative to reference sequence SEQ ID NO: 1, amino acid substitutions Q585M, S586M, A587T, Q588T, and Q590R; and amino acid N at position 452.

In some embodiments, the capsid protein comprises, relative to reference sequence SEQ ID NO: 1, at least two, three, four or more of the amino acid substitutions Q585N, A587T, Q588Y, A589L, and/or Q590G (or any combination of these substitutions). In some embodiments, the capsid protein comprises, relative to reference sequence SEQ ID NO: 1, amino acid substitutions Q585N, A587T, Q588Y, A589L, and Q590G. In some embodiments, the capsid protein comprises, relative to reference sequence SEQ ID NO: 1, amino acid substitutions Q585N, A587T, Q588Y, A589L, and Q590G; and amino acid N at position 452.

In some embodiments, the capsid protein comprises, relative to reference sequence SEQ ID NO: 1, at least two, three, four or more of the amino acid substitutions Q585C, A587T, Q588S, A589I, and/or Q590R (or any combination of these substitutions). In some embodiments, the capsid protein comprises, relative to reference sequence SEQ ID NO: 1, amino acid substitutions Q585C, A587T, Q588S, A589I, and Q590R. In some embodiments, the capsid protein comprises, relative to reference sequence SEQ ID NO: 1, amino acid substitutions Q585C, A587T, Q588S, A589I, and Q590R; and amino acid N at position 452.

In some embodiments, the capsid protein comprises, relative to reference sequence SEQ ID NO: 1, at least two, three, four or more of the amino acid substitutions Q585E, S586D, A587N, Q588I, A589R, and/or Q590S (or any combination of these substitutions). In some embodiments, the capsid protein comprises, relative to reference sequence SEQ ID NO: 1, amino acid substitutions Q585E, S586D, A587N, Q588I, A589R, and Q590S. In some embodiments, the capsid protein comprises, relative to reference sequence SEQ ID NO: 1, amino acid substitutions Q585E, S586D, A587N, Q588I, A589R, and Q590S; and amino acid N at position 452.

In some embodiments, the capsid protein comprises, relative to reference sequence SEQ ID NO: 1, at least two, three, four or more of the amino acid substitutions Q585E, S586D, A587N, Q588I, A589R, Q590S, and/or N452K (or any combination of these substitutions). In some embodiments, the capsid protein comprises, relative to reference sequence SEQ ID NO: 1, amino acid substitutions Q585E, S586D, A587N, Q588I, A589R, Q590S, and N452K.

In some embodiments, the capsid protein comprises, relative to reference sequence SEQ ID NO: 1, amino acid S586G and/or Q588Y. In some embodiments, the capsid protein comprises, relative to reference sequence SEQ ID NO: 1, amino acid substitutions S586G and Q588Y; and amino acid N at position 452.

In some embodiments, the capsid protein comprises, relative to reference sequence SEQ ID NO: 1, at least two, three, four or more of the amino acid substitutions substitutions S586A, A587N, Q588Y, A589G, and/or N452K (or any combination of these substitutions). In some embodiments, the capsid protein comprises, relative to reference sequence SEQ ID NO: 1, amino acid substitutions substitutions S586A, A587N, Q588Y, A589G, and N452K.

In some embodiments, the capsid protein of any of the embodiments described herein comprises, relative to reference sequence SEQ ID NO:1, amino acids ATN at positions 581-583, and amino acids AQTG at positions 591-594.

In some embodiments, the capsid protein of any of the embodiemnts described herein comprises, relative to reference sequence SEQ ID NO:1, amino acids ATNH at positions 581-584, and amino acids AQTG at positions 591-594.

In some embodiments, the capsid protein described herein comprises, relative to reference sequence SEQ ID NO:1, any one of the following:
(i) amino acid sequence ATNHENTVSIAQTG at the VR-VIII positions 581-594, and amino acid K at the VR-IV position 452;
(ii) amino acid sequence ATNHQTLFNSAQTG at the VR-VIII positions 581-594, and amino acid K at the VR-IV position 452;
(iii) amino acid sequence ATNHNSTYLGAQTG at the VR-VIII positions 581-594, and amino acid K at the VR-IV position 452;
(iv) amino acid sequence ATNHGSILTHAQTG at the VR-VIII positions 581-594, and amino acid K at the VR-IV position 452;
(v) amino acid sequence ATNHMMTTARAQTG at the VR-VIII positions 581-594, and amino acid N at the VR-IV position 452;
(vi) amino acid sequence ATNHNSTYLGAQTG at the VR-VIII positions 581-594, and amino acid N at the VR-IV position 452;
(vii) amino acid sequence ATNHCSTSIRAQTG at the VR-VIII positions 581-594, and amino acid N at the VR-IV position 452;
(viii) amino acid sequence ATNHEDNIRSAQTG at the VR-VIII positions 581-594, and amino acid N at the VR-IV position 452;
(ix) amino acid sequence ATNHEDNIRSAQTG at the VR-VIII positions 581-594, and amino acid K at the VR-IV position 452;
(x) amino acid sequence ATNHNNVISGAQTG at the VR-VIII positions 581-594, and amino acid K at the VR-IV position 452;
(xi) amino acid sequence ATNHQGAYAQAQTG at the VR-VIII positions 581-594, and amino acid N at the VR-IV position 452;
(xii) amino acid sequence ATNHQANYGQAQTG at the VR-VIII positions 581-594, and amino acid K at the VR-IV position 452;
(xiii) amino acid sequence ATNHNMNRVNAQTG at the VR-VIII positions 581-594, and amino acid N at the VR-IV position 452;
(xiv) amino acid sequence ATNHNNVISGAQTG at the VR-VIII positions 581-594, and amino acid N at the VR-IV position 452;
(xv) amino acid sequence ATNHSNSVQSAQTG at the VR-VIII positions 581-594, and amino acid N at the VR-IV position 452;
(xvi) amino acid sequence ATNHSSTFQGAQTG at the VR-VIII positions 581-594, and amino acid N at the VR-IV position 452;
(xvii) amino acid sequence ATNHVSSFTSAQTG at the VR-VIII positions 581-594, and amino acid N at the VR-IV position 452;
(xviii) amino acid sequence ATNHSTTNFRAQTG at the VR-VIII positions 581-594, and amino acid N at the VR-IV position 452;
(xix) amino acid sequence ATNHSSIFNSAQTG at the VR-VIII positions 581-594, and amino acid N at the VR-IV position 452;
(xx) amino acid sequence ATNHAGNYNNAQTG at the VR-VIII positions 581-594, and amino acid N at the VR-IV position 452;
(xxi) amino acid sequence ATNHTSVISIAQTG at the VR-VIII positions 581-594, and amino acid N at the VR-IV position 452;
(xxii) amino acid sequence ATNHHSRVEIAQTG at the VR-VIII positions 581-594, and amino acid N at the VR-IV position 452;
(xxiii) amino acid sequence ATNHSSIIYSAQTG at the VR-VIII positions 581-594, and amino acid N at the VR-IV position 452;
(xxiv) amino acid sequence ATNHSGRDSYAQTG at the VR-VIII positions 581-594, and amino acid N at the VR-IV position 452;
(xxv) amino acid sequence ATNHSSSYNNAQTG at the VR-VIII positions 581-594, and amino acid N at the VR-IV position 452;
(xxvi) amino acid sequence ATNHHNPSINAQTG at the VR-VIII positions 581-594, and amino acid N at the VR-IV position 452;
(xxvii) amino acid sequence ATNHNRNGLLAQTG at the VR-VIII positions 581-594, and amino acid N at the VR-IV position 452;
(xxviii) amino acid sequence ATNHESTSVRAQTG at the VR-VIII positions 581-594, and amino acid N at the VR-IV position 452;
(xxix) amino acid sequence ATNHNIRTEMAQTG at the VR-VIII positions 581-594, and amino acid N at the VR-IV position 452;
(xxx) amino acid sequence ATNHQTLFNSAQTG at the VR-VIII positions 581-594, and amino acid N at the VR-IV position 452;
(xxxi) amino acid sequence ATNHLSVSSIAQTG at the VR-VIII positions 581-594, and amino acid N at the VR-IV position 452;
(xxxii) amino acid sequence ATNHEDIIRSAQTG at the VR-VIII positions 581-594, and amino acid N at the VR-IV position 452;
(xxxiii) amino acid sequence ATNRQTAQAQAQTG at the VR-VIII positions 581-594, and amino acid N at the VR-IV position 452; or
(xxxiv) amino acid sequence ATNRQIAQAQAQTG at the VR-VIII positions 581-594, and amino acid N at the VR-IV position 452.

In some embodiments, the capsid protein of any of the embodiments described herein comprises any substitution and/or insertion motif described herein, e.g., described in any one of the tables and/or sequences provided herein. In some embodiments, the capsid protein of any of the embodiments described herein comprises a substitution motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any substitution motif described herein, e.g., described in any one of the tables and/or sequences provided herein. In some embodiments, the capsid protein of any of the embodiments described herein comprises an insertion motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any insertion motif described herein, e.g., described in any one of the tables and/or sequences provided herein.

In some embodiments, the capsid protein of any of the embodiments described herein shares, or comprises a sequence sharing, at least 85%, at least 90%, at least 95%, at least 96%, at least 97%, at least 99%, or 100% amino acid sequence identity to an AAV9 VP3 sequence according to SEQ ID NO: 487, except for the specified modifications.

In some embodiments, the capsid protein of any of the embodiments described herein shares, or comprises a sequence sharing, at least 85%, at least 90%, at least 95%, at least 96%, at least 97%, at least 99%, or 100% amino acid sequence identity to an AAV9 VP2 sequence according to SEQ ID NO: 486, except for the specified modifications.

In some embodiments, the capsid protein of any of the embodiments described herein shares, or comprises a sequence sharing, at least 85%, at least 90%, at least 95%, at least 96%, at least 97%, at least 99%, or 100% amino acid sequence identity to an AAV9 VP1 sequence according to SEQ ID NO: 1, except for the specified modifications.

In some embodiments, the capsid protein of any of the embodiments described herein comprises, consists essentially of, or consists of an amino acid sequence at least 85%, 90%, 95%, 96%, 97%, 98%, 99%, or 100% identical to any one of the modified capsid protein sequences disclosed herein (e.g., VP1, VP2, or VP3), or a functional fragment thereof.

In some embodiments, the capsid protein described herein comprises, consists essentially of, or consists of a polypeptide sequence of any one of the modified capsid protein sequences disclosed herein (e.g., VP1, VP2, or VP3).

In some embodiments, the capsid protein of any of the embodiments described herein comprises, consists essentially of, or consists of an amino acid sequence having at least 80%, 85%, 90%, 95%, 96%, 97%, 98%, 99%, or 100% sequence identity to any sequence selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

In some embodiments, the capsid protein comprises, consists essentially of, or consists of an amino acid sequence having at least 80%, 85%, 90%, 95%, 96%, 97%, 98%, 99%, or 100% sequence identity to any capsid protein sequence provided herein (e.g., any capsid protein sequence in any one of the tables and/or sequences provided herein).

In some embodiments, the capsid protein described herein comprises, consists essentially of, or consists of a polypeptide sequence of any one selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774.

In some embodiments, the capsid protein is any capsid protein described in any one of the tables and/or sequences provided herein. In some embodiments, the capsid protein comprises, consists essentially of, or consists of an amino acid sequence of any capsid protein described in any one of the tables and/or sequences provided herein.

In some embodiments, the capsid is not a chimeric capsid and/or not a combinatory capsid.

In some aspects, the disclosure provides a recombinant adeno-associated virus (rAAV) virion comprising any one of the capsid proteins described herein. In some aspects, the disclosure provides recombinant adeno-associated virus (rAAV) virions comprising any of the capsid proteins described herein and a vector genome. The vector genome may comprise a polynucleotide cassette flanked by inverted terminal repeats (ITRs). In some embodiments, the polynucleotide cassette encodes any one or more of the proteins (or gene products) described herein. In some embodiments, the polynucleotide cassette comprises any of the transgenes described herein.

In some embodiments, the rAAV virion specifically transduces heart cells.

In some embodiments, the rAAV virion specifically transduces cardiomyocytes.

In some embodiments, the rAAV virion traffics to the heart.

In some embodiments, the rAAV virion traffics to at least one organ other than the liver.

In some embodiments, the rAAV virion exhibits a higher heart transduction efficiency than an rAAV virion having an AAV9 VP1 capsid protein according to SEQ ID NO: 1.

In some embodiments, administration of the rAAV virion to a subject leads to a lower liver viral load than administration of an rAAV virion having an AAV9 VP1 capsid protein according to SEQ ID NO: 1. In some embodiments, administration of the rAAV virion to a subject leads to a lower liver viral load in a primate or as assessed in a primate, than administration of an rAAV virion having an AAV9 VP1 capsid protein according to SEQ ID NO: 1. In some embodiments, administration of the rAAV virion to a subject leads to at least 2, 3, 4, 5, 6, 7, 8, 9 or 10 times lower liver viral load than administration of an rAAV virion having an AAV9 VP1 capsid protein according to SEQ ID NO: 1 (e.g., in a primate or as assessed in a primate).

In some embodiments, the rAAV virion exhibits a higher heart-to-liver transduction ratio than an rAAV virion having an AAV9 VP1 capsid protein according to SEQ ID NO: 1. In some embodiments, the rAAV virion exhibits a heart-to-liver transduction ratio which is at least 2, 3, 4, 5, 6, 7, 8, 9 or 10 times higher than an rAAV virion having an AAV9 VP1 capsid protein according to SEQ ID NO: 1.

In some embodiments, the rAAV virion exhibits a higher transduction efficiency than an rAAV virion having an AAV9 VP1 capsid protein according to SEQ ID NO: 1, assessed in a primate.

In some embodiments, the rAAV virion exhibits a higher heart transduction efficiency than an rAAV virion having an AAV9 VP1 capsid protein according to SEQ ID NO: 1 (e.g., as assessed in a primate).

In some embodiments, the rAAV virion exhibits a higher heart-to-liver transduction ratio than an rAAV virion having an AAV9 VP1 capsid protein according to SEQ ID NO: 1, assessed in a primate.

In some embodiments, the rAAV virion exhibits at least 2, 3, 4, 5, 6, 7, 8, 9 or 10 times higher heart-to-liver transduction ratio than an rAAV virion having an AAV9 VP1 capsid protein according to SEQ ID NO: 1 (e.g., as assessed in a primate).

In some embodiments, the polynucleotide cassette comprises a polynucleotide sequence encoding MYBPC3, DWORF, PKP2, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, JPH2, PLN, ATP2A2, CACNA1C, DMD, DMPK, EPG5, EVC, EVC2, FBN1, NF1, SCN5A, SOS1, NPR1, ERBB4, VIP, MYH6, MYH7, Cas9, RBM20, MYOCD, ASCL1, GATA4, MEF2C, TBX5, miR-133, and/or MESP1.

In some embodiments, the polynucleotide cassette comprises a polynucleotide sequence encoding MYBPC3, DWORF, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, and/or ATP2A2. In some embodiments, the polynucleotide cassette comprises a polynucleotide sequence encoding JPH2 and/or PLN. In some embodiments, the polynucleotide cassette comprises a polynucleotide sequence encoding Lamin A isoform of LMNA, Lamin C isoform of LMNA, LAMP2a, LAMP2b, LAMP2c, DPI isoform of DSP, or DPII isoform of DSP. In some embodiments, the polynucleotide cassette comprises a polynucleotide sequence encoding MMP11, SYNPO2L (e.g., SYNPO2LA or SYNPO2LA), or an inhibitory oligonucleotide targeting MTSS1.

In some embodiments, the polynucleotide cassette comprises a polynucleotide sequence which encodes a protein selected from the group consisting of: MYBPC3, DWORF, PKP2, LMNA, LAMP2, BAG3, CRYAB, JPH2, PLN, TTNI3, MYOCD, ASCL1, DSP, JUP, DSP, MYH6, MYH7, RBM20, Cas9. In some embodiments, the polynucleotide cassette comprises a polynucleotide sequence encoding saCas9. In some embodiments, the polynucleotide cassette comprises a polynucleotide sequence encoding a C151R mutant form of BAG3 polypeptide. In some embodiments, the polynucleotide cassette comprises a polynucleotide sequence encoding a guide RNA targeting a mutant PLN (such as a deletious mutant of PLN, e.g., PLN-R14Del).

In some embodiments, the polynucleotide cassette comprises a polynucleotide sequence encoding CACNA1C, DMD, DMPK, EPG5, EVC, EVC2, FBN1, NF1, SCN5A, SOS1, NPR1, ERBB4, VIP, MYH7, and/or Cas9.

In some embodiments, the polynucleotide cassette comprises a polynucleotide sequence encoding MYOCD, ASCL1, GATA4, MEF2C, TBX5, miR-133, and/or MESP1.

In another aspect, the disclosure provides pharmaceutical compositions comprising any rAAV virion described herein and a pharmaceutically acceptable carrier.

In another aspect, the disclosure provides a polynucleotide encoding any of the capsid proteins described herein.

In another aspect, the disclosure provides a method of transducing a cell, comprising contacting the cell with a polynucleotide encoding any of the capsid proteins described herein.

In another aspect, the disclosure provides methods of transducing a cardiac cell, comprising contacting the cardiac cell with any rAAV virion described herein, wherein the rAAV virion transduces the cardiac cell.

In some embodiments, the cardiac cell is a cardiomyocyte.
efficiency in the cell than an rAAV virion having an AAV9 VP1 capsid protein according to SEQ ID NO: 1.

In another aspect, the disclosure provides methods of delivering one or more gene products to a cardiac cell, comprising contacting the cardiac cell with any rAAV virion described herein.

In some embodiments, the cardiac cell is a cardiomyocyte.

In another aspect, the disclosure provides methods of treating a cardiac pathology in a subject in need thereof, comprising administering a therapeutically effective amount of any rAAV virion or any pharmaceutical composition described herein, wherein the rAAV virion transduces cardiac tissue.

In another aspect, the disclosure provides methods of treating a heart disease or condition in a subject in need thereof, comprising administering a therapeutically effective amount of any rAAV virion or any pharmaceutical composition described herein, optionally whrein the heart disease or condition is a cardiomyopathy (e.g., DCM or HCM) or a heart failure (e.g., heart failure with reduced ejection fraction).

In another aspect, the disclosure provides kits comprising a vector or plasmid encoding any AAV capsid protein described herein.

In another aspect, the disclosure provides kits comprising any pharmaceutical composition described herein and instructions for use.

### BRIEF DESCRIPTION OF THE DRAWINGS

FIG. 1 depicts the AAV9 capsid highlighting amino acids in selected AAV9 variable regions (VR-IV and VR-VIII site).

FIG. 2 shows a schematic of directed evolution selection strategy and variant characterization. Following library generation, each library was subjected to two rounds of selection in a primates.

FIG. 3. shows a vector map for the vector genomes used in screening for capsid protein variants.

FIG. 4. shows a plot of data from the second-round screening. Liver viral genome abundance is plotted against heart mRNA transcript abundance (“Heart transduction”) on a log₂ scale. In each case, the values are normalized against the values for a reference AAV9 virion.

FIGS. 5A-5C plot 102 variants selected as having the desired cell properties (high heart transduction relative to AAV9, high heart-to-liver ratio relative to AAV9, or both).

FIG. 5A plots heart transduction measurements of the 102 selected variants on x-axis and heart-to-liver ratios on y-axis.

FIG. 5B shows the subset of variants from the sub-library no. 1 in Table 4 with both randomized VR-IV (amino acids 452 to 458 of AAV9 VP1) and substituted VR-VIII (amino acids 586 to 589 of AAV9 VP1).

FIG. 5C shows novel variants with modified VR-VIII (amino acids 581 to 594 on AAV9 VP1).

FIG. 6 shows a schematic of re-testing rAAV virions having engineered capsid proteins in a mouse model.

FIGS. 7A-7C show heart transduction (FIG. 7A), liver viral load (FIG. 7B), and heart-to-liver ratio (FIG. 7C) measurements of the selected variants and AAV9 reference.

FIGS. 8A-8B show schematics of the modified viral capsids (FIG. 8A) and screening strategy for evaluating transduction efficiency in various organs and tissues of animal models transduced with barcoded modified viral capsids (FIG. 8B).

FIG. 9 shows graphs measuring transduction/viral load levels of novel capsids without an N452K mutation (ZC404, ZC470, ZC428, and ZC416) and with an N452K mutation (ZC373, ZC374, ZC375, and ZC376) in cynomolgus monkey heart and liver, mouse heart and liver, and human iPSCs.

FIG. 10 shows a schematic of a screening strategy for evaluating transduction efficiency in various organs and tissues of animal models transduced with modified viral capsids.

FIGS. 11A-11B show a heatmap of transduction efficiency of modified AAV capsids. Each column represents one capsid and each row is one sample type. The average measurements of 4 animals, 3 animals, 6 animals, or 2 multiplicities of infection are shown for cynomolgus monkey, mouse, pig, and iPSC-CMs, respectively. The capsids are ordered in columns from left to right ranked by their heart-to-liver ratio in cynomolgus monkey. AAV9-1, AAV9-2, and AAV9-3 are all wildtype AAV9 capsid serve as control replicates.

FIG. 12 provides graphs showing transduction in heart, liver viral load, and the heart-to-liver transduction ratio in cynomolgus monkey, mouse, and pig using four novel AAV capsids. Results show fold change relative to wild-type AAV9 control.

FIG. 13 provides a graph showing heart-to-liver ratio, heart transduction, and liver viral load of four novel capsids compared to AAV9 wild-type control in Cynomolgus monkeys. Animals were administered 1E+13 vg/kg via intravenous bolus administration. Tissue was collected 4-weeks post injection. The figure shows fold change relative to wildtype AAV9 control.

FIG. 14 provides graphs showing heart-to-liver ratio, heart transduction, and liver viral load of ZC375, ZC401, ZC428, and ZC478 capsids compared to AAV9 wild-type control in CD-1 mice. Virus was administered at 2E+13 vg/kg for ZC375, ZC401, and ZC428, and 1.45E+13 vg/kg for ZC478 through retro-orbital injection. Dosage matched AAV9 controls were included. Tissue was collected 18 days post injection. Results show fold change relative to AAV9 control.

FIG. 15 provides graphs showing heart-to-liver ratio, heart transduction, and liver viral load of ZC401 capsid compared to AAV9 wild-type control in C57BL/6NCrl mice. The viruses were administered at 2E+13 vg/kg through retro-orbital injection. Tissue was collected 18 days post injection. Results show fold change relative to AAV9.

FIG. 16 provides a graph showing heart and liver transduction by ZC401 capsid compared to AAV9 wild-type control in CD-1 mice. Viruses were administered at 2E+13 vg/kg (AAV9 and ZC401) or 1.2E+14 vg/kg (ZC401) through retro-orbital injection. Tissue was collected 18 days post injection. Results show fold change relative to AAV9.

FIG. 17 shows incorporation of N452K substitution into AAV9-based capsid variants. The figure provides an image of capsid structure illustrating the location of VR-VIII region and N452 (Asn452) on the wildtype AAV9 capsid and tables showing the names of sequences of parental capsids (on the left) and new N452K capsids (on the right) for AAV9-based VR-VIII substitution variants.

FIG. 18 shows testing N452K variants in multiple models. The figure shows a heatmap of transduction efficiency of modified AAV capsids from FIG. 17. Each column represents one capsid and each row is one sample type. The N452K variants were tested in Cynomolgus monkeys, mice, and human iPSC-CMs using pooled barcode-based methodology. Heart transduction and iPSC-CM transduction were measured by NGS-based quantification of RNA samples. Liver viral load was measured by NGS-based quantification of DNA samples. Heart-to-liver ratio was calculated by dividing heart transduction by liver viral load. All the measurements were normalized to AAV9 control.

FIG. 19 is a graph showing iPSC-CM transduction efficiency improvements of N452K variants compared to matched parental capsids without the N452 substitution (in fold change). N452K substitution consistently enhanced transduction efficiency.

FIG. 20 provides graphs showing heart-to-liver ratio, heart transduction, and liver viral load of select capsids from FIG. 18 compared to AAV9 wild-type control in Cynomolgus monkey (a non-human primate or “NHP”). All the values are relative to the performance of wildtype AAV9 control. ZC533, ZC536, and ZC538 showed improved heart-to-liver ratio and/or improved heart transduction in NHPs relative to AAV9.

FIG. 21 shows a schematic of experiment comparing biodistribution and transduction of new capsids and AAV9 in NHPs. In this experiment, performance of top capsids was measured in NHPs injected individually (one test article per animal) at a therapeutic relevant dose. AAV9, ZC375, and ZC428 were administered at 6E+13 vg/kg systemically. This study was divided to two phases and in each phase, one novel capsid and AAV9 control were tested with 4 Cynomolgus Monkeys per test article. Animals were sacrificed at 28-day post injection. RNA and DNA were extracted from heart and liver tissues, followed by RT-qPCR based quantification of viral.

FIG. 22 is a graph showing viral transgene expression (“Heart RNA”) levels in the heart from the NHP biodistribtion and transduction study depicted in FIG. 21. Viral transgene expression levels were measured by RT-qPCR analysis on RNA samples and normalized to the average of all AAV9 data points. Each dot on the figure represents one individual animal for which 4 heart biopsy samples were analyzed and averaged. Both ZC375 and ZC428 showed comparable transgene expression in the heart compared to their matched AAV9 control.

FIGS. 23A-23B are graphs showing reduced liver tropism compared to AAV9. The figure shows viral transgene expression (“Liver RNA”; FIG. 23A) and viral genome load (“Liver DNA”; FIG. 23B) levels in the liver from the NHP biodistribution and transduction study in FIG. 21 (with animals systemically administered ZC375, ZC428, or wild-type control AAV9 at 6E+13 vg/kg). Viral transgene expression levels were measured by RT-qPCR analysis on RNA samples and normalized to the average of all AAV9 data points. Viral genome load levels were measured by qPCR analysis on DNA samples and normalized to the average of all AAV9 data points. Each dot on the figure represents one individual animal for which 2 liver biopsy samples were analyzed and averaged. ZC375 and ZC428 showed reduced transduction in the liver at both RNA and DNA levels compared to their matched AAV9 control.

FIGS. 24A-24B are graphs showing heart transduction to liver transduction ratios from the NHP biodistribution and transduction study depicted in FIG. 21, calculated by either heart RNA-based and liver RNA-based measurements (FIG. 24A), or heart RNA-based and liver DNA-based measurements (FIG. 24B). The ratios were individually calculated to each animal. ZC375 and ZC428 showed improved heart-to-liver ratio compared to their matched AAV9 control.

### DETAILED DESCRIPTION

The disclosure provides engineered capsid proteins and recombinant adeno-associated virus (rAAV) virions. In particular, the disclosure provides engineered capsid proteins (including chimeric capsid proteins), methods of identifying them, and methods of using them. The methods of identifying new capsid proteins disclosed herein have wide applicability for any serotype of AAV, including chimeric capsid proteins. In addition, they can be applied to iteratively improve capsid proteins that have mutations from this or other methods. In general, the methods of the disclosure relate to preparation of randomized or semi-randomized libraries of AAV capsids in the form of cap gene polynucleotides, preparation of AAV virions comprising such capsids (either by incorporating the cap gene library into an AAV genome or providing it in trans such as on a plasmid transfected into the packaging line), positively or negatively selecting the AAV virions, and recovering the cap gene for sequencing. In some embodiments, the recovery and sequencing include nanopore sequencing. Other high-throughput or next-generation-sequencing (NGS) methods can be used.

In some embodiments, the present disclosure provides recombinant adeno-associated virus (rAAV) virions comprising:
a) a capsid protein as described herein; and
b) a heterologous nucleic acid comprising a nucleotide sequence encoding one or more gene products.

In some embodiments, the rAAV virions disclosed herein comprise an AAV9 capsid protein as disclosed herein. In some embodiments, the rAAV virions disclosed herein comprise a chimeric AAV5/AAV9 capsid protein as disclosed herein. In some embodiments, the rAAV virions disclosed herein comprise a combinatory capsid protein as disclosed herein.

In some embodiments, the AAV9 capsid protein described herein comprises a sequence that shares at least about 80%, 85%, 90%, 95%, 96%, 97%, 98%, 99%, 99.5%, or 100% identity to SEQ ID NO: 1, as shown below. In some embodiments, the AAV9 capsid protein described herein comprises a sequence that shares at least about 80%, 85%, 90%, 95%, 96%, 97%, 98%, 99%, 99.5%, or 100% identity to SEQ ID NO: 487. The N-terminal residue of VP1, VP2, and VP3, as well as the VR sites (VR-IV, VR-V, VR-VII and VR-VIII), are indicated (in bold, and underlined) in the sequence of full-length VP1 (SEQ ID NO: 1) below. The wild type AAV9 VP1 has the amino acid sequence of SEQ ID NO:1. The wild type AAV9 VP2 has the amino acid sequence of SEQ ID NO:486. The wild type AAV9 VP3 has the amino acid sequence of SEQ ID NO:487.

```
(SEQ ID NO: 1)
VP1--> (SEQ ID NO: 1)
MAADGYLPDWLEDNLSEGIREWWALKPGAPQPKANQQHQDNARGLVLPGYKYLGPGNGLDKGE
PVNAADAAALEHDKAYDQQLKAGDNPYLKYNHADAEFQERLKEDTSFGGNLGRAVFQAKKRLL
                      VP2--> (SEQ ID NO: 486)
EPLGLVEEAAKTAPGKKRPVEQSPQEPDSSAGIGKSGAQPAKKRLNFGQTGDTESVPDPQPIG
                          VP3--> (SEQ ID NO: 487)
EPPAAPSGVGSLTMASGGGAPVADNNEGADGVGSSSGNWHCDSQWLGDRVITTSTRIWALPTY
NNHLYKQISNSTSGGSSNDNAYFGYSTPWGYFDFNRFHCHFSPRDWQRLINNNWGFRPKRLNF
KLFNIQVKEVTDNNGVKTIANNLTSTVQVFTDSDYQLPYVLGSAHEGCLPPFPADVFMIPQYG
YLTLNDGSQAVGRSSFYCLEYFPSQMLRTGNNFQFSYEFENVPFHSSYAHSQSLDRLMNPLID
                    VR-IV VR-V
QYLYYLSKTINGSGQNQQTLKFSVAGPSNMAVQGRNYIPGPSYRQQRVSTTVTQNNNSEFAWP
                                                                                      VR-VII
GASSWALNGRNSLMNPGPAMASHKEGEDRFFPLSGSLIFGKQGTGRDNVDADKVMITNEEEIK
                                  VR-VIII
TTNPVATESYGQVATNHQSAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSK
RWNPEIQYTSNYYKSNNVEFAVNTEGVYSEPRPIGTRYLTRNL
```

Capsid Proteins with Variant Polypeptide Sequences

In one aspect, the present disclosure provides AAV9 capsid proteins, wherein the capsid protein comprises variant polypeptide sequences with respect to the parental sequence at one or more sites of the parental sequence. In some embodiments, the one or more sites of the parental sequence are selected from the group consisting of VR-IV site, VR-V site, VR-VII site and VR-VIII site. As labeled in the SEQ ID NO: 1 above, the VR-IV site is between residues 452 and 460 in the parental sequence (“NGSGQNQ”, SEQ ID NO: 2); the VR-V site is between residues 497 and 502 in the parental sequence (“NNSEFA”, SEQ ID NO: 3); the VR-VII site is between residues 549 and 553 in the parental sequence (“GRDNV”, SEQ ID NO: 4); the VR-VIII site is between residues 581 and 594 in the parental sequence (“ATNHQSAQAQAQTG”, SEQ ID NO: 5). In some embodiments, the AAV9 capsid protein comprises a sequence that shares at least about 80%, 85%, 90%, 95%, 96%, 97%, 98%, 99%, 99.5%, or 100% identity to SEQ ID NO: 1, excluding the VR-IV site, VR-V site, VR-VII site and/or the VR-VIII site. In some embodiments, the AAV9 capsid protein comprises a sequence that shares at least about 80%, 85%, 90%, 95%, 96%, 97%, 98%, 99%, 99.5%, or 100% identity to SEQ ID NO: 1, excluding the VR-VIII site. In some embodiments, the AAV9 capsid protein comprises a sequence that shares at least about 80%, 85%, 90%, 95%, 96%, 97%, 98%, 99%, 99.5%, or 100% identity to SEQ ID NO: 487, excluding the VR-IV site, VR-V site, VR-VII site and/or the VR-VIII site. In some embodiments, the AAV9 capsid protein comprises a sequence that shares at least about 80%, 85%, 90%, 95%, 96%, 97%, 98%, 99%, 99.5%, or 100% identity to SEQ ID NO: 487, excluding the VR-VIII site. In some embodiments, the AAV9 capsid protein comprises a variant polypeptide sequence at one or more of a VR-IV site, a VR-V site, a VR-VII site, and a VR-VIII site of a parental sequence, wherein the parental sequence comprises a sequence at least 80%, at least 85%, at least 90%, at least 95%, at least 98%, at least 99%, or 100% identical to SEQ ID NO: 463. (In SEQ ID NO:463, the amino acids residues labeled “X” are excluded from sequence identity calculation.)

In some embodiments, a capsid protein described herein comprises an amino acid substitution or insertion in the VR-IV site (between residues 452 and 460 in SEQ ID NO: 1 or in the sequence of SEQ ID NO:2 (NGSGQNQ)). In some embodiments, a capsid protein described herein comprises an amino acid substitution in the VR-IV site (between residues 452 and 460 in SEQ ID NO:1 or in the sequence of SEQ ID NO:2 (NGSGQNQ)). In some embodiments, the amino acid substitution or insertion in the VR-IV site is any amino acid substitution or insertion described herein. In some embodiments, a capsid protein described herein comprises an amino acid substitution at position 452 of SEQ ID NO: 1 or the first amino acid of SEQ ID NO:2 (NGSGQNQ) in the VR-IV site. In some embodiments, a capsid protein described herein comprises an amino acid substitution N452K in SEQ ID NO:1 or comprises the sequence KGSGQNQ in the VR-IV site.

In some embodiments, a capsid protein described herein comprises an amino acid substitution or insertion in the VR-V site (between residues 497 and 502 in SEQ ID NO: 1 or in the sequence of SEQ ID NO:3 (NNSEFA)). In some embodiments, a capsid protein described herein comprises an amino acid substitution in the VR-V site (between residues 497 and 502 in SEQ ID NO:1 or in the sequence of SEQ ID NO:3 (NNSEFA)). In some embodiments, the amino acid substitution or insertion in the VR-V site is any amino acid substitution or insertion described herein.

In some embodiments, a capsid protein described herein comprises an amino acid substitution or insertion in the VR-VII site (between residues 549 and 553 in SEQ ID NO:1 or in the sequence of SEQ ID NO:4 (GRDNV)). In some embodiments, a capsid protein described herein comprises an amino acid substitution in the VR-VII site (between residues 549 and 553 in SEQ ID NO:1 or in the sequence of SEQ ID NO:4 (GRDNV)). In some embodiments, the amino acid substitution or insertion in the VR-VII site is any amino acid substitution or insertion described herein.

In some embodiments, a capsid protein described herein comprises an amino acid substitution or insertion in the VR-VIII site (between residues 581 and 594 in SEQ ID NO:1 or in the sequence of SEQ ID NO:5 (ATNHQSAQAQAQTG)). In some embodiments, a capsid protein described herein comprises an amino acid substitution in the VR-VIII site (between residues 581 and 594 in SEQ ID NO:1 or in the sequence of SEQ ID NO:5 (ATNHQSAQAQAQTG)). In some embodiments, the amino acid substitution or insertion in the VR-VIII site is any amino acid substitution or insertion described herein.

In some embodiments, the AAV9 capsid protein comprises a variant polypeptide sequence that are either rationally designed; introduced by mutagenesis; or randomized through generating a library of sequences with random codon usage at one or more sites. The capsid proteins of the disclosure include any variant polypeptide sequences identified as enriched by directed evolution followed by sequencing, as shown in, but not limited to, the Examples. Without being limited to any particular substitution site, in some embodiments, one or more sites selected from the group consisting of the VR-IV site, the VR-V site, the VR-VII site and VR-VIII site have the amino acid substitutions as described herein.

Various amino acid substitutions, insertions, and deletion are provided herein. Any of these modifications may be combined with any of the others, though where the modifications overlap one must be selected or an insertion maybe used to put two modifications adjacent to or near one another. The combination of modifications may be tested, using methods described herein (e.g., in vitro test in iPSC-CMs, in vivo testing in model organisms individually, and in vivo re-screening of pooled rAAV virions) or other known methods to identify combinations having desired packing efficiency, tropism, or other desired properties. Similarly, modifications may be made at exactly the position desired herein, or the same modification may be may at any position near to the described position. Structural modeling of the capsid protein may be used to select modification for testing.

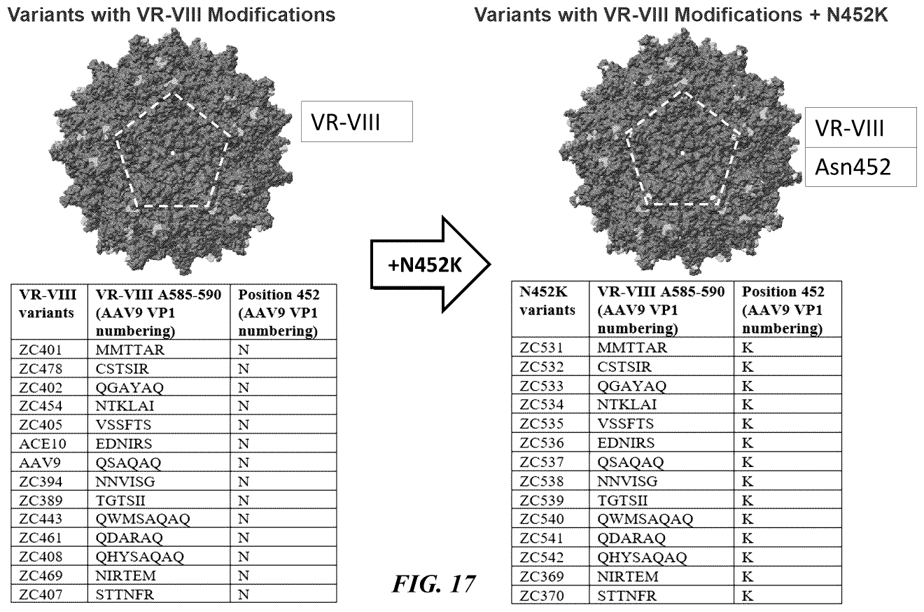

In some embodiments, the engineered capsid provided herein is any one of the capsids described herein. In some embodiments, the engineered capsid provided herein is any one of the VR-VIII-modified capsids described herein. In some embodiments, the engineered capsid provided herein is any one of the VR-IV-modified capsids described herein. In some embodiments, the engineered capsid provided herein is any one of the VR-VIII and VR-IV-modified capsids described herein. In some embodiments, the engineered capsid provided herein is any of the capsids described in any of the examples, tables or figures provided herein. In some embodiments, the engineered capsid provided herein is any of the capsids described in FIG. 17.

### Modifications Identified in Primate Screening

In one aspect, the present disclosure provides recombinant adeno-associated virus (rAAV) capsid proteins, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1, one or more of the modifications described herein.

Any of the modifications described herein may be used either alone or in combination with other modifications (e.g., in combination with other modifications described herein).

Solely for purposes of clarity and without limitation, it is noted that reference to amino acid position numbers at which modifications occur is relative to position of corresponding amino acids in SEQ ID NO:1. In some embodiments, the capsid protein does not comprise the full-length sequence corresponding to SEQ ID NO:1, but comprises a shorter variant of this sequence (e.g., comprises only a variant of SEQ ID NO:487, or a variant of SEQ ID NO: 486). In such embodiments, the modifications described herein may not occur at the same numerical positions as in SEQ ID NO:1 but occur at the same site or consensus sequence relative to reference sequence SEQ ID NO: 1. In some embodiments, the capsid protein is a variant of SEQ ID NO:1, and the modifications described herein occur at the same numerical positions as in SEQ ID NO:1.

The capsid protein may comprise an amino acid insertion at position 584 comprising one or more of an asparagine (N), a threonine (T), a tyrosine (Y), phenylalanine (F), and an alanine (A).

The capsid protein may comprise an amino acid insertion at position 585 comprising one or more of a histidine (H) and a methionine (M).

The capsid protein may comprise an amino acid insertion at position 586 comprising one or more of a histidine (H), a tyrosine (Y), a valine (V), a threonine (T), an alanine (A), an isoleucine (I), a tryptophan (W), a methionine (M), and a leucine.

The capsid protein may comprise an amino acid insertion at position 587 comprising one or more of an isoleucine (I) and a proline (P).

The capsid protein may comprise an amino acid insertion at position 588 comprising one or more of an isoleucine (I), a threonine (T), and a proline (P).

The capsid protein may comprise one or more amino acid substitutions selected from the group consisting of N452K, N452A, N452V, G453A, G453N, S454T, S454D, G455N, Q456L, Q456K, N457L, N457V, Q458I, and Q458H.

The capsid protein may comprise an amino acid substitution N452K.

The capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

The capsid protein may comprise an amino acid insertion at position 584 consisting of a TY, FN, or AT.

The capsid protein may comprise an amino acid insertion at position 585 consisting of MH.

The capsid protein may comprise an amino acid insertion at position 586 consisting of HY, VT, AI, WM, or ML.

The capsid protein may comprise an amino acid insertion at position 587 consisting of PI.

The capsid protein may comprise an amino acid insertion at position 588 consisting of IT or PT.

The capsid protein may comprise one or more amino acid substitutions selected from the group consisting of T582D, T582E, N583V, H584Q, S586K, A587P, A587S, Q588G, Q588M, A589S, A591I, G594Q, and G594D.

The capsid protein may comprise one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R.

The capsid protein may comprise one or more amino acid substitutions selected from the group consisting of Q585C, Q585S, and S586I.

The capsid protein may comprise one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

The capsid protein may comprise one or more amino acid substitutions selected from the group consisting of A587V and A587G.

The capsid protein may comprise an amino acid sequence selected from SEQ ID NOs: 599-692 and wherein the capsid protein shares at least 80%, at least 90%, at least 95%, at least 98%, or 100% identity to SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, and 710.

The capsid protein may comprise an amino acid sequence selected from SEQ ID NOs: 599-692 and wherein the capsid protein shares at least 80%, at least 90%, at least 95%, at least 98%, or 100% identity to SEQ ID NOs: 496-589.

The capsid protein may comprise the amino acid sequence ANYG at positions 586-589 or at about positions 586-589.

The capsid protein may comprise two or more amino acid substitutions selected from the group consisting of N452K, N452A, N452V, G453A, G453N, S454T, S454D, G455N, Q456L, Q456K, N457L, N457V, Q458I, and Q458H.

The capsid protein may comprise the amino acid substitution N452K, N452A, or N452V.

The capsid protein may comprise the amino acid substitution N452K.

The capsid protein may comprise the amino acid substitution G453A or G453N.

The capsid protein may comprise the amino acid substitution S454T or S454D.

The capsid protein may comprise the amino acid substitution G455N.

The capsid protein may comprise the amino acid substitution Q456L or Q456K.

The capsid protein may comprise the amino acid substitution N457L or N457V.

The capsid protein may comprise the amino acid substitution Q458I or Q458H.

The capsid protein may comprise an amino acid sequence selected from KGSGQNQ (SEQ ID NO: 590), NASGQNQ (SEQ ID NO: 591), NGTGQNQ (SEQ ID NO: 592), NGSGLNQ (SEQ ID NO: 593), ANDNKLI (SEQ ID NO: 594), VNDNKVI (SEQ ID NO: 595), NGSGQNH (SEQ ID NO: 596), or ANDNKVI (SEQ ID NO: 597) at positions 452-458 or at about positions 452-458 and wherein the capsid protein shares at least 80%, at least 90%, at least 95%, at least 98%, or 100% identity to SEQ ID NOs: 488-495.

The capsid protein may comprise an amino acid sequence selected from NTVS (SEQ ID NO: 712), TLFN (SEQ ID NO: 713), STYL (SEQ ID NO: 714), SILT (SEQ ID NO: 715), MTTA (SEQ ID NO: 716), and STSI (SEQ ID NO: 717) at positions 586-589 or at about positions 586-589 relative to reference sequence SEQ ID NO: 1. In some of these embodiments, the capsid protein comprises N452K substitution relative to reference sequence SEQ ID NO: 1.

The capsid protein may comprise an amino acid sequence selected from GAYA (SEQ ID NO: 741), TKLA (SEQ ID NO: 742), SSFT (SEQ ID NO: 743), DNIR (SEQ ID NO: 744), NVIS (SEQ ID NO: 745), GTSI (SEQ ID NO: 746), ANYG (SEQ ID NO: 305) and DARA (SEQ ID NO: 747) at positions 586-589 or at about positions 586-589 relative to reference sequence SEQ ID NO: 1. In some of these embodiments, the capsid protein comprises N452K substitution relative to reference sequence SEQ ID NO: 1.

The capsid protein may comprise an amino acid sequence SAQA (SEQ ID NO: 748) at positions 586-589 or at about positions 586-589 relative to reference sequence SEQ ID NO: 1 or comprise the same sequence at the corresponding positions relative to reference sequence SEQ ID NO:1. In some of these embodiments, the capsid protein comprises N452K substitution relative to reference sequence SEQ ID NO: 1.

The capsid protein may comprise an amino acid sequence selected from ENTVSI (SEQ ID NO: 719), QTLFNS (SEQ ID NO: 720), NSTYLG (SEQ ID NO: 721), GSILTH (SEQ ID NO: 722), MMTTAR (SEQ ID NO: 723), and CSTSIR (SEQ ID NO: 724) at positions 585-590 or at about positions 585-590 relative to reference sequence SEQ ID NO: 1. In some of these embodiments, the capsid protein comprises N452K substitution relative to reference sequence SEQ ID NO: 1.

The capsid protein may comprise an amino acid sequence selected from QGAYAQ (SEQ ID NO: 749), NTKLAI (SEQ ID NO: 750), VSSFTS (SEQ ID NO: 751), EDNIRS (SEQ ID NO: 725), NNVISG (SEQ ID NO: 752), TGTSII (SEQ ID NO: 753), QANYGQ (SEQ ID NO: 754), and QDARAQ (SEQ ID NO: 755) at positions 585-590 or at about positions 585-590 relative to reference sequence SEQ ID NO: 1. In some of these embodiments, the capsid protein comprises N452K substitution relative to reference sequence SEQ ID NO: 1.

The capsid protein may comprise an amino acid sequence QSAQAQ (SEQ ID NO: 756) at positions 585-590 or at about positions 585-590 relative to reference sequence SEQ ID NO: 1 or comprise the same sequence at the corresponding positions relative to reference sequence SEQ ID NO:1. In some of these embodiments, the capsid protein comprises N452K substitution relative to reference sequence SEQ ID NO: 1.

The capsid protein may comprise AAV9 wild type amino acid sequence at positions 581-584 (i.e., ATNH) and/or at positions 591-594 (i.e., AQTG). The capsid protein may comprise AAV9 wild type amino acid sequence at positions 581-583 (i.e., ATN) and/or at positions 591-594 (i.e., AQTG).

### Modifications in VR-IV, VR-V and VR-VII Sites

In some embodiments, the capsid protein of the present disclosure comprises a variant polypeptide sequence at the VR-IV site. In some embodiments, the entire VR-IV site (“NGSGQNQQT”, SEQ ID NO: 2) is substituted by a peptide of formula:

-(X)ₙ-
wherein n is 7-11, and X represents any of the 20 standard amino acids (SEQ ID NO: 478).

In some embodiments, the variant polypeptide sequence at the VR-IV site is:

X₁-X₂-X₃-X₄-X₅-X₆-X₇-X₈-X₉- (SEQ ID NO: 478).

In some embodiments, the variant polypeptide sequence at the VR-IV site is:

-X₁-X₂-X₃-X₄-X₅-X₆-X₇-X₈-X₉-
wherein X₁ is G, S or V; X₂ is Y, Q or I; X₃ is H, W, V, or I; X₄ is K or N; X₅ is S, G or I; X₆ is G or R; X₇ is A, P or V; X₈ is A or R; and/or X₉ is Q or D (SEQ ID NO: 477).

In some embodiments, the variant polypeptide sequence at the VR-IV site is:

-X₁-X₂-X₃-X₄-X₅-X₆-X₇-X₈-X₉-
wherein X₁ is K, G, S or V; X₂ is Y, Q or I; X₃ is H, W, V, or I; X₄ is K or N; X₅ is S, G or I; X₆ is G or R; X₇ is A, P or V; X₈ is A or R; and/or X₉ is Q or D (SEQ ID NO: 729).

In some embodiments, the variant polypeptide sequence at the VR-IV site is:

-X₁-X₂-X₃-X₄-X₅-X₆-X₇-X₈-X₉-
wherein X₁ is K (SEQ ID NO: 730).

In some embodiments, the variant polypeptide sequence at the VR-IV site comprises or consists of the sequence KGSGQNQQT (SEQ ID NO:727).

In some embodiments, the capsid protein of the present disclosure comprises a variant polypeptide sequence with N452K substitution at the VR-IV site. In some embodiments, the capsid protein of the present disclosure comprises a variant polypeptide sequence with N452K substitution at the VR-IV site relative to reference SEQ ID NO:1 or comprises the sequence of KGSGQNQQT (SEQ ID NO:727). In some embodiments, such substitution is the only substitution in an AAV9 capsid protein. In some embodiments, such substitution is the only substitution in the capsid protein of the present disclosure relative to reference SEQ ID NO:1. In some embodiments, the capsid protein comprises amino acid substitution N452K as the only substitution in a wild type AAV9 capsid protein (such as in the parental sequence of SEQ ID NO:487 or SEQ ID NO: 1). In some embodiments, such substitution is the only substitution in the AAV9 capsid protein's VR-IV and/or VR-III sites. In some embodiments, the capsid protein of the present disclosure (such as an AAV9 capsid protein) comprises amino acid substitution N452K at the VR-IV site in addition to any other substitution or insertion described herein or known in the art (including, but not limited to, any other substitution or insertion at the VR-IV site, VR-V site, VR-VII site and/or VR-VIII site). In some embodiments, the capsid protein of the present disclosure comprises amino acid substitution N452K at the VR-IV site relative to reference SEQ ID NO:1 or the sequence KGSGQNQQT (SEQ ID NO:727) in addition to any other substitution, insertion, or chimeric modification described herein or known in the art. In some embodiments, the capsid protein of the present disclosure comprises the sequence KGSGQNQQT (SEQ ID NO:727) in addition to any chimeric modification described herein or known in the art. In some embodiments, N452K substitution is combined with any other substitution(s) or insertion(s) described herein (e.g., in the VR-IV site and/or the VR-VIII site), and/or any chimeric modification(s) described herein. In some embodiments, such substitution is combined with any substitution(s) or insertion(s) in the VR-IV site described herein or known in the art. In some embodiments, such substitution is combined with any substitution(s) or insertion(s) in the VR-V site described herein or known in the art. In some embodiments, such substitution is combined with any substitution(s) or insertion(s) in the VR-VII site described herein or known in the art. In some embodiments, such substitution is combined with any substitution(s) or insertion(s) in the VR-VIII site described herein or known in the art. In some embodiments, the capsid protein of the present disclosure comprises amino acid substitution N452K at the VR-IV site in addition to any one, two, three or more substitutions or insertions at the VR-VIII site. In some embodiments, the capsid protein of the present disclosure comprises amino acid substitution N452K, relative to reference sequence SEQ ID NO: 1, in addition to one, two, three or more substitutions or insertions at the VR-VIII site described herein. In some embodiments, the capsid protein, such as the capsid protein with N452K substitution at the VR-IV site relative to reference SEQ ID NO:1, increases transduction efficiency (e.g., of any tissue, such as muscle, heart, skeletal muscle, brain, etc.). In some embodiments, the capsid protein of the present disclosure, such as the capsid protein with N452K substitution at the VR-IV site relative to reference SEQ ID NO:1, increases transduction efficiency of the heart.

In some embodiments, the capsid protein of the present disclosure comprises wild type AAV9 amino acid (which is N) at position 452 of the VR-IV site relative to reference SEQ ID NO: 1.

In some embodiments, the engineered capsid protein of the present disclosure comprises N or K at position 452 of the VR-IV site relative to reference SEQ ID NO: 1.

In some embodiments, the variant polypeptide sequence at the VR-IV site comprises or consists of a sequence selected from GYHKSGAAQ (SEQ ID NO: 6), VIIKSGAAQ (SEQ ID NO: 7), GYHKIGAAQ (SEQ ID NO: 8), GYHKSGVAQ (SEQ ID NO: 9), VYHKSGAAQ (SEQ ID NO: 10), GYHKISAAQ (SEQ ID NO: 11), TTVPSSSRY (SEQ ID NO: 12), VIIRVVRLS (SEQ ID NO: 13), TVLGQNQQT (SEQ ID NO: 14), IYHKSGAAQ (SEQ ID NO: 15), TVLDKNQQT (SEQ ID NO: 16), YSGTDVRYK (SEQ ID NO: 17), VTASGKEHR (SEQ ID NO: 18), GYRKSGAAQ (SEQ ID NO: 19), NRTVSNGSE (SEQ ID NO: 20), TVLDRINKT (SEQ ID NO: 21), TGVGHLTSA (SEQ ID NO: 22), GYHKGGAAQ (SEQ ID NO: 23), VIAKSGAAQ (SEQ ID NO: 24), GYHKSGAAH (SEQ ID NO: 25), FIIKSGAAQ (SEQ ID NO: 26), GYHKVVRLS (SEQ ID NO: 27), GATRSAVES (SEQ ID NO: 28), TVSGQNQQT (SEQ ID NO: 29), LSHKSGAAQ (SEQ ID NO: 30), SSSGQNQQT (SEQ ID NO: 31), SGSGQNQQT (SEQ ID NO: 32), SQVNGRPRD (SEQ ID NO: 33), GYHKEWCGS (SEQ ID NO: 34), VVSSKSLNS (SEQ ID NO: 35), GYHKSGAAP (SEQ ID NO: 36), DASSREKVR (SEQ ID NO: 37), SYHKSGAAQ (SEQ ID NO: 38), TANGSQKYL (SEQ ID NO: 39), VIIRVGAAQ (SEQ ID NO: 40), SSTNKISTA (SEQ ID NO: 41), TVLDRIQQT (SEQ ID NO: 42), GYHKSGAVQ (SEQ ID NO: 43), TVLDQNQQT (SEQ ID NO: 44), VNMSSPIKT (SEQ ID NO: 45), AAYNSNSAF (SEQ ID NO: 46), GYHKSGAAR (SEQ ID NO: 47), VIIRVVRLQ (SEQ ID NO: 48), RFWTQNQQT (SEQ ID NO: 49), SSPRASSAL (SEQ ID NO: 50), IIIRVVRLS (SEQ ID NO: 51), KSSNLTAMP (SEQ ID NO: 52), NLNSDRHSA (SEQ ID NO: 53), LSLKSGAAQ (SEQ ID NO: 54), TVLDRNQQT (SEQ ID NO: 55), GSERVSNSG (SEQ ID NO: 56), VIAKIGAAQ (SEQ ID NO: 57), VYHKIGAAQ (SEQ ID NO: 58), LSYKSGAAQ (SEQ ID NO: 59), STVSQPVRT (SEQ ID NO: 60), GHHKSGAAQ (SEQ ID NO: 61), YAGIDPRYH (SEQ ID NO: 62), DRSRKSMCD (SEQ ID NO: 63), VIIRSGAAQ (SEQ ID NO: 64), GYHKSGGSA (SEQ ID NO: 65), VIIKIGAAQ (SEQ ID NO: 66), GYHKVVQLS (SEQ ID NO: 67), VIIKLVAAQ (SEQ ID NO: 68), KVSSHSVCD (SEQ ID NO: 69), GYHKRVRLS (SEQ ID NO: 70), GYHKSSAAQ (SEQ ID NO: 71), GYRKIGAAQ (SEQ ID NO: 72), GYHKSGAAC (SEQ ID NO: 73), GYRQSGAAQ (SEQ ID NO: 74), VIIKLIAAQ (SEQ ID NO: 75), VIIRVVRAQ (SEQ ID NO: 76), GYHKSGAAW (SEQ ID NO: 77), GYHKSGAVS (SEQ ID NO: 78), GYHKEWCSS (SEQ ID NO: 79), SSSSNRLAD (SEQ ID NO: 80), SNNSSSAKF (SEQ ID NO: 81), VKLSSTSSS (SEQ ID NO: 82), GYHKEWCAQ (SEQ ID NO: 83), AGSGQNQQT (SEQ ID NO: 84), NPHGTATYL (SEQ ID NO: 85), NGSGQNQHT (SEQ ID NO: 86), GYHKVGAAQ (SEQ ID NO: 87), VIIRVVRLK (SEQ ID NO: 88), NSIPSTSKW (SEQ ID NO: 89), VIIRVVQLQ (SEQ ID NO: 90), SQVNGRPQD (SEQ ID NO: 91), NGSGQDQQT (SEQ ID NO: 92), GLNSSDRRL (SEQ ID NO: 93), IYHKIGAAQ (SEQ ID NO: 94), YHKSGAAQL (SEQ ID NO: 95), YSGTDVQYK (SEQ ID NO: 96), LGSGQNQQT (SEQ ID NO: 97), PVSSGADRR (SEQ ID NO: 98), EHSTKLNAC (SEQ ID NO: 99), NGSDRINKR (SEQ ID NO: 100), VIIKGGAAQ (SEQ ID NO: 101), GYHRVVRLS (SEQ ID NO: 102), VIIRVVRLL (SEQ ID NO: 103), and VILKSGAAQ (SEQ ID NO: 104). In some of any of these embodiments, the first amino acid is substituted with K instead of any other amino acid at this position (or has N452K substitution relative to reference sequence SEQ ID NO: 1).

In some embodiments, the variant polypeptide sequence at the VR-IV site comprises, consists essentially of, or consists of a polypeptide sequence at least about 60%, 70%, 80%, 90%, or 100% identical to one of SEQ ID NOs: 6-104.

In some embodiments, the variant polypeptide sequence at the VR-IV site comprises, consists essentially of, or consists of a sequence at least about 60%, 70%, 77%, 80%, 88%, 90%, or 100% identical to KGSGQNQQT (SEQ ID NO:727). In some embodiments, the variant polypeptide sequence at the VR-IV site comprises, consists essentially of, or consists of a sequence consisting of at most 1, 2, 3, or 4 amino-acid substitutions relative to KGSGQNQQT (SEQ ID NO:727). In some embodiments, the variant polypeptide sequence at the VR-IV site comprises, consists essentially of, or consists of a sequence consisting of at most 1, 2, 3, or 4 conservative amino-acid substitutions relative KGSGQNQQT (SEQ ID NO:727). In some embodiments, the variant polypeptide sequence at the VR-IV site is KGSGQNQQT (SEQ ID NO: 727).

In some embodiments, the variant polypeptide sequence at the VR-IV site comprises, consists essentially of, or consists of a sequence at least about 60%, 70%, 77%, 80%, 88%, 90%, or 100% identical to GYHKSGAAQ (SEQ ID NO: 6). In some embodiments, the variant polypeptide sequence at the VR-IV site comprises, consists essentially of, or consists of a sequence consisting of at most 1, 2, 3, or 4 amino-acid substitutions relative to GYHKSGAAQ (SEQ ID NO: 6). In some embodiments, the variant polypeptide sequence at the VR-IV site comprises, consists essentially of, or consists of a sequence consisting of at most 1, 2, 3, or 4 conservative amino-acid substitutions relative GYHKSGAAQ (SEQ ID NO: 6). In some embodiments, the variant polypeptide sequence at the VR-IV site is GYHKSGAAQ (SEQ ID NO: 6). In some of any of these embodiments, the first amino acid is substituted with K (KYHKSGAAQ; SEQ ID NO: 757).

In some embodiments, the variant polypeptide sequence at the VR-IV site comprises, consists essentially of, or consists of a sequence at least about 60%, 70%, 77%, 80%, 88%, 90%, or 100% identical to SQVNGRPRD (SEQ ID NO: 33). In some embodiments, the variant polypeptide sequence at the VR-IV site comprises, consists essentially of, or consists of a sequence consisting of at most 1, 2, 3, or 4 amino-acid substitutions relative to SQVNGRPRD (SEQ ID NO: 33). In some embodiments, the variant polypeptide sequence at the VR-IV site comprises, consists essentially of, or consists of a sequence consisting of at most 1, 2, 3, or 4 conservative amino-acid substitutions relative SQVNGRPRD (SEQ ID NO: 33). In some embodiments, the variant polypeptide sequence at the VR-IV site is SQVNGRPRD (SEQ ID NO: 33). In some of any of these embodiments, the first amino acid is substituted with K (KQVNGRPRD; SEQ ID NO: 758).

In some embodiments, the capsid protein of the present disclosure comprises a variant polypeptide sequence at the VR-V site. In some embodiments, the entire VR-V site (“NNSEFA”, SEQ ID NO: 3) is substituted by a peptide of formula:

-(X)ₙ-
wherein n is 4-8, and X represents any of the 20 standard amino acids (SEQ ID NO: 479).

In some embodiments, the variant polypeptide sequence at the VR-V site is:

(SEQ ID NO: 479)
-X₁-X₂-X₃-X₄-X₅-X₆-

In some embodiments, the variant polypeptide sequence at the VR-V site is:

-X₁-X₂-X₃-X₄-X₅-X₆-
Wherein X₁ is S, L, H, N, or A; X₂ is T, M, K, G, or N; X₃ is S, T, M or I; X₄ is S, P, F, M, or N; X₅ is F, S, P or L; and X₆ is I, V, or T (SEQ ID NO: 474).

In some embodiments, the variant polypeptide sequence at the VR-V site comprises or consists of a sequence selected from LNSMLI (SEQ ID NO: 105), NGMSFT (SEQ ID NO: 106), HKTFSI (SEQ ID NO: 107), SMSNFV (SEQ ID NO: 108), ATIPPI (SEQ ID NO: 109), SSTHFD (SEQ ID NO: 110), NNQFSY (SEQ ID NO: 111), NMGHYS (SEQ ID NO: 112), SKQMFQ (SEQ ID NO: 113), WPSAGV (SEQ ID NO: 114), NGGYQC (SEQ ID NO: 115), STSPIV (SEQ ID NO: 116), SQSGLW (SEQ ID NO: 117), VNSQFS (SEQ ID NO: 118), SGIEFR (SEQ ID NO: 119), SASKFT (SEQ ID NO: 120), QLNWTS (SEQ ID NO: 121), SMGFPV (SEQ ID NO: 122), SSFMGL (SEQ ID NO: 123), GSNFHV (SEQ ID NO: 124), DMTLYA (SEQ ID NO: 125), MGCLFT (SEQ ID NO: 126), ALAFNS (SEQ ID NO: 127), SKFLFA (SEQ ID NO: 128), QDAGLL (SEQ ID NO: 129), QDASLL (SEQ ID NO: 130), RDDMFS (SEQ ID NO: 131), LSRCFQ (SEQ ID NO: 132), LSRDFQ (SEQ ID NO: 133), QGLTPV (SEQ ID NO: 134), QWDVFT (SEQ ID NO: 135), PRVSFA (SEQ ID NO: 136), QSYYNP (SEQ ID NO: 137), RASHLG (SEQ ID NO: 138), IILFVP (SEQ ID NO: 139), IISFSY (SEQ ID NO: 140), LDSMLI (SEQ ID NO: 141), NIGHYS (SEQ ID NO: 142), NRMSFT (SEQ ID NO: 143), NGMSFA (SEQ ID NO: 144), IILLLP (SEQ ID NO: 145), RMRSLL (SEQ ID NO: 146), RRRCRF (SEQ ID NO: 147), PKQMFQ (SEQ ID NO: 148), LMSNFV (SEQ ID NO: 149), GASHLG (SEQ ID NO: 150), CASISW (SEQ ID NO: 151), SMTTFR (SEQ ID NO: 152), AAIPPI (SEQ ID NO: 153), PGCESL (SEQ ID NO: 154), SMGFAC (SEQ ID NO: 155), FLPSLM (SEQ ID NO: 156), NGISFT (SEQ ID NO: 157), ESSRWA (SEQ ID NO: 158), QLYFVP (SEQ ID NO: 159), SSNFHV (SEQ ID NO: 160), LEFMLI (SEQ ID NO: 161), QFDSFD (SEQ ID NO: 162), SPVFAC (SEQ ID NO: 163), VRLIFD (SEQ ID NO: 164), NGMSFI (SEQ ID NO: 165), LLFPPI (SEQ ID NO: 166), GAGVTG (SEQ ID NO: 167), QWMSFT (SEQ ID NO: 168), SIGFPV (SEQ ID NO: 169), RMQSLL (SEQ ID NO: 170), TSALQV (SEQ ID NO: 171), SLTHFD (SEQ ID NO: 172), QELPFL (SEQ ID NO: 173), LYFLLP (SEQ ID NO: 174), LSFFFA (SEQ ID NO: 175), LSRIFQ (SEQ ID NO: 176), DEVILF (SEQ ID NO: 177), RAGVAG (SEQ ID NO: 178), NGMSLP (SEQ ID NO: 179), PFEDFQ (SEQ ID NO: 180), QYGSLF (SEQ ID NO: 181), NYTFVL (SEQ ID NO: 182), MSGYQC (SEQ ID NO: 183), NYAFVP (SEQ ID NO: 184), RAGVTG (SEQ ID NO: 185), WNSMLI (SEQ ID NO: 186), IRRFSI (SEQ ID NO: 187), NGMSFY (SEQ ID NO: 188), IIQFSY (SEQ ID NO: 189), NGCLFT (SEQ ID NO: 190), RDASLL (SEQ ID NO: 191), ADSMLI (SEQ ID NO: 192), VDSQFS (SEQ ID NO: 193), SIGNFV (SEQ ID NO: 194), NGMSLL (SEQ ID NO: 195), NYTFVP (SEQ ID NO: 196), IRRLVF (SEQ ID NO: 197), PMSNFV (SEQ ID NO: 198), LWVFPV (SEQ ID NO: 199), VRLHFD (SEQ ID NO: 200), SMSNLF (SEQ ID NO: 201), STSLIV (SEQ ID NO: 202), and HKTFGI (SEQ ID NO: 203).

In some embodiments, the variant polypeptide sequence at the VR-V site comprises, consists essentially of, or consists of a polypeptide sequence at least about 60%, 70%, 80%, 90%, 95%, or 100% identical to one of SEQ ID NOs: 105-203.

In some embodiments, the variant polypeptide sequence at the VR-V site comprises, consists essentially of, or consists of a sequence at least about 60%, 70%, 80%, 83%, 90%, or 100% identical to LNSMLI (SEQ ID NO: 105). In some embodiments, the variant polypeptide sequence at the VR-V site comprises, consists essentially of, or consists of a sequence consisting of at most 1, 2, 3, or 4 amino-acid substitutions relative to LNSMLI (SEQ ID NO: 105). In some embodiments, the variant polypeptide sequence at the VR-V site comprises, consists essentially of, or consists of a sequence consisting of at most 1, 2, 3, or 4 conservative amino-acid substitutions relative LNSMLI (SEQ ID NO: 105). In some embodiments, the variant polypeptide sequence at the VR-V site is LNSMLI (SEQ ID NO: 105).

In some embodiments, the capsid protein of the present disclosure comprises a variant polypeptide sequence at the VR-VII site. In some embodiments, the entire VR-VII site (“GRDNV”, SEQ ID NO: 4) is substituted by a peptide of formula:

-(X)ₙ-
wherein n is 3-7, and X represents any of the 20 standard amino acids (SEQ ID NO: 480).

In some embodiments, the variant polypeptide sequence at the VR-VII site is:

(SEQ ID NO: 480)
-X₁-X₂-X3-X₄-X₅-

In some embodiments, the variant polypeptide sequence at the VR-VII site is:

-X₁-X₂-X₃-X₄-X₅-
Wherein X₁ is V, L, Q, C, or R; X₂ is S, H, G, C, or D; X₃ is Y, S, L, G, or N; X₄ is S, L, H, Q, or N; and X₅ is V, I, or R (SEQ ID NO: 475).

In some embodiments, the variant polypeptide sequence at the VR-VII site comprises or consists of a sequence selected from RGNQV (SEQ ID NO: 204), VSLNR (SEQ ID NO: 205), CDYSV (SEQ ID NO: 206), QHGHI (SEQ ID NO: 207), LCSLV (SEQ ID NO: 208), PTIYV (SEQ ID NO: 209), DVIHI (SEQ ID NO: 210), AEFYA (SEQ ID NO: 211), NSVVC (SEQ ID NO: 212), VRSNC (SEQ ID NO: 213), LANNI (SEQ ID NO: 214), NLQFM (SEQ ID NO: 215), EFRDL (SEQ ID NO: 216), DFGSL (SEQ ID NO: 217), VTNYC (SEQ ID NO: 218), WNTNA (SEQ ID NO: 219), TESTC (SEQ ID NO: 220), SGAAV (SEQ ID NO: 221), GGCDI (SEQ ID NO: 222), SGSVV (SEQ ID NO: 223), SSNAC (SEQ ID NO: 224), YNTTV (SEQ ID NO: 225), SKCLA (SEQ ID NO: 226), SAYTV (SEQ ID NO: 227), VRDTV (SEQ ID NO: 228), WRSMV (SEQ ID NO: 229), AYHGV (SEQ ID NO: 230), GMNTI (SEQ ID NO: 231), AETSL (SEQ ID NO: 232), TLVYV (SEQ ID NO: 233), NHDWI (SEQ ID NO: 234), TVGIV (SEQ ID NO: 235), SLPTV (SEQ ID NO: 236), TGILC (SEQ ID NO: 237), TDTYI (SEQ ID NO: 238), LPVTY (SEQ ID NO: 239), GDVYI (SEQ ID NO: 240), LYGTV (SEQ ID NO: 241), GCEFI (SEQ ID NO: 242), SAGLL (SEQ ID NO: 243), IKSNI (SEQ ID NO: 244), VTTSL (SEQ ID NO: 245), AVTSV (SEQ ID NO: 246), RDIHI (SEQ ID NO: 247), SAISL (SEQ ID NO: 248), VASTC (SEQ ID NO: 249), IKGLL (SEQ ID NO: 250), GSYHT (SEQ ID NO: 251), RIGFV (SEQ ID NO: 252), NDIYI (SEQ ID NO: 253), AVSCV (SEQ ID NO: 254), QHNLL (SEQ ID NO: 255), VSSCV (SEQ ID NO: 256), LNLDV (SEQ ID NO: 257), LGATI (SEQ ID NO: 258), PVLCV (SEQ ID NO: 259), SARHI (SEQ ID NO: 260), RATLI (SEQ ID NO: 261), PYNHA (SEQ ID NO: 262), IGDSI (SEQ ID NO: 263), SPMLC (SEQ ID NO: 264), YDSTL (SEQ ID NO: 265), ALKHV (SEQ ID NO: 266), ADLLT (SEQ ID NO: 267), NNGHL (SEQ ID NO: 268), INSEV (SEQ ID NO: 269), SNKTT (SEQ ID NO: 270), GSTGL (SEQ ID NO: 271), DSDMI (SEQ ID NO: 272), TSNFI (SEQ ID NO: 273), RNFTT (SEQ ID NO: 274), SHKYS (SEQ ID NO: 275), VSDIV (SEQ ID NO: 276), RVVQA (SEQ ID NO: 277), AACAV (SEQ ID NO: 278), RGRQI (SEQ ID NO: 279), AVANI (SEQ ID NO: 280), AGYDL (SEQ ID NO: 281), LSEAA (SEQ ID NO: 282), MSNYL (SEQ ID NO: 283), NFSDN (SEQ ID NO: 284), SCCDV (SEQ ID NO: 285), LASSV (SEQ ID NO: 286), PDHAV (SEQ ID NO: 287), KFDII (SEQ ID NO: 288), NSSSA (SEQ ID NO: 289), HTMHV (SEQ ID NO: 290), TLSYC (SEQ ID NO: 291), ADTHR (SEQ ID NO: 292), SMYSV (SEQ ID NO: 293), SVNLV (SEQ ID NO: 294), MSGHL (SEQ ID NO: 295), KISDT (SEQ ID NO: 296), TGLLA (SEQ ID NO: 297), AWTTS (SEQ ID NO: 298), GGALI (SEQ ID NO: 299), SCIEV (SEQ ID NO: 300), PPVIC (SEQ ID NO: 301), and GTYNL (SEQ ID NO: 302).

In some embodiments, the variant polypeptide sequence at the VR-VII site comprises, consists essentially of, or consists of a polypeptide sequence at least about 60%, 70%, 80%, 90%, or 100% identical to one of SEQ ID NOs: 204-302.

In some embodiments, the variant polypeptide sequence at the VR-VII site comprises, consists essentially of, or consists of a sequence at least about 60%, 70%, 80%, 90%, or 100% identical to RGNQV (SEQ ID NO: 204). In some embodiments, the variant polypeptide sequence at the VR-VII site comprises, consists essentially of, or consists of a sequence consisting of at most 1, 2, 3, or 4 amino-acid substitutions relative to RGNQV (SEQ ID NO: 204). In some embodiments, the variant polypeptide sequence at the VR-VII site comprises, consists essentially of, or consists of a sequence consisting of at most 1, 2, 3, or 4 conservative amino-acid substitutions relative RGNQV (SEQ ID NO: 204). In some embodiments, the variant polypeptide sequence at the VR-VII site is RGNQV (SEQ ID NO: 204).

### Modifications in VR-VIII Site

In some embodiments, the capsid protein of the present disclosure comprises a variant polypeptide sequence at the VR-VIII site.

In some embodiments, the amino acids at positions 586 to 589 (relative to reference sequence SEQ ID NO:1) of the VR-VIII site (“SAQA”) are substituted by a peptide of formula:

-(X)ₙ-
wherein n is 2-6, and X represents any of the 20 standard amino acids (SEQ ID NO: 481).

In some embodiments, the variant polypeptide sequence at the VR-VIII site is:

(SEQ ID NO: 481)
-X₁-X₂-X₃-X₄-

In some embodiments, the variant polypeptide sequence at the VR-VIII site is:

-X₁-X₂-X₃-X₄-
wherein X₁ is S, N, or A; X₂ is V, M, N, or A; X₃ is Y, V, S, or G; and X₄ is Y, T, M, G, or N (SEQ ID NO: 476).

In some embodiments, the variant polypeptide sequence at the VR-VIII site comprises:

-X₁-X₂-X₃-X₄-
wherein X₁ is S, N, T, M, G, or D; X₂ is A, T, L, I, K, S, Nor V; X₃ is Q, V, F, Y, L, T, S, I, R, or Q; and X₄ is A, S, N, L, T, I, or R (SEQ ID NO: 731).

In some embodiments, the variant polypeptide sequence at the VR-VIII site comprises:

-X₁-X₂-X₃-X₄-
wherein X₁ is S, N, T, M, G, or D; X₂ is T, L, I, K, S, N or V; X₃ is V, F, Y, L, T, S, I, R, or Q; and X₄ is A, S, N, L, T, I, or R (SEQ ID NO: 732).

In some embodiments, the variant polypeptide sequence at the VR-VIII site comprises:

-X₁-X₂-X₃-X₄-
wherein X₁ is S, N, M, or T; X₂ is A, T, L, or I; X₃ is Q, V, F, Y, T, S, or L; and X₄ is A, S, N, L, I, or T (SEQ ID NO: 733).

In some embodiments, the variant polypeptide sequence at the VR-VIII site comprises:

-X₁-X₂-X₃-X₄-
wherein X₁ is S, N, M, or T; X₂ is T, L, or I; X₃ is V, F, Y, T, S, or L; and X₄ is A, S, N, L, I, or T (SEQ ID NO: 734).

In some embodiments, the variant polypeptide sequence at the VR-VIII site comprises:

-X₁-X₂-X₃-X₄-
wherein X₁ is S, M, D, N, G, A, T, R, or I; X₂ is T, N, V, A, L, I, S, R, or P; X₃ is Y, T, S, I, V, F, L, R, N, D, G, or Q; and X₄ is L, A, I, R, S, G, N, T, V, Q, F, E, or Y (SEQ ID NO: 760).

In some embodiments, the variant polypeptide sequence at the VR-VIII site comprises:

-X₁-X₂-X₃-X₄-
wherein X₁ is S, M, D, N, G, or A; X₂ is T, N, V, or A; X₃ is Y, T, S, I, or V; and X₄ is L, A, I, R, S, or G (SEQ ID NO: 761).

In some embodiments, the variant polypeptide sequence at the VR-VIII site comprises or consists of a sequence selected from NVSY (SEQ ID NO: 303), SMVN (SEQ ID NO: 304), ANYG (SEQ ID NO: 305), NVGT (SEQ ID NO: 306), SAYM (SEQ ID NO: 307), EKVT (SEQ ID NO: 308), TTPG (SEQ ID NO: 309), GVYS (SEQ ID NO: 310), SYVG (SEQ ID NO: 311), LQYN (SEQ ID NO: 312), DPAK (SEQ ID NO: 313), THFS (SEQ ID NO: 314), IGGV (SEQ ID NO: 315), SSWN (SEQ ID NO: 316), SVYV (SEQ ID NO: 317), TLNG (SEQ ID NO: 318), NTSN (SEQ ID NO: 319), VQYA (SEQ ID NO: 320), DQYR (SEQ ID NO: 321), MPVS (SEQ ID NO: 322), SAQA (SEQ ID NO: 323), MTVA (SEQ ID NO: 324), TVMG (SEQ ID NO: 325), FSSI (SEQ ID NO: 326), SLRL (SEQ ID NO: 327), SAMG (SEQ ID NO: 328), YIKL (SEQ ID NO: 329), LMTM (SEQ ID NO: 330), QVHL (SEQ ID NO: 331), YNSV (SEQ ID NO: 332), CVIS (SEQ ID NO: 333), RLDG (SEQ ID NO: 334), AIMV (SEQ ID NO: 335), GTTG (SEQ ID NO: 336), ASYT (SEQ ID NO: 337), LHVG (SEQ ID NO: 338), LQFA (SEQ ID NO: 339), VRGD (SEQ ID NO: 340), NVMI (SEQ ID NO: 341), SLYG (SEQ ID NO: 342), GTVG (SEQ ID NO: 343), FNSV (SEQ ID NO: 344), TRLG (SEQ ID NO: 345), LKVL (SEQ ID NO: 346), SIRV (SEQ ID NO: 347), KIQG (SEQ ID NO: 348), QILG (SEQ ID NO: 349), QRDA (SEQ ID NO: 350), EAVR (SEQ ID NO: 351), AITV (SEQ ID NO: 352), KESI (SEQ ID NO: 353), LMVN (SEQ ID NO: 354), INLS (SEQ ID NO: 355), GQVS (SEQ ID NO: 356), TSLL (SEQ ID NO: 357), SSTL (SEQ ID NO: 358), YEKF (SEQ ID NO: 359), DGKL (SEQ ID NO: 360), QVYS (SEQ ID NO: 361), QKEG (SEQ ID NO: 362), ARDM (SEQ ID NO: 363), DNFR (SEQ ID NO: 364), SHGL (SEQ ID NO: 365), VSVN (SEQ ID NO: 366), GLKD (SEQ ID NO: 367), QPVF (SEQ ID NO: 368), VYSM (SEQ ID NO: 369), VMAQ (SEQ ID NO: 370), FVGM (SEQ ID NO: 371), WSTP (SEQ ID NO: 372), SYPV (SEQ ID NO: 373), TTYS (SEQ ID NO: 374), TVTT (SEQ ID NO: 375), KDKT (SEQ ID NO: 376), YREL (SEQ ID NO: 377), LSHF (SEQ ID NO: 378), SPGT (SEQ ID NO: 379), LMGT (SEQ ID NO: 380), AASL (SEQ ID NO: 381), FSNN (SEQ ID NO: 382), QARL (SEQ ID NO: 383), YHIA (SEQ ID NO: 384), ARQD (SEQ ID NO: 385), VAYT (SEQ ID NO: 386), TPSY (SEQ ID NO: 387), MILH (SEQ ID NO: 388), LGNV (SEQ ID NO: 389), TSIS (SEQ ID NO: 390), TMVY (SEQ ID NO: 391), LVVG (SEQ ID NO: 392), SPLY (SEQ ID NO: 393), YKSE (SEQ ID NO: 394), FTRL (SEQ ID NO: 395), VSYN (SEQ ID NO: 396), ERTP (SEQ ID NO: 397), FRSE (SEQ ID NO: 398), NYTE (SEQ ID NO: 399), QTIN (SEQ ID NO: 400), and DVHR (SEQ ID NO: 401). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO: 487 except for the specific substitutions at the VR-VIII site and, optionally, position 452 described herein.

In some embodiments, the variant polypeptide sequence at the VR-VIII site comprises or consists of a sequence selected from NTVS (SEQ ID NO: 712), TLFN (SEQ ID NO: 713), STYL (SEQ ID NO: 714), SILT (SEQ ID NO: 715), MTTA (SEQ ID NO: 716), and STSI (SEQ ID NO: 717). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence STYL (SEQ ID NO: 714). In some embodiments, a capsid described herein comprises the variant polypeptide sequence at the VR-VIII site comprising the sequence STYL (SEQ ID NO: 714), and further comprises N452K substitution (relative to reference sequence SEQ ID NO:1) in the VR-IV site. In some embodiments, a capsid described herein comprises the variant polypeptide sequence at the VR-VIII site comprising the sequence STYL (SEQ ID NO: 714), and does not comprise N452K substitution (relative to reference sequence SEQ ID NO:1) in the VR-IV site. In some embodiments, the variant polypeptide sequence at the VR-VIII site comprises the sequence STYL (SEQ ID NO: 714). In some embodiments, a capsid described herein comprises the variant polypeptide sequence at the VR-VIII site comprising the sequence NSTYLG (SEQ ID NO: 721), and further comprises N452K substitution (relative to reference sequence SEQ ID NO:1) in the VR-IV site. In some embodiments, a capsid described herein comprises the variant polypeptide sequence at the VR-VIII site comprising the sequence NSTYLG (SEQ ID NO: 721), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence MTTA (SEQ ID NO: 716). In some embodiments, a capsid described herein comprises the variant polypeptide sequence at the VR-VIII site comprising the sequence MTTA (SEQ ID NO: 716), and further comprises N452K substitution (relative to reference sequence SEQ ID NO:1) in the VR-IV site. In some embodiments, a capsid described herein comprises the variant polypeptide sequence at the VR-VIII site comprising the sequence MTTA (SEQ ID NO: 716), and does not comprise N452K substitution (relative to reference sequence SEQ ID NO:1) in the VR-IV site. In some embodiments, the variant polypeptide sequence at the VR-VIII site comprises the sequence MMTTAR (SEQ ID NO: 723). In some embodiments, a capsid described herein comprises the variant polypeptide sequence at the VR-VIII site comprising the sequence MMTTAR (SEQ ID NO: 723), and further comprises N452K substitution (relative to reference sequence SEQ ID NO:1) in the VR-IV site. In some embodiments, a capsid described herein comprises the variant polypeptide sequence at the VR-VIII site comprising the sequence MMTTAR (SEQ ID NO: 723), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence STSI (SEQ ID NO: 717). In some embodiments, a capsid described herein comprises the variant polypeptide sequence at the VR-VIII site comprising the sequence STSI (SEQ ID NO: 717), and further comprises N452K substitution (relative to reference sequence SEQ ID NO:1) in the VR-IV site. In some embodiments, a capsid described herein comprises the variant polypeptide sequence at the VR-VIII site comprising the sequence STSI (SEQ ID NO: 717), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence NVIS (SEQ ID NO: 745). In some embodiments, a capsid described herein comprises the variant polypeptide sequence at the VR-VIII site comprising the sequence NVIS (SEQ ID NO: 745), and further comprises N452K substitution (relative to reference sequence SEQ ID NO:1) in the VR-IV site. In some embodiments, a capsid described herein comprises the variant polypeptide sequence at the VR-VIII site comprising the sequence NVIS (SEQ ID NO: 745), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence DNIR (SEQ ID NO: 744). In some embodiments, a capsid described herein comprises the variant polypeptide sequence at the VR-VIII site comprising the sequence DNIR (SEQ ID NO: 744), and further comprises N452K substitution (relative to reference sequence SEQ ID NO:1) in the VR-IV site. In some embodiments, a capsid described herein comprises the variant polypeptide sequence at the VR-VIII site comprising the sequence DNIR (SEQ ID NO: 744), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a polypeptide sequence at least about 60%, 70%, 80%, 90%, 95%, or 100% identical to one of SEQ ID NOs: 303-401.

In some embodiments, the variant polypeptide sequence at the VR-VIII site comprises, consists essentially of, or consists of a sequence at least about 60%, 70%, 80%, 90%, or 100% identical to ANYG (SEQ ID NO: 305). In some embodiments, the variant polypeptide sequence at the VR-VIII site comprises, consists essentially of, or consists of a sequence consisting of at most 1, 2, or 3 amino-acid substitutions relative to ANYG (SEQ ID NO: 305). In some embodiments, the variant polypeptide sequence at the VR-VIII site comprises, consists essentially of, or consists of a sequence consisting of at most 1, 2, or 3 conservative amino-acid substitutions relative ANYG (SEQ ID NO: 305). In some embodiments, the variant polypeptide sequence at the VR-VIII site is ANYG (SEQ ID NO: 305).

In some embodiments, the variant polypeptide sequence at the VR-VIII site comprises, consists essentially of, or consists of a sequence at least about 60%, 70%, 80%, 90%, or 100% identical to NVSY (SEQ ID NO: 303). In some embodiments, the variant polypeptide sequence at the VR-VIII site comprises, consists essentially of, or consists of a sequence consisting of at most 1, 2, or 3 amino-acid substitutions relative to NVSY (SEQ ID NO: 303). In some embodiments, the variant polypeptide sequence at the VR-VIII site comprises, consists essentially of, or consists of a sequence consisting of at most 1, 2, or 3 conservative amino-acid substitutions relative NVSY (SEQ ID NO: 303). In some embodiments, the variant polypeptide sequence at the VR-VIII site is NVSY (SEQ ID NO: 303).

In some embodiments, the variant polypeptide sequence at the VR-VIII site comprises, consists essentially of, or consists of a polypeptide sequence at least about 60%, 70%, 80%, 90%, 95%, or 100% identical to one of SEQ ID NOs: 712-717.

In some embodiments, the variant polypeptide sequence at the VR-VIII site comprises, consists essentially of, or consists of a sequence at least about 60%, 70%, 80%, 90%, or 100% identical to NTVS (SEQ ID NO: 712). In some embodiments, the variant polypeptide sequence at the VR-VIII site comprises, consists essentially of, or consists of a sequence consisting of at most 1, 2, or 3 amino-acid substitutions relative to NTVS (SEQ ID NO: 712). In some embodiments, the variant polypeptide sequence at the VR-VIII site comprises, consists essentially of, or consists of a sequence consisting of at most 1, 2, or 3 conservative amino-acid substitutions relative NTVS (SEQ ID NO: 712). In some embodiments, the variant polypeptide sequence at the VR-VIII site is NTVS (SEQ ID NO: 712).

In some embodiments, the variant polypeptide sequence at the VR-VIII site comprises, consists essentially of, or consists of a sequence at least about 60%, 70%, 80%, 90%, or 100% identical to TLFN (SEQ ID NO: 713). In some embodiments, the variant polypeptide sequence at the VR-VIII site comprises, consists essentially of, or consists of a sequence consisting of at most 1, 2, or 3 amino-acid substitutions relative to TLFN (SEQ ID NO: 713). In some embodiments, the variant polypeptide sequence at the VR-VIII site comprises, consists essentially of, or consists of a sequence consisting of at most 1, 2, or 3 conservative amino-acid substitutions relative TLFN (SEQ ID NO: 713). In some embodiments, the variant polypeptide sequence at the VR-VIII site is TLFN (SEQ ID NO: 713).

In some embodiments, the variant polypeptide sequence at the VR-VIII site comprises, consists essentially of, or consists of a sequence at least about 60%, 70%, 80%, 90%, or 100% identical to STYL (SEQ ID NO: 714). In some embodiments, the variant polypeptide sequence at the VR-VIII site comprises, consists essentially of, or consists of a sequence consisting of at most 1, 2, or 3 amino-acid substitutions relative to STYL (SEQ ID NO: 714). In some embodiments, the variant polypeptide sequence at the VR-VIII site comprises, consists essentially of, or consists of a sequence consisting of at most 1, 2, or 3 conservative amino-acid substitutions relative STYL (SEQ ID NO: 714). In some embodiments, the variant polypeptide sequence at the VR-VIII site is STYL (SEQ ID NO: 714).

In some embodiments, the variant polypeptide sequence at the VR-VIII site comprises, consists essentially of, or consists of a sequence at least about 60%, 70%, 80%, 90%, or 100% identical to SILT (SEQ ID NO: 715). In some embodiments, the variant polypeptide sequence at the VR-VIII site comprises, consists essentially of, or consists of a sequence consisting of at most 1, 2, or 3 amino-acid substitutions relative to SILT (SEQ ID NO: 715). In some embodiments, the variant polypeptide sequence at the VR-VIII site comprises, consists essentially of, or consists of a sequence consisting of at most 1, 2, or 3 conservative amino-acid substitutions relative SILT (SEQ ID NO: 715). In some embodiments, the variant polypeptide sequence at the VR-VIII site is SILT (SEQ ID NO: 715).

In some embodiments, the variant polypeptide sequence at the VR-VIII site comprises, consists essentially of, or consists of a sequence at least about 60%, 70%, 80%, 90%, or 100% identical to MTTA (SEQ ID NO: 716). In some embodiments, the variant polypeptide sequence at the VR-VIII site comprises, consists essentially of, or consists of a sequence consisting of at most 1, 2, or 3 amino-acid substitutions relative to MTTA (SEQ ID NO: 716). In some embodiments, the variant polypeptide sequence at the VR-VIII site comprises, consists essentially of, or consists of a sequence consisting of at most 1, 2, or 3 conservative amino-acid substitutions relative MTTA (SEQ ID NO: 716). In some embodiments, the variant polypeptide sequence at the VR-VIII site is MTTA (SEQ ID NO: 716).

In some embodiments, the variant polypeptide sequence at the VR-VIII site comprises, consists essentially of, or consists of a sequence at least about 60%, 70%, 80%, 90%, or 100% identical to STSI (SEQ ID NO: 717). In some embodiments, the variant polypeptide sequence at the VR-VIII site comprises, consists essentially of, or consists of a sequence consisting of at most 1, 2, or 3 amino-acid substitutions relative to STSI (SEQ ID NO: 717). In some embodiments, the variant polypeptide sequence at the VR-VIII site comprises, consists essentially of, or consists of a sequence consisting of at most 1, 2, or 3 conservative amino-acid substitutions relative STSI (SEQ ID NO: 717). In some embodiments, the variant polypeptide sequence at the VR-VIII site is STSI (SEQ ID NO: 717).

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 712-717, or a functional fragment thereof.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 402-410 and 464-468, or a functional fragment thereof.

TABLE 1
Capsid Protein Sequences

| Name/Alternate Name | SEQ ID NO: |
|---|---|
| CR9-01/TN1 | 402 |
| CR9-07 | 403 |
| CR9-07-A/TN5 | 482 |
| CR9-07-E/ TN6 | 483 |
| CR9-08 | 464 |
| CR9-09 | 465 |
| CR9-10/ TN3 | 404 |
| CR9-11 | 466 |
| CR9-13 | 405 |
| CR9-14/TN4 | 406 |
| CR9-15 | 467 |
| CR9-16 | 468 |
| CR9-17 | 407 |
| CR9-20 | 408 |
| CR9-21 | 409 |
| CR9-22 | 410 |
| HV1/TN7 | 484 |
| HV2/TN11 | 485 |

In some embodiments, the capsid protein of the present disclosure comprises a variant polypeptide sequence at the VR-VIII site. In some embodiments, the entire VR-VIII site comprises or consists of amino acids ATNHQSAQAQAQTG (SEQ ID NO: 5), wherein amino acids QSAQAQ (SEQ ID NO: 756) are substituted by a peptide of formula:

-(X)ₙ-
wherein n is 4-8, and X represents any of the 20 standard amino acids (SEQ ID NO: 481).

In some embodiments, the variant polypeptide sequence at the VR-VIII site is or comprises:

(SEQ ID NO: 481)
-X₁-X₂-X₃-X₄-X₅-X₆-

In some embodiments, the variant polypeptide sequence at the VR-VIII site is or comprises:

-X₁-X₂-X₃-X₄-X₅-X₆
wherein X₁ is N, M, C, E, G, S, V, A, T, H, L, or Q; X₂ is M, D, N, G, A, T, R, I, or S; X₃ is T, N, V, L, I, S, R, P, or A; X₄ is Y, T, S, I, V, F, L, R, N, D, G, or Q; X₅ is L, I, R, S, G, N, T, V, Q, F, E, Y, or A, and X₆ is G, R, S, I, H, N, Y, L, M, or Q (SEQ ID NO: 762).

In some embodiments, the variant polypeptide sequence at the VR-VIII site is or comprises:

-X₁-X₂-X₃-X₄-X₅-X₆-X₇-
wherein X₁ is R or H; X₂ is N, M, C, E, G, S, V, A, T, H, L, or Q; X₃ is M, D, N, G, A, T, R, I, or S; X₄ is T, N, V, L, I, S, R, P, or A; X₅ is Y, T, S, I, V, F, L, R, N, D, G, or Q; X₆ is L, I, R, S, G, N, T, V, Q, F, E, Y, or A, and X₇ is G, R, S, I, H, N, Y, L, M, or Q (SEQ ID NO: 781).

In some embodiments, the variant polypeptide sequence at the VR-VIII site is or comprises:

-X₁-X₂-X₃-X₄-X₅-X₆
wherein X₁ is N, M, C, E, G, S, V, A, T, H, or L; X₂ is M, D, N, G, A, T, R, or I; X₃ is T, N, V, L, I, S, R, or P; X₄ is Y, T, S, I, V, F, L, R, N, D, or G; X₅ is L, I, R, S, G, N, T, V, Q, F, E, or Y, and X₆ is G, R, S, I, H, N, Y, L, or M (SEQ ID NO: 763).

In some embodiments, the variant polypeptide sequence at the VR-VIII site is or comprises:

-X₁-X₂-X₃-X₄-X₅-X₆
wherein X₁ is Q, E, N, G, M, C, V, or T; X₂ is S, N, T, M, G, or D; X₃ is A, T, L, I, K, S, N or V; X₄ is Q, V, F, Y, L, T, S, I, or R; X₅ is A, S, N, L, T, I, or R, and X₆ is Q, I, S, G, H or R (SEQ ID NO: 735).

In some embodiments, the variant polypeptide sequence at the VR-VIII site is or comprises:

-X₁-X₂-X₃-X₄-X₅-X₆
wherein X₁ is Q, E, N, G, M, C, V, or T; X₂ is S, N, T, M, G, or D; X₃ is T, L, I, K, S, Nor V; X₄ is V, F, Y, L, T, S, I, R, or Q; X₅ is A, S, N, L, T, I, or R, and X₆ is I, S, G, H or R (SEQ ID NO: 736).

In some embodiments, the variant polypeptide sequence at the VR-VIII site is or comprises:

-X₁-X₂-X₃-X₄-X₅-X₆
wherein X₁ is Q, E, N, M, C, or G; X₂ is S, N, M, or T; X₃ is A, T, L, or I; X₄ is Q, V, F, Y, T, S, or L; X₅ is A, S, N, L, I, or T; and X₆ is I, S, G, R, or H (SEQ ID NO: 737).

In some embodiments, the variant polypeptide sequence at the VR-VIII site is or comprises:

-X₁-X₂-X₃-X₄-X₅-X₆
wherein X₁ is E, N, M, C, or G; X₂ is S, N, M, or T; X₃ is T, L, or I; X₄ is V, F, Y, T, S, or L; X₅ is A, S, N, L, I, or T; and X₆ is I, S, G, R, or H (SEQ ID NO: 738).

In some embodiments, the variant polypeptide sequence at the VR-VIII site is or comprises:

-X₁-X₂-X₃-X₄-X₅-X₆
wherein X₁ is Q, E, N, G, M, or C; X₂ is S, N, T, or M; X₃ is A, T, L, I, or S; X₄ is Q, V, F, Y, L, or I; X₅ is A, S, N, L, T, or I; and X₆ is I, S, Q, G, H, or R (SEQ ID NO: 718).

In some embodiments, the variant polypeptide sequence at the VR-VIII site is or comprises:

-X₁-X₂-X₃-X₄-X₅-X₆
wherein X₁ is E, N, G, M, C, V, or T; X₂ is N, T, M, G, or D; X₃ is T, L, I, K, S, N or V; X₄ is V, F, Y, L, T, S, I, R; X₅ is S, N, L, T, I, or R, and X₆ is I, S, G, H or R (SEQ ID NO: 764).

In some embodiments, the variant polypeptide sequence at the VR-VIII site is or comprises:

-X₁-X₂-X₃-X₄-X₅-X₆
wherein X₁ is E, N, M, C, or Q; X₂ is A, M, G, D, N, or S; X₃ is T, N, V, or A; X₄ is V, Y, T, S, I, or Q; X₅ is S, G, L, I, R, or A; and X₆ is I, S, G, R, or Q (SEQ ID NO: 765).

In some embodiments, the variant polypeptide sequence at the VR-VIII site is or comprises:

-X₁-X₂-X₃-X₄-X₅-X₆
wherein X₁ is E, N, M, or C; X₂ is A, M, G, D, or N; X₃ is T, N, or V; X₄ is V, Y, T, S, or I; X₅ is S, G, L, I, or R; and X₆ is I, S, G, or R (SEQ ID NO: 766).

In some embodiments, the capsid protein of the present disclosure comprises a variant polypeptide sequence at the VR-VIII site. In some embodiments, the entire VR-VIII site comprises the following peptide of formula:

ATNH-(X)ₙ-AQTG
wherein n is 4-8, and X represents any of the 20 standard amino acids (SEQ ID NO: 740).

In some embodiments, the entire VR-VIII site comprises the following peptide of formula:

(SEQ ID NO: 740)
ATNH-X₁-X₂-X₃-X₄-X₅-X₆-AQTG.

In some embodiments, X₁-X₂-X₃-X₄-X₅-X₆ are as described above. For example, in some embodiments, X₁ is Q, E, N, G, M, C, V, or T; X₂ is S, N, T, M, G, or D; X₃ is A, T, L, I, K, S, Nor V; X₄ is Q, V, F, Y, L, T, S, I, R, or Q; X₅ is A, S, N, L, T, I, or R, and X₆ is Q, I, S, G, H or R (SEQ ID NO: 728). In some embodiments, X₁ is Q, E, N, G, M, or C; X₂ is S, N, T, or M; X₃ is A, T, L, I, or S; X₄ is Q, V, F, Y, L, or I; X₅ is A, S, N, L, T, or I; and X₆ is I, S, Q, G, H, or R (SEQ ID NO: 739).

In some of these embodiments, the capsid protein comprises N or K at position 452 relative to reference sequence SEQ ID NO: 1 (in addition to the variant polypeptide sequence described herein).

In some of these embodiments, the capsid protein may further comprise N452K substitution relative to reference sequence SEQ ID NO: 1 (in addition to the variant polypeptide sequence described herein).

In some embodiments, the variant polypeptide sequence at the VR-VIII site comprises or consists of a sequence selected from ENTVSI (SEQ ID NO: 719), QTLFNS (SEQ ID NO: 720), NSTYLG (SEQ ID NO: 721), GSILTH (SEQ ID NO: 722), MMTTAR (SEQ ID NO: 723), and CSTSIR (SEQ ID NO: 724). In some of these embodiments, the capsid protein may further comprise N452K substitution relative to reference sequence SEQ ID NO: 1 (in addition to the variant polypeptide sequence). In some of these embodiments, the capsid protein comprises the sequence at least 85%, 90%, 95%, 98%, 99% or 100% identical to VP3 of SEQ ID NO: 487 except for the specific substitutions at the VR-VIII site and, optionally, position 452 described herein.

In some embodiments, the variant polypeptide sequence at the VR-VIII site comprises or consists of a sequence selected from NSTYLG (SEQ ID NO: 721), MMTTAR (SEQ ID NO: 723), CSTSIR (SEQ ID NO: 724), EDNIRS (SEQ ID NO: 725), NNVISG (SEQ ID NO: 752), QGAYAQ (SEQ ID NO: 749), VSSFTS (SEQ ID NO: 751), TGTSII (SEQ ID NO: 753), and QHYSAQAQ (SEQ ID NO: 759). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises or consists of a sequence selected from NSTYLG (SEQ ID NO: 721), MMTTAR (SEQ ID NO: 723), CSTSIR (SEQ ID NO: 724), EDNIRS (SEQ ID NO: 725), and NNVISG (SEQ ID NO: 752). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO: 487 except for the specific substitutions at the VR-VIII site and, optionally, position 452 described herein.

In some embodiments, the variant polypeptide sequence at the VR-VIII site comprises the sequence NSTYLG (SEQ ID NO: 721). In some embodiments, the variant polypeptide sequence at the VR-VIII site comprises, consists essentially of, or consists of a sequence at least about 60%, 70%, 80%, 83%, 90%, or 100% identical to NSTYLG (SEQ ID NO: 721). In some embodiments, a capsid described herein comprises the variant polypeptide sequence at the VR-VIII site comprising the sequence NSTYLG (SEQ ID NO: 721), and further comprises N452K substitution (relative to reference sequence SEQ ID NO: 1) in the VR-IV site. In some embodiments, a capsid described herein comprises the variant polypeptide sequence at the VR-VIII site comprising the sequence NSTYLG (SEQ ID NO: 721), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence MMTTAR (SEQ ID NO: 723). In some embodiments, the variant polypeptide sequence at the VR-VIII site comprises, consists essentially of, or consists of a sequence at least about 60%, 70%, 80%, 83%, 90%, or 100% identical to MMTTAR (SEQ ID NO: 723). In some embodiments, a capsid described herein comprises the variant polypeptide sequence at the VR-VIII site comprising the sequence MMTTAR (SEQ ID NO: 723), and further comprises N452K substitution (relative to reference sequence SEQ ID NO:1) in the VR-IV site. In some embodiments, a capsid described herein comprises the variant polypeptide sequence at the VR-VIII site comprising the sequence MMTTAR (SEQ ID NO: 723), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence CSTSIR (SEQ ID NO: 724). In some embodiments, the variant polypeptide sequence at the VR-VIII site comprises, consists essentially of, or consists of a sequence at least about 60%, 70%, 80%, 83%, 90%, or 100% identical to CSTSIR (SEQ ID NO: 724). In some embodiments, a capsid described herein comprises the variant polypeptide sequence at the VR-VIII site comprising the sequence CSTSIR (SEQ ID NO: 724), and further comprises N452K substitution (relative to reference sequence SEQ ID NO:1) in the VR-IV site. In some embodiments, a capsid described herein comprises the variant polypeptide sequence at the VR-VIII site comprising the sequence CSTSIR (SEQ ID NO: 724), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence NNVISG (SEQ ID NO: 752). In some embodiments, the variant polypeptide sequence at the VR-VIII site comprises, consists essentially of, or consists of a sequence at least about 60%, 70%, 80%, 83%, 90%, or 100% identical to NNVISG (SEQ ID NO: 752). In some embodiments, a capsid described herein comprises the variant polypeptide sequence at the VR-VIII site comprising the sequence NNVISG (SEQ ID NO: 752), and further comprises N452K substitution (relative to reference sequence SEQ ID NO:1) in the VR-IV site. In some embodiments, a capsid described herein comprises the variant polypeptide sequence at the VR-VIII site comprising the sequence NNVISG (SEQ ID NO: 752), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence EDNIRS (SEQ ID NO: 725). In some embodiments, the variant polypeptide sequence at the VR-VIII site comprises, consists essentially of, or consists of a sequence at least about 60%, 70%, 80%, 83%, 90%, or 100% identical to EDNIRS (SEQ ID NO: 725). In some embodiments, a capsid described herein comprises the variant polypeptide sequence at the VR-VIII site comprising the sequence EDNIRS (SEQ ID NO: 725), and further comprises N452K substitution (relative to reference sequence SEQ ID NO: 1) in the VR-IV site. In some embodiments, a capsid described herein comprises the variant polypeptide sequence at the VR-VIII site comprising the sequence EDNIRS (SEQ ID NO: 725), and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a polypeptide sequence at least about 60%, 70%, 80%, 90%, 95%, or 100% identical to one of SEQ ID NOs: 719-724.

In some embodiments, the variant polypeptide sequence at the VR-VIII site comprises, consists essentially of, or consists of a sequence at least about 60%, 70%, 80%, 83%, 90%, or 100% identical to ENTVSI (SEQ ID NO: 719). In some embodiments, the variant polypeptide sequence at the VR-VIII site comprises, consists essentially of, or consists of a sequence consisting of at most 1, 2, or 3 amino-acid substitutions relative to ENTVSI (SEQ ID NO: 719). In some embodiments, the variant polypeptide sequence at the VR-VIII site comprises, consists essentially of, or consists of a sequence consisting of at most 1, 2, or 3 conservative amino-acid substitutions ENTVSI (SEQ ID NO: 719). In some embodiments, the variant polypeptide sequence at the VR-VIII site is NTVS ENTVSI (SEQ ID NO: 719). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to QTLFNS (SEQ ID NO: 720). In some embodiments, the variant polypeptide sequence at the VR-VIII site comprises, consists essentially of, or consists of a sequence consisting of at most 1, 2, or 3 amino-acid substitutions relative to QTLENS (SEQ ID NO: 720). In some embodiments, the variant polypeptide sequence at the VR-VIII site comprises, consists essentially of, or consists of a sequence consisting of at most 1, 2, or 3 conservative amino-acid substitutions relative QTLFNS (SEQ ID NO: 720). In some embodiments, the variant polypeptide sequence at the VR-VIII site is QTLENS (SEQ ID NO: 720). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to NSTYLG (SEQ ID NO: 721). In some embodiments, the variant polypeptide sequence at the VR-VIII site comprises, consists essentially of, or consists of a sequence consisting of at most 1, 2, or 3 amino-acid substitutions relative to NSTYLG (SEQ ID NO: 721). In some embodiments, the variant polypeptide sequence at the VR-VIII site comprises, consists essentially of, or consists of a sequence consisting of at most 1, 2, or 3 conservative amino-acid substitutions relative NSTYLG (SEQ ID NO: 721). In some embodiments, the variant polypeptide sequence at the VR-VIII site is NSTYLG (SEQ ID NO: 721). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to GSILTH (SEQ ID NO: 722). In some embodiments, the variant polypeptide sequence at the VR-VIII site comprises, consists essentially of, or consists of a sequence consisting of at most 1, 2, or 3 amino-acid substitutions relative to GSILTH (SEQ ID NO: 722). In some embodiments, the variant polypeptide sequence at the VR-VIII site comprises, consists essentially of, or consists of a sequence consisting of at most 1, 2, or 3 conservative amino-acid substitutions relative GSILTH (SEQ ID NO: 722). In some embodiments, the variant polypeptide sequence at the VR-VIII site is GSILTH (SEQ ID NO: 722). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to MMTTAR (SEQ ID NO: 723). In some embodiments, the variant polypeptide sequence at the VR-VIII site comprises, consists essentially of, or consists of a sequence consisting of at most 1, 2, or 3 amino-acid substitutions relative to MMTTAR (SEQ ID NO: 723). In some embodiments, the variant polypeptide sequence at the VR-VIII site comprises, consists essentially of, or consists of a sequence consisting of at most 1, 2, or 3 conservative amino-acid substitutions relative MMTTAR (SEQ ID NO: 723). In some embodiments, the variant polypeptide sequence at the VR-VIII site is MMTTAR (SEQ ID NO: 723). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to CSTSIR (SEQ ID NO: 724). In some embodiments, the variant polypeptide sequence at the VR-VIII site comprises, consists essentially of, or consists of a sequence consisting of at most 1, 2, or 3 amino-acid substitutions relative to CSTSIR (SEQ ID NO: 724). In some embodiments, the variant polypeptide sequence at the VR-VIII site comprises, consists essentially of, or consists of a sequence consisting of at most 1, 2, or 3 conservative amino-acid substitutions relative CSTSIR (SEQ ID NO: 724). In some embodiments, the variant polypeptide sequence at the VR-VIII site is CSTSIR (SEQ ID NO: 724). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to QGAYAQ (SEQ ID NO: 749). In some embodiments, the variant polypeptide sequence at the VR-VIII site comprises, consists essentially of, or consists of a sequence consisting of at most 1, 2, or 3 amino-acid substitutions relative to QGAYAQ (SEQ ID NO: 749). In some embodiments, the variant polypeptide sequence at the VR-VIII site comprises, consists essentially of, or consists of a sequence consisting of at most 1, 2, or 3 conservative amino-acid substitutions relative QGAYAQ (SEQ ID NO: 749). In some embodiments, the variant polypeptide sequence at the VR-VIII site is QGAYAQ (SEQ ID NO: 749). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to QANYGQ (SEQ ID NO: 754). In some embodiments, the variant polypeptide sequence at the VR-VIII site comprises, consists essentially of, or consists of a sequence consisting of at most 1, 2, or 3 amino-acid substitutions relative to QANYGQ (SEQ ID NO: 754). In some embodiments, the variant polypeptide sequence at the VR-VIII site comprises, consists essentially of, or consists of a sequence consisting of at most 1, 2, or 3 conservative amino-acid substitutions relative QANYGQ (SEQ ID NO: 754). In some embodiments, the variant polypeptide sequence at the VR-VIII site is QANYGQ (SEQ ID NO: 754). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 719-724, or a functional fragment thereof.

In some embodiments, the variant polypeptide sequence at the VR-VIII site comprises amino acid R or H at position 584 relative to reference sequence SEQ ID NO: 1. In some embodiments, the variant polypeptide sequence at the VR-VIII site comprises R at position 584.

In some embodiments, the variant polypeptide sequence at the VR-VIII site comprises A587T substitution (i.e., T at position 587) relative to reference sequence SEQ ID NO: 1.

In some embodiments, the variant polypeptide sequence at the VR-VIII site comprises amino acid N or R at one, two, three or more positions selected from the group consisting of: 584, 585, 586, 588, 589, and 590 (or amino acid N or R within-3 to +3 positions from position 587), relative to reference sequence SEQ ID NO: 1. In some embodiments, the variant polypeptide sequence at the VR-VIII site comprises amino acid N or R at two, three or more positions selected from the group consisting of: 584, 585, 586, 588, 589, and 590 (or amino acid N or R within-3 to +3 positions from position 587), relative to reference sequence SEQ ID NO: 1.

In some embodiments, the variant polypeptide sequence at the VR-VIII site comprises A587T substitution (i.e., T at position 587), and comprises amino acid N or R at one, two, three or more positions selected from the group consisting of: 584, 585, 586, 588, 589, and 590 (or amino acid N or R within-3 to +3 positions from position 587), relative to reference sequence SEQ ID NO: 1.

In some embodiments, the variant polypeptide sequence at the VR-VIII site comprises amino acid S at two, three or more positions selected from the group consisting of: 585, 586, 587, 588, 589 and 590 (or, two or more amino acids S at positions in the region 585-590), relative to reference sequence SEQ ID NO: 1.

In some embodiments, the variant polypeptide sequence at the VR-VIII site comprises, at three, four or more positions in the region 585-590, relative to reference sequence SEQ ID NO: 1, one, two or more amino acids (in any combination) selected from the group consisting of: N, S, T, R and I. In some embodiments, the variant polypeptide sequence at the VR-VIII site comprises, at three, four or more positions in the region 585-590, relative to reference sequence SEQ ID NO: 1, one, two or more amino acids (in any combination) selected from the group consisting of: N, S, T, and R.

In some embodiments, the variant polypeptide sequence at the VR-VIII site comprises any one or more amino acids (e.g., any 2, 3, 4 or more, in any combination) selected from the group consisting of: N, S, T, R and I, at three, four or more positions in the region 585-590 (i.e., position 585, 586, 587, 588, 589, and/or 590), relative to reference sequence SEQ ID NO: 1. In some embodiments, the variant polypeptide sequence at the VR-VIII site comprises any one or more amino acids (e.g., any 2, 3, 4 or more, in any combination) selected from the group consisting of: N, S, T and R, at three, four or more positions in the region 585-590 (i.e., positions 585, 586, 587, 588, 589, and 590), relative to reference sequence SEQ ID NO: 1. For example, and without limitation, in the region 585-590, there may be three or more N, three or more S, three or more T, etc., or all three of N, S and T, or two or three of one referenced amino acid (e.g., N) and one or more of any other referenced amino acid (e.g., T), or one of each of these amino acids (i.e., all five of N, S, T, R and I), and so on, in any combination.

In some of these embodiments, the capsid protein comprises N or K at position 452 relative to reference sequence SEQ ID NO: 1 (in addition to the variant polypeptide sequence described herein).

In some embodiments, the capsid protein may comprise N452K substitution relative to reference sequence SEQ ID NO: 1 (either by itself, or in addition to the variant polypeptide having one or more substitutions described herein, such as any substitution or substitution pattern at the VR-VIII site described herein).

In some embodiments, the capsid protein comprises N452K substitution relative to reference sequence SEQ ID NO: 1 (and, optionally, comprises 80%, 85%, 90%, 95%, 98%, 99% or 100% identity to VP3 of SEQ ID NO:487 and/or VP1 of SEQ ID NO:1 at positions other than 452).

In some embodiments, the variant VP1 capsid protein of SEQ ID NO: 1 comprises one of the substitution patterns at the VR-VIII site positions 581-594 or 585-590 and/or position 452 of AAV9 VP1 presented in the below tables. In some embodiments, the variant VP1 capsid protein of SEQ ID NO:1 comprises a substitution pattern at the VR-VIII site positions 581-594 of AAV9 VP1 that has at least about 75%, 78.5%, 80%, 85%, 90%, 93% or 100% sequence identity to that presented in the below tables.

| | VR-VIII | SEQ ID |
|---|---|---|
| Position 452 | Alignment (581-594) | NO |
| N or K | ATNHENTVSIAQTG | 618 |
| N or K | ATNHQTLFNSAQTG | 684 |
| N or K | ATNHNSTYLGAQTG | 642 |
| N or K | ATNHGSILTHAQTG | 630 |
| N or K | ATNHMMTTARAQTG | 615 |
| N or K | ATNHCSTSIRAQTG | 692 |
| N or K | ATNHQGAYAQAQTG | 616 |
| N or K | ATNHNTKLAIAQTG | 668 |
| N or K | ATNHVSSFTSAQTG | 619 |
| N or K | ATNHEDNIRSAQTG | 726 |
| K | ATNHQSAQAQAQTG | 5 |
| N or K | ATNHNNVISGAQTG | 608 |
| N or K | ATNHTGTSIIAQTG | 603 |
| N or K | ATNHQWMSAQAQAQTG | 657 |
| N or K | ATNHQDARAQAQTG | 675 |
| N or K | ATNHQHYSAQAQAQTG | 622 |
| N or K | ATNHQSAQAQAQTG | 5 |
| N or K | ATNHNIRTEMAQTG | 683 |
| N or K | ATNHSTTNFRAQTG | 621 |

| Position | Position | Position | Position | Position | Position | Position |
|---|---|---|---|---|---|---|
| 585 | 586 | 587 | 588 | 589 | 590 | 452 |
| Q585E | S586N | A587T | Q588V | A589S | Q590I | N or N452K |
| Q | S586T | A587L | Q588F | A589N | Q590S | N or N452K |
| Q585N | S | A587T | Q588Y | A589L | Q590G | N or N452K |
| Q585G | S | A587I | Q588L | A589T | Q590H | N or N452K |
| Q585E | S586N | A587T | Q588V | A589S | Q590I | N or N452K |
| Q | S586T | A587L | Q588F | A589N | Q590S | N or N452K |
| Q585N | S | A587T | Q588Y | A589L | Q590G | N or N452K |
| Q585G | S | A587I | Q588L | A589T | Q590H | N or N452K |
| Q585M | S586M | A587T | Q588T | A | Q590R | N or N452K |
| Q585C | S | A587T | Q588S | A589I | Q590R | N or N452K |
| Q | S586G | A | Q588Y | A | Q | N or N452K |
| Q585N | S586T | A587K | Q588L | A | Q590I | N or N452K |
| Q585V | S | A587S | Q588F | A589T | Q590S | N or N452K |
| Q585E | S586D | A587N | Q588I | A589R | Q590S | N or N452K |
| Q | S | A | Q | A | Q | N or N452K |
| Q585N | S586N | A587V | Q588I | A589S | Q590G | N or N452K |
| Q585T | S586G | A587T | Q588S | A589I | Q590I | N or N452K |
| Q | S586D | A | Q588R | A | Q | N or N452K |
| Q585N | S586I | A587R | Q588T | A589E | Q590M | N or N452K |
| Q585S | S586T | A587T | Q588N | A589F | Q590R | N or N452K |
| Q | S | A | Q | A | Q | N or N452K |

In some embodiments, the capsids in the above table have: (i) ATNH at positions 581, 582, 583 and 584, respectively, and/or (ii) AQTG at positions 591, 592, 593 and 594, respectively.

In some embodiments, the variant VP1 capsid protein of SEQ ID NO:1 comprises one of the following amino acids at the VR-VIII site positions 581-594 or 585-590:

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, | N, | T, | V, | S, | I, | A | Q | T | G |
| | | | | N, | T, | L, | F, | N, | S, | | | | |
| | | | | G, | M, | I, | Y, | L, | G, | | | | |
| | | | | M, | G, | K. | L, | T, | H, | | | | |
| | | | | C, | D, | S, | T, | I, | R, | | | | |
| | | | | V, | S, | N, | S, | R, | Q, | | | | |
| | | | | T, | I | V, | I, | A, | M | | | | |
| | | | | Q, | | A, | R, | E, | | | | | |
| | | | | S | | R | Q, | F | | | | | |
| | | | | | | | N | | | | | | |

In some embodiments, the variant VP1 capsid protein of SEQ ID NO:1 comprises one of the substitution patterns at the VR-VIII site positions 581-594 or 585-590 and/or position 452 of AAV9 VP1 presented in the below tables. In some embodiments, the variant VP1 capsid protein of SEQ ID NO:1 comprises a substitution pattern at the VR-VIII site positions 581-594 of AAV9 VP1 that has at least about 75%, 78.5%, 80%, 85%, 90%, 93% or 100% sequence identity to that presented in the below tables.

| | VR-VIII | SEQ |
|---|---|---|
| Position 452 | Alignment (581-594) | ID NO |
| N or K | ATNHNSTYLGAQTG | 642 |
| N or K | ATNHMMTTARAQTG | 615 |
| N or K | ATNHCSTSIRAQTG | 692 |
| N or K | ATNHQGAYAQAQTG | 616 |
| N or K | ATNHVSSFTSAQTG | 619 |
| N or K | ATNHEDNIRSAQTG | 726 |
| N or K | ATNHNNVISGAQTG | 608 |
| N or K | ATNHTGTSIIAQTG | 603 |
| N or K | ATNHQHYSAQAQAQTG | 622 |

| Position | Position | Position | Position | Position | Position | Position |
|---|---|---|---|---|---|---|
| 585 | 586 | 587 | 588 | 589 | 590 | 452 |
| Q585N | S | A587T | Q588Y | A589L | Q590G | N or N452K |
| Q585M | S586M | A587T | Q588T | A | Q590R | N or N452K |
| Q585C | S | A587T | Q588S | A589I | Q590R | N or N452K |
| Q | S586G | A | Q588Y | A | Q | N or N452K |
| Q585V | S | A587S | Q588F | A589T | Q590S | N or N452K |
| Q585E | S586D | A587N | Q588I | A589R | Q590S | N or N452K |
| Q585N | S586N | A587V | Q588I | A589S | Q590G | N or N452K |
| Q585T | S586G | A587T | Q588S | A589I | Q590I | N or N452K |

In some embodiments, the capsids in the above table have: (i) ATNH at positions 581, 582, 583 and 584, respectively, and/or (ii) AQTG at positions 591, 592, 593 and 594, respectively.

In some embodiments, the variant VP1 capsid protein of SEQ ID NO:1 comprises one of the following amino acids at the VR-VIII site positions 581-594 or 585-590:

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, | N, | T, | F, | S, | I, | A | Q | T | G |
| | | | | N, | M, | S, | Y, | L, | S, | | | | |
| | | | | M, | G, | N, | T, | T, | G, | | | | |
| | | | | C, | D, | V, | S, | I, | R, | | | | |
| | | | | V, | S | A | I, | R, | Q | | | | |
| | | | | T, | | | | A | | | | | |
| | | | | Q | | | | | | | | | |

In some embodiments, the disclosure provides a recombinant adeno-associated virus (rAAV) capsid protein, wherein the capsid protein comprises, relative to reference sequence SEQ ID NO: 1, amino acid substitutions Q585E, S586N, A587T, Q588V, A589S, Q590I, and N452K.

In some embodiments, the disclosure provides a recombinant adeno-associated virus (rAAV) capsid protein, wherein the capsid protein comprises, relative to reference sequence SEQ ID NO: 1, amino acid substitutions S586T, A587L, Q588F, A589N, Q590S, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585N, A587T, Q588Y, A589L, Q590G, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585N, A587T, Q588Y, A589L, and Q590G.

In some embodiments, the disclosure provides a recombinant adeno-associated virus (rAAV) capsid protein, wherein the capsid protein comprises, relative to reference sequence SEQ ID NO: 1, amino acid substitutions Q585G, A587I, Q588L, A589T, Q590H, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585M, S586M, A587T, Q588T, A589A, and Q590R.

In some embodiments, the disclosure provides a recombinant adeno-associated virus (rAAV) capsid protein, wherein the capsid protein comprises, relative to reference sequence SEQ ID NO: 1, amino acid substitutions Q585C, A587T, Q588S, A589I, and Q590R.

In some embodiments, the disclosure provides a recombinant adeno-associated virus (rAAV) capsid protein, wherein the capsid protein comprises the amino acid sequence of SEQ ID NO: 488. In some embodiments, the disclosure provides a recombinant adeno-associated virus (rAAV) capsid protein, wherein the capsid protein comprises the amino acid sequence of SEQ ID NO: 499. In some embodiments, the disclosure provides a recombinant adeno-associated virus (rAAV) capsid protein, wherein the capsid protein comprises the amino acid sequence of SEQ ID NO: 504. In some embodiments, the disclosure provides a recombinant adeno-associated virus (rAAV) capsid protein, wherein the capsid protein comprises the amino acid sequence of SEQ ID NO: 505. In some embodiments, the disclosure provides a recombinant adeno-associated virus (rAAV) capsid protein, wherein the capsid protein comprises the amino acid sequence of SEQ ID NO: 506. In some embodiments, the disclosure provides a recombinant adeno-associated virus (rAAV) capsid protein, wherein the capsid protein comprises the amino acid sequence of SEQ ID NO: 510. In some embodiments, the disclosure provides a recombinant adeno-associated virus (rAAV) capsid protein, wherein the capsid protein comprises the amino acid sequence of SEQ ID NO: 512. In some embodiments, the disclosure provides a recombinant adeno-associated virus (rAAV) capsid protein, wherein the capsid protein comprises the amino acid sequence of SEQ ID NO: 513. In some embodiments, the disclosure provides a recombinant adeno-associated virus (rAAV) capsid protein, wherein the capsid protein comprises the amino acid sequence of SEQ ID NO: 516. In some embodiments, the disclosure provides a recombinant adeno-associated virus (rAAV) capsid protein, wherein the capsid protein comprises the amino acid sequence of SEQ ID NO: 518. In some embodiments, the disclosure provides a recombinant adeno-associated virus (rAAV) capsid protein, wherein the capsid protein comprises the amino acid sequence of SEQ ID NO: 521. In some embodiments, the disclosure provides a recombinant adeno-associated virus (rAAV) capsid protein, wherein the capsid protein comprises the amino acid sequence of SEQ ID NO: 522. In some embodiments, the disclosure provides a recombinant adeno-associated virus (rAAV) capsid protein, wherein the capsid protein comprises the amino acid sequence of SEQ ID NO: 533. In some embodiments, the disclosure provides a recombinant adeno-associated virus (rAAV) capsid protein, wherein the capsid protein comprises the amino acid sequence of SEQ ID NO: 536. In some embodiments, the disclosure provides a recombinant adeno-associated virus (rAAV) capsid protein, wherein the capsid protein comprises the amino acid sequence of SEQ ID NO: 539. In some embodiments, the disclosure provides a recombinant adeno-associated virus (rAAV) capsid protein, wherein the capsid protein comprises the amino acid sequence of SEQ ID NO: 558. In some embodiments, the disclosure provides a recombinant adeno-associated virus (rAAV) capsid protein, wherein the capsid protein comprises the amino acid sequence of SEQ ID NO: 562. In some embodiments, the disclosure provides a recombinant adeno-associated virus (rAAV) capsid protein, wherein the capsid protein comprises the amino acid sequence of SEQ ID NO: 566. In some embodiments, the disclosure provides a recombinant adeno-associated virus (rAAV) capsid protein, wherein the capsid protein comprises the amino acid sequence of SEQ ID NO: 571. In some embodiments, the disclosure provides a recombinant adeno-associated virus (rAAV) capsid protein, wherein the capsid protein comprises the amino acid sequence of SEQ ID NO: 576. In some embodiments, the disclosure provides a recombinant adeno-associated virus (rAAV) capsid protein, wherein the capsid protein comprises the amino acid sequence of SEQ ID NO: 578. In some embodiments, the disclosure provides a recombinant adeno-associated virus (rAAV) capsid protein, wherein the capsid protein comprises the amino acid sequence of SEQ ID NO: 579. In some embodiments, the disclosure provides a recombinant adeno-associated virus (rAAV) capsid protein, wherein the capsid protein comprises the amino acid sequence of SEQ ID NO: 580. In some embodiments, the disclosure provides a recombinant adeno-associated virus (rAAV) capsid protein, wherein the capsid protein comprises the amino acid sequence of SEQ ID NO: 581. In some embodiments, the disclosure provides a recombinant adeno-associated virus (rAAV) capsid protein, wherein the capsid protein comprises the amino acid sequence of SEQ ID NO: 585. In some embodiments, the disclosure provides a recombinant adeno-associated virus (rAAV) capsid protein, wherein the capsid protein comprises the amino acid sequence of SEQ ID NO: 588. In some embodiments, the disclosure provides a recombinant adeno-associated virus (rAAV) capsid protein, wherein the capsid protein comprises the amino acid sequence of SEQ ID NO: 589.

In some embodiments, the disclosure provides a recombinant adeno-associated virus (rAAV) capsid protein, wherein the capsid protein comprises the amino acid sequence of SEQ ID NO: 705. In some embodiments, the disclosure provides a recombinant adeno-associated virus (rAAV) capsid protein, wherein the capsid protein comprises the amino acid sequence of SEQ ID NO: 706. In some embodiments, the disclosure provides a recombinant adeno-associated virus (rAAV) capsid protein, wherein the capsid protein comprises the amino acid sequence of SEQ ID NO: 707. In some embodiments, the disclosure provides a recombinant adeno-associated virus (rAAV) capsid protein, wherein the capsid protein comprises the amino acid sequence of SEQ ID NO: 708. In some embodiments, the disclosure provides a recombinant adeno-associated virus (rAAV) capsid protein, wherein the capsid protein comprises the amino acid sequence of SEQ ID NO: 710. In some embodiments, the disclosure provides a recombinant adeno-associated virus (rAAV) capsid protein, wherein the capsid protein comprises the amino acid sequence of SEQ ID NO: 767. In some embodiments, the disclosure provides a recombinant adeno-associated virus (rAAV) capsid protein, wherein the capsid protein comprises the amino acid sequence of SEQ ID NO: 768. In some embodiments, the disclosure provides a recombinant adeno-associated virus (rAAV) capsid protein, wherein the capsid protein comprises the amino acid sequence of SEQ ID NO: 769. In some embodiments, the disclosure provides a recombinant adeno-associated virus (rAAV) capsid protein, wherein the capsid protein comprises the amino acid sequence of SEQ ID NO: 770. In some embodiments, the disclosure provides a recombinant adeno-associated virus (rAAV) capsid protein, wherein the capsid protein comprises the amino acid sequence of SEQ ID NO: 771. In some embodiments, the disclosure provides a recombinant adeno-associated virus (rAAV) capsid protein, wherein the capsid protein comprises the amino acid sequence of SEQ ID NO: 772. In some embodiments, the disclosure provides a recombinant adeno-associated virus (rAAV) capsid protein, wherein the capsid protein comprises the amino acid sequence of SEQ ID NO: 773. In some embodiments, the disclosure provides a recombinant adeno-associated virus (rAAV) capsid protein, wherein the capsid protein comprises the amino acid sequence of SEQ ID NO: 774. In some embodiments, the disclosure provides a recombinant adeno-associated virus (rAAV) capsid protein, wherein the capsid protein comprises the amino acid sequence of SEQ ID NO: 775. In some embodiments, the disclosure provides a recombinant adeno-associated virus (rAAV) capsid protein, wherein the capsid protein comprises the amino acid sequence of SEQ ID NO: 776. In some embodiments, the disclosure provides a recombinant adeno-associated virus (rAAV) capsid protein, wherein the capsid protein comprises the amino acid sequence of SEQ ID NO: 777. In some embodiments, the disclosure provides a recombinant adeno-associated virus (rAAV) capsid protein, wherein the capsid protein comprises the amino acid sequence of SEQ ID NO: 778.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 767, 768, 769, 770, 771, 772, 773, 774, 775, 776, 777, 778, or a functional fragment thereof. In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 705-708, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 705, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 706, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 707, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 708, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 710, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 772, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 774, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 488, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 512, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 513, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 539, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 589, or a functional fragment thereof.

In some embodiments, the capsid protein comprises, at amino acid positions 581-594 relative to reference sequence SEQ ID NO:1, the amino acid sequence of any one of SEQ ID NOs: 618, 684, 642, 630, 615, 692, 616, 668, 726, 608, 603, 657, 675, and 622, and optimally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 618, and optionally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 684, and optionally wherein the capsid protein further comprises an amino acid substitution of N452K. In some embodiments, the capsid protein comprises, at amino acid positions 581-594 relative to reference sequence SEQ ID NO: 1, the amino acid sequence of SEQ ID NO: 642, and optionally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 630, and optionally wherein the capsid protein further comprises an amino acid substitution of N452K.

In some embodiments, the capsid protein comprises, at amino acid positions 581-594 relative to reference sequence SEQ ID NO:1, the amino acid sequence of any one of SEQ ID NOs: 598, 602, 607, 608, 609, 613, 615, 616, 618, 619, 621, 624, 625, 630, 636, 639, 642, 661, 665, 669, 674, 679, 681, 682, 683, 684, 688, 691, 692, and 726. In some of these embodiments, the capsid comprises at amino acid position 452, relative to reference sequence SEQ ID NO:1, amino acid N or K. In some of these embodiments, the capsid protein comprises an amino acid substitution N452K.

In some embodiments, the capsid protein comprises, at amino acid positions 581-594 relative to reference sequence SEQ ID NO:1, the amino acid sequence of any one of SEQ ID NOs: 598, 608, 615, 616, 618, 642, 692, and 726. In some of these embodiments, the capsid comprises at amino acid position 452, relative to reference sequence SEQ ID NO:1, amino acid Nor K. In some of these embodiments, the capsid protein comprises an amino acid substitution N452K.

In some embodiments, the capsid protein of the present disclosure comprises a variant polypeptide sequence at the VR-VIII site, wherein the VR-VIII site (e.g., the entire VR-VIII site) comprises, consists essentially of, or consists of, a sequence having at least about 60%, 65%, 70%, 71%, 74%, 75%, 78%, 78.5%, 79%, 80%, 83%, 85%, 86%, 90%, 92%, 93% or 100% identity to any one of the following sequences (e.g., with at most 1, 2, or 3 amino acid substitutions relative to any one of the following sequences):

| VR-VIII Alignment (581-594) | SEQ ID NO |
|---|---|
| ATNHENTVSIAQTG | 618 |
| ATNHQTLFNSAQTG | 684 |
| ATNHNSTYLGAQTG | 642 |
| ATNHGSILTHAQTG | 630 |
| ATNHMMTTARAQTG | 615 |
| ATNHCSTSIRAQTG | 692 |
| ATNHQGAYAQAQTG | 616 |
| ATNHNTKLAIAQTG | 668 |
| ATNHVSSFTSAQTG | 619 |
| ATNHEDNIRSAQTG | 726 |
| ATNHQSAQAQAQTG | 5 |
| ATNHNNVISGAQTG | 608 |
| ATNHTGTSIIAQTG | 603 |
| ATNHQWMSAQAQAQTG | 657 |
| ATNHQDARAQAQTG | 675 |
| ATNHQHYSAQAQAQTG | 622 |
| ATNHQSAQAQAQTG | 5 |
| ATNHNIRTEMAQTG | 683 |
| ATNHSTTNFRAQTG | 621 |
| ATNHQANYGQAQTG | 598 |
| ATNHNMNRVNAQTG | 607 |
| ATNHSNSVQSAQTG | 609 |
| ATNHSSTFQGAQTG | 613 |
| ATNHSTTNFRAQTG | 621 |
| ATNHSSIFNSAQTG | 624 |
| ATNHAGNYNNAQTG | 625 |
| ATNHTSVISIAQTG | 636 |
| ATNHHSRVEIAQTG | 639 |
| ATNHSSIIYSAQTG | 661 |
| ATNHSGRDSYAQTG | 665 |
| ATNHSSSYNNAQTG | 669 |
| ATNHHNPSINAQTG | 674 |
| ATNHNRNGLLAQTG | 681 |
| ATNHESTSVRAQTG | 682 |
| ATNHLSVSSIAQTG | 688 |
| ATNHEDIIRSAQTG | 691 |
| ATNRQTAQAQAQTG | 602 |
| ATNRQIAQAQAQTG | 679 |

In some embodiments, the capsid protein of the present disclosure comprises a variant polypeptide sequence at the VR-VIII site, wherein the entire VR-VIII site comprises amino acids ATNHQSAQAQAQTG (SEQ ID NO: 5), and wherein there is an insertion of one, two or more amino acids in this site. In some embodiments, the insertion is within the variant polypeptide of sequence QSAQAQ (SEQ ID NO: 756), within SEQ ID NO:5. In some embodiments, the insertion is between amino acids ATNHQ and amino acids SAQAQAQTG of SEQ ID NO:5. In other words, in some embodiments, the insertion at the VR-VIII site is between position 585 and position 586 relative to reference sequence SEQ ID NO:1. In some embodiments, the insertion is insertion of amino acids WM (e.g., between positions 585 and 586 relative to reference sequence SEQ ID NO:1). In some embodiments, the insertion is insertion of amino acids HY (e.g., between positions 585 and 586 relative to reference sequence SEQ ID NO:1). In some of these embodiments, the capsid protein may further comprise N452K substitution relative to reference sequence SEQ ID NO: 1 (in addition to the variant polypeptide at VR-VIII site described herein).

### Chimeric AAV5/AAV9 Capsid

The present disclosure also provides recombinant adeno-associated virus (rAAV) capsid proteins comprising a sequence at least 80%, at least 85%, at least 90%, at least 95%, at least 98%, at least 99%, or 100% identical to SEQ ID NO: 463. (In SEQ ID NO:463, the amino acids residues labeled “X” are excluded from sequence identity calculation.) In some embodiments, the capsid protein is an AAV5/AAV9 chimeric capsid protein. In some embodiments, the AAV5/AAV9 chimeric capsid protein sequence is more than about 50%, 55%, 60%, 65%, 70%, 75%, 80%, 85%, 90%, 95%, 96%, 97%, 98%, 99%, or 99.5% identical to the AAV9 capsid protein sequence (SEQ ID NO: 1). In some embodiments, the C-terminal 500 residues of the AAV5/AAV9 chimeric capsid protein sequence is at least about 80%, 85%, 90%, 95%, 96%, 97%, 98%, 99%, 99.5%, or 100% identical to the C-terminal 500 residues of the AAV9 capsid protein sequence (SEQ ID NO: 1). In some embodiments, the residue at the position equivalent to Q688 of the AAV9 capsid protein sequence (SEQ ID NO: 1) is a lysine (K) in the chimeric capsid protein.

In some embodiments, the chimeric capsid protein comprises at least 1, 2, 3, 4, 5 or more polypeptide segments that are derived from AAV5 capsid protein. In some embodiments, the chimeric capsid protein comprises at least 1, 2, 3, 4, 5 or more polypeptide segments that are derived from AAV9 capsid protein. In some embodiments, at least one polypeptide segment is derived from the AAV5 capsid protein and at least one polypeptide segment is derived from the AAV9 capsid protein.

In some embodiments, the first 250 residues at the N-terminus of the chimeric capsid protein comprise one or more AAV5 capsid derived polypeptide segments. In some embodiments, the first 225 residues at the N-terminus of the chimeric capsid protein comprise one or more AAV5 capsid derived polypeptide segments. In some embodiments, the first 200 residues at the N-terminus of the chimeric capsid protein comprise one or more AAV5 capsid derived polypeptide segments. In some embodiments, the first 150 residues at the N-terminus of the chimeric capsid protein comprise one or more AAV5 capsid derived polypeptide segments. In some embodiments, the first 100 residues at the N-terminus of the chimeric capsid protein comprise one or more AAV5 capsid derived polypeptide segments. In some embodiments, the first 50 residues at the N-terminus of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% sequence identity to the corresponding AAV5 capsid sequence.

In some embodiments, residues 50-250 of the chimeric capsid protein comprise one or more AAV5 capsid derived polypeptide segments. In some embodiments, residues 50-200 of the chimeric capsid protein comprise one or more AAV5 capsid derived polypeptide segments. In some embodiments, residues 50-150 of the chimeric capsid protein comprise one or more AAV5 capsid derived polypeptide segments. In some embodiments, residues 100-250 of the chimeric capsid protein comprise one or more AAV5 capsid derived polypeptide segments. In some embodiments, residues 100-200 of the chimeric capsid protein comprise one or more AAV5 capsid derived polypeptide segments. In some embodiments, residues 150-250 of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% sequence identity to the corresponding AAV5 capsid sequence.

In some embodiments, the last 100 residues at the C-terminus of the chimeric capsid protein comprise one or more AAV5 capsid derived polypeptide segments. In some embodiments, the last 50 residues at the C-terminus of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% sequence identity to the corresponding AAV5 capsid sequence. In some embodiments, the chimeric capsid protein comprises one or more AAV5 capsid derived polypeptide segments at or near the N-terminus of the chimeric capsid protein, as described above, and one or more AAV5 capsid derived polypeptide segments at or near the C-terminus of the chimeric capsid protein, as described in this paragraph.

In some embodiments, the chimeric capsid protein comprises, in N-terminal to C-terminal order, a first polypeptide segment having sequence at least about 80%, 85%, 90%, 95%, 96%, 97%, 98%, 99%, 99.5%, or 100% identical to SEQ ID NO: 411 or at least about 80%, 85%, 90%, 95%, 96%, 97%, 98%, 99%, 99.5%, or 100% identical to SEQ ID NO: 412; a second polypeptide segment having sequence at least about 80%, 85%, 90%, 95%, 96%, 97%, 98%, 99%, 99.5%, or 100% identical to SEQ ID NO: 413 or at least about 80%, 85%, 90%, 95%, 96%, 97%, 98%, 99%, 99.5%, or 100% identical to SEQ ID NO: 414; a third polypeptide segment having sequence at least about 80%, 85%, 90%, 95%, 96%, 97%, 98%, 99%, 99.5%, or 100% identical to SEQ ID NO: 415 or at least about 80%, 85%, 90%, 95%, 96%, 97%, 98%, 99%, 99.5%, or 100% identical to SEQ ID NO: 416; a fourth polypeptide segment having sequence at least about 80%, 85%, 90%, 95%, 96%, 97%, 98%, 99%, 99.5%, or 100% identical to SEQ ID NO: 417 or at least about 80%, 85%, 90%, 95%, 96%, 97%, 98%, 99%, 99.5%, or 100% identical to SEQ ID NO: 418; and a fifth polypeptide segment having sequence at least about 80%, 85%, 90%, 95%, 96%, 97%, 98%, 99%, 99.5%, or 100% identical to SEQ ID NO: 419 or at least about 80%, 85%, 90%, 95%, 96%, 97%, 98%, 99%, 99.5%, or 100% identical to SEQ ID NO: 420. In some embodiments, at least one polypeptide segment is derived from the AAV5 capsid protein and at least one polypeptide segment is derived from the AAV9 capsid protein.

AAV9 derived polypeptide segment 1:
(SEQ ID NO: 411)
MAADGYLPDWLEDNLSEGIREWWALKPGAPQPKANQQHQDNARGLVLPG
Y
Sequence of AAV5 derived polypeptide segment 1:
(SEQ ID NO: 412)
MSFVDHPPDWLEEVGEGLREFLGLEAGPPKPKPNQQHQDQARGLVLPGY
Sequence of AAV9 derived polypeptide segment 2:
(SEQ ID NO: 413)
KYLGPGNGLDKGEPVNAADAAALEHDKAYDQQLK
Sequence of AAV5 derived polypeptide segment 2:
(SEQ ID NO: 414)
NYLGPGNGLDRGEPVNRADEVAREHDISYNEQLE
Sequence of AAV9 derived polypeptide segment 3:
(SEQ ID NO: 415)
AGDNPYLKYNHADAEFQERLKEDTSFGGNLGRAVFQAKKRLLEP
Sequence of AAV5 derived polypeptide segment 3:
(SEQ ID NO: 416)
AGDNPYLKYNHADAEFQEKLADDTSFGGNLGKAVFQAKKRVLEP
Sequence of AAV9 derived polypeptide segment 4:
(SEQ ID NO: 417)
LGLVEEAAKTAPGKKRPVEQSPQEPDSSAGIGKSGAQPAKKRLNFGQTG
DTESVPDPQPIGEPPAAPSGVGSLTMASGGGAPVA
Sequence of AAV5 derived polypeptide segment 4:
(SEQ ID NO: 418)
FGLVEEGAKTAPTGKRIDDHFPKRKKARTEEDSKPSTSSDAEAGPSGSQ
QLQIPAQPASSLGADTMSAGGGGPLG
Sequence of AAV9 derived polypeptide segment 5:
(SEQ ID NO: 419)
DNNEGADGVGSSSGNWHCDSQWLGDRVITTSTRTWALPTYNNHLYKQIS
NSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSA
HEGCLPPFPADVFMIPQYGYLTLNDGSQAVGRSSFYCLEYFPSQMLRTG
NNFQFSYEFENVPFHSSYAHSQSLDRLMNPLIDQYLYYLSKTINGSGQN
QQTLKFSVAGPSNMAVQGRNYIPGPSYRQQRVSTTVTQNNNSEFAWPGA
SSWALNGRNSLMNPGPAMASHKEGEDRFFPLSGSLIFGKQGTgrdnvDA
DKVMITNEEEIKTTNPVATESYGQVATNHQSAQAQAQTGWVQNQGILPG
MVWQDRDVYLQGPIWAKIPHTDGNFHPSPLMGGFGMKHPPPQILIKNTP
VPADPPTAFNKDKLNSFITQYSTGQVSVEIEWELQKENSKRWNPEIQYT
SNYYKSNNVEFAVNTEGVYSEPRPIGTRYLTRNL

Sequence of AAV9 derived polypeptide segment 5 with Q688K mutation:

(SEQ ID NO: 420)
DNNEGADGVGSSSGNWHCDSQWLGDRVITTSTRTWALPTYNNHLYKQIS
NSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSA
HEGCLPPFPADVFMIPQYGYLTLNDGSQAVGRSSFYCLEYFPSQMLRTG
NNFQFSYEFENVPFHSSYAHSQSLDRLMNPLIDQYLYYLSKTINGSGQN
QQTLKFSVAGPSNMAVQGRNYIPGPSYRQQRVSTTVTQNNNSEFAWPGA
SSWALNGRNSLMNPGPAMASHKEGEDRFFPLSGSLIFGKQGTgrdnvDA
DKVMITNEEEIKTTNPVATESYGQVATNHQSAQAQAQTGWVQNQGILPG
MVWQDRDVYLQGPIWAKIPHTDGNFHPSPLMGGFGMKHPPPQILIKNTP
VPADPPTAFNKDKLNSFITQYSTGQVSVEIEWELKKENSKRWNPEIQYT
SNYYKSNNVEFAVNTEGVYSEPRPIGTRYLTRNL

In some embodiments, the chimeric capsid protein comprises, consists essentially of, or consists of a polypeptide sequence at least about 80%, 85%, 90%, 95%, 96%, 97%, 98%, 99%, 99.5%, or 100% identical to one of SEQ ID NOs: 421-444, or a functional fragment thereof.

TABLE 2
Capsid Protein Sequences

| Name/Alternate Name | SEQ ID NO: |
|---|---|
| ZC23 | 421 |
| ZC24 | 422 |
| ZC25 | 423 |
| ZC26 | 424 |
| ZC27 | 425 |
| ZC28 | 426 |
| ZC29 | 427 |
| ZC30 | 428 |
| ZC31 | 429 |
| ZC32 | 430 |
| ZC33 | 431 |
| ZC34 | 432 |
| ZC35 | 433 |
| ZC40/TN8 | 434 |
| ZC41 | 435 |
| ZC42 | 436 |
| ZC43 | 437 |
| ZC44/TN10 | 438 |
| ZC45 | 439 |
| ZC46 | 440 |
| ZC47/TN14 | 441 |
| ZC48 | 442 |
| ZC49 | 443 |
| ZC50 | 444 |

In some embodiments, the chimeric capsid protein of the present disclosure comprises the sequence KGSGQNQQT (SEQ ID NO:727), optionally in addition to any chimeric modification described herein. In some embodiments, N452K substitution, relative to reference SEQ ID NO:1, is combined with any other chimeric modification(s) described herein.

### Combinatory Capsid Protein

In one aspect, the present disclosure provides combinatory capsid proteins. As used herein, “combinatory capsid protein” refers to a AAV5/AAV9 chimeric capsid protein as described in the present disclosure, which further comprises amino acid variations with respect to the chimeric parental sequence at one or more sites. In some embodiments, the one or more sites of the chimeric parental sequence are selected from those equivalent to the VR-IV site, the VR-V site, the VR-VII site and the VR-VIII site of the AAV9 capsid protein.

The combinatory capsid proteins of the present disclosure include any variant polypeptide sequences identified as shown in, but not limited to, the Examples. Without being limited to any particular example, in some embodiments, the combinatory capsid protein comprises a chimeric AAV5/AAV9 capsid protein backbone, and further comprises the variant polypeptide sequence at one or more sites selected from the group consisting of those equivalent to the VR-IV site, the VR-V site, the VR-VII site and the VR-VIII site of the AAV9 capsid protein as described herein.

In some embodiments, the combinatory capsid protein comprises, in N-terminal to C-terminal order, a first polypeptide segment having sequence at least about 80%, 85%, 90%, 95%, 96%, 97%, 98%, 99%, 99.5%, or 100% identical to SEQ ID NO: 411 or at least about 80%, 85%, 90%, 95%, 96%, 97%, 98%, 99%, 99.5%, or 100% identical to SEQ ID NO: 412; a second polypeptide segment having sequence at least about 80%, 85%, 90%, 95%, 96%, 97%, 98%, 99%, 99.5%, or 100% identical to SEQ ID NO: 413 or at least about 80%, 85%, 90%, 95%, 96%, 97%, 98%, 99%, 99.5%, or 100% identical to SEQ ID NO: 414; a third polypeptide segment having sequence at least about 80%, 85%, 90%, 95%, 96%, 97%, 98%, 99%, 99.5%, or 100% identical to SEQ ID NO: 415 or at least about 80%, 85%, 90%, 95%, 96%, 97%, 98%, 99%, 99.5%, or 100% identical to SEQ ID NO: 416; a fourth polypeptide segment having sequence at least about 80%, 85%, 90%, 95%, 96%, 97%, 98%, 99%, 99.5%, or 100% identical to SEQ ID NO: 417 or at least about 80%, 85%, 90%, 95%, 96%, 97%, 98%, 99%, 99.5%, or 100% identical to SEQ ID NO: 418; and a fifth polypeptide segment having sequence at least about 80%, 85%, 90%, 95%, 96%, 97%, 98%, 99%, 99.5%, or 100% identical to SEQ ID NO: 419 or at least about 80%, 85%, 90%, 95%, 96%, 97%, 98%, 99%, 99.5%, or 100% identical to SEQ ID NO: 420 (here, regions equivalent to the VR-IV site, the VR-V site, the VR-VII site and the VR-VIII site of the AAV9 capsid protein are excluded in the sequence identity calculation of the fifth polypeptide segment). In some embodiments, the combinatory capsid protein comprises a variant polypeptide sequence at one or more of a VR-IV site, a VR-V site, a VR-VII site, and a VR-VIII site of a parental sequence, wherein the parental sequence comprises a sequence at least 80%, at least 85%, at least 90%, at least 95%, at least 98%, at least 99%, or 100% identical to SEQ ID NO: 463. (In SEQ ID NO:463, the amino acids residues labeled “X” are excluded from sequence identity calculation.)

In some embodiments, at least one polypeptide segment is derived from the AAV5 capsid protein and at least one polypeptide segment is derived from the AAV9 capsid protein.

In some embodiments, the combinatory capsid protein further comprises variant polypeptide sequence at one or more sites selected from those equivalent to the VR-IV site, the VR-V site, the VR-VII site and the VR-VIII site of the AAV9 capsid protein.

In some embodiments, the combinatory capsid protein has a variant polypeptide sequence at the site equivalent to the VR-IV site of the AAV9 capsid protein, which comprises, consists essentially of, or consists of a sequence at least about 60%, 70%, 80%, 90%, or 100% identical to GYHKSGAAQ (SEQ ID NO: 6). In some embodiments, the variant polypeptide sequence at the site equivalent to the VR-IV site of the AAV9 capsid protein comprises, consists essentially of, or consists of a sequence consisting of at most 1, 2, 3, or 4 conservative amino-acid substitutions relative GYHKSGAAQ (SEQ ID NO: 6).

In some embodiments, the combinatory capsid protein has a variant polypeptide sequence at the site equivalent to the VR-V site of the AAV9 capsid protein, which comprises, consists essentially of, or consists of a sequence at least about 60%, 70%, 80%, 90%, or 100% identical to LNSMLI (SEQ ID NO: 105). In some embodiments, the variant polypeptide sequence at the site equivalent to the VR-V site of the AAV9 capsid protein comprises, consists essentially of, or consists of a sequence consisting of at most 1, 2, 3, or 4 conservative amino-acid substitutions relative LNSMLI (SEQ ID NO: 105).

In some embodiments, the combinatory capsid protein has a variant polypeptide sequence at the site equivalent to the VR-VIII site of the AAV9 capsid protein, which comprises, consists essentially of, or consists of a sequence at least about 60%, 70%, 80%, 90%, or 100% identical to ANYG (SEQ ID NO: 305) or NVSY (SEQ ID NO: 303). In some embodiments, the variant polypeptide sequence at the site equivalent to the VR-VIII site of the AAV9 capsid protein comprises, consists essentially of, or consists of a sequence consisting of at most 1, 2, 3, or 4 conservative amino-acid substitutions relative ANYG (SEQ ID NO: 305) or NVSY (SEQ ID NO: 303).

In some embodiments, the residue at the position equivalent to Q688 of the AAV9 capsid protein sequence (SEQ ID NO: 1) is a lysine (K) in the combinatory capsid protein.

In some embodiments, the combinatory capsid protein comprises, consists essentially of, or consists of a polypeptide sequence at least about 80%, 85%, 90%, 95%, 96%, 97%, 98%, 99%, 99.5%, or 100% identical to one of SEQ ID NOs: 445-462, or a functional fragment thereof.

TABLE 3
Capsid Protein Sequences

| Name/Alternate Name | SEQ ID NO: |
|---|---|
| TN47-07 | 445 |
| TN47-10/TN12 | 446 |
| TN47-13 | 447 |
| TN47-14 | 448 |
| TN47-17 | 449 |
| TN47-22 | 450 |
| TN40-07 | 451 |
| TN40-10 | 452 |
| TN40-13 | 453 |
| TN40-14 | 454 |
| TN40-17 | 455 |
| TN40-22 | 456 |
| TN44-07/TN13 | 457 |
| TN44-10 | 458 |
| TN44-13 | 459 |
| TN44-14 | 460 |
| TN44-17 | 461 |
| TN44-22 | 462 |

In some embodiments, the combinatory capsid protein of the present disclosure comprises the sequence KGSGQNQQT (SEQ ID NO:727), optionally in addition to any combinatory modification described herein. In some embodiments, N452K substitution, relative to reference SEQ ID NO:1, is combined with any other combinatory modification(s) described herein.

In some embodiments, the disclosure provides an AAV9, AAV5/AAV9 chimeric, or combinatory capsid protein comprising a sequence at least 80%, 85%, 90%, 95%, 99%, or 100% identical to a modified capsid selected from SEQ ID NOs: 402-410, 421-462, 464-468, wherein the amino acid substitutions, optionally conservative substitutions, with the specified percent identity level are tolerated.

### Additional Mutations

Additional amino acid substitutions may be incorporated, for example, to further improve transduction efficiency or tissue selectivity.

Exemplary non-limiting substitutions include, but are not limited to, S651A, T578A or T582A relative to the sequence of AAV5, in either an AAV5 or AAV9-based capsid.

In some embodiments, the capsid protein comprises a mutation selected from S651A, T578A, T582A, K251R, Y709F, Y693F, or S485A relative to the sequence of AAV5, in either an AAV5 or AAV9-based capsid. In some embodiments, the capsid protein comprises a mutation selected from K251R, Y709F, Y693F, or S485A relative to the sequence of AAV5, in either an AAV5 or AAV9-based capsid.

### Transduction Efficiency, Tropism, and NAb Evasion

Transduction efficiency can be determined using methods known in the art or those described in the Examples. In some embodiments, the rAAV virion with engineered capsid protein exhibits increased transduction efficiency in cardiac cells compared to an AAV virion comprising the parental sequence. The rAAV virion referenced in this section is any rAAV virion with modified or engineered capsid protein described herein.

In some embodiments, the rAAV virion exhibits increased transduction efficiency in human cardiac fibroblast (hCF) cells compared to an AAV virion comprising the parental sequence. In some embodiments, the human cardiac fibroblasts are located in the left ventricle of the heart.

In some embodiments, the rAAV virion exhibits at least 2-, 3-, 4-, 5-, 6, 7-, 8-, 9-, 10-, 11-, 12-, 13-, 14, or 15-fold increased transduction efficiency in hCF cells at a multiplicity of infection (MOI) of 100,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in hCF cells at a multiplicity of infection (MOI) of 100,000. In some embodiments, the rAAV virion exhibits at least 2-, 3-, 4-, 5-, 6, 7-, 8-, 9-, 10, 11-, 12-, 13-, 14, or 15-fold increased transduction efficiency in hCF cells at a multiplicity of infection (MOI) of 100,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in hCF cells at a multiplicity of infection (MOI) of 100,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in hCF cells at a multiplicity of infection (MOI) of 1,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in hCF cells at a multiplicity of infection (MOI) of 1,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in hCF cells at a multiplicity of infection (MOI) of 1,000.

In some embodiments, the rAAV virion exhibits increased transduction efficiency in induced pluripotent stem cell-derived cardiomyocyte (iPS-CM) cells compared to an AAV virion comprising the parental sequence. Accordingly, the fold improvement discussed in this section is as compared to an AAV virion comprising the parental sequence (e.g., AAV9).

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 100,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 100,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 100,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 75,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 75,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 75,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 1,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 1,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 1,000.

In some embodiments, the rAAV virion comprising the engineered capsid protein of the present disclosure exhibits increased transduction efficiency in heart compared to an AAV virion comprising the parental sequence. In some embodiments, transduction efficiency in heart is monitored by injecting C57BL/6J mice with either AAV9: CAG-GFP or CAG-GFP encapsulated by the engineered capsid protein of the present disclosure. In some embodiments, the injection dosage is 2.5E+11 vg/mouse. In some embodiments, the injection dosage is 2E+11 vg/mouse. In some embodiments, the injection dosage is 1E+11 vg/mouse. In some embodiments, the rAAV virion exhibits at least 2-, 3-, 4-, 5-, 6, 7-, 8-, 9-, 10-, 11-, 12-, 13-, 14, or 15-fold increased transduction efficiency in heart. In some embodiments, the rAAV virion exhibits at least 2-, 3-, 4-, 5-, 6, 7-, 8-, 9-, 10-, 11-, 12-, 13-, 14, or 15-fold increased transduction efficiency in heart relative to wild-type AAV9. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart relative to wild-type AAV9. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart relative to wild-type AAV9.

In some embodiments, the rAAV virion comprising the engineered capsid protein of the present disclosure exhibits decreased transduction efficiency in liver cells compared to an AAV virion comprising the parental sequence. In some embodiments, liver transduction efficiency is monitored by injecting C57BL/6J mice with either AAV9: CAG-GFP or CAG-GFP encapsulated by the engineered capsid protein of the present disclosure. In some embodiments, the injection dosage is 2.5E+11 vg/mouse. In some embodiments, the injection dosage is 2E+11 vg/mouse. In some embodiments, the injection dosage is 1E+11 vg/mouse. In some embodiments, the rAAV virion exhibits at least 2-, 3-, 4-, 5-, 6, 7-, 8-, 9-, 10-, 11-, 12-, 13-, 14, or 15-fold decreased transduction efficiency in liver. In some embodiments, the injection dosage is 1E+11 vg/mouse. In some embodiments, the rAAV virion exhibits at least 2-, 3-, 4-, 5-, 6, 7-, 8-, 9-, 10-, 11-, 12-, 13-, 14, or 15-fold decreased transduction efficiency in liver relative to wild-type AAV9. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver relative to wild-type AAV9. In some embodiments, the rAAV virion exhibits about 20% to 30%, about 30% to 40%, about 40% to 50%, about 50% to 80%, or about 80% to 100 decreased transduction efficiency in liver. In some embodiments, the rAAV virion exhibits about 20% to 30%, about 30% to 40%, about 40% to 50%, about 50% to 80%, or about 80% to 100 decreased transduction efficiency in liver relative to wild-type AAV9.

Selectivity for a cell type and/or a tissue/organ type is increased when the ratio of the transduction efficiencies for one cell/tissue/organ type over another is increased for rAAV virions comprising the engineered capsid protein of the present disclosure compared to an AAV virion comprising the parental sequence. In some embodiments, the rAAV virion comprising the engineered capsid protein exhibits increased selectivity for iPS-CM cells over liver cells. In some embodiments, the rAAV virion comprising the engineered capsid protein exhibits increased selectivity for heart over liver when injected in vivo. In some embodiments, the rAAV virion comprising the engineered capsid protein exhibits increased selectivity for the left ventricle of the heart over liver when injected in vivo.

In some embodiments, the rAAV virion comprising the engineered capsid protein exhibits at least 2-, 3-, 4-, 5-, 6, 7-, 8-, 9-, 10-, 11-, 12-, 13-, 14, or 15-fold increased selectivity of iPS-CM cells over liver cells and/or heart over liver. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased selectivity of iPS-CM cells over liver cells and/or heart over liver. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% increased selectivity of iPS-CM cells over liver cells and/or heart over liver.

In some embodiments, the rAAV virion comprising the engineered capsid protein exhibits at least 2-, 3-, 4-, 5-, 6, 7-, 8-, 9-, 10-, 11-, 12-, 13-, 14, or 15-fold increased selectivity of heart tissue over liver tissue. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased selectivity of heart tissue over liver tissue. In some embodiments, the rAAV virion comprising the engineered capsid protein exhibits at least or more than 30%, 40%, 50%, 80%, 100%, 125%, 150%, 175%, 200%, 250%, 300%, 400%, 500%, 600%, 700%, 800% or 1000% increased selectivity of heart tissue over liver tissue. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% increased selectivity of heart tissue over liver tissue.

In some embodiments, the rAAV virion comprising the engineered capsid protein of the present disclosure exhibits improved ability to evade human NAb (neutralizing antibodies) compared to an AAV virion comprising the parental sequence. In some embodiments, the ability to evade human NAb is measured via an NAb inhibition assay. Non-limiting examples of NAb inhibition assays are described in the Example section of the present disclosure. In some embodiments, NAb inhibition assays are performed by incubating AAV virions with pooled human NAb (e.g., IgG) before treating a target cell at a pre-determined MOI and measure the decrease of transduction efficiency compared to AAV virions not incubated with pooled human NAb. Less NAb inhibition indicates improved ability of the AAV virion to evade human NAb. In some embodiments, the rAAV virion comprising the engineered capsid protein exhibits at least 2-, 3-, 4-, 5-, 6, 7-, 8-, 9-, 10, 11-, 12-, 13-, 14, or 15-fold improved ability to evade human NAb. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold improved ability to evade human NAb. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% improved ability to evade human NAb.

In some embodiments, any rAAV comprising N452K mutation as described herein exhibits at least 2-, 3-, 4-, 5-, 6, 7-, 8-, 9-, 10-, 11-, 12-, 13-, 14, or 15-fold increased transduction efficiency in heart relative to wild-type AAV9 and/or relative to transduction of liver. In some embodiments, any rAAV comprising N452K mutation as described herein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart relative to wild-type AAV9 and/or relative to transduction of liver. In some embodiments, any rAAV virion comprising N452K mutation as described herein exhibits at least or more than 30%, 40%, 50%, 80%, 100%, 125%, 150%, 175%, 200%, 250%, 300%, 400%, 500%, 600%, 700%, 800% or 1000% increased transduction efficiency in heart relative to wild-type AAV9 and/or relative to transduction of liver. In some embodiments, any rAAV comprising N452K mutation as described herein exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart relative to wild-type AAV9 and/or relative to transduction of liver.

In some embodiments, any rAAV comprising N452K mutation as described herein exhibits at least 2-, 3-, 4-, 5-, 6, 7-, 8-, 9-, 10-, 11-, 12-, 13-, 14, or 15-fold decreased transduction efficiency in liver relative to wild-type AAV9. In some embodiments, any rAAV comprising N452K mutation as described herein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver relative to wild-type AAV9. In some embodiments, any rAAV virion comprising N452K mutation as described herein exhibits at least or more than 30%, 40%, 50%, 80%, 100%, 125%, 150%, 175%, 200%, 250%, 300%, 400%, 500%, 600%, 700%, 800% or 1000% decreased transduction efficiency in liver relative to wild-type AAV9. In some embodiments, any rAAV comprising N452K mutation as described herein exhibits about 20% to 30%, about 30% to 40%, about 40% to 50%, about 50% to 80%, or about 80% to 100 decreased transduction efficiency in liver relative to wild-type AAV9.

### Gene Products/Transgenes

The transgenes and gene products described herein are non-limiting. Any transgene encoding any gene product may be used in the rAAV virions described herein.

In some embodiments, the rAAV virion of the present disclosure comprises a viral vector comprising a transgene.

A transgene can be a gene or nucleotide sequence that encodes a product, or a functional fragment thereof. A product can be, for example, a polypeptide or a non-coding nucleotide. By non-coding nucleotide, it is meant that the sequence transcribed from the transgene or nucleotide sequence is not translated into a polypeptide. In some embodiments, the product encoded by the transgene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (InRNA), and/or a short interfering RNA (siRNA). In some embodiments, the transgene encodes a product natively expressed by a cardiac cell, e.g., a cardiomyocyte.

In some embodiments, the transgene encodes a polypeptide. In some embodiments, the transgene encodes a non-coding polynucleotide such as, for example, a microRNA (miRNA or mIR).

In some embodiments, the transgene comprises a nucleotide sequence encoding a human protein. In some embodiments, the transgene comprises a human nucleotide sequence (a human DNA sequence). In some embodiments, the transgene comprises a DNA sequence that has been codon-optimized. In some embodiments, the transgene comprises a nucleotide sequence encoding a wild-type protein, or a functionally active fragment thereof. In some embodiments, the transgene comprises a nucleotide sequence encoding a variant of a wild-type protein, such as a functionally active variant thereof.

In some embodiments, the transgene comprises a sequence encoding a product selected from vascular endothelial growth factor (VEGF), a VEGF isoform, VEGF-A, VEGF-B, VEGF-C, VEGF-D, VEGF-D^{dNdC}, VEGF-A_{116A}, VEGF-A₁₆₅, VEGF-A₁₂₁, VEGF-2, placenta growth factor (PIGF), fibroblast growth factor 4 (FGF-4), human growth factor (HGF), human granulocyte colony-stimulating factor (hGCSF), and hypoxia inducible factor 1α (HIF-1α).

In some embodiments, the transgene comprises a sequence encoding a product selected from SERCA2a, stromal cell-derived factor-1 (SDF-1), adenylyl cyclase type 6, S100A1, miRNA-17-92, miR-302-367, anti-miR-29a, anti-miR-30a, antimiR-141, cyclin A2, cyclin-dependent kinase 2, Tbx20, miRNA-590, miRNA-199, anti-sense oligonucleotide against Lp(a), interfering RNA against PCSK9, anti-sense oligonucleotide against apolipoprotein C-III, lipoprotein lipase^{S447X}, anti-sense oligonucleotide against apolipoprotein B, anti-sense oligonucleotide against c-myc, and E2F oligonucleotide decoy.

In some embodiments, the transgene encodes a gene product whose expression complements a defect in a gene responsible for a genetic disorder. In some embodiments, the disclosure provides, without limitation, polynucleotides encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOSIs (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, a polynucleotide encodes one or more of: cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the transgene comprises a nucleotide sequence encoding a protein selected from DWORF, junctophilin (e.g., JPH2), BAG family molecular chaperone regulator 3 (BAG3), phospholamban (PLN), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), junction plakoglobin (JUP), and plakophilin-2 (PKP2). In some embodiments, the transgene comprises a nucleotide sequence encoding a matrix metallopeptidase 11 (MMP11) protein, a synaptopodin 2 like (SYNPO2L) protein (e.g., SYNPO2LA or SYNPO2LA), or an RNA binding motif protein 20 (RBM20). In some embodiments, the transgene comprises a nucleotide sequence encoding an inhibitory oligonucleotide targeting metastasis suppressor protein 1 (MTSS1).

In some embodiments, the transgene in the viral vector (such as that in the rAAV virion of the present disclosure) is selected from DWORF, JPH2, BAG3, CRYAB, LMNA (e.g., Lamin A isoform of LMNA, or Lamin C isoform of LMNA), TNNI3, PLN, LAMP2 (e.g., LAMP2a, LAMP2b, or LAMP2c), DSP (e.g., DPI isoform of DSP or DPII isoform of DSP), DSG2 and JUP.

In some embodiments, the transgene comprises a polynucleotide sequence encoding a MYBPC3 polypeptide. In some embodiments, the transgene comprises a polynucleotide sequence encoding a human MYBPC3 polypeptide. In some embodiments, the transgene comprises, essentially consists of, or consists of SEQ ID NO: 811. In some embodiments, a polynucleotide sequence is a codon-optimized sequence encoding MYBPC3, e.g., human MYBPC3. In some embodiments, the transgene comprises a polynucleotide sequence that has at least 70%, 75%, 80%, 85%, 90%, 95%, 98%, 99% or 100% sequence identity to SEQ ID NO: 811. In some embodiments, the MYBPC3 polypeptide comprises, essentially consists of, or consists of SEQ ID NO: 815. In some embodiments, the MYBPC3 polypeptide has least 75%, 80%, 85%, 90%, 95%, 98%, 99% or 100% sequence identity to SEQ ID NO: 815.

In some embodiments, the transgene comprises a polynucleotide sequence encoding a MYBPC3-delC3 variant polypeptide. In some embodiments, the transgene comprises, essentially consists of, or consists of SEQ ID NO: 812. In some embodiments, a polynucleotide sequence is a codon-optimized sequence encoding MYBPC3-delC3. In some embodiments, the transgene comprises a polynucleotide sequence that has at least 70%, 75%, 80%, 85%, 90%, 95%, 98%, 99% or 100% sequence identity to SEQ ID NO: 812. In some embodiments, the MYBPC3-delC3 variant polypeptide comprises, essentially consists of, or consists of SEQ ID NO: 816. In some embodiments, the MYBPC3-delC3 variant polypeptide has least 75%, 80%, 85%, 90%, 95%, 98%, 99% or 100% sequence identity to SEQ ID NO: 816.

In some embodiments, the transgene comprises a polynucleotide sequence encoding a MYBPC3-delC4 variant polypeptide. In some embodiments, the transgene comprises, essentially consists of, or consists of SEQ ID NO: 813. In some embodiments, a polynucleotide sequence is a codon-optimized sequence encoding MYBPC3-delC4. In some embodiments, the transgene comprises a polynucleotide sequence that has at least 70%, 75%, 80%, 85%, 90%, 95%, 98%, 99% or 100% sequence identity to SEQ ID NO: 813. In some embodiments, the MYBPC3-delC4 variant polypeptide comprises, essentially consists of, or consists of SEQ ID NO: 817. In some embodiments, the MYBPC3-delC4 variant polypeptide has least 75%, 80%, 85%, 90%, 95%, 98%, 99% or 100% sequence identity to SEQ ID NO: 817.

In some embodiments, the transgene comprises a polynucleotide sequence encoding a MYBPC3-delC4b variant polypeptide. In some embodiments, the transgene comprises, essentially consists of, or consists of SEQ ID NO: 814. In some embodiments, a polynucleotide sequence is a codon-optimized sequence encoding MYBPC3-delC4b. In some embodiments, the transgene comprises a polynucleotide sequence that has at least 70%, 75%, 80%, 85%, 90%, 95%, 98%, 99% or 100% sequence identity to SEQ ID NO: 814. In some embodiments, the MYBPC3-delC4b variant polypeptide comprises, essentially consists of, or consists of SEQ ID NO: 818. In some embodiments, the MYBPC3-delC4b variant polypeptide has least 75%, 80%, 85%, 90%, 95%, 98%, 99% or 100% sequence identity to SEQ ID NO: 818.

In some embodiments, the transgene comprises a polynucleotide sequence encoding a DWORF polypeptide. In some embodiments, the transgene comprises a polynucleotide sequence encoding a human DWORF polypeptide. In some embodiments, the transgene comprises, essentially consists of, or consists of SEQ ID NO: 827 or SEQ ID NO:828. In some embodiments, a polynucleotide sequence is a codon-optimized sequence encoding DWORF, e.g., human DWORF. In some embodiments, the transgene comprises a polynucleotide sequence that has at least 70%, 75%, 80%, 85%, 90%, 95%, 98%, 99% or 100% sequence identity to SEQ ID NO: 827 or SEQ ID NO:828. In some embodiments, the DWORF polypeptide comprises, essentially consists of, or consists of SEQ ID NO: 826. In some embodiments, the DWORF polypeptide has least 75%, 80%, 85%, 90%, 95%, 98%, 99% or 100% sequence identity to SEQ ID NO: 826.

In some embodiments, the transgene comprises a polynucleotide sequence encoding a junctophilin 2 (JPH2) polypeptide. In some embodiments, the transgene comprises a polynucleotide sequence encoding a full-length JPH2 polypeptide. In some embodiments, the transgene comprises a polynucleotide sequence encoding a human JPH2 polypeptide. In some embodiments, the transgene comprises, essentially consists of, or consists of SEQ ID NO: 782. In some embodiments, a polynucleotide sequence is a codon-optimized sequence encoding JPH2, e.g., human JPH2. In some embodiments, the transgene comprises a polynucleotide sequence that has at least 70%, 75%, 80%, 85%, 90%, 95%, 98%, 99% or 100% sequence identity to SEQ ID NO: 782. In some embodiments, the JPH2 polypeptide comprises, essentially consists of, or consists of SEQ ID NO: 783. In some embodiments, the JPH2 polypeptide has least 75%, 80%, 85%, 90%, 95%, 98%, 99% or 100% sequence identity to SEQ ID NO: 783.

In some embodiments, the transgene comprises a polynucleotide sequence encoding an N-terminal fragment of the JPH2 polypeptide. In some embodiments, the transgene comprises a polynucleotide sequence encoding an N-terminal fragment of the JPH2 polypeptide, which retains the JPH2 activity. In some embodiments, the transgene comprises, essentially consists of, or consists of SEQ ID NO: 809. In some embodiments, a polynucleotide sequence is a codon-optimized sequence encoding N-terminal fragment of JPH2. In some embodiments, the transgene comprises a polynucleotide sequence that has at least 70%, 75%, 80%, 85%, 90%, 95%, 98%, 99% or 100% sequence identity to SEQ ID NO: 809. In some embodiments, the N-terminal fragment of JPH2 polypeptide comprises, essentially consists of, or consists of SEQ ID NO: 808. In some embodiments, the N-terminal fragment of JPH2 polypeptide has least 75%, 80%, 85%, 90%, 95%, 98%, 99% or 100% sequence identity to SEQ ID NO: 808.

In some embodiments, the transgene comprises a polynucleotide sequence encoding a BAG3 polypeptide. In some embodiments, the transgene comprises a polynucleotide sequence encoding a human BAG3 polypeptide. In some embodiments, the transgene comprises, essentially consists of, or consists of SEQ ID NO: 785. In some embodiments, a polynucleotide sequence is a codon-optimized sequence encoding BAG3, e.g., human BAG3. In some embodiments, the transgene comprises a polynucleotide sequence that has at least 70%, 75%, 80%, 85%, 90%, 95%, 98%, 99% or 100% sequence identity to SEQ ID NO: 785. In some embodiments, the BAG3 polypeptide comprises, essentially consists of, or consists of SEQ ID NO: 784. In some embodiments, the BAG3 polypeptide has least 75%, 80%, 85%, 90%, 95%, 98%, 99% or 100% sequence identity to SEQ ID NO: 784.

In some embodiments, the transgene comprises a polynucleotide sequence encoding a C151R mutant form of BAG3 polypeptide. In some embodiments, a polynucleotide sequence is a codon-optimized sequence encoding a C151R mutant form of BAG3 polypeptide. In some embodiments, a C151R mutant form of BAG3 polypeptide comprises, essentially consists of, or consists of SEQ ID NO: 829. In some embodiments, a C151R mutant form of BAG3 polypeptide has least 75%, 80%, 85%, 90%, 95%, 98%, 99% or 100% sequence identity to SEQ ID NO: 829.

In some embodiments, the transgene comprises a polynucleotide sequence encoding a CRYAB polypeptide. In some embodiments, the transgene comprises a polynucleotide sequence encoding a human CRYAB polypeptide. In some embodiments, the transgene comprises, essentially consists of, or consists of SEQ ID NO: 787. In some embodiments, a polynucleotide sequence is a codon-optimized sequence encoding CRYAB, e.g., human CRYAB. In some embodiments, the transgene comprises a polynucleotide sequence that has at least 70%, 75%, 80%, 85%, 90%, 95%, 98%, 99% or 100% sequence identity to SEQ ID NO: 787. In some embodiments, the CRYAB polypeptide comprises, essentially consists of, or consists of SEQ ID NO: 786. In some embodiments, the CRYAB polypeptide has least 75%, 80%, 85%, 90%, 95%, 98%, 99% or 100% sequence identity to SEQ ID NO: 786.

In some embodiments, the transgene comprises a polynucleotide sequence encoding a LMNA polypeptide. In some embodiments, the transgene comprises a polynucleotide sequence encoding a human LMNA polypeptide. In some embodiments, the transgene comprises a polynucleotide sequence encoding the LaminA isoform of LMNA. In some embodiments, the transgene comprises, essentially consists of, or consists of SEQ ID NO: 789. In some embodiments, a polynucleotide sequence is a codon-optimized sequence encoding LaminA isoform of LMNA, e.g., human. In some embodiments, the transgene comprises a polynucleotide sequence that has at least 70%, 75%, 80%, 85%, 90%, 95%, 98%, 99% or 100% sequence identity to SEQ ID NO: 789. In some embodiments, the LaminA isoform of LMNA polypeptide comprises, essentially consists of, or consists of SEQ ID NO: 788. In some embodiments, the LMNA polypeptide has least 75%, 80%, 85%, 90%, 95%, 98%, 99% or 100% sequence identity to SEQ ID NO: 788.

In some embodiments, the transgene comprises a polynucleotide sequence encoding the LaminC isoform of LMNA. In some embodiments, the transgene comprises, essentially consists of, or consists of SEQ ID NO: 791. In some embodiments, a polynucleotide sequence is a codon-optimized sequence encoding LaminC isoform of LMNA, e.g., human. In some embodiments, the transgene comprises a polynucleotide sequence that has at least 70%, 75%, 80%, 85%, 90%, 95%, 98%, 99% or 100% sequence identity to SEQ ID NO: 791. In some embodiments, the LaminC isoform of LMNA polypeptide comprises, essentially consists of, or consists of SEQ ID NO: 790. In some embodiments, the LMNA polypeptide has least 75%, 80%, 85%, 90%, 95%, 98%, 99% or 100% sequence identity to SEQ ID NO: 790.

In some embodiments, the transgene comprises a polynucleotide sequence encoding a TNNI3 polypeptide. In some embodiments, the transgene comprises a polynucleotide sequence encoding a human TNNI3 polypeptide. In some embodiments, the transgene comprises, essentially consists of, or consists of SEQ ID NO: 793. In some embodiments, a polynucleotide sequence is a codon-optimized sequence encoding TNNI3, e.g., human TNNI3. In some embodiments, the transgene comprises a polynucleotide sequence that has at least 70%, 75%, 80%, 85%, 90%, 95%, 98%, 99% or 100% sequence identity to SEQ ID NO: 793. In some embodiments, the TNNI3 polypeptide comprises, essentially consists of, or consists of SEQ ID NO: 792. In some embodiments, the TNNI3 polypeptide has least 75%, 80%, 85%, 90%, 95%, 98%, 99% or 100% sequence identity to SEQ ID NO: 792.

In some embodiments, the transgene comprises a polynucleotide sequence encoding a PLN polypeptide. In some embodiments, the transgene comprises a polynucleotide sequence encoding a human PLN polypeptide. In some embodiments, the transgene comprises, essentially consists of, or consists of SEQ ID NO: 810. In some embodiments, a polynucleotide sequence is a codon-optimized sequence encoding PLN, e.g., human PLN. In some embodiments, the transgene comprises a polynucleotide sequence that has at least 70%, 75%, 80%, 85%, 90%, 95%, 98%, 99% or 100% sequence identity to SEQ ID NO: 810. In some embodiments, the PLN polypeptide comprises, essentially consists of, or consists of SEQ ID NO: 830. In some embodiments, the PLN polypeptide has least 75%, 80%, 85%, 90%, 95%, 98%, 99% or 100% sequence identity to SEQ ID NO: 830.

In some embodiments, the transgene comprises a polynucleotide sequence encoding a guide RNA targeting a mutant PLN gene (such as a deletious mutant of PLN, e.g., PLN-R14Del).

In some embodiments, the transgene comprises a polynucleotide sequence encoding a LAMP2 polypeptide. In some embodiments, the transgene comprises a polynucleotide sequence encoding a human LAMP2 polypeptide. In some embodiments, the transgene comprises a polynucleotide sequence encoding the LAMP2a isoform. In some embodiments, the transgene comprises, essentially consists of, or consists of SEQ ID NO: 795. In some embodiments, a polynucleotide sequence is a codon-optimized sequence encoding LAMP2a, e.g., human LAMP2a. In some embodiments, the transgene comprises a polynucleotide sequence that has at least 70%, 75%, 80%, 85%, 90%, 95%, 98%, 99% or 100% sequence identity to SEQ ID NO: 795. In some embodiments, the LAMP2a polypeptide comprises, essentially consists of, or consists of SEQ ID NO: 794. In some embodiments, the LAMP2a polypeptide has least 75%, 80%, 85%, 90%, 95%, 98%, 99% or 100% sequence identity to SEQ ID NO: 794.

In some embodiments, the transgene comprises a polynucleotide sequence encoding the LAMP2b isoform. In some embodiments, the transgene comprises, essentially consists of, or consists of SEQ ID NO: 797. In some embodiments, a polynucleotide sequence is a codon-optimized sequence encoding LAMP2b, e.g., human LAMP2b. In some embodiments, the transgene comprises a polynucleotide sequence that has at least 70%, 75%, 80%, 85%, 90%, 95%, 98%, 99% or 100% sequence identity to SEQ ID NO: 797. In some embodiments, the LAMP2b polypeptide comprises, essentially consists of, or consists of SEQ ID NO: 796. In some embodiments, the LAMP2b polypeptide has least 75%, 80%, 85%, 90%, 95%, 98%, 99% or 100% sequence identity to SEQ ID NO: 796.

In some embodiments, the transgene comprises a polynucleotide sequence encoding the LAMP2c isoform. In some embodiments, the transgene comprises, essentially consists of, or consists of SEQ ID NO: 799. In some embodiments, a polynucleotide sequence is a codon-optimized sequence encoding LAMP2c, e.g., human LAMP2c. In some embodiments, the transgene comprises a polynucleotide sequence that has at least 70%, 75%, 80%, 85%, 90%, 95%, 98%, 99% or 100% sequence identity to SEQ ID NO: 799. In some embodiments, the LAMP2c polypeptide comprises, essentially consists of, or consists of SEQ ID NO: 798. In some embodiments, the LAMP2c polypeptide has least 75%, 80%, 85%, 90%, 95%, 98%, 99% or 100% sequence identity to SEQ ID NO: 798.

In some embodiments, the transgene comprises a polynucleotide sequence encoding a DSP polypeptide. In some embodiments, the transgene comprises a polynucleotide sequence encoding a human DSP polypeptide. In some embodiments, the transgene comprises a polynucleotide sequence encoding the DPI isoform of DSP. In some embodiments, the transgene comprises, essentially consists of, or consists of SEQ ID NO: 801. In some embodiments, a polynucleotide sequence is a codon-optimized sequence encoding DPI isoform of DSP, e.g., human. In some embodiments, the transgene comprises a polynucleotide sequence that has at least 70%, 75%, 80%, 85%, 90%, 95%, 98%, 99% or 100% sequence identity to SEQ ID NO: 801. In some embodiments, the DPI isoform of DSP polypeptide comprises, essentially consists of, or consists of SEQ ID NO: 800. In some embodiments, the DPI isoform of DSP polypeptide has least 75%, 80%, 85%, 90%, 95%, 98%, 99% or 100% sequence identity to SEQ ID NO: 800.

In some embodiments, the transgene comprises a polynucleotide sequence encoding the DPII isoform of DSP. In some embodiments, the transgene comprises, essentially consists of, or consists of SEQ ID NO: 803. In some embodiments, a polynucleotide sequence is a codon-optimized sequence encoding DPII isoform of DSP, e.g., human. In some embodiments, the transgene comprises a polynucleotide sequence that has at least 70%, 75%, 80%, 85%, 90%, 95%, 98%, 99% or 100% sequence identity to SEQ ID NO: 803. In some embodiments, the DPII isoform of DSP polypeptide comprises, essentially consists of, or consists of SEQ ID NO: 802. In some embodiments, the DPII isoform of DSP polypeptide has least 75%, 80%, 85%, 90%, 95%, 98%, 99% or 100% sequence identity to SEQ ID NO: 802.

In some embodiments, the transgene comprises a polynucleotide sequence encoding a DSG2 polypeptide. In some embodiments, the transgene comprises a polynucleotide sequence encoding a human DSG2 polypeptide. In some embodiments, the transgene comprises, essentially consists of, or consists of SEQ ID NO: 805. In some embodiments, a polynucleotide sequence is a codon-optimized sequence encoding DSG2, e.g., human DSG2. In some embodiments, the transgene comprises a polynucleotide sequence that has at least 70%, 75%, 80%, 85%, 90%, 95%, 98%, 99% or 100% sequence identity to SEQ ID NO: 805. In some embodiments, the DSG2 polypeptide comprises, essentially consists of, or consists of SEQ ID NO: 804. In some embodiments, the DSG2 polypeptide has least 75%, 80%, 85%, 90%, 95%, 98%, 99% or 100% sequence identity to SEQ ID NO: 804.

In some embodiments, the transgene comprises a polynucleotide sequence encoding a JUP polypeptide. In some embodiments, the transgene comprises a polynucleotide sequence encoding a human JUP polypeptide. In some embodiments, the transgene comprises, essentially consists of, or consists of SEQ ID NO: 807. In some embodiments, a polynucleotide sequence is a codon-optimized sequence encoding JUP, e.g., human JUP. In some embodiments, the transgene comprises a polynucleotide sequence that has at least 70%, 75%, 80%, 85%, 90%, 95%, 98%, 99% or 100% sequence identity to SEQ ID NO: 807. In some embodiments, the JUP polypeptide comprises, essentially consists of, or consists of SEQ ID NO: 806. In some embodiments, the JUP polypeptide has least 75%, 80%, 85%, 90%, 95%, 98%, 99% or 100% sequence identity to SEQ ID NO: 806.

In some embodiments, the transgene comprises a polynucleotide sequence encoding MMP11. In some embodiments, the transgene comprises a polynucleotide sequence encoding a human MMP11 polypeptide. In some embodiments, the transgene comprises, essentially consists of, or consists of SEQ ID NO: 819. In some embodiments, a polynucleotide sequence is a codon-optimized sequence encoding MMP11, e.g., human MMP11. In some embodiments, the transgene comprises a polynucleotide sequence that has at least 70%, 75%, 80%, 85%, 90%, 95%, 98%, 99% or 100% sequence identity to SEQ ID NO: 819. In some embodiments, the MMP11 polypeptide comprises, essentially consists of, or consists of SEQ ID NO: 822. In some embodiments, the MMP11 polypeptide has least 75%, 80%, 85%, 90%, 95%, 98%, 99% or 100% sequence identity to SEQ ID NO: 822.

In some embodiments, the transgene comprises a polynucleotide sequence encoding SYNPO2L (e.g., SYNPO2LA or SYNPO2LA). In some embodiments, the transgene comprises a polynucleotide sequence encoding a human SYNPO2L (e.g., SYNPO2LA or SYNPO2LA). In some embodiments, a polynucleotide sequence is a codon-optimized sequence encoding SYNPO2LA, e.g., human. In some embodiments, the transgene comprises, essentially consists of, or consists of SEQ ID NO: 820. In some embodiments, the transgene comprises a polynucleotide sequence that has at least 70%, 75%, 80%, 85%, 90%, 95%, 98%, 99% or 100% sequence identity to SEQ ID NO: 820. In some embodiments, the SYNPO2LA polypeptide comprises, essentially consists of, or consists of SEQ ID NO: 823. In some embodiments, the SYNPO2LA polypeptide has least 75%, 80%, 85%, 90%, 95%, 98%, 99% or 100% sequence identity to SEQ ID NO: 823. In some embodiments, a polynucleotide sequence is a codon-optimized sequence encoding SYNPO2LB, e.g., human. In some embodiments, the transgene comprises, essentially consists of, or consists of SEQ ID NO: 821. In some embodiments, the transgene comprises a polynucleotide sequence that has at least 70%, 75%, 80%, 85%, 90%, 95%, 98%, 99% or 100% sequence identity to SEQ ID NO: 821. In some embodiments, the SYNPO2LB polypeptide comprises, essentially consists of, or consists of SEQ ID NO: 824. In some embodiments, the SYNPO2LB polypeptide has least 75%, 80%, 85%, 90%, 95%, 98%, 99% or 100% sequence identity to SEQ ID NO: 824.

In some embodiments, the transgene comprises a polynucleotide sequence encoding an inhibitory oligonucleotide (e.g., siRNA) targeting MTSS1. In some embodiments, the transgene comprises a polynucleotide sequence encoding an inhibitory oligonucleotide (e.g., siRNA) targeting SEQ ID NO: 831.

In some embodiments, the transgene comprises a polynucleotide sequence encoding saCas9. In some embodiments, the transgene comprises, essentially consists of, or consists of SEQ ID NO: 832. In some embodiments, a polynucleotide sequence is a codon-optimized sequence encoding saCas9. In some embodiments, the transgene comprises a polynucleotide sequence that has at least 70%, 75%, 80%, 85%, 90%, 95%, 98%, 99% or 100% sequence identity to SEQ ID NO: 832. In some embodiments, the saCas9 polypeptide comprises, essentially consists of, or consists of SEQ ID NO: 833. In some embodiments, the saCas9 polypeptide has least 75%, 80%, 85%, 90%, 95%, 98%, 99% or 100% sequence identity to SEQ ID NO: 833.

In some embodiments, the rAAV virion of the present disclosure comprises a heterologous nucleic acid comprising a nucleotide sequence that encodes one or more gene products selected from MYBPC3, KCNH2, TRPM4, DSG2, ATP2A2, CACNA1C, DMD, DMPK, EPG5, EVC, EVC2, FBN1, NF1, SCN5A, SOS1, NPR1, ERBB4, VIP, MYH6, MYH7, or a mutant, variant, or fragment thereof. In some embodiments, the rAAV virion of the present disclosure comprises a heterologous nucleic acid comprising a nucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3 and ACTN2. In some embodiments, the rAAV virion of the present disclosure comprises a heterologous nucleic acid comprising a nucleotide sequence that encodes one or more gene products selected from MYBPC3, DWORF, JPH2, BAG3, CRYAB, Lamin A isoform of LMNA, Lamin C isoform of LMNA, TNNI3, PLN, LAMP2a, LAMP2b, LAMP2c, DPI isoform of DSP, DPII isoform of DSP, DSG2, MYH6, MYH7, RBM20, and JUP.

In some embodiments, the rAAV virion of the present disclosure comprises a heterologous nucleic acid comprising a nucleotide sequence that encodes one or more gene products selected from ASCL1, MYOCD, MEF2C, and TBX5. In some embodiments, the rAAV virion of the present disclosure comprises a heterologous nucleic acid comprising a nucleotide sequence that encodes one or more gene products selected from ASCL1, MYOCD, MEF2C, AND TBX5, CCNB1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and MIR-133.

In some embodiments, the rAAV virion of the present disclosure comprises a heterologous nucleic acid comprising a nucleotide sequence that encodes one or more gene products selected from MYBPC3, DWORF, KCNH2, TRPM4, DSG2, and ATP2A2.

In some embodiments, the rAAV virion of the present disclosure comprises a heterologous nucleic acid comprising a nucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3 and ACTN2.

In some embodiments, the rAAV virion of the present disclosure comprises a heterologous nucleic acid comprising a nucleotide sequence that encodes one or more gene products selected from CACNA1C, DMD, DMPK, EPG5, EVC, EVC2, FBN1, NF1, SCN5A, SOS1, NPR1, ERBB4, VIP, MYH6, MYH7, and Cas9. In some embodiments, the rAAV virion of the present disclosure comprises a heterologous nucleic acid comprising a nucleotide sequence that encodes saCas9.

In some embodiments, the rAAV virion of the present disclosure comprises a heterologous nucleic acid comprising a nucleotide sequence that encodes one or more gene products selected from MYOCD, ASCL1, GATA4, MEF2C, TBX5, miR-133, and MESP1.

In some embodiments, the rAAV virion of the present disclosure comprises a heterologous nucleic acid comprising a nucleotide sequence that encodes one or more gene products selected from MMP11, SYNPO2L (e.g., SYNPO2LA or SYNPO2LA), and an inhibitory oligonucleotide targeting MTSS1.

In some embodiments, the transgene in the rAAV virion of the present disclosure encodes any of the above-identified gene products.

In some embodiments, the capsids described herein improve heart transduction efficiency, liver viral load, and/or heart-to-liver transduction ratio of the rAAV virions carrying any of the transgenes described herein (and encoding, and resulting in the expression of, any of the gene products described herein).

### Additional Embodiments of Capsids, Transgenes and Virions

Efforts to identify capsid variants with properties useful for gene therapy have included shuffling the DNA of AAV2 and AAV5 cap genes as described in U.S. Pat. No. 9,233,131; as well as directed evolution as described in Int'l Pat. Appl. Nos. WO2012/145601A2 and WO2018/222503A1. The disclosures of these documents are incorporated here for all purposes, and particularly for the methods of making and using AAV virions and for the polynucleotide sequences and gene products therein disclosed, as well as for the combinations of transcription factors useful in treating cardiac diseases or disorders.

The AAV capsid is encoded by the cap gene of AAV, which is also termed the right open-reading frame (ORF) (in contrast to the left ORF, rep). The structures of representative AAV capsids are described in various publications including Xie et al. (2002) Proc. Natl. Acad. Sci USA 99:10405-1040 (AAV2); Govindasamy et al. (2006) J. Virol. 80:11556-11570 (AAV4); Nam et a. (2007) J. Virol. 81:12260-12271 (AAV8) and Govindasamy et al. (2013) J. Virol. 87:11187-11199 (AAV5).

The AAV capsid contain 60 copies (in total) of three viral proteins (VPs), VP1, VP2, and VP3, in a predicted ratio of 1:1:10, arranged with T=1 icosahedral symmetry. The three VPs are translated from the same mRNA, with VP1 containing a unique N-terminal domain in addition to the entire VP2 sequence at its C-terminal region. VP2 contains an extra N-terminal sequence in addition to VP3 at its C terminus. In most crystal structures, only the C-terminal polypeptide sequence common to all the capsid proteins (˜530 amino acids) is observed. The N-terminal unique region of VP1, the VP1-VP2 overlapping region, and the first 14 to 16 N-terminal residues of VP3 are thought to be primarily disordered. Cryo-electron microscopy and image reconstruction data suggest that in intact AAV capsids, the N-terminal regions of the VP1 and VP2 proteins are located inside the capsid and are inaccessible for receptor and antibody binding. Thus, receptor attachment and transduction phenotypes are, generally, determined by the amino acid sequences within the common C-terminal domain of VP1, VP2 and VP3

In some embodiments, the one or more amino acid insertions, substitutions, or deletions is/are in the GH loop, or loop IV, of the AAV capsid protein, e.g., in a solvent-accessible portion of the GH loop, or loop IV, of the AAV capsid protein. For the GH loop/loop IV of AAV capsid, see, e.g., van Vliet et al. (2006) Mol. Ther. 14:809; Padron et al. (2005) Virol. 79:5047; and Shen et al. (2007) Mol. Ther. 15:1955. In some embodiments, a “parental” AAV capsid protein is a wild-type AAV9 capsid protein. In some embodiments, a “parental” AAV capsid protein is a wild-type AAV5 capsid protein. In some embodiments, a “parental” AAV capsid protein is a chimeric AAV capsid protein. Amino acid sequences of various AAV capsid proteins are known in the art. See, e.g., GenBank Accession No. NP_049542 for AAV1; GenBank Accession No. NP_044927 for AAV4; GenBank Accession No. AAD13756 for AAV5; GenBank Accession No. AAB95450 for AAV6; GenBank Accession No. YP_077178 for AAV7; GenBank Accession No. YP_077180 for AAV 8; GenBank Accession No. AAS99264 for AAV9 and GenBank Accession No. AAT46337 for AAV10. See, e.g., Santiago-Ortiz et al. (2015) Gene Ther. 22:934 for a predicted ancestral AAV capsid.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the AAV5 genome is provided in GenBank Accession No. AF085716. The life cycle and genetics of AAV are reviewed in Muzyczka, Current Topics in Microbiology and Immunology, 158:97-129 (1992). Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). Illustrative AAV vectors are provided in U.S. Pat. No. 7,105,345; U.S. Ser. No. 15/782,980; U.S. Pat. Nos. 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety for all purposes.

The rAAV virions of the disclosure comprise a heterologous nucleic acid comprising a nucleotide sequence encoding one or more gene product. The gene product(s) may be either a polypeptide or an RNA, or both. When the gene product is a polypeptide, the nucleotide sequence encodes a messenger RNA, optionally with one or more introns, which is translated into the gene product polypeptide. The nucleotide sequence may encode one, two, three, or more gene products (though the number is limited by the packaging capacity of the rAAV virion, typically about 5.2 kb). The gene products may be operatively linked to one promoter (for a single transcriptional unit) or more than one. Multiple gene products may also be produced using internal ribosome entry signal (IRES) or a self-cleaving peptide (e.g., a 2A peptide).

In some embodiments, the gene product is a polypeptide. In some embodiments, the polypeptide gene product is a polypeptide that induces reprogramming of a cardiac fibroblast, to generate an induced cardiomyocyte-like cell (iCM). In some embodiments, the polypeptide gene product is a polypeptide that enhances the function of a cardiac cell. In some embodiments, the polypeptide gene product is a polypeptide that provides a function that is missing or defective in the cardiac cell. In some embodiments, the polypeptide gene product is a genome-editing endonuclease.

In some embodiments, the gene product comprises a fusion protein that is fused to a heterologous polypeptide. In some embodiments, the gene product comprises a genome editing nuclease fused to an amino acid sequence that provides for subcellular localization, i.e., the fusion partner is a subcellular localization sequence (e.g., one or more nuclear localization signals (NLSs) for targeting to the nucleus, two or more NLSs, three or more NLSs, etc.).

In general, a viral vector is produced by introducing a viral DNA or RNA construct into a “producer cell” or “packaging cell” line. Packaging cell lines include but are not limited to any easily-transfectable cell line. Packaging cell lines can be based on HEK291, 293T cells, NIH3T3, COS, HeLa or Sf9 cell lines. Examples of packaging cell lines include but are not limited to: Sf9 (ATCC® CRL-1711™). Exemplary packing cell lines and methods for generating rAAV virions are provided by Int'l Pat. Pub. Nos. WO2017075627, WO2015/031686, WO2013/063379, WO2011/020710, WO2009/104964, WO2008/024998, WO2003/042361, and WO1995/013392; U.S. Pat. Nos. 9,441,206B2, 8,679,837, and 7,091,029B2.

In some embodiments, the gene product is a functional cardiac protein. In some embodiments, the gene product is a genome-editing endonuclease (optionally with a guide RNA, single-guide RNA, and/or repair template) that replaces or repairs a non-functional cardiac protein into a functional cardiac protein. Functional cardiac proteins include, but are not limited to cardiac troponin T; a cardiac sarcomeric protein; β-myosin heavy chain; myosin ventricular essential light chain 1; myosin ventricular regulatory light chain 2; cardiac a-actin; a-tropomyosin; cardiac troponin I; cardiac myosin binding protein C; four-and-a-half LIM protein 1; titin; 5′-AMP-activated protein kinase subunit gamma-2; troponin I type 3, myosin light chain 2, actin alpha cardiac muscle 1; cardiac LIM protein; caveolin 3 (CAV3); galactosidase alpha (GLA); lysosomal-associated membrane protein 2 (LAMP2); mitochondrial transfer RNA glycine (MTTG); mitochondrial transfer RNA isoleucine (MTTI); mitochondrial transfer RNA lysine (MTTK); mitochondrial transfer RNA glutamine (MTTQ); myosin light chain 3 (MYL3); troponin C (TNNC1); transthyretin (TTR); sarcoendoplasmic reticulum calcium-ATPase 2a (SERCA2a); stromal-derived factor-1 (SDF-1); adenylate cyclase-6 (AC6); beta-ARKct (β-adrenergic receptor kinase C terminus); fibroblast growth factor (FGF); platelet-derived growth factor (PDGF); vascular endothelial growth factor (VEGF); hepatocyte growth factor; hypoxia inducible growth factor; thymosin beta 4 (TMSB4X); nitric oxide synthase-3 (NOS3); unocartin 3 (UCN3); melusin; apoplipoprotein-E (ApoE); superoxide dismutase (SOD); and S100A1 (a small calcium binding protein; see, e.g., Ritterhoff and Most (2012) Gene Ther. 19:613; Kraus et al. (2009) Mol. Cell. Cardiol. 47:445).

In some embodiments, the gene product is a gene product whose expression complements a defect in a gene responsible for a genetic disorder. The disclosure provides rAAV virions comprising a polynucleotide encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOSIs (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, the rAAV virion comprises a polynucleotide encoding one or more of cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the gene products of the disclosure are polypeptide reprogramming factors. Reprogramming factors are desirable as means to convert one cell type into another. Non-cardiomyocytes cells can be differentiated into cardiomyocytes cells in vitro or in vivo using any method available to one of skill in the art. For example, see methods described in Ieda et al. (2010) Cell 142:375-386; Christoforou et al. (2013) PLoS ONE 8: e63577; Addis et al. (2013) J. Mol. Cell Cardiol. 60:97-106; Jayawardena et al. (2012) Circ. Res. 110:1465-1473; Nam Y et al. (2003) PNAS USA 110:5588-5593; Wada R et al. (2013) PNAS USA 110:12667-12672; and Fu J et al. (2013) Stem Cell Reports 1:235-247.

In cardiac context, the reprogramming factors may be capable of converting a cardiac fibroblast to a cardiac myocyte either directly or through an intermediate cell type. In particular, direct reprogramming is possible, or reprogramming by first converting the fibroblast to a pluripotent or totipotent stem cell. Such a pluripotent stem cell is termed an induced pluripotent stem (iPS) cell. An iPS cell that is subsequently converted to a cardiac myocyte (CM) cell is termed an iPS-CM cell. In the examples, iPS-CM derived in vitro from cardiac fibroblasts are used in vivo to select capsid proteins of interest. The disclosure also envisions using the capsid proteins disclosure to in turn generate iPS-CM cells in vitro but, particular, in vivo, as part of a therapeutic gene therapy regimen. Induced cardiomyocyte-like (iCM) cells refer to cells directly reprogrammed into cardiomyocytes.

Induced cardiomyocytes express one or more cardiomyocyte-specific markers, where cardiomyocyte-specific markers include, but are not limited to, cardiac troponin I, cardiac troponin-C, tropomyosin, caveolin-3, myosin heavy chain, myosin light chain-2a, myosin light chain-2v, ryanodine receptor, sarcomeric a-actinin, Nkx2.5, connexin 43, and atrial natriuretic factor. Induced cardiomyocytes can also exhibit sarcomeric structures. Induced cardiomyocytes exhibit increased expression of cardiomyocyte-specific genes ACTC1 (cardiac a-actin), ACTN2 (actinin a2), MYH6 (a-myosin heavy chain), RYR2 (ryanodine receptor 2), MYL2 (myosin regulatory light chain 2, ventricular isoform), MYL7 (myosin regulatory light chain, atrial isoform), TNNT2 (troponin T type 2, cardiac), and NPPA (natriuretic peptide precursor type A), PLN (phospholamban). Expression of fibroblasts markers such as Col1a2 (collagen 1a2) is downregulated in induced cardiomyocytes, compared to fibroblasts from which the iCM is derived.

Reprogramming methods involving polypeptide reprogramming factors (in some cases supplemented by small-molecule reprogramming factors supplied in conjunction with the rAAV) include those described in US2018/0112282A1, WO2018/005546, WO2017/173137, US2016/0186141, US2016/0251624, US2014/0301991, and US2013/0216503A1, which are incorporated in their entirety, particularly for the reprogramming methods and factors disclosed.

In some embodiments, cardiac cells are reprogrammed into induced cardiomyocyte-like (iCM) cells using one or more reprogramming factors that modulate the expression of one or more polynucleotides or proteins of interest, such as Achaete-scute homolog 1 (ASCL1), Myocardin (MYOCD), myocyte-specific enhancer factor 2C (MEF2C), and/or T-box transcription factor 5 (TBX5). In some embodiments, the one or more reprogramming factors are provided as a polynucleotide (e.g., an RNA, an mRNA, or a DNA polynucleotide) that encode one or more polynucleotides or proteins of interest. In some embodiments, the one or more reprogramming factors are provided as a protein.

In some embodiments, the reprogramming factors are microRNAs or microRNA antagonists, siRNAs, or small molecules that are capable of increasing the expression of one or more polynucleotides or proteins of interest. In some embodiments, expression of a polynucleotides or proteins of interest is increased by expression of a microRNA or a microRNA antagonist. For example, endogenous expression of an Oct polypeptide can be increased by introduction of microRNA-302 (miR-302), or by increased expression of miR-302. See, e.g., Hu et al., Stem Cells 31 (2): 259-68 (2013), which is incorporated herein by reference in its entirety. Hence, miRNA-302 can be an inducer of endogenous Oct polypeptide expression. The miRNA-302 can be introduced alone or with a nucleic acid that encodes the Oct polypeptide. In some embodiments, a suitable nucleic acid gene product is a microRNA. Suitable microRNAs include, e.g., mir-1, mir-133, mir-208, mir-143, mir-145, and mir-499.

In some embodiments, the methods of the disclosure comprise administering an rAAV virion of the disclosure before, during, or after administration of the small-molecule reprogramming factor. In some embodiments, the small-molecule reprogramming factor is a small molecule selected from the group consisting of SB431542, LDN-193189, dexamethasone, LY364947, D4476, myricetin, IWR1, XAV939, docosahexaenoic acid (DHA), S-Nitroso-TV-acetylpenicillamine (SNAP), Hh-Ag1.5, alprostadil, cromakalim, MNITMT, A769662, retinoic acid p-hydoxyanlide, decamethonium dibromide, nifedipine, piroxicam, bacitracin, aztreonam, harmalol hydrochloride, amide-C2 (A7), Ph-C12 (CIO), mCF3-C-7 (J5), G856-7272 (A473), 5475707, or any combination thereof.

In some embodiments, the gene products comprise reprogramming factors that modulate the expression of one or more proteins of interest selected from ASCL1, MYOCD, MEF2C, and TBX5. In some embodiments, the gene products comprise one or more reprogramming factors selected from ASCL1, MYOCD, MEF2C, AND TBX5, CCNB1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and miR-133.

In some embodiments, the gene products comprise GATA4, MEF2C, and TBX5 (i.e., GMT). In some embodiments, the gene products comprise MYOCD, MEF2C, and TBX5 (i.e., MyMT). In some embodiments, the gene products comprise MYOCD, ASCL1, MEF2C, and TBX5 (i.e., MyAMT). In some embodiments, the gene products comprise MYOCD and ASCL1 (i.e., MyA). In some embodiments, the gene products comprise GATA4, MEF2C, TBX5, and MYOCD (i.e., 4F). In other embodiments, the gene products comprise GATA4, MEF2C, TBX5, ESSRG, MYOCD, ZFPM2, and MESP1 (i.e., 7F). In some embodiments, the gene products comprise one or more of ASCL1, MEF2C, GATA4, TBX5, MYOCD, ESRRG, AND MESPL.

In some embodiments, the rAAV virions generate cardiac myocytes in vitro or in vivo. Cardiomyocytes or cardiac myocytes are the muscle cells that make up the cardiac muscle. Each myocardial cell contains myofibrils, which are long chains of sarcomeres, the contractile units of muscle cells. Cardiomyocytes show striations similar to those on skeletal muscle cells, but unlike multinucleated skeletal cells, they contain only one nucleus. Cardiomyocytes have a high mitochondrial density, which allows them to produce ATP quickly, making them highly resistant to fatigue. Mature cardiomyocytes can express one or more of the following cardiac markers: α-Actinin, MLC2v, MY20, cMHC, NKX2-5, GATA4, cTNT, cTNI, MEF2C, MLC2a, or any combination thereof. In some embodiments, the mature cardiomyocytes express NKX2-5, MEF2C or a combination thereof. In some embodiments, cardiac progenitor cells express early stage cardiac progenitor markers such as GATA4, ISL1 or a combination thereof.

In some embodiments, the gene product is a polynucleotide. In some embodiments, as described below, the gene product is a guide RNA capable of binding to an RNA-guided endonuclease. In some embodiments, the gene product is an inhibitory nucleic acid capable of reducing the level of an mRNA and/or a polypeptide gene product, e.g., in a cardiac cell. For example, in some embodiments, the polynucleotide gene product is an interfering RNA capable of selectively inactivating a transcript encoded by an allele that causes a cardiac disease or disorder. As an example, the allele is a myosin heavy chain 7, cardiac muscle, beta (MYH7) allele that comprises a hypertrophic cardiomyopathy-causing mutation. Other examples include, e.g., interfering RNAs that selectively inactivate a transcript encoded by an allele that causes hypertrophic cardiomyopathy (HCM), dilated cardiomyopathy (DCM) or Left Ventricular Non-Compaction (LVNC), where the allele is a MYL3 (myosin light chain 3, alkali, ventricular, skeletal slow), MYH7, TNNI3 (troponin I type 3 (cardiac)), TNNT2 (troponin T type 2 (cardiac)), TPM1 (tropomyosin 1 (alpha)) or ACTC1 allele comprising an HCM-causing, a DCM-causing or a LVNC-causing mutation. See, e.g., U.S. Pat. Pub. No. 2016/0237430 for examples of cardiac disease-causing mutations.

In some embodiments, the gene product is a polypeptide-encoding RNA. In some embodiments, the gene product is an interfering RNA. In some embodiments, the gene product is an aptamer. In some embodiments, the gene product is a polypeptide. In some embodiments, the gene product is a therapeutic polypeptide, e.g., a polypeptide that provides clinical benefit. In some embodiments, the gene product is a site-specific nuclease that provide for site-specific knock-down of gene function. In some embodiments, the gene product is an RNA-guided endonuclease that provides for modification of a target nucleic acid. In some embodiments, the gene products are: i) an RNA-guided endonuclease that provides for modification of a target nucleic acid; and ii) a guide RNA that comprises a first segment that binds to a target sequence in a target nucleic acid and a second segment that binds to the RNA-guided endonuclease. In some embodiments, the gene products are: i) an RNA-guided endonuclease that provides for modification of a target nucleic acid; ii) a first guide RNA that comprises a first segment that binds to a first target sequence in a target nucleic acid and a second segment that binds to the RNA-guided endonuclease; and iii) a first guide RNA that comprises a first segment that binds to a second target sequence in the target nucleic acid and a second segment that binds to the RNA-guided endonuclease.

A nucleotide sequence encoding a heterologous gene product in an rAAV virion of the present disclosure can be operably linked to a promoter. For example, a nucleotide sequence encoding a heterologous gene product in an rAAV virion of the present disclosure can be operably linked to a constitutive promoter, a regulatable promoter, or a cardiac cell-specific promoter. Suitable constitutive promoters include a human elongation factor 1 α subunit (EF1α) promoter, a β-actin promoter, an α-actin promoter, a β-glucuronidase promoter, CAG promoter, super core promoter, and a ubiquitin promoter. In some embodiments, a nucleotide sequence encoding a heterologous gene product in an rAAV virion of the present disclosure is operably linked to a cardiac-specific transcriptional regulator element (TRE), where cardiac-specific TREs include promoters and enhancers. Suitable cardiac-specific TREs include, but are not limited to, TREs derived from the following genes: myosin light chain-2 (MLC-2), a-myosin heavy chain (a-MHC), desmin, AE3, cardiac troponin C (cTnC), and cardiac actin. Franz et al. (1997) Cardiovasc. Res. 35:560-566; Robbins et al. (1995) Ann. NY. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13. In some embodiments, the promoter is an α-MHC promoter, an MLC-2 promoter, or cTnT promoter.

The polynucleotide encoding a gene product is operably linked to a promoter and/or enhancer to facilitate expression of the gene product. Depending on the host/vector system utilized, any of a number of suitable transcription and translation control elements, including constitutive and inducible promoters, transcription enhancer elements, transcription terminators, etc. may be used in the rAAV virion (e.g., Bitter et al. (1987) Methods in Enzymology, 153:516-544).

Separate promoters and/or enhancers can be employed for each of the polynucleotides. In some embodiments, the same promoter and/or enhance is used for two or more polynucleotides in a single open reading frame. Vectors employing this configuration of genetic elements are termed “polycistronic.” An illustrative example of a polycistronic vector comprises an enhancer and a promoter operatively linked to a single open-reading frame comprising two or more polynucleotides linked by 2A region(s), whereby expression of the open-reading frame result in multiple polypeptides being generated co-translationally. The 2A region is believed to mediate generation of multiple polypeptide sequences through codon skipping; however, the present disclosure relates also to polycistronic vectors that employ post-translational cleavage to generate two or more proteins of interest from the same polynucleotide. Illustrative 2A sequences, vectors, and associated methods are provided in US20040265955A1, which is incorporated herein by reference.

Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring cardiac specific expression will be used. Non-limiting examples of suitable cardiac specific promoters include desmin (Des), alpha-myosin heavy chain (a-MHC), myosin light chain 2 (MLC-2), cardiac troponin T (cTnT) and cardiac troponin C (cTnC). Non-limiting examples of suitable neuron specific promoters include synapsin I (SYN), calcium/calmodulin-dependent protein kinase II, tubulin alpha I, neuron-specific enolase and platelet-derived growth factor beta chain promoters and hybrid promoters by fusing cytomegalovirus enhancer (E) to those neuron-specific promoters.

Examples of suitable promoters for driving expression reprogramming factors include, but are not limited to, retroviral long terminal repeat (LTR) elements; constitutive promoters such as CMV, HSV1-TK, SV40, EF-1a, β-actin, phosphoglycerol kinase (PGK); inducible promoters, such as those containing Tet-operator elements; cardiac specific promoters, such as desmin (DES), alpha-myosin heavy chain (a-MHC), myosin light chain 2 (MLC-2), cardiac troponin T (cTnT) and cardiac troponin C (cTnC); neural specific promoters, such as nestin, neuronal nuclei (NeuN), microtubule-associate protein 2 (MAP2), beta III tubulin, neuron specific enolase (NSE), oligodendrocyte lineage (Olig1/2), and glial fibrillary acidic protein (GFAP); and pancreatic specific promoters, such as Pax4, Nkx2.2, Ngn3, insulin, glucagon, and somatostatin.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, a-myosin heavy chain, AE3, cardiac troponin C, and cardiac actin. Franz et al. (1997) Cardiovasc. Res. 35:560-566; Robbins et al. (1995) Ann. N. Y. Acad. Sci. 752:492-505; Linn et al. (1995) Circ. Res. 76:584-591; Parmacek et al. (1994) Cell. Biol. 14:1870-1885; Hunter et al. (1993) Hypertension 22:608-617; and Sartorelli et al. (1992) PNAS USA 89:4047-4051.

The promoter can be one naturally associated with a gene or nucleic acid segment. Similarly, for RNAs (e.g., microRNAs), the promoter can be one naturally associated with a microRNA gene (e.g., an miRNA-302 gene). Such a naturally associated promoter can be referred to as the “natural promoter” and may be obtained by isolating the 5′ non-coding sequences located upstream of the coding segment and/or exon. Similarly, an enhancer may be one naturally associated with a nucleic acid sequence. However, the enhancer can be located either downstream or upstream of that sequence.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers can include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

The promoters employed may be constitutive, inducible, developmentally-specific, tissue-specific, and/or useful under the appropriate conditions to direct high level expression of the nucleic acid segment. For example, the promoter can be a constitutive promoter such as, a CMV promoter, a CMV cytomegalovirus immediate early promoter, a CAG promoter, an EF-1α promoter, a HSV1-TK promoter, an SV40 promoter, a β-actin promoter, a PGK promoter, or a combination thereof. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. In certain embodiments, cell type-specific promoters are used to drive expression of reprogramming factors in specific cell types. Examples of suitable cell type-specific promoters useful for the methods described herein include, but are not limited to, the synthetic macrophage-specific promoter described in He et al (2006), Human Gene Therapy 17:949-959; the granulocyte and macrophage-specific lysozyme M promoter (see, e.g., Faust et al (2000), Blood 96 (2): 719-726); and the myeloid-specific CD11b promoter (see, e.g., Dziennis et al (1995), Blood 85 (2): 319-329). Other examples of promoters that can be employed include a human EF1α elongation factor promoter, a CMV cytomegalovirus immediate early promoter, a CAG chicken albumin promoter, a viral promoter associated with any of the viral vectors described herein, or a promoter that is homologous to any of the promoters described herein (e.g., from another species). Examples of prokaryotic promoters that can be used include, but are not limited to, SP6, T7, T5, tac, bla, trp, gal, lac, or maltose promoters.

In some embodiments, an internal ribosome entry sites (IRES) element can be used to create multigene, or polycistronic, messages. IRES elements are able to bypass the ribosome scanning model of 5′-methylated Cap dependent translation and begin translation at internal sites (Pelletier and Sonenberg, Nature 334 (6180): 320-325 (1988)). IRES elements from two members of the picornavirus family (polio and encephalomyocarditis) have been described (Pelletier and Sonenberg, Nature 334 (6180): 320-325 (1988)), as well an IRES from a mammalian message (Macejak & Samow, Nature 353:90-94 (1991)). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

In some embodiments, a nucleotide sequence is operably linked to a polyadenylation sequence. Suitable polyadenylation sequences include bovine growth hormone polyA signal (bGHpolyA) and short poly A signal. Optionally the rAAV vectors of the disclosure comprise the Woodchuck Post-transcriptional Regulatory Element (WPRE). In some embodiments, the polynucleotide encoding gene products are join by sequences include so-called self-cleaving peptide, e.g. P2A peptides.

In some embodiments, the gene product comprises a site-specific endonuclease that provides for site-specific knock-down of gene function, e.g., where the endonuclease knocks out an allele associated with a cardiac disease or disorder. For example, where a dominant allele encodes a defective copy of a gene that, when wild-type, is a cardiac structural protein and/or provides for normal cardiac function, a site-specific endonuclease can be targeted to the defective allele and knock out the defective allele. In some embodiments, a site-specific endonuclease is an RNA-guided endonuclease.

In addition to knocking out a defective allele, a site-specific nuclease can also be used to stimulate homologous recombination with a donor DNA that encodes a functional copy of the protein encoded by the defective allele. For example, a subject rAAV virion can be used to deliver both a site-specific endonuclease that knocks out a defective allele a functional copy of the defective allele (or fragment thereof), resulting in repair of the defective allele, thereby providing for production of a functional cardiac protein (e.g., functional troponin, etc.). In some embodiments, a subject rAAV virion comprises a heterologous nucleotide sequence that encodes a site-specific endonuclease and a heterologous nucleotide sequence that encodes a functional copy of a defective allele, where the functional copy encodes a functional cardiac protein. Functional cardiac proteins include, e.g., troponin, a chloride ion channel, and the like.

Site-specific endonucleases that are suitable for use include, e.g., zinc finger nucleases (ZFNs); meganucleases; and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Pat. Pub. No. 2011/0301073. Suitable site-specific endonucleases include engineered meganuclease re-engineered homing endonucleases. Suitable endonucleases include an I-Tev1 nuclease. Suitable meganucleases include I-Sce1 (see, e.g., Bellaiche et al. (1999) Genetics 152:1037); and I-Cre1 (see, e.g., Heath et al. (1997) Nature Sructural Biology 4:468). Site-specific endonucleases that are suitable for use include CRISPRi systems and the Cas9-based SAM system.

In some embodiments, the gene product is an RNA-guided endonuclease. In some embodiments, the gene product comprises an RNA comprising a nucleotide sequence encoding an RNA-guided endonuclease. In some embodiments, the gene product is a guide RNA, e.g., a single-guide RNA. In some embodiments, the gene products are: 1) a guide RNA; and 2) an RNA-guided endonuclease. The guide RNA can comprise: a) a protein-binding region that binds to the RNA-guided endonuclease; and b) a region that binds to a target nucleic acid. An RNA-guided endonuclease is also referred to herein as a “genome editing nuclease.”

Examples of suitable genome editing nucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some embodiments, the gene product comprises a class 2 CRISPR/Cas endonuclease. In some embodiments, the gene product comprises a class 2 type II CRISPR/Cas endonuclease (e.g., a Cas9 protein such as saCas9). In some embodiments, the gene product comprises a class 2 type V CRISPR/Cas endonuclease (e.g., a Cpf1 protein, a C2c1 protein, or a C2c3 protein). In some embodiments, the gene product comprises a class 2 type VI CRISPR/Cas endonuclease (e.g., a C2c2 protein; also referred to as a “Cas13a” protein). In some embodiments, the gene product comprises a CasX protein. In some embodiments, the gene product comprises a CasY protein.

Nucleic Acids, Vectors, Cells and Generation of rAAV Virions

In some embodiments, the disclosure provides nucleic acids encoding any AAV capsid protein described herein (such as AAV capsid proteins comprising one or more of the modifications described herein).

The polynucleotide encoding the capsid protein can comprise a sequence comprising either the native codons of the wild-type cap gene, or alternative codons selected to encode the same protein. The codon usage of the insertion can be varied. It is within the skill of those in the art to select appropriate nucleotide sequences and to derive alternative nucleotide sequences to encode any capsid protein of the disclosure. Reverse translation of the protein sequence can be performed using the codon usage table of the host organism, i.e. Eukaryotic codon usage for humans.

In some embodiments, the disclosure provides a polynucleotide encoding an AAV9 derived capsid protein comprising a sequence at least 80%, 85%, 90%, 95%, 99%, or 100% identical to any one of SEQ ID NOs: 402-410 and 464-468.

In some embodiments, the disclosure provides a polynucleotide encoding an AAV5/AAV9 chimeric capsid protein comprising a sequence at least 80%, 85%, 90%, 95%, 99%, or 100% identical to any one of SEQ ID NOs: 421-444.

In some embodiments, the disclosure provides a polynucleotide encoding an combinatory capsid protein comprising a sequence at least 80%, 85%, 90%, 95%, 99%, or 100% identical to any one of SEQ ID NO: 445-462.

In some embodiments, the disclosure provides a polynucleotide encoding an AAV9 derived capsid protein comprising a sequence having at least or more than 75%, 80%, 85%, 90%, 95%, 99%, or 100% sequence identity to any one selected from the group consisting of: SEQ ID NOs: 488-589, 705-710, and 767-780.

In some embodiments, the disclosure provides a polynucleotide encoding an AAV9 derived capsid protein comprising a sequence having at least or more than 75%, 80%, 85%, 90%, 95%, 99%, or 100% sequence identity to any one selected from the group consisting of: SEQ ID NOs: 512, 589, 772, 774, 705, 513, 710, 488, 707, and 539.

In some embodiments, the disclosure provides a polynucleotide encoding an AAV9 derived capsid protein comprising a sequence having at least or more than 80%, 85%, 90%, 95%, 99%, or 100% sequence identity to any one of SEQ ID NOs: 705-708.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 515, 581, 539 and 527.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 707, 512, 539 and 589.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 707, 512, 539 and 589. In some embodiments, the disclosure provides a polynucleotide encoding an AAV9 derived capsid protein comprising a sequence having at least 80%, 85%, 90%, 95%, 99%, or 100% sequence identity to SEQ ID NO: 707. In some embodiments, the disclosure provides a polynucleotide encoding an AAV9 derived capsid protein comprising a sequence having at least 80%, 85%, 90%, 95%, 99%, or 100% sequence identity to SEQ ID NO: 512. In some embodiments, the disclosure provides a polynucleotide encoding an AAV9 derived capsid protein comprising a sequence having at least 80%, 85%, 90%, 95%, 99%, or 100% sequence identity to SEQ ID NO: 539. In some embodiments, the disclosure provides a polynucleotide encoding an AAV9 derived capsid protein comprising a sequence having at least 80%, 85%, 90%, 95%, 99%, or 100% sequence identity to SEQ ID NO: 589.

In some embodiments, the disclosure provides a polynucleotide encoding an AAV9 derived capsid protein comprising a sequence at least 80%, 85%, 90%, 95%, 97%, 98%, 99%, or 100% identical to any one of SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, and 710.

In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 488. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 499. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 504. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 505. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 506. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 510. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 512. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 513. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 516. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 518. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 521. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 522. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 533. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 536. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 539. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 558. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 562. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 566. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 571. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 576. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 578. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 579. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 580. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 581. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 585. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 588. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 589. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 705. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 706. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 707. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 708. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 710.

In some embodiments, the disclosure provides a vector or a plasmid comprising a nucleic acid encoding any AAV capsid protein described herein. In some embodiments, the vector or plasmid further comprises a promoter operably linked to the nucleic acid encoding the AAV capsid proteins. In some embodiments, the promoter is any promoter active in a cell to be used for expressing the capsid protein (e.g., a producer or host cell). In some embodiments, the promoter is P40 promoter. In some embodiments, the promoter is a polyhedrin promoter.

In some embodiments, the vector or plasmid comprising a nucleic acid encoding any AAV capsid protein described herein further comprises a nucleic acid encoding a replication (Rep) protein. In some embodiments, the Rep protein is a Rep protein from the same serotype of AAV as the inverted terminal repeats (ITRs) used to flank the transgene (to be packaged into virions using any of the AAV capsid proteins described herein). In some embodiments, the Rep protein is an AAV2 Rep protein. In some embodiments, the Rep protein is an AAV8 Rep protein. In some embodiments, the vector or plasmid comprising a nucleic acid encoding any AAV capsid protein described herein does not further comprise a nucleic acid encoding a Rep protein.

In some embodiments, the disclosure provides a cell comprising a nucleic acid encoding any AAV capsid protein described herein. In some embodiments, the disclosure provides a cell comprising a vector or a plasmid comprising a nucleic acid encoding any AAV capsid protein described herein. In some embodiments, the cell further comprises a vector or plasmid comprising a nucleic acid encoding a Rep protein, wherein the Rep protein may be expressed by the same or different vector or plasmid as the AAV capsid protein described herein.

In some embodiments, the disclosure provides a host cell comprising a nucleic acid encoding any AAV capsid protein described herein. In some embodiments, the disclosure provides a host cell comprising a vector or a plasmid comprising a nucleic acid encoding any AAV capsid protein described herein.

In some embodiments, a host cell comprising a nucleic acid encoding any AAV capsid protein described herein is for producing an rAAV virion described herein (such as an rAAV virion comprising a modified AAV capsid protein as described herein). In some embodiments, the nucleic acid encoding any AAV capsid protein is transiently transfected into a cell. In some embodiments, the nucleic acid encoding any AAV capsid protein is stably inserted into the cell genome.

In some embodiments, the host cell is a mammalian cell. In some embodiments, the host cell is selected from the group consisting of: are HEK293, HEK293T, HeLa, Vero, MDCK, MRC-5, PER.C6, BHK21 and CHO. In some embodiments, the host cell is HEK293 cell.

In some embodiments, the host cell is an insect cell. In some embodiments, the host cell is Sf9 insect cell. In some embodiments where the insect cells are used as host cells, the vectors or plasmids described herein are first introduced into a recombinant baculovirus and then carried into insect cells by baculovirus infection.

In some embodiments, the host cells are further transfected with one or more vectors or plasmids comprising helper functions and/or viral structural proteins necessary for replication and/or encapsidation of the vector(s) carrying the transgene.

In some embodiments, the host cells are further transfected with a viral vector carrying a transgene (such as any transgene described herein). In some embodiments, the transgene is flanked by inverted terminal repeats (ITRs). In some embodiments, the ITRs are of the same serotype as the Rep protein expressed in the host cells. In some embodiments, the ITRs are AAV2 ITRs. In some embodiments, the ITRs are AAV8 ITRs. Any combinations of Rep proteins and ITRs known in the art can be used in the cells and methods described herein.

In some embodiments, a host cell (e.g., a mammalian or an insect cell) further comprises a helper plasmid expression Adenovirus helper genes.

In some embodiments, a host cell comprises one or more packaging factors stably integrated into cell genome. In some embodiments, the host cell comprises a nucleic acid encoding any of the AAV capsid proteins described herein stably integrated into its genome. In some embodiments, the host cell comprises a nucleic acid encoding a Rep protein stably integrated into its genome. In some embodiments, the host cell comprises an Adenovirus helper gene stably integrated into its genome. In some embodiments, the host cell comprises a nucleic acid encoding an AAV capsid protein described herein, a nucleic acid encoding a Rep protein, and an Adenovirus helper gene(s) stably integrated into its genome.

The methods of production of rAAV virions are known in the art. In some embodiments, an rAAV virion can be generated using the host cells as described herein.

In some embodiments, the method of producing an rAAV virion in cell comprises:
i. introducing (e.g., by transient transfection or stable integration techniques) a nucleic acid encoding any of the AAV capsid proteins described herein, a nucleic acid encoding a Rep protein (such as any AAV Rep protein known in the art or described herein), an Adenovirus helper gene(s) (such as any Adenovirus helper genes known in the art), and/or a transgene cassette comprising a transgene flanked by ITRs (e.g., wherein the transgene expresses a therapeutic protein) into the cell (e.g., via DNA transfection, viral infection, and/or stable integration), wherein each of the introduced nucleic acids or genes is operably linked to a promoter active in the cell;
ii culturing the cell (e.g., using a suspension cell culture or an adherent cell culture) under conditions suitable for production of an rAAV virion (e.g., suitable for packaging protein expression and/or suitable for viral packaging), and
iii. collecting the produced rAAV virion (e.g, from media supernatant and/or from cell lysate following cell lysis), and
iv. optionally further purifying the rAAV virion, e.g., by density gradient ultracentrifugation and/or chromatography-based methods.

In some embodiments, the vectors, promoters, packaging factors, packaging systems, host cells, and/or methods of rAAV virion production are any of those known in the art.

### Methods of Use

In some embodiments, the disclosure provides methods of identifying AAV capsid proteins that confer on rAAV virions increased transduction efficiency in target cells. The methods comprise providing a population of rAAV virions whose rAAV genomes comprise a library of cap polynucleotides encoding variant AAV capsid proteins; optionally contacting the population with non-target cells for a time sufficient to permit attachment of undesired rAAV virions to the non-target cells; contacting the population with target cells for a time sufficient to permit transduction of the cap polynucleotide into the target cells by the rAAV virions; and sequencing the cap polynucleotides from the target cells, thereby identifying AAV capsid proteins that confer increased transduction efficiency in the target cells. In some embodiments, the method further comprises depleting the population of rAAV virions by contacting the population with non-target cells for time sufficient to permit attachment of the rAAV virions to the non-target cells. Non-limiting examples of such identifications methods are provided in the Examples.

The disclosure provides methods for generating cardiomyocytes and/or cardiomyocyte-like cells in vitro using an rAAV virion. Selected starting cells are transduced with an rAAV and optionally exposed to small-molecule reprogramming factors (before, during, or after transduction) for a time and under conditions sufficient to convert the starting cells across lineage and/or differentiation boundaries to form cardiac progenitor cells and/or cardiomyocytes. In some embodiments, the starting cells are fibroblast cells. In some embodiments, the starting cells express one or more markers indicative of a differentiated phenotype. The time for conversion of starting cells into cardiac progenitor and cardiomyocyte cells can vary. For example, the starting cells can be incubated after treatment with one or more polynucleotides or proteins of interest until cardiac or cardiomyocyte cell markers are expressed. Such cardiac or cardiomyocyte cell markers can include any of the following markers: α-GATA4, TNNT2, MYH6, RYR2, NKX2-5, MEF2C, ANP, Actinin, MLC2v, MY20, CMHC, ISL1, cTNT, cTNI, and MLC2a, or any combination thereof. In some embodiments, the induced cardiomycocyte cells are negative for one or more neuronal cells markers. Such neuronal cell markers can include any of the following markers: DCX, TUBB3, MAP2, and ENO2.

Incubation can proceed until cardiac progenitor markers are expressed by the starting cells. Such cardiac progenitor markers include GATA4, TNNT2, MYH6, RYR2, or a combination thereof. The cardiac progenitor markers such as GATA4, TNNT2, MYH6, RYR2, or a combination thereof can be expressed by about 8 days, or by about 9 days, or by about 10 days, or by about 11 days, or by about 12 days, or by about 14 days, or by about 15 days, or by about 16 days, or by about 17 days, or by about 18 days, or by about 19 days, or by about 20 days after starting incubation of cells in the compositions described herein. Further incubation of the cells can be performed until expression of late stage cardiac progenitor markers such as NKX2-5, MEF2C or a combination thereof occurs.

Reprogramming efficiency may be measured as a function of cardiomyocyte markers. Such pluripotency markers include, but are not limited to, the expression of cardiomyocyte marker proteins and mRNA, cardiomyocyte morphology and electrophysiological phenotype. Non-limiting examples of cardiomyocyte markers include, a-sarcoglycan, atrial natriuretic peptide (ANP), bone morphogenetic protein 4 (BMP4), connexin 37, connexin 40, crypto, desmin, GATA4, GATA6, MEF2C, MYH6, myosin heavy chain, NKX2.5, TBX5, and Troponin T.

The expression of various markers specific to cardiomyocytes may be detected by conventional biochemical or immunochemical methods (e.g., enzyme-linked immunosorbent assay, immunohistochemical assay, and the like). Alternatively, expression of a nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public databases such as GenBank. Thus, marker-specific sequences needed for use as primers or probes are easily determined.

Cardiomyocytes exhibit some cardiac-specific electrophysiological properties. One electrical characteristic is an action potential, which is a short-lasting event in which the difference of potential between the interior and the exterior of each cardiac cell rises and falls following a consistent trajectory. Another electrophysiological characteristic of cardiomyocytes is the cyclic variations in the cytosolic-free Ca²⁺ concentration, named as Ca²⁺ transients, which are employed in the regulation of the contraction and relaxation of cardiomyocytes. These characteristics can be detected and evaluated to assess whether a population of cells has been reprogrammed into cardiomyocytes.

The present disclosure provides a method of delivering a gene product to a cardiac cell, e.g., a cardiac fibroblast. The methods generally involve infecting a cardiac cell (e.g., a cardiac fibroblast) with an rAAV virion, where the gene product(s) encoded by the heterologous nucleic acid present in the rAAV virion is/are produced in the cardiac cell (e.g., cardiac fibroblast). Delivery of gene product(s) to a cardiac cell (e.g., cardiac fibroblast) can provide for treatment of a cardiac disease or disorder. Delivery of gene product(s) to a cardiac cell (e.g., cardiac fibroblast) can provide for generation of an induced cardiomyocyte-like (iCM) cell from the cardiac fibroblast. Delivery of gene product(s) to a cardiac cell (e.g., cardiac fibroblast) can provide for editing of the genome of the cardiac cell (e.g., cardiac fibroblast).

In some embodiments, infecting or transducing a cardiac cell (e.g., cardiac fibroblast) is carried out in vitro. In some embodiments, infecting or transducing a cardiac cell (e.g., cardiac fibroblast) is carried out in vitro; and the infected/transduced cardiac cell (e.g., cardiac fibroblast) is introduced into (e.g., transfused into or implanted into) an individual in need thereof, e.g., directly into cardiac tissue of an individual in need thereof. For in vitro transduction, an effective amount of rAAV virions to be delivered to cells is from about 10⁵ to about 10¹³ of the rAAV virions. Other effective dosages can be readily established by one of ordinary skill in the art through routine trials establishing dose response curves.

In some embodiments, infecting a cardiac cell (e.g., cardiac fibroblast) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in vivo injection, i.e., injection directly into cardiac tissue, a therapeutically effective dose will be on the order of from about 10⁶ to about 10¹⁵ of the rAAV virions, e.g., from about 10⁵ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁴ to about 10⁵, from about 10⁵ to about 10⁶, from about 10⁶ to about 10⁷, from about 10⁶ to about 10⁷, from about 10⁷ to about 10⁸, from about 10⁸ to about 10⁹, from about 10⁹ to about 10¹⁰, from about 10¹⁰ to about 10¹¹ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual, or are administered via another route. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, the rAAV viron is administered at, at least at, or at no more than, 10² vg/kg, 10³ vg/kg, 10⁴ vg/kg, 10⁵ vg/kg, 10⁶ vg/kg, 10⁸ vg/kg, 10⁹ vg/kg, 10¹⁰ vg/kg, 10¹¹ vg/kg, 10¹² vg/kg, 10¹³ vg/kg, 2×10¹³ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹³ vg/kg, 10¹⁴ vg/kg, 2×10¹⁴ vg/kg, 3×10¹⁴ vg/kg, 4×10¹⁴ vg/kg, 5×10¹⁴ vg/kg, 6×10¹⁴ vg/kg, 7×10¹⁴ vg/kg, 8×10¹⁴ vg/kg, 9×10¹⁴ vg/kg, 10¹⁵ vg/kg, 10¹⁶ vg/kg, 10¹⁷ vg/kg, or 10¹⁸ vg/kg (or at any range of amounts in between these values). In some embodiments, the rAAV virion is administered at 2×10¹³ vg/kg. In some embodiments, the rAAV virion is administered at 1.43×10¹³ vg/kg. In some embodiments, the rAAV virion is administered at 1.2×10¹⁴ vg/kg.

In some embodiments, an effective amount of an rAAV virion of the present disclosure is administered locally to the heart. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery, e.g., intravenously. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, more than one administration (e.g., two, three, four or more administrations) may be employed to achieve the desired level of gene expression. In some embodiments, the more than one administration is administered at various intervals, e.g., daily, weekly, twice monthly, monthly, every 3 months, every 6 months, yearly, etc. In some embodiments, multiple administrations are administered over a period of time of from 1 month to 2 months, from 2 months to 4 months, from 4 months to 8 months, from 8 months to 12 months, from 1 year to 2 years, from 2 years to 5 years, or more than 5 years.

The present disclosure provides a method of reprogramming a cardiac fibroblast to generate an induced cardiomyocyte-like cell (iCM). The method generally involves infecting a cardiac fibroblast with an rAAV virion of the present disclosure, wherein the rAAV virion comprises a heterologous nucleic acid comprising a nucleotide sequence encoding one or more reprogramming factors.

The expression of various markers specific to cardiomyocytes is detected by conventional biochemical or immunochemical methods (e.g., enzyme-linked immunosorbent assay; immunohistochemical assay; and the like). Alternatively, expression of nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public data bases such as GenBank; thus, marker-specific sequences needed for use as primers or probes is easily determined.

Induced cardiomyocytes can also exhibit spontaneous contraction. Whether an induced cardiomyocyte exhibits spontaneous contraction can be determined using standard electrophysiological methods (e.g., patch clamp).

In some embodiments, induced cardiomyocytes can exhibit spontaneous Ca²⁺ oscillations. Ca²⁺ oscillations can be detected using standard methods, e.g., using any of a variety of calcium-sensitive dyes, intracellular Ca²⁺ ion-detecting dyes include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, rhod-3, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes, and others (see, e.g., the catalog or Internet site for Molecular Probes, Eugene, see, also, Nuccitelli, ed., Methods in Cell Biology, Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed, Academic Press (1999); Calcium Signaling Protocols (Methods in Molecular Biology), 2005, D. G. Lamber, ed., Humana Press.).

In some embodiments, an iCM is generated in vitro; and the iCM is introduced into an individual, e.g., the iCM is implanted into a cardiac tissue of an individual in need thereof. A method of the present disclosure can comprise infecting a population of cardiac fibroblasts in vitro, to generate a population of iCMs; and the population of iCMs is implanted into a cardiac tissue of an individual in need thereof.

In some embodiments, an iCM is generated in vivo. For example, in some embodiments, an rAAV virion of the present disclosure that comprises a heterologous nucleic acid comprising a nucleotide sequence encoding one or more reprogramming factors is administered to an individual. In some embodiments, the rAAV virion is administered directly into cardiac tissue of an individual in need thereof. In some embodiments, from about 10⁶ to about 10⁵, from about 10⁵ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure that comprises a heterologous nucleic acid comprising a nucleotide sequence encoding one or more reprogramming factors are administered to an individual, e.g., are administered directly into cardiac tissue in the individual or via another route of administration. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁴ vg/kg, from about 10¹⁴ vg/kg to about 10¹⁶ vg/kg, or more than 10¹⁶ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

The present disclosure provides a method of modifying (“editing”) the genome of a cardiac cell. The present disclosure provides a method of modifying (“editing”) the genome of a cardiac fibroblast. The present disclosure provides a method of modifying (“editing”) the genome of a cardiomyocyte. The methods generally involve infecting a cardiac cell (e.g., a cardiac fibroblast or a cardiomyocyte) with an rAAV virion of the present disclosure, wherein the rAAV virion comprises a heterologous nucleic acid comprising a nucleotide sequence encoding a genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding an RNA-guided genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; and ii) one or more guide RNAs. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; ii) a guide RNAs; and iii) a donor template DNA. Suitable RNA-guided genome-editing endonucleases are described above.

In some embodiments, infecting a cardiac cell (e.g., cardiac fibroblast; a cardiomyocyte) is carried out in vitro. In some embodiments, infecting a cardiac cell (e.g., cardiac fibroblast; a cardiomyocyte) is carried out in vitro; and the infected cardiac cell (e.g., cardiac fibroblast) is introduced into (e.g., implanted into) an individual in need thereof, e.g., directly into cardiac tissue of an individual in need thereof. For in vitro transduction, an effective amount of rAAV virions to be delivered to cells will be on the order of from about 10^{s} to about 10¹³ of the rAAV virions. Other effective dosages can be readily established by one of ordinary skill in the art through routine trials establishing dose response curves.

In some embodiments, infecting a cardiac cell (e.g., cardiac fibroblast; a cardiomyocyte) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in vivo injection, i.e., injection directly into cardiac tissue, a therapeutically effective dose will be on the order of from about 10⁶ to about 10¹⁵ of the rAAV virions, e.g., from about 10¹¹ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁶ to about 10⁷, from about 10⁷ to about 10⁸, from about 10⁸ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, the genome editing comprises homology-directed repair (HDR). In some embodiments, the HDR corrects a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

In some embodiments, the genome editing comprises non-homologous end joining (NHEJ). In some embodiments, the NHEJ deletes a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

A method of the present disclosure for editing the genome of a cardiac cell can be used to correct any of a variety of genetic defects that give rise to a cardiac disease or disorder. Mutations of interest include mutations in one or more of the following genes: cardiac troponin T (TNNT2); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3). Specific mutations of interest include, without limitation, MYH7 R663H mutation; TNNT2 R173W; and KCNQ1 G269S missense mutation. Mutations of interest include mutations in one or more of the following genes: MYH6, ACTN2, SERCA2, GATA4, TBX5, MYOCD, NKX2-5, NOTCH1, MEF2C, HAND2, and HAND1. In some embodiments, the mutations of interest include mutations in the following genes: MEF2C, TBX5, and MYOCD. Cardiac diseases and disorders that can be treated with a method of the present disclosure include coronary heart disease, cardiomyopathy, endocarditis, congenital cardiovascular defects, and congestive heart failure. Cardiac diseases and disorders that can be treated with a method of the present disclosure include hypertrophic cardiomyopathy; a valvular heart disease; myocardial infarction; congestive heart failure; long QT syndrome; atrial arrhythmia; ventricular arrhythmia; diastolic heart failure; systolic heart failure; cardiac valve disease; cardiac valve calcification; left ventricular non-compaction; ventricular septal defect; and ischemia.

In some embodiments, the disclosure provides a method of transducing a cardiac cell. In some embodiments, the disclosure provides a method of transducing a cardiac cell, comprising contacting the cardiac cell with an rAAV virion described herein, wherein the rAAV virion transduces the cardiac cell. In some embodiments, the cardiac cell is a cardiomyocyte.

In some embodiments, the disclosure provides a method of transducing a cardiac cell, comprising contacting the cardiac cell with an rAAV virion, wherein the rAAV virion comprises a capsid protein, wherein the capsid protein is any capsid protein described herein.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
an amino acid insertion at position 584 comprising one or more of an asparagine (N), a threonine (T), a tyrosine (Y), phenylalanine (F), and an alanine (A);
an amino acid insertion at position 585 comprising one or more of a histidine (H) and a methionine (M);
an amino acid insertion at position 586 comprising one or more of a histidine (H), a tyrosine (Y), a valine (V), a threonine (T), an alanine (A), an isoleucine (I), a tryptophan (W), a methionine (M), and a leucine;
an amino acid insertion between positions 585 and 586 comprising one or two of histidine (H), tyrosine (Y), tryptophan (W), and methionine (M) (e.g., an insertion of WM or HY);
an amino acid insertion at position 587 comprising one or more of an isoleucine (I) and a proline (P);
an amino acid insertion at position 588 comprising one or more of an isoleucine (I), a threonine (T), and a proline (P);
an amino acid insertion at position 589 comprising one or more of a glycine (G) and a glutamine (Q);
one or more amino acid substitutions selected from the group consisting of N452K, N452A, N452V, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585E, S586N, A587T, Q588V, A589S, Q590I, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions S586T, A587L, Q588F, A589N, Q590S, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585N, A587T, Q588Y, A589L, Q590G, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585G, A587I, Q588L, A589T, Q590H, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585M, S586M, A587T, Q588T, A589A, and Q590R.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585C, A587T, Q588S, A589I, and Q590R.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585N, A587T, Q588Y, A589L, and Q590G.

In some embodiments, the disclosure provides a method of delivering one or more gene products to a cardiac cell. In some embodiments, the method of delivering one or more gene products to a cardiac cell comprises contacting the cardiac cell with an rAAV virion described herein. In some embodiments, the cardiac cell is a cardiomyocyte.

In some embodiments, the disclosure provides a method of delivering one or more gene products to a cardiac cell with an rAAV virion comprising a capsid protein, wherein the capsid protein is any capsid protein described herein.

In some embodiments, the disclosure provides a method of delivering one or more gene products to a cardiac cell with an rAAV virion comprising a capsid protein, wherein the capsid protein comprises a variant polypeptide sequence at the VR-VIII site, wherein the VR-VIII site (e.g., the entire VR-VIII site) comprises, consists essentially of, or consists of, a sequence having at least about 60%, 65%, 70%, 71%, 74%, 75%, 78%, 78.5%, 79%, 80%, 83%, 85%, 86%, 90%, 92%, 93% or 100% identity to any one of the following sequences (e.g., with at most 1, 2, or 3 amino acid substitutions relative to any one of the following sequences):

| VR-VIII Alignment (581-594) | SEQ ID NO |
|---|---|
| ATNHENTVSIAQTG | 618 |
| ATNHQTLFNSAQTG | 684 |
| ATNHNSTYLGAQTG | 642 |
| ATNHGSILTHAQTG | 630 |
| ATNHMMTTARAQTG | 615 |
| ATNHCSTSIRAQTG | 692 |
| ATNHQGAYAQAQTG | 616 |
| ATNHNTKLAIAQTG | 668 |
| ATNHVSSFTSAQTG | 619 |
| ATNHEDNIRSAQTG | 726 |
| ATNHQSAQAQAQTG | 5 |
| ATNHNNVISGAQTG | 608 |
| ATNHTGTSIIAQTG | 603 |
| ATNHQWMSAQAQAQTG | 657 |
| ATNHQDARAQAQTG | 675 |
| ATNHQHYSAQAQAQTG | 622 |
| ATNHQSAQAQAQTG | 5 |
| ATNHNIRTEMAQTG | 683 |
| ATNHSTTNFRAQTG | 621 |
| ATNHQANYGQAQTG | 598 |
| ATNHNMNRVNAQTG | 607 |
| ATNHSNSVQSAQTG | 609 |
| ATNHSSTFQGAQTG | 613 |
| ATNHSTTNFRAQTG | 621 |
| ATNHSSIFNSAQTG | 624 |
| ATNHAGNYNNAQTG | 625 |
| ATNHTSVISIAQTG | 636 |
| ATNHHSRVEIAQTG | 639 |
| ATNHSSIIYSAQTG | 661 |
| ATNHSGRDSYAQTG | 665 |
| ATNHSSSYNNAQTG | 669 |
| ATNHHNPSINAQTG | 674 |
| ATNHNRNGLLAQTG | 681 |
| ATNHESTSVRAQTG | 682 |
| ATNHLSVSSIAQTG | 688 |
| ATNHEDIIRSAQTG | 691 |
| ATNRQTAQAQAQTG | 602 |
| ATNRQIAQAQAQTG | 679 |

In some embodiments, the disclosure provides a method of delivering one or more gene products to a cardiac cell with an rAAV virion comprising a capsid protein, wherein the capsid protein comprises, consists essentially of, or consists of an amino acid sequence having at least 80%, 85%, 90%, 95%, 96%, 97%, 98%, 99%, or 100% sequence identity to any sequence selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

In some embodiments, the disclosure provides a method of delivering one or more gene products to a cardiac cell with an rAAV virion comprising a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
(a) amino acid substitutions Q585E, S586N, A587T, Q588V, A589S, Q590I, and N452K;
(b) amino acid substitutions S586T, A587L, Q588F, A589N, Q590S, and N452K;
(c) amino acid substitutions Q585N, A587T, Q588Y, A589L, Q590G, and N452K;
(d) amino acid substitutions Q585G, A587I, Q588L, A589T, Q590H, and N452K;
(e) amino acid substitutions Q585M, S586M, A587T, Q588T, A589A, and Q590R;
(f) amino acid substitutions Q585C, A587T, Q588S, A589I, and Q590R, or
(g) amino acid substitutions Q585N, A587T, Q588Y, A589L, and Q590G.

### Methods of Treatment

The disclosure provides a methods of treating a cardiac pathology in a subject in need thereof, comprising administering a therapeutically effective amount of a pharmaceutical composition comprising an rAAV virion to the subject, wherein the rAAV virion transduces cardiac tissue.

Subjects in need of treatment using compositions and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects suitable for treatment using the compositions, cells and methods of the present disclosure include individuals (e.g., mammalian subjects, such as humans, non-human primates, domestic mammals, experimental non-human mammalian subjects such as mice, rats, etc.) having a cardiac condition including but limited to a condition that results in ischemic heart tissue (e.g., individuals with coronary artery disease) and the like.

In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For example, the cardiac pathology can be selected from the group consisting of congestive heart failure, myocardial infarction, cardiac ischemia, myocarditis and arrhythmia. In some embodiments, the subject is diabetic. In some embodiments, the subject is non-diabetic. In some embodiments, the subject suffers from diabetic cardiomyopathy.

For therapy, the rAAV virions of the disclosure and/or pharmaceutical compositions thereof can be administered locally or systemically. An rAAV virion can be introduced by injection, catheter, implantable device, or the like. An rAAV virion can be administered in any physiologically acceptable excipient or carrier that does not adversely affect the cells. For example, rAAV virions of the disclosure and/or pharmaceutical compositions thereof can be administered intravenously or through an intracardiac route (e.g., epicardially or intramyocardially). Methods of administering rAAV virions of the disclosure and/or pharmaceutical compositions thereof to subjects, particularly human subjects include injection or infusion of the pharmaceutical compositions (e.g., compositions comprising rAAV virions). Injection may include direct muscle injection and infusion may include intravascular infusion. The rAAV virions or pharmaceutical compositions can be inserted into a delivery device which facilitates introduction by injection into the subjects. Such delivery devices include tubes, e.g., catheters, for injecting cells and fluids into the body of a recipient subject. The tubes can additionally include a needle, e.g., a syringe, through which the cells of the invention can be introduced into the subject at a desired location.

In some embodiments, the rAAV virion is administered by subcutaneous, intravenous, intramuscular, intraperitoneal, or intracardiac injection or by intracardiac catheterization. In some embodiments, the rAAV virion is administered by direct intramyocardial injection or transvascular administration. In some embodiments, the rAAV virion is administered by direct intramyocardial injection, antegrade intracoronary injection, retrograde injection, transendomyocardial injection, or molecular cardiac surgery with recirculating delivery (MCARD).

The rAAV virions can be inserted into such a delivery device, e.g., a syringe, in different forms. The rAAV virion can be supplied in the form of a pharmaceutical composition. Such a composition can include an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The choice of the excipient and any accompanying constituents of the composition can be adapted to optimize administration by the route and/or device employed.

Recombinant AAV may be administered locally or systemically. Recombinant AAV may be engineered to target specific cell types by selecting the appropriate capsid protein of the disclosure. To determine the suitability of various therapeutic administration regimens and dosages of AAV virion compositions, the rAAV virions can first be tested in a suitable animal model. At one level, recombinant AAV are assessed for their ability to infect target cells in vivo. Recombinant AAV can also be assessed to ascertain whether it migrates to target tissues, whether they induce an immune response in the host, or to determine an appropriate number, or dosage, of rAAV virions to be administered. It may be desirable or undesirable for the recombinant AAV to generate an immune response, depending on the disease to be treated. Generally, if repeated administration of a virion is required, it will be advantageous if the virion is not immunogenic. For testing purposes, rAAV virion compositions can be administered to immunodeficient animals (such as nude mice, or animals rendered immunodeficient chemically or by irradiation). Target tissues or cells can be harvested after a period of infection and assessed to determine if the tissues or cells have been infected and if the desired phenotype (e.g. induced cardiomyocyte) has been induced in the target tissue or cells.

Recombinant AAV virions can be administered by various routes, including without limitation direct injection into the heart or cardiac catheterization. Alternatively, the rAAV virions can be administered systemically such as by intravenous infusion. When direct injection is used, it may be performed either by open-heart surgery or by minimally invasive surgery. In some embodiments, the recombinant viruses are delivered to the pericardial space by injection or infusion. Injected or infused recombinant viruses can be traced by a variety of methods. For example, recombinant AAV labeled with or expressing a detectable label (such as green fluorescent protein, or beta-galactosidase) can readily be detected. The recombinant AAV may be engineered to cause the target cell to express a marker protein, such as a surface-expressed protein or a fluorescent protein. Alternatively, the infection of target cells with recombinant AAV can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

In some embodiments, the disclosure provides a method of treating a cardiac pathology in a subject in need thereof, comprising administering a therapeutically effective amount of an rAAV virion described herein.

In some embodiments, the disclosure provides a method of treating a cardiac pathology in a subject in need thereof, comprising administering a therapeutically effective amount of an rAAV virion comprising a capsid protein, wherein the capsid protein is any capsid protein described herein.

In some embodiments, the disclosure provides a method of treating a cardiac pathology in a subject in need thereof, comprising administering a therapeutically effective amount of an rAAV virion comprising a capsid protein, wherein the capsid protein comprises a variant polypeptide sequence at the VR-VIII site, wherein the VR-VIII site (e.g., the entire VR-VIII site) comprises, consists essentially of, or consists of, a sequence having at least about 60%, 65%, 70%, 71%, 74%, 75%, 78%, 78.5%, 79%, 80%, 83%, 85%, 86%, 90%, 92%, 93% or 100% identity to any one of the following sequences (e.g., with at most 1, 2, or 3 amino acid substitutions relative to any one of the following sequences):

| VR-VIII Alignment (581-594) | SEQ ID NO |
|---|---|
| ATNHENTVSIAQTG | 618 |
| ATNHQTLFNSAQTG | 684 |
| ATNHNSTYLGAQTG | 642 |
| ATNHGSILTHAQTG | 630 |
| ATNHMMTTARAQTG | 615 |
| ATNHCSTSIRAQTG | 692 |
| ATNHQGAYAQAQTG | 616 |
| ATNHNTKLAIAQTG | 668 |
| ATNHVSSFTSAQTG | 619 |
| ATNHEDNIRSAQTG | 726 |
| ATNHQSAQAQAQTG | 5 |
| ATNHNNVISGAQTG | 608 |
| ATNHTGTSIIAQTG | 603 |
| ATNHQWMSAQAQAQTG | 657 |
| ATNHQDARAQAQTG | 675 |
| ATNHQHYSAQAQAQTG | 622 |
| ATNHQSAQAQAQTG | 5 |
| ATNHNIRTEMAQTG | 683 |
| ATNHSTTNFRAQTG | 621 |
| ATNHQANYGQAQTG | 598 |
| ATNHNMNRVNAQTG | 607 |
| ATNHSNSVQSAQTG | 609 |
| ATNHSSTFQGAQTG | 613 |
| ATNHSTTNFRAQTG | 621 |
| ATNHSSIFNSAQTG | 624 |
| ATNHAGNYNNAQTG | 625 |
| ATNHTSVISIAQTG | 636 |
| ATNHHSRVEIAQTG | 639 |
| ATNHSSIIYSAQTG | 661 |
| ATNHSGRDSYAQTG | 665 |
| ATNHSSSYNNAQTG | 669 |
| ATNHHNPSINAQTG | 674 |
| ATNHNRNGLLAQTG | 681 |
| ATNHESTSVRAQTG | 682 |
| ATNHLSVSSIAQTG | 688 |
| ATNHEDIIRSAQTG | 691 |
| ATNRQTAQAQAQTG | 602 |
| ATNRQIAQAQAQTG | 679 |

In some embodiments, the disclosure provides a method of treating a cardiac pathology in a subject in need thereof, comprising administering a therapeutically effective amount of an rAAV virion comprising a capsid protein, wherein the capsid protein comprises, consists essentially of, or consists of an amino acid sequence having at least 80%, 85%, 90%, 95%, 96%, 97%, 98%, 99%, or 100% sequence identity to any sequence selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

In some embodiments, the disclosure provides a method of treating a cardiac pathology in a subject in need thereof, comprising administering a therapeutically effective amount of an rAAV virion comprising a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
(a) amino acid substitutions Q585E, S586N, A587T, Q588V, A589S, Q590I, and N452K;
(b) amino acid substitutions S586T, A587L, Q588F, A589N, Q590S, and N452K;
(c) amino acid substitutions Q585N, A587T, Q588Y, A589L, Q590G, and N452K;
(d) amino acid substitutions Q585G, A587I, Q588L, A589T, Q590H, and N452K;
(e) amino acid substitutions Q585M, S586M, A587T, Q588T, A589A, and Q590R;
(f) amino acid substitutions Q585C, A587T, Q588S, A589I, and Q590R, or
(g) amino acid substitutions Q585N, A587T, Q588Y, A589L, and Q590G.

### Pharmaceutical Compositions

The present disclosure provides pharmaceutical composition comprising an rAAV virion of the disclosure. The pharmaceutical composition may include one or more of a pharmaceutically acceptable carrier, diluent, excipient, and buffer. In some embodiments, the pharmaceutically acceptable carrier, diluent, excipient, or buffer is suitable for use in a human. Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as pH buffering substances may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

To prepare the composition, rAAV virion is generated and purified as necessary or desired. The rAAV can be mixed with or suspended in a pharmaceutically acceptable carrier. These rAAV can be adjusted to an appropriate concentration, and optionally combined with other agents. The concentration of rAAV virion and/or other agent included in a unit dose can vary widely. The dose and the number of administrations can be optimized by those skilled in the art. For example, about 10²-10¹⁰ vector genomes (vg) may be administered. In some embodiments, the dose is at least about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes. Daily doses of the compounds can vary as well. Such daily doses can range, for example, from at least about 10² vg/day, about 10³ vg/day, about 10⁴ vg/day, to about 10⁵ vg/day, about 10⁶ vg/day, about 10⁷ vg/day, about 10⁸ vg/day, about 10⁹ vg/day, about 10¹⁰ vg/day, or more vector genomes per day.

In certain embodiments, the method of treatment is enhanced by the administration of one or more anti-inflammatory agents, e.g., an anti-inflammatory steroid or a nonsteroidal anti-inflammatory drug (NSAID).

Anti-inflammatory steroids for use in the invention include the corticosteroids, and in particular those with glucocorticoid activity, e.g., dexamethasone and prednisone. Nonsteroidal anti-inflammatory drugs (NSAIDs) for use in the invention generally act by blocking the production of prostaglandins that cause inflammation and pain, cyclooxygenase-1 (COX-1) and/or cyclooxygenase-2 (COX-2). Traditional NSAIDs work by blocking both COX-1 and COX-2. The COX-2 selective inhibitors block only the COX-2 enzyme. In certain embodiment, the NSAID is a COX-2 selective inhibitor, e.g., celecoxib (Celebrex®), rofecoxib (Vioxx), and valdecoxib (B extra). In certain embodiments, the anti-inflammatory is an NSAID prostaglandin inhibitor, e.g., Piroxicam.

The amount of rAAV virion for use in treatment will vary not only with the particular carrier selected but also with the route of administration, the nature of the condition being treated and the age and condition of the patient. Ultimately, the attendant health care provider may determine proper dosage. A pharmaceutical composition may be formulated with the appropriate ratio of each compound in a single unit dosage form for administration with or without cells. Cells or vectors can be separately provided and either mixed with a liquid solution of the compound composition, or administered separately.

Recombinant AAV can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions can take the form of suspensions, solutions, or emulsions in oily or aqueous vehicles, and can contain formulatory agents such as suspending, stabilizing and/or dispersing agents. Suitable carriers include saline solution, phosphate buffered saline, and other materials commonly used in the art.

The compositions can also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g, Amiloride (Midamor), Bumetanide (Bumex), Chlorothiazide (Diuril), Chlorthalidone (Hygroton), Furosemide (Lasix), Hydrochlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof.

Additional agents can also be included such as antibacterial agents, antimicrobial agents, anti-viral agents, biological response modifiers, growth factors; immune modulators, monoclonal antibodies and/or preservatives. The compositions of the invention may also be used in conjunction with other forms of therapy.

The rAAV virions described herein can be administered to a subject to treat a disease or disorder. Such a composition may be in a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compounds and compositions of the invention may be essentially continuous over a preselected period of time or may be in a series of spaced doses. Both local and systemic administration is contemplated. In some embodiments, localized delivery of TAAV virion is achieved. In some embodiments, localized delivery of rAAV virions is used to generate a population of cells within the heart. In some embodiments, such a localized population operates as “pacemaker cells” for the heart. In some embodiments, the rAAV virions are used to generate, regenerate, repair, replace, and/or rejuvenate one or more of a sinoatrial (SA) node, an atrioventricular (AV) node, a bindle of His, and/or Purkinje fibers.

To control tonicity, an aqueous pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8, e.g. between 6.5 and 7.5, or between 7.0 and 7.8.

The composition is preferably sterile. The composition is preferably gluten free. The composition is preferably non-pyrogenic.

In some embodiments, a composition comprising cells may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

One or more of the following types of compounds can also be present in the composition with the rAAV virions: a WNT agonist, a GSK3 inhibitor, a TGF-beta signaling inhibitor, an epigenetic modifier, LSD1 inhibitor, an adenylyl cyclase agonist, or any combination thereof.

### Kits

A variety of kits are described herein that include any of composition (e.g. rAAV virions) described herein. The kit can include any of compositions described herein, either mixed together or individually packaged, and in dry or hydrated form. The rAAV virions and/or other agents described herein can be packaged separately into discrete vials, bottles or other containers. Alternatively, any of the rAAV virions and/or agents described herein can be packaged together as a single composition, or as two or more compositions that can be used together or separately. The compounds and/or agents described herein can be packaged in appropriate ratios and/or amounts to facilitate conversion of selected cells across differentiation boundaries to form cardiac progenitor cells and/or cardiomyocytes.

The kit can include instructions for administering those compositions, compounds and/or agents. Such instructions can provide the information described throughout this application. The rAAV virion or pharmaceutical composition can be provided within any of the kits in the form of a delivery device. Alternatively a delivery device can be separately included in the kits, and the instructions can describe how to assemble the delivery device prior to administration to a subject.

Any of the kits can also include syringes, catheters, scalpels, sterile containers for sample or cell collection, diluents, pharmaceutically acceptable carriers, and the like. The kits can provide other factors such as any of the supplementary factors or drugs described herein for the compositions in the preceding section or other parts of the application.

### Exemplary Embodiments

In some embodiments, a recombinant adeno-associated virus (rAAV) capsid protein, wherein the capsid protein shares, or comprises a sequence sharing, at least 80%, at least 85%, at least 90%, or at least 95% amino acid sequence identity to an AAV9 VP3 reference sequence according to SEQ ID NO: 487, and wherein the capsid protein comprises one or more modifications in the VR-III site and/or one more modifications in the VR-IV site of SEQ ID NO: 487.

In some embodiments, the disclosure provides a recombinant adeno-associated virus (rAAV) capsid protein. In some embodiments, the disclosure provides an rAAV virion comprising an rAAV capsid protein described herein. In some embodiments, the rAAV capsid protein shares at least 80%, at least 85%, at least 90%, or at least 95% amino acid sequence identity to an AAV9 VP3 reference sequence according to SEQ ID NO: 487, and comprises, relative to reference sequence SEQ ID NO: 1, amino acid substitution N452K.

In some embodiments, the disclosure provides a recombinant adeno-associated virus (rAAV) capsid protein. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585E, S586N, A587T, Q588V, A589S, Q590I, and N452K. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions S586T, A587L, Q588F, A589N, Q590S, and N452K. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585N, A587T, Q588Y, A589L, Q590G, and N452K. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585G, A587I, Q588L, A589T, Q590H, and N452K. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585N, A587T, Q588Y, A589L, and Q590G. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585M, S586M, A587T, Q588T, A589A, and Q590R. In some embodiments, the capsid protein shares at least 80%, at least 85%, at least 90%, or at least 95% amino acid sequence identity to an AAV9 VP3 reference sequence according to SEQ ID NO: 487, and wherein the capsid protein comprises, relative to reference sequence SEQ ID NO: 1, amino acid substitutions Q585C, Q586S, A587T, Q588S, A589I, and Q590R.

In some embodiments, the capsid protein comprises an amino acid sequence having at least 80%, at least 85%, at least 90%, at least 95%, at least 98%, at least 99%, or 100% sequence identity to the amino acid sequence selected from the group consisting of: SEQ ID NOs: 488-589, 705-710, and 767-780. In some embodiments, the capsid protein comprises an amino acid sequence having at least 80%, at least 85%, at least 90%, at least 95%, at least 98%, at least 99%, or 100% sequence identity to the amino acid sequence selected from the group consisting of: SEQ ID NOs: 488-589, 705-710, and 767-780, without any changes in the VR-VIII site (or in the VR-VIII and VR-IV sites) other than those already provided in the respective sequences.

In some embodiments, the capsid protein comprises SEQ ID NO: 705. In some embodiments, the capsid protein comprises SEQ ID NO: 706. In some embodiments, the capsid protein comprises SEQ ID NO: 707. In some embodiments, the capsid protein comprises SEQ ID NO: 708. In some embodiments, the capsid protein comprises SEQ ID NO: 512. In some embodiments, the capsid protein comprises SEQ ID NO: 539. In some embodiments, the capsid protein comprises SEQ ID NO: 589. In some embodiments, the capsid protein comprises SEQ ID NO: 488. In some embodiments, the capsid protein comprises SEQ ID NO: 499. In some embodiments, the capsid protein comprises SEQ ID NO: 504. In some embodiments, the capsid protein comprises SEQ ID NO: 505. In some embodiments, the capsid protein comprises SEQ ID NO: 506. In some embodiments, the capsid protein comprises SEQ ID NO: 510. In some embodiments, the capsid protein comprises SEQ ID NO: 513. In some embodiments, the capsid protein comprises SEQ ID NO: 516. In some embodiments, the capsid protein comprises SEQ ID NO: 518. In some embodiments, the capsid protein comprises SEQ ID NO: 521. In some embodiments, the capsid protein comprises SEQ ID NO: 522. In some embodiments, the capsid protein comprises SEQ ID NO: 533. In some embodiments, the capsid protein comprises SEQ ID NO: 536. In some embodiments, the capsid protein comprises SEQ ID NO: 558. In some embodiments, the capsid protein comprises SEQ ID NO: 562. In some embodiments, the capsid protein comprises SEQ ID NO: 566. In some embodiments, the capsid protein comprises SEQ ID NO: 571. In some embodiments, the capsid protein comprises SEQ ID NO: 576. In some embodiments, the capsid protein comprises SEQ ID NO: 578. In some embodiments, the capsid protein comprises SEQ ID NO: 579. In some embodiments, the capsid protein comprises SEQ ID NO: 580. In some embodiments, the capsid protein comprises SEQ ID NO: 581. In some embodiments, the capsid protein comprises SEQ ID NO: 585. In some embodiments, the capsid protein comprises SEQ ID NO: 588. In some embodiments, the capsid protein comprises SEQ ID NO: 710. In some embodiments, the capsid protein comprises SEQ ID NO: 772. In some embodiments, the capsid protein comprises SEQ ID NO: 774. In some embodiments, the capsid protein referenced herein may further comprise up to one, two, three, four, five, six, seven, eight, nine, ten, fifteen, twenty, twenty-five, thirty, thirty-five, forty or fifty substitutions or insertions (e.g., as described herein or conservative substitutions).

In some embodiments, the capsid protein comprises the same substitution motif (e.g., the same VR-VIII, and/or the same VR-IV substitution motif) as any one of the capsid proteins selected from the group consisting of: SEQ ID NOs: 512, 589, 772, 774, 705, 513, 710, 488, 707, and 539. In some embodiments, the capsid protein comprises the same substitution motif (e.g., the same VR-VIII, and/or the same VR-IV substitution motif) as any one of the capsid proteins selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774.

In some embodiments, the capsid protein is ZC377 as provided herein. In some embodiments, the capsid protein is ZC388 as provided herein. In some embodiments, the capsid protein is ZC393 as provided herein. In some embodiments, the capsid protein is ZC394 as provided herein. In some embodiments, the capsid protein is ZC395 as provided herein. In some embodiments, the capsid protein is ZC399 as provided herein. In some embodiments, the capsid protein is ZC401 as provided herein. In some embodiments, the capsid protein is ZC402 as provided herein. In some embodiments, the capsid protein is ZC405 as provided herein. In some embodiments, the capsid protein is ZC407 as provided herein. In some embodiments, the capsid protein is ZC410 as provided herein. In some embodiments, the capsid protein is ZC411 as provided herein. In some embodiments, the capsid protein is ZC422 as provided herein. In some embodiments, the capsid protein is ZC425 as provided herein. In some embodiments, the capsid protein is ZC428 as provided herein. In some embodiments, the capsid protein is ZC447 as provided herein. In some embodiments, the capsid protein is ZC451 as provided herein. In some embodiments, the capsid protein is ZC455 as provided herein. In some embodiments, the capsid protein is ZC460 as provided herein. In some embodiments, the capsid protein is ZC465 as provided herein. In some embodiments, the capsid protein is ZC467 as provided herein. In some embodiments, the capsid protein is ZC468 as provided herein. In some embodiments, the capsid protein is ZC469 as provided herein. In some embodiments, the capsid protein is ZC470 as provided herein. In some embodiments, the capsid protein is ZC474 as provided herein. In some embodiments, the capsid protein is ZC477 as provided herein. In some embodiments, the capsid protein is ZC478 as provided herein. In some embodiments, the capsid protein is ZC373 as provided herein. In some embodiments, the capsid protein is ZC374 as provided herein. In some embodiments, the capsid protein is ZC375 as provided herein. In some embodiments, the capsid protein is ZC376 as provided herein. In some embodiments, the capsid protein is ACE10 as provided herein. In some embodiments, the capsid protein is ZC536 as provided herein. In some embodiments, the capsid protein is ZC538 as provided herein. In some embodiments, the capsid protein referenced herein may further comprise up to one, two, three, four, five, six, seven, eight, nine, ten, fifteen, twenty, twenty-five, thirty, thirty-five, forty or fifty substitutions or insertions (e.g., as described herein or conservative substitutions).

In some embodiments, the capsid protein comprises the same substitution motif (e.g., the same VR-VIII, and/or the same VR-IV substitution motif) as any one of the capsid proteins selected from the group consisting of: ZC401, ZC478, ZC536, ZC538, ZC373, ZC402, ACE10, ZC377, ZC375, and ZC428. In some embodiments, the capsid protein comprises the same substitution motif (e.g., the same VR-VIII, and/or the same VR-IV substitution motif) as any one of the capsid proteins selected from the group consisting of: ZC401, ZC478, ZC536, ZC538, ZC373, ZC402, ACE10, ZC377, ZC375, ZC428, ZC374, ZC376, ZC393, ZC394, ZC395, ZC399, ZC405, ZC407, ZC410, ZC411, ZC422, ZC425, ZC447, ZC451, ZC455, ZC460, ZC467, ZC468, ZC469, ZC470, ZC474, ZC477, ZC388, and ZC465.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1, amino acid substitutions of SEQ ID NO: 719 (ENTVSI) at positions 585-590.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1: amino acid substitutions of SEQ ID NO: 720 (QTLFNS) at positions 585-590.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1: amino acid substitutions of SEQ ID NO: 721 (NSTYLG) at positions 585-590.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1: amino acid substitutions of SEQ ID NO: 722 (GSILTH) at positions 585-590.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1: amino acid substitutions of SEQ ID NO: 723 (MMTTAR) at positions 585-590.

In some embodiments, the capsid protein shares at least 80%, at least 85%, at least 90%, or at least 95% amino acid sequence identity to an AAV9 VP3 reference sequence according to SEQ ID NO: 487, and the capsid protein comprises, relative to reference sequence SEQ ID NO: 1: amino acid substitutions of SEQ ID NO: 724 (CSTSIR) at positions 585-590.

The capsid proteins described in this exemplary embodiments section and in the numbered embodiments sections can be used in any of the embodiments (e.g., any of the virions, compositions, cells, and methods) and in combination with any other features specified herein.

### Numbered Embodiments I

1. A recombinant adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion at position 584 comprising one or more of an asparagine (N), a threonine (T), a tyrosine (Y), phenylalanine (F), and an alanine (A);
an amino acid insertion at position 585 comprising one or more of a histidine (H) and a methionine (M);
an amino acid insertion at position 586 comprising one or more of a histidine (H), a tyrosine (Y), a valine (V), a threonine (T), an alanine (A), an isoleucine (I), a tryptophan (W), a methionine (M), and a leucine;
an amino acid insertion at position 587 comprising one or more of an isoleucine (I) and a proline (P);
an amino acid insertion at position 588 comprising one or more of an isoleucine (I), a threonine (T), and a proline (P);
an amino acid insertion at position 589 comprising one or more of a glycine (G) and a glutamine (Q);
one or more amino acid substitutions selected from the group consisting of N452K, N452A, N452V, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

2. The capsid protein of embodiment 1, wherein the capsid protein comprises, relative to reference sequence SEQ ID NO: 1, an amino acid insertion at position 584 comprising one or more of an asparagine (N), a threonine (T), a tyrosine (Y), phenylalanine (F), and an alanine (A).

3. The capsid protein of embodiment 1 or embodiment 2, wherein the capsid protein comprises, relative to reference sequence SEQ ID NO: 1, an amino acid insertion at position 585 comprising one or more of a histidine (H) and a methionine (M).

4. The capsid protein of any one of embodiments 1-3, wherein the capsid protein comprises, relative to reference sequence SEQ ID NO: 1, an amino acid insertion at position 586 comprising one or more of a histidine (H), a tyrosine (Y), a valine (V), a threonine (T), an alanine (A), an isoleucine (I), a tryptophan (W), a methionine (M), and a leucine.

5. The capsid protein of any one of embodiments 1-4, wherein the capsid protein comprises, relative to reference sequence SEQ ID NO: 1, an amino acid insertion at position 587 comprising one or more of an isoleucine (I) and a proline (P).

6. The capsid protein of any one of embodiments 1-5, wherein the capsid protein comprises, relative to reference sequence SEQ ID NO: 1, an amino acid insertion at position 588 comprising one or more of an isoleucine (I), a threonine (T), and a proline (P)

7. The capsid protein of any one of embodiments 1-6, wherein the capsid protein comprises, relative to reference sequence SEQ ID NO: 1, an amino acid insertion at position 589 comprising one or more of a glycine (G) and a glutamine (Q).

8. The capsid protein of any one of embodiments 1-7, wherein the capsid protein comprises, relative to reference sequence SEQ ID NO: 1, an amino acid insertion at position 584 consisting of a TY, FN, or AT.

9. The capsid protein of any one of embodiments 1-8, wherein the capsid protein comprises, relative to reference sequence SEQ ID NO: 1, an amino acid insertion at position 585 consisting of MH.

10. The capsid protein of any one of embodiments 1-9, wherein the capsid protein comprises, relative to reference sequence SEQ ID NO: 1, an amino acid insertion at position 586 consisting of HY, VT, AI, WM, or ML.

11. The capsid protein of any one of embodiments 1-10, wherein the capsid protein comprises, relative to reference sequence SEQ ID NO: 1, an amino acid insertion at position 587 consisting of PI.

12. The capsid protein of any one of embodiments 1-11, wherein the capsid protein comprises, relative to reference sequence SEQ ID NO: 1, an amino acid insertion at position 588 consisting of IT or PT.

13. The capsid protein of any one of embodiments 1-12, wherein the capsid protein comprises, relative to reference sequence SEQ ID NO: 1, one or more amino acid substitutions selected from the group consisting of N452K, N452A, N452V, G453A, G453N, S454T, S454D, G455N, Q456L, Q456K, N457L, N457V, Q458I, and Q458H.

14. The capsid protein of any one of embodiments 1-13, wherein the capsid protein comprises, relative to reference sequence SEQ ID NO: 1, one or more amino acid substitutions selected from the group consisting of T582D, T582E, N583V, H584Q, S586K, A587P, A587S, Q588G, Q588M, A589S, A591I, G594Q, and G594D.

15. The capsid protein of any one of embodiments 1-14, wherein the capsid protein comprises, relative to reference sequence SEQ ID NO: 1, one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R.

16. The capsid protein of any one of embodiments 1-15, wherein the capsid protein comprises, relative to reference sequence SEQ ID NO: 1, one or more amino acid substitutions selected from the group consisting of Q585C, Q585S, S586I, A587V and A587G.

17. The capsid protein of any one of embodiments 1-16, wherein the capsid protein comprises, relative to reference sequence SEQ ID NO: 1, one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

18. The capsid protein of any one of embodiments 1-17, wherein the capsid protein comprises, relative to reference sequence SEQ ID NO: 1, an amino acid sequence selected from SEQ ID NOs: 599-692 and wherein the capsid protein shares at least 80%, at least 90%, at least 95%, at least 98%, or 100% identity to SEQ ID NOs: 496-589.

19. The capsid protein of any one of embodiments 1-18, wherein the capsid protein comprises, relative to reference sequence SEQ ID NO: 1, the amino acid sequence ANYG at positions 586-589 or at about positions 586-589.

20. The capsid protein of any one of embodiments 1-19, wherein the capsid protein comprises, relative to reference sequence SEQ ID NO: 1, two or more amino acid substitutions selected from the group consisting of N452K, N452A, N452V, G453A, G453N, S454T, S454D, G455N, Q456L, Q456K, N457L, N457V, Q458I, and Q458H.

21. The capsid protein of any one of embodiments 1-20, wherein the capsid protein comprises, relative to reference sequence SEQ ID NO: 1, the amino acid substitution N452K, N452A, or N452V.

22. The capsid protein of embodiment 21, wherein the capsid protein comprises, relative to reference sequence SEQ ID NO: 1, the amino acid substitution N452K.

23. The capsid protein of any one of embodiments 1-22, wherein the capsid protein comprises, relative to reference sequence SEQ ID NO: 1, the amino acid substitution G453A or G453N.

24. The capsid protein of any one of embodiments 1-23, wherein the capsid protein comprises, relative to reference sequence SEQ ID NO: 1, the amino acid substitution S454T or S454D.

25. The capsid protein of any one of embodiments 1-24, wherein the capsid protein comprises, relative to reference sequence SEQ ID NO: 1, the amino acid substitution G455N.

26. The capsid protein of any one of embodiments 1-25, wherein the capsid protein comprises, relative to reference sequence SEQ ID NO: 1, the amino acid substitution Q456L or Q456K.

27. The capsid protein of any one of embodiments 1-26, wherein the capsid protein comprises, relative to reference sequence SEQ ID NO: 1, the amino acid substitution N457L or N457V.

28. The capsid protein of any one of embodiments 1-27, wherein the capsid protein comprises, relative to reference sequence SEQ ID NO: 1, the amino acid substitution Q458I or Q458H.

29. The capsid protein of any one of embodiments 1-28, wherein the capsid protein comprises, relative to reference sequence SEQ ID NO: 1, an amino acid sequence selected from KGSGQNQ (SEQ ID NO: 590), NASGQNQ (SEQ ID NO: 591), NGTGQNQ (SEQ ID NO: 592), NGSGLNQ (SEQ ID NO: 593), ANDNKLI (SEQ ID NO: 594), VNDNKVI (SEQ ID NO: 595), NGSGQNH (SEQ ID NO: 596), and ANDNKVI (SEQ ID NO: 597) at positions 452-458 or at about positions 452-458, and wherein the capsid protein shares at least 80%, at least 90%, at least 95%, at least 98%, or 100% identity to SEQ ID NOs: 488-495, and wherein optionally the capsid protein comprises the amino acid sequence ANYG at positions 586-589 or at about positions 586-589.

30. A recombinant adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1, amino acid substitution N452K.

31. The capsid protein of any one of embodiments 1-30, wherein the capsid protein comprises, relative to reference sequence SEQ ID NO: 1:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and/or
at position 590 an amino acid selected from: I, S, G, H, R and Q.

32. The capsid protein of any one of embodiments 1-31, wherein the capsid protein comprises, relative to reference sequence SEQ ID NO: 1:
at position 585 an amino acid selected from: E, N, G, M, C, V and T;
at position 586 an amino acid selected from: N, T, M, G, D and N;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and/or
at position 590 an amino acid selected from: I, S, G, H and R.

33. The capsid protein of any one of embodiments 1-32, wherein the capsid protein comprises, relative to reference sequence SEQ ID NO: 1, amino acid substitutions Q585E, S586N, A587T, Q588V, A589S, Q590I, and N452K.

34. The capsid protein of any one of embodiments 1-32, wherein the capsid protein comprises, relative to reference sequence SEQ ID NO: 1, amino acid substitutions S586T, A587L, Q588F, A589N, Q590S, and N452K.

35. The capsid protein of any one of embodiments 1-32, wherein the capsid protein comprises, relative to reference sequence SEQ ID NO: 1, amino acid substitutions Q585N, A587T, Q588Y, A589L, Q590G, and N452K.

36. The capsid protein of any one of embodiments 1-32, wherein the capsid protein comprises, relative to reference sequence SEQ ID NO: 1, amino acid substitutions Q585G, A587I, Q588L, A589T, Q590H, and N452K.

37. The capsid protein of any one of embodiments 1-32, wherein the capsid protein comprises, relative to reference sequence SEQ ID NO: 1, amino acid substitutions Q585M, S586M, A587T, Q588T, and Q590R.

38. The capsid protein of any one of embodiments 1-32, wherein the capsid protein comprises, relative to reference sequence SEQ ID NO: 1, amino acid substitutions Q585N, A587T, Q588Y, A589L, and Q590G.

39. The capsid protein of any one of embodiments 1-32, wherein the capsid protein comprises, relative to reference sequence SEQ ID NO: 1, amino acid substitutions Q585C, A587T, Q588S, A589I, and Q590R.

40. A recombinant adeno-associated virus (rAAV) virion, comprising a capsid protein according to any one of embodiments 1-39 and a vector genome comprising a polynucleotide cassette flanked by inverted terminal repeats (ITRs).

41. The rAAV virion of embodiment 40, wherein the rAAV virion transduces heart cells.

42. The rAAV virion of embodiment 40 or embodiment 41, wherein the rAAV virion transduces cardiomyocytes.

43. The rAAV virion of any one of embodiments 40-42, wherein the rAAV virion traffics to at least one organ other than the liver.

44. The rAAV virion of any one of embodiments 40-43, wherein the rAAV virion traffics to the heart.

45. The rAAV virion of any one of embodiments 40-44, wherein the rAAV virion exhibits a higher heart transduction efficiency than an rAAV virion having an AAV9 VP1 capsid protein according to SEQ ID NO: 1.

46. The rAAV virion of any one of embodiments 40-45, wherein the rAAV virion exhibits a higher heart-to-liver transduction ratio than an rAAV virion having an AAV9 VP1 capsid protein according to SEQ ID NO: 1, optionally at least 2, 3, 4, 5, 6, 7, 8, 9 or 10 times higher.

47. The rAAV virion of any one of embodiments 40-46, wherein administration of the rAAV virion to a subject leads to a lower liver viral load than administration of an rAAV virion having an AAV9 VP1 capsid protein according to SEQ ID NO: 1, optionally at least 2, 3, 4, 5, 6, 7, 8, 9 or 10 times lower.

48. The rAAV virion of any one of embodiments 40-47, wherein the rAAV virion exhibits a higher transduction efficiency, optionally higher heart transduction efficiency, than an rAAV virion having an AAV9 VP1 capsid protein according to SEQ ID NO: 1, assessed in a primate.

49. The rAAV virion of any one of embodiments 40-48, wherein the rAAV virion exhibits a higher heart-to-liver transduction ratio than an rAAV virion having an AAV9 VP1 capsid protein according to SEQ ID NO: 1, assessed in a primate, optionally at least 2, 3, 4, 5, 6, 7, 8, 9 or 10 times higher.

50. The rAAV virion of any one of embodiments 40-49, wherein administration of the rAAV virion to a subject leads to a lower liver viral load than administration of an rAAV virion having an AAV9 VP1 capsid protein according to SEQ ID NO: 1, assessed in a primate, optionally at least 2, 3, 4, 5, 6, 7, 8, 9 or 10 times lower.

51. The rAAV virion of any one of embodiments 40-50, wherein the polynucleotide cassette comprises a polynucleotide sequence encoding MYBPC3, DWORF, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, JPH2, PLN, and/or ATP2A2.

52. The rAAV virion of any one of embodiments 40-50, wherein the polynucleotide cassette comprises a polynucleotide sequence encoding CACNA1C, DMD, DMPK, EPG5, EVC, EVC2, FBN1, NF1, SCN5A, SOS1, NPR1, ERBB4, VIP, MYH7, and/or Cas9.

53. The rAAV virion of any one of embodiments 40-50, wherein the polynucleotide cassette comprises a polynucleotide sequence encoding MYOCD, ASCL1, GATA4, MEF2C, TBX5, miR-133, and/or MESP1.

54. A pharmaceutical composition comprising an rAAV virion according to any one of embodiments 40-53 and a pharmaceutically acceptable carrier.

55. A polynucleotide encoding the capsid protein of any one of embodiments 1-39.

56. A method of transducing a cardiac cell, comprising contacting the cardiac cell with an rAAV virion according to any one of embodiments 40-53, wherein the rAAV virion transduces the cardiac cell.

57. The method of embodiment 56, wherein the cardiac cell is a cardiomyocyte.

58. The method of embodiment 56 or embodiment 57, wherein the rAAV virion exhibits higher transduction efficiency in the cell than an rAAV virion having an AAV9 VP1 capsid protein according to SEQ ID NO: 1.

59. A method of delivering one or more gene products to a cardiac cell, comprising contacting the cardiac cell with an rAAV virion according to any one of embodiments 40-53.

60. The method of embodiment 59, wherein the cardiac cell is a cardiomyocyte.

61. A method of treating a cardiac pathology in a subject in need thereof, comprising administering a therapeutically effective amount of an rAAV virion according to any one of embodiments 40-53 to the subject, wherein the rAAV virion transduces cardiac tissue.

62. A kit comprising a pharmaceutical composition according to embodiment 54 and instructions for use.

### Numbered Embodiments II

1. An engineered polynucleotide encoding an adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion at position 584, or between positions 583 and 584, comprising one or more of an asparagine (N), a threonine (T), a tyrosine (Y), phenylalanine (F), and an alanine (A);
an amino acid insertion at position 585, or between positions 584 and 585, comprising one or more of a histidine (H) and a methionine (M);
an amino acid insertion at position 586, or between positions 585 and 586, comprising one or more of a histidine (H), a tyrosine (Y), a valine (V), a threonine (T), an alanine (A), an isoleucine (I), a tryptophan (W), a methionine (M), and a leucine (L);
an amino acid insertion at position 587, or between positions 586 and 587, comprising one or more of an isoleucine (I) and a proline (P);
an amino acid insertion at position 588, or between positions 587 and 588, comprising one or more of an isoleucine (I), a threonine (T), and a proline (P);
an amino acid insertion at position 589, or between positions 588 and 589, comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

2. The polynucleotide of embodiment 1, wherein the capsid protein comprises one, two, three, four or more substitutions or insertions in the VR-VIII site.

3. The polynucleotide of embodiment 2, wherein the capsid protein comprises comprises, relative to reference SEQ ID NO:1, one, two, three, four or more substitutions or insertions at positions from 584 to 590 in the VR-VIII site, or one, two, three, four or more substitutions or insertions at positions from 585 to 590 in the VR-VIII site.

4. The polynucleotide of any one of embodiments 1-3, wherein the capsid protein comprises, relative to reference sequence SEQ ID NO: 1:
(i) one or more amino acid substitutions selected from the group consisting of T582D, T582E, N583V, H584Q, S586K, A587P, A587S, Q588G, Q588M, A589S, A591I, G594Q, and G594D;
(ii) one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R;
(iii) one or more amino acid substitutions selected from the group consisting of Q585C, Q585S, S586I, A587V and A587G; or
(iv) one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

5. The polynucleotide of any one of embodiments 1-4, wherein the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence.

6. The polynucleotide of any one of embodiments 1-5, wherein the capsid protein comprises, relative to reference sequence SEQ ID NO: 1, one or more amino acid substitutions selected from the group consisting of N452K, N452A, N452V, N452I, G453A, G453N, S454T, S454D, G455N, Q456L, Q456K, N457L, N457V, Q458I, and Q458H.

7. The polynucleotide of any one of embodiments 1-5, wherein the capsid protein comprises, relative to reference sequence SEQ ID NO: 1, at position 452 an amino acid selected from the group consisting of: K and N.

8. The polynucleotide of any one of embodiments 1-5, wherein the capsid protein comprises, relative to reference sequence SEQ ID NO: 1, an amino acid substitution N452K.

9. The polynucleotide of any one of embodiments 1-8, wherein the capsid protein comprises, relative to reference sequence SEQ ID NO: 1:
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q.

10. The polynucleotide of any one of embodiments 1-5, wherein the capsid protein comprises, relative to reference sequence SEQ ID NO: 1:
at position 452 an amino acid selected from the group consisting of: K and N;
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

11. The polynucleotide of any one of embodiments 1-8, wherein the capsid protein comprises, relative to reference sequence SEQ ID NO: 1:
at position 584 amino acid R;
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G;
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M.

12. The polynucleotide of any one of embodiments 1-5, wherein the capsid protein comprises, relative to reference sequence SEQ ID NO: 1, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M

13. The polynucleotide of any one of embodiments 1-8, wherein the capsid protein comprises, relative to reference sequence SEQ ID NO: 1:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q.

14. The polynucleotide of any one of embodiments 1-5, wherein the capsid protein comprises, relative to reference sequence SEQ ID NO: 1:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

15. The polynucleotide of any one of embodiments 1-8, wherein the capsid protein comprises, relative to reference sequence SEQ ID NO: 1:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or at position 590 an amino acid selected from the group consisting of: I, S, G, H and R.

16. The polynucleotide of any one of embodiments 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, G, M, C, V and T;
(iii) at position 586 an amino acid selected from the group consisting of: N, T, M, G, and D;
(iv) at position 587 an amino acid selected from the group consisting of: T, L, I, K, S, N and V;
(v) at position 588 an amino acid selected from the group consisting of: V, F, Y, L, T, S, I and R;
(vi) at position 589 an amino acid selected from the group consisting of: S, N, L, T, I and R; and
(vii) at position 590 an amino acid selected from the group consisting of: I, S, G, H and R.

17. The polynucleotide of any one of embodiments 1-8, wherein the capsid protein comprises, relative to reference sequence SEQ ID NO: 1:
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q.

18. The polynucleotide of any one of embodiments 1-5, wherein the capsid protein comprises, relative to reference sequence SEQ ID NO: 1:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

19. The polynucleotide of any one of embodiments 1-8, wherein the capsid protein comprises, relative to reference sequence SEQ ID NO: 1:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or
at position 590 an amino acid selected from the group consisting of: I, S, G, and R.

20. The polynucleotide of any one of embodiments 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N;
(iv) at position 587 an amino acid selected from the group consisting of: T, N, and V;
(v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

21. The polynucleotide of any one of embodiments 1-20, wherein the capsid protein comprises, relative to reference sequence SEQ ID NO: 1:
at position 452 an amino acid selected from the group consisting of: K and N; and
at position 587 amino acid substitution A587T; and optionally comprises amino acid N or R at one, two or more positions selected from the group consisting of: 584, 585, 586, 588, 589, and 590.

22. The polynucleotide of any one of embodiments 1-21, wherein the capsid protein comprises, relative to reference sequence SEQ ID NO: 1:
at position 452 an amino acid selected from the group consisting of: K and N; and
amino acid N or R at one, two or more positions selected from the group consisting of: 584, 585, 586, 588, 589, and 590.

23. The polynucleotide of any one of embodiments 1-22, wherein the capsid protein comprises, relative to reference sequence SEQ ID NO: 1:
at position 452 an amino acid selected from the group consisting of: K and N; and amino acid S at two or more positions selected from the group consisting of: 585, 586, 587, 588, 589 and 590.

24. The polynucleotide of any one of embodiments 1-23, wherein the capsid protein comprises, relative to reference sequence SEQ ID NO: 1:
at position 452 an amino acid selected from the group consisting of: K and N; and
at three, four or more positions in the region 585-590 of the VR-VIII site, amino acids selected from the group consisting of: N, S, T, R and I.

25. The polynucleotide of embodiment 24, wherein the capsid protein comprises, relative to reference sequence SEQ ID NO: 1:
at three, four or more positions in the region 585-590 of the VR-VIII site, amino acids selected from the group consisting of: N, S, T, and R.

26. The polynucleotide of any one of embodiments 1-5, wherein the capsid protein comprises, relative to reference sequence SEQ ID NO: 1, amino acid substitutions Q585E, S586N, A587T, Q588V, A589S, Q590I, and N452K.

27. The polynucleotide of any one of embodiments 1-5, wherein the capsid protein comprises, relative to reference sequence SEQ ID NO: 1, amino acid substitutions S586T, A587L, Q588F, A589N, Q590S, and N452K.

28. The polynucleotide of any one of embodiments 1-5, wherein the capsid protein comprises, relative to reference sequence SEQ ID NO: 1, amino acid substitutions Q585N, A587T, Q588Y, A589L, Q590G, and N452K.

29. The polynucleotide of any one of embodiments 1-5, wherein the capsid protein comprises, relative to reference sequence SEQ ID NO: 1, amino acid substitutions Q585G, A587I, Q588L, A589T, Q590H, and N452K.

30. The polynucleotide of any one of embodiments 1-5, wherein the capsid protein comprises, relative to reference sequence SEQ ID NO: 1, amino acid substitutions Q585M, S586M, A587T, Q588T, and Q590R; and amino acid N at position 452.

31. The polynucleotide of any one of embodiments 1-5, wherein the capsid protein comprises, relative to reference sequence SEQ ID NO: 1, amino acid substitutions Q585N, A587T, Q588Y, A589L, and Q590G; and amino acid N at position 452.

32. The polynucleotide of any one of embodiments 1-5, wherein the capsid protein comprises, relative to reference sequence SEQ ID NO: 1, amino acid substitutions Q585C, A587T, Q588S, A589I, and Q590R; and amino acid N at position 452.

33. The polynucleotide of any one of embodiments 1-5, wherein the capsid protein comprises, relative to reference sequence SEQ ID NO: 1, amino acid substitutions Q585E, S586D, A587N, Q588I, A589R, and Q590S; and amino acid N at position 452.

34. The polynucleotide of any one of embodiments 1-5, wherein the capsid protein comprises, relative to reference sequence SEQ ID NO: 1, amino acid substitutions Q585E, S586D, A587N, Q588I, A589R, Q590S, and N452K.

35. The polynucleotide of any one of embodiments 1-5, wherein the capsid protein comprises, relative to reference sequence SEQ ID NO: 1, amino acid substitutions Q585N, S586N, A587V, Q588I, A589S, Q590G, and N452K.

36. The polynucleotide of any one of embodiments 1-5, wherein the capsid protein comprises, relative to reference sequence SEQ ID NO: 1, amino acid substitutions S586G and Q588Y; and amino acid N at position 452.

37. The polynucleotide of any one of embodiments 1-5, wherein the capsid protein comprises, relative to reference sequence SEQ ID NO: 1, amino acid substitutions S586A, A587N, Q588Y, A589G, and N452K.

38. The polynucleotide of any one of embodiments 1-37, wherein the capsid protein comprises, relative to reference sequence SEQ ID NO:1, amino acids ATN at positions 581-583, and amino acids AQTG at positions 591-594.

39. The polynucleotide of any one of embodiments 1-37, wherein the capsid protein comprises, relative to reference sequence SEQ ID NO: 1, amino acids ATNH at positions 581-584, and amino acids AQTG at positions 591-594.

40. The polynucleotide of any one of embodiments 1-5, wherein the capsid protein comprises, relative to reference sequence SEQ ID NO:1:
(i) amino acid sequence ATNHENTVSIAQTG at the VR-VIII positions 581-594, and amino acid K at the VR-IV position 452;
(ii) amino acid sequence ATNHQTLFNSAQTG at the VR-VIII positions 581-594, and amino acid K at the VR-IV position 452;
(iii) amino acid sequence ATNHNSTYLGAQTG at the VR-VIII positions 581-594, and amino acid K at the VR-IV position 452;
(iv) amino acid sequence ATNHGSILTHAQTG at the VR-VIII positions 581-594, and amino acid K at the VR-IV position 452;
(v) amino acid sequence ATNHMMTTARAQTG at the VR-VIII positions 581-594, and amino acid N at the VR-IV position 452;
(vi) amino acid sequence ATNHNSTYLGAQTG at the VR-VIII positions 581-594, and amino acid N at the VR-IV position 452;
(vii) amino acid sequence ATNHCSTSIRAQTG at the VR-VIII positions 581-594, and amino acid N at the VR-IV position 452;
(viii) amino acid sequence ATNHEDNIRSAQTG at the VR-VIII positions 581-594, and amino acid N at the VR-IV position 452;
(ix) amino acid sequence ATNHEDNIRSAQTG at the VR-VIII positions 581-594, and amino acid K at the VR-IV position 452;
(x) amino acid sequence ATNHNNVISGAQTG at the VR-VIII positions 581-594, and amino acid K at the VR-IV position 452;
(xi) amino acid sequence ATNHQGAYAQAQTG at the VR-VIII positions 581-594, and amino acid N at the VR-IV position 452;
(xii) amino acid sequence ATNHQANYGQAQTG at the VR-VIII positions 581-594, and amino acid K at the VR-IV position 452;
(xiii) amino acid sequence ATNHNMNRVNAQTG at the VR-VIII positions 581-594, and amino acid N at the VR-IV position 452;
(xiv) amino acid sequence ATNHNNVISGAQTG at the VR-VIII positions 581-594, and amino acid N at the VR-IV position 452;
(xv) amino acid sequence ATNHSNSVQSAQTG at the VR-VIII positions 581-594, and amino acid N at the VR-IV position 452;
(xvi) amino acid sequence ATNHSSTFQGAQTG at the VR-VIII positions 581-594, and amino acid N at the VR-IV position 452;
(xvii) amino acid sequence ATNHVSSFTSAQTG at the VR-VIII positions 581-594, and amino acid N at the VR-IV position 452;
(xviii) amino acid sequence ATNHSTTNFRAQTG at the VR-VIII positions 581-594, and amino acid N at the VR-IV position 452;
(xix) amino acid sequence ATNHSSIFNSAQTG at the VR-VIII positions 581-594, and amino acid N at the VR-IV position 452;
(xx) amino acid sequence ATNHAGNYNNAQTG at the VR-VIII positions 581-594, and amino acid N at the VR-IV position 452;
(xxi) amino acid sequence ATNHTSVISIAQTG at the VR-VIII positions 581-594, and amino acid N at the VR-IV position 452;
(xxii) amino acid sequence ATNHHSRVEIAQTG at the VR-VIII positions 581-594, and amino acid N at the VR-IV position 452;
(xxiii) amino acid sequence ATNHSSIIYSAQTG at the VR-VIII positions 581-594, and amino acid N at the VR-IV position 452;
(xxiv) amino acid sequence ATNHSGRDSYAQTG at the VR-VIII positions 581-594, and amino acid N at the VR-IV position 452;
(xxv) amino acid sequence ATNHSSSYNNAQTG at the VR-VIII positions 581-594, and amino acid N at the VR-IV position 452;
(xxvi) amino acid sequence ATNHHNPSINAQTG at the VR-VIII positions 581-594, and amino acid N at the VR-IV position 452;
(xxvii) amino acid sequence ATNHNRNGLLAQTG at the VR-VIII positions 581-594, and amino acid N at the VR-IV position 452;
(xxviii) amino acid sequence ATNHESTSVRAQTG at the VR-VIII positions 581-594, and amino acid N at the VR-IV position 452;
(xxix) amino acid sequence ATNHNIRTEMAQTG at the VR-VIII positions 581-594, and amino acid N at the VR-IV position 452;
(xxx) amino acid sequence ATNHQTLFNSAQTG at the VR-VIII positions 581-594, and amino acid N at the VR-IV position 452;
(xxxi) amino acid sequence ATNHLSVSSIAQTG at the VR-VIII positions 581-594, and amino acid N at the VR-IV position 452;
(xxxii) amino acid sequence ATNHEDIIRSAQTG at the VR-VIII positions 581-594, and amino acid N at the VR-IV position 452;
(xxxiii) amino acid sequence ATNRQTAQAQAQTG at the VR-VIII positions 581-594, and amino acid N at the VR-IV position 452; or
(xxxiv) amino acid sequence ATNRQIAQAQAQTG at the VR-VIII positions 581-594, and amino acid N at the VR-IV position 452.

41. The polynucleotide of any one of embodiments 1-8, wherein the capsid protein comprises, relative to reference sequence SEQ ID NO: 1:
(i) an amino acid insertion at position 584 comprising one or more of an asparagine (N), a threonine (T), a tyrosine (Y), phenylalanine (F), and an alanine (A);
(ii) an amino acid insertion at position 585 comprising one or more of a histidine (H) and a methionine (M);
(iii) an amino acid insertion at position 586 comprising one or more of a histidine (H), a tyrosine (Y), a valine (V), a threonine (T), an alanine (A), an isoleucine (I), a tryptophan (W), a methionine (M), and a leucine;
(iv) an amino acid insertion at position 587 comprising one or more of an isoleucine (I) and a proline (P);
(v) an amino acid insertion at position 588 comprising one or more of an isoleucine (I), a threonine (T), and a proline (P); and/or
(vi) an amino acid insertion at position 589 comprising one or more of a glycine (G) and a glutamine (Q).

42. The polynucleotide of embodiment 41, wherein the capsid protein comprises, relative to reference sequence SEQ ID NO: 1:
(i) an amino acid insertion at position 584 consisting of a TY, FN, or AT;
(ii) an amino acid insertion at position 585 consisting of MH;
(iii) an amino acid insertion at position 586 consisting of HY, VT, AI, WM, or ML;
(iv) an amino acid insertion at position 587 consisting of PI; and/or
(v) an amino acid insertion at position 588 consisting of IT or PT.

43. The polynucleotide of any one of embodiments 1-42, wherein the capsid protein shares, or comprises a sequence sharing, at least 90%, at least 95%, at least 96%, at least 97%, at least 99%, or 100% amino acid sequence identity to an AAV9 VP3 sequence according to SEQ ID NO: 487, except for the specified modifications.

44. The polynucleotide of any one of embodiments 1-43, wherein the capsid protein shares, or comprises a sequence sharing, at least 90%, at least 95%, at least 96%, at least 97%, at least 99%, or 100% amino acid sequence identity to an AAV9 VP2 sequence according to SEQ ID NO: 486, except for the specified modifications.

45. The polynucleotide of any one of embodiments 1-44, wherein the capsid protein shares, or comprises a sequence sharing, at least 90%, at least 95%, at least 96%, at least 97%, at least 99%, or 100% amino acid sequence identity to an AAV9 VP1 sequence according to SEQ ID NO: 1, except for the specified modifications.

46. The polynucleotide of any one of embodiments 1-45, wherein the capsid protein comprises, consists essentially of, or consists of an amino acid sequence at least 85%, 90%, 95%, 96%, 97%, 98%, 99%, or 100% identical to any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

47. The polynucleotide of any one of embodiment 1, wherein the capsid protein comprises, consists essentially of, or consists of a polypeptide sequence of any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774.

48. A plasmid or vector comprising the polynucleotide of any one of embodiments 1-47.

49. The plasmid or vector of embodiment 48, which comprises a promoter operably linked to the polynucleotide, optionally wherein the promoter is a promoter suitable for expression in insect cells (e.g., a polyhedrin promoter) or a promoter suitable for expression in mammalian cells.

50. The plasmid or vector of embodiment 48 or 49, which further comprises a polynucleotide encoding a rep protein.

51. A method of generating a producer cell comprising introducing into a cell (e.g., by transfecting the cell) the polynucleotide of any one of embodiments 1-47 or the plasmid or vector of any one of embodiments 48-50, optionally wherein the cell is a mammalian cell or an insect cell, optionally wherein the producer cell is for AAV virion production.

52. The method of embodiment 51, comprising introducing into the cell a polynucleotide, a plasmid or a vector comprising an Adenovirus helper gene.

53. The method of embodiment 51 or 52, comprising introducing into the cell a polynucleotide, a plasmid or a vector comprising a polynucleotide encoding a rep protein.

54. The method of any one of embodiments 51-53, comprising introducing into the cell a polynucleotide, a plasmid or a vector comprising a transgene cassette, wherein the transgene cassette comprises a transgene and inverted terminal repeats or ITRs (e.g., the transgene flanked by the ITRs), optionally wherein the transgene encodes a therapeutic protein.

55. The method of embodiment 54, wherein the ITRs are AAV2 ITRs and wherein the cell comprises a polynucleotide encoding an AAV2 rep protein.

56. A cell comprising the polynucleotide of any one of embodiments 1-47 or the vector of any one of embodiments 48-50.

57. The cell of embodiment 56, which is a mammalian cell, optionally wherein the mammalian cell is a HEK293 cell.

58. The cell of embodiment 56, which is an insect cell, optionally wherein the insect cell is an Sf9 cell.

59. The cell of any one of embodiments 56-58, further comprising one or more of: (i) a polynucleotide, plasmid or vector encoding a rep protein, (ii) a polynucleotide, plasmid or vector comprising an Adenovirus helper gene, and (iii) a polynucleotide, plasmid or vector comprising a transgene cassette comprising a transgene flanked by ITRs (optionally wherein the transgene encodes a therapeutic protein).

60. A method of producing an AAV virion comprising introducing in a cell: (i) the polynucleotide of any one of embodiments 1-47 or the plasmid or vector of any one of embodiments 48-50, (ii) a polynucleotide, plasmid or vector encoding a rep protein, (iii) a polynucleotide, plasmid or vector comprising an Adenovirus helper gene, and (iv) a polynucleotide, plasmid or vector comprising a transgene cassette comprising a transgene flanked by ITRs (optionally wherein the transgene encodes a therapeutic protein); optionally wherein the cell is a mammalian cell or an insect cell, optionally wherein the cell is for AAV virion production.

61. The method of embodiment 59, wherein any of (i)-(iv) are introduced by genomic integration, DNA transfection or viral infection.

62. The method of embodiment 60 or 61, which further comprises culturing the cell under conditions suitable for production and/or packaging of an AAV virion.

63. The method of any one of embodiments 60-62, comprising (i) collecting the produced AAV virion from cell supernatant, and/or (ii) lysing the cell and collecting the produced AAV virion from cell lysate.

64. The method of embodiment 63, comprising purifying the produced AAV virion, optionally wherein the purifying is by density gradient centrifugation and/or a chromatography-based method.

### Definitions

Unless the context indicates otherwise, the features of the invention can be used in any combination. Any feature or combination of features set forth can be excluded or omitted. Certain features of the invention, which are described in separate embodiments may also be provided in combination in a single embodiment. Features of the invention, which are described in a single embodiment may also be provided separately or in any suitable sub-combination. All combinations of the embodiments are disclosed herein as if each and every combination were individually disclosed. All sub-combinations of the embodiments and elements are disclosed herein as if every such sub-combination were individually disclosed.

Unless defined otherwise, all technical and scientific terms used herein have the same meaning as commonly understood by one of ordinary skill in the art to which this invention belongs. The detailed description is divided into sections only for the reader's convenience and disclosure found in any section may be combined with that in another section. Although any methods and materials similar or equivalent to those described herein can also be used in the practice or testing of the present invention, the exemplary methods and materials are now described. All publications mentioned herein are incorporated by reference to disclose and describe the methods and/or materials in connection with which the publications are cited. Reference to a publication is not an admission that the publication is prior art.

The singular forms “a,” “an,” and “the” include plural referents unless the context clearly dictates otherwise. For example, reference to “a recombinant AAV virion” includes a plurality of such virions and reference to “the cardiac cell” includes one or more cardiac cells.

The conjunction “and/or” means both “and” and “or,” and lists joined by “and/or” encompasses all possible combinations of one or more of the listed items.

The term “vector” refers to a macromolecule or complex of molecules comprising a polynucleotide or protein to be delivered to a cell.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9). Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999) J. Virol. 73:3994; Muramatsu et al. (1996) Virol. 221:208; Shade et al. (1986) J. Virol. 58:921; Gao et al. (2002) Proc. Nat. Acad. Sci. USA 99:11854; Moris et al. (2004) Virology 33:375-383; Int'l Pat. Publ Nos. WO2018/222503A1, WO2012/145601A2, WO2000/028061A2, WO1999/61601A2, and WO1998/11244A2; U.S. patent application Ser. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “AAV vector” or “rAAV vector” as used in the art to refer either to the DNA packaged into in the rAAV virion or to the rAAV virion itself, depending on context. As used herein, unless otherwise apparent from context, rAAV vector refers to a nucleic acid (typically a plasmid) comprising a polynucleotide sequence capable of being packaged into an rAAV virion, but with the capsid or other proteins of the rAAV virion. Generally an rAAV vector comprises a heterologous polynucleotide sequence (i.e., a polynucleotide not of AAV origin) and one or two AAV inverted terminal repeat sequences (ITRs) flanking the heterologous polynucleotide sequence. Only one of the two ITRs may be packaged into the rAAV and yet infectivity of the resulting rAAV virion may be maintained. See Wu et al. (2010) Mol Ther. 18:80. An rAAV vector may be designed to generate either single-stranded (ssAAV) or self-complementary (scAAV). See McCarty D. (2008) Mo. Ther. 16:1648-1656; WO2001/11034; WO2001/92551; WO2010/129021.

An “rAAV virion” refers to an extracellular viral particle including at least one viral capsid protein (e.g. VP1) and an encapsidated rAAV vector (or fragment thereof), including the capsid proteins.

For brevity and clarity, the disclosure refers to “capsid protein” or “capsid proteins.” Those skilled in the art understand that such references refer to VP1, VP2, or VP3, or combinations of VP1, VP2, and VP3. As in wild-type AAV and most recombinant expression systems VP1, VP2, and VP3 are expressed from the same open reading frame, engineering of the sequence that encodes VP3 inevitably alters the sequences of the C-terminal domain of VP1 and VP2. One may also express the capsid proteins from different open reading frames, in which case the capsid of the resulting rAAV virion could contain a mixture of wild-type and engineered capsid proteins, and mixtures of different engineered capsid proteins.

Positions with a sequence alignment are generally denotes in terms of a reference sequence. Unless otherwise specified, amino acid positions in the engineered capsid proteins disclosed herein are numbered according to the VP1 sequence of AAV9 provided as SEQ ID NO: 1. Positions may be determined using a best fit alignment of a sequence of interest to a reference sequence. An insertion “at” a position means inserting sequence between that amino acid position and the preceding position in the alignment. The term “about” allows for substitutions or insertions in positions near to the reference position. Those of skill in the art can used techniques such as structural modeling to determine suitable nearby positions (e.g., by identifying the residues in the loop region exposed on the surface of the capsid).

The term “inverted terminal repeats” or “ITRs” as used herein refers to AAV viral cis-elements named so because of their symmetry. These elements are essential for efficient multiplication of an AAV genome. Without being bound by theory, it is believed that the minimal elements indispensable for ITR function are a Rep-binding site and a terminal resolution site plus a variable palindromic sequence allowing for hairpin formation. The disclosure contemplates that alternative means of generating an AAV genome may exist or may be prospectively developed to be compatible with the capsid proteins of the disclosure.

“Helper virus functions” refers to functions encoded in a helper virus genome which allow AAV replication and packaging.

“Packaging” refers to a series of intracellular events that result in the assembly of an rAAV virion including encapsidation of the rAAV vector. AAV “rep” and “cap” genes refer to polynucleotide sequences encoding replication and encapsidation proteins of adeno-associated virus. AAV rep and cap are referred to herein as AAV “packaging genes.” Packaging requires either a helper virus itself or, more commonly in recombinant systems, helper virus function supplied by a helper-free system (i.e. one or more helper plasmids).

A “helper virus” for AAV refers to a virus that allows AAV (e.g. wild-type AAV) to be replicated and packaged by a mammalian cell. The helper viruses may be an adenovirus, herpesvirus or poxvirus, such as vaccinia.

An “infectious” virion or viral particle is one that comprises a competently assembled viral capsid and is capable of delivering a polynucleotide component into a cell for which the virion is tropic. The term does not necessarily imply any replication capacity of the virus.

“Infectivity” refers to a measurement of the ability of a virion to inflect a cell. Infectivity can be expressed as the ratio of infectious viral particles to total viral particles. Infectivity is general determined with respect to a particular cell type. It can be measured both in vivo or in vitro. Methods of determining the ratio of infectious viral particle to total viral particle are known in the art. See, e.g., Grainger et al. (2005) Mol. Ther. 11: S337 (describing a TCID₅₀ infectious titer assay); and Zolotukhin et al. (1999) Gene Ther. 6:973.

The terms “parental capsid” or “parental sequence” refer to a reference sequence from which a particle capsid or sequence is derived. Unless otherwise specified, parental sequence refers to the sequence of the wild-type capsid protein of the same serotype as the engineered capsid protein.

A “replication-competent” virus (e.g. a replication-competent AAV) refers to a virus that is infectious, and is also capable of being replicated in an infected cell (i.e. in the presence of a helper virus or helper virus functions). In some embodiments, the rAAV virion of the disclosure comprises a genome that lacks the rep gene, or both the rep and cap genes, and therefore is replication incompetent.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3^{rd} edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); and Sell (2013) Stem Cells Handbook.

The terms “nucleic acid” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers. Unless otherwise specified or required, any embodiment of the invention described herein that is a polynucleotide encompasses both the double-stranded form and each of two complementary single-stranded forms known or predicted to make up the double-stranded form.

The terms “polypeptide” and “protein,” are used interchangeably herein and refer to a polymeric form of amino acids of any length, which can include genetically coded and non-genetically coded amino acids, chemically or biochemically modified or derivatized amino acids, and polypeptides having modified peptide backbones. The terms also encompass an amino acid polymer that has been modified; for example, disulfide bond formation, glycosylation, lipidation, phosphorylation, or conjugation with a labeling component.

The term “peptide” refers to a short polypeptide, e.g. a peptide having between about 4 and 30 amino acid residues.

The term “isolated” means separated from constituents, cellular and otherwise, in which the virion, cell, tissue, polynucleotide, peptide, polypeptide, or protein is normally associated in nature. For example, an isolated cell is a cell that is separated form tissue or cells of dissimilar phenotype or genotype.

As used herein, “sequence identity” or “identity” refers to the percentage of number of amino acids that are identical between a sequence of interest and a reference sequence. Generally identity is determined by aligning the sequence of interest to the reference sequence, determining the number of amino acids that are identical between the aligned sequences, dividing that number by the total number of amino acids in the reference sequence, and multiplying the result by 100 to yield a percentage. Sequences can be aligned using various computer programs, such BLAST, available at ncbi.nlm.nih.gov. Other techniques for alignment are described in Methods in Enzymology, vol. 266: Computer Methods for Macromolecular Sequence Analysis (1996); and Meth. Mol. Biol. 70:173-187 (1997); J. Mol. Biol. 48:44. Skill artisans are capable of choosing an appropriate alignment method depending on various factors including sequence length, divergence, and the presence of absence of insertions or deletions with respect to the reference sequence.

“Recombinant,” as applied to a polynucleotide means that the polynucleotide is the product of various combinations of cloning, restriction or ligation steps, and other procedures that result in a construct that is distinct from a polynucleotide found in nature, or that the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g. a recombinant capsid protein.

A “gene” refers to a polynucleotide containing at least one open reading frame that is capable of encoding a particular protein after being transcribed and translated. A “gene product” is a molecule resulting from expression of a particular gene. Gene products may include, without limitation, a polypeptide, a protein, an aptamer, an interfering RNA, or an mRNA. Gene-editing systems (e.g. a CRISPR/Cas system) may be described as one gene product or as the several gene products required to make the system (e.g. a Cas protein and a guide RNA).

A “short hairpin RNA,” or shRNA, is a polynucleotide construct used to express an siRNA.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements include transcriptional regulatory sequences such as promoters and/or enhancers.

A “promoter” is a DNA sequence capable under certain conditions of binding RNA polymerase and initiating transcription of a coding region usually located downstream (in the 3′ direction) from the promoter. The term “tissue-specific promoter” as used herein refers to a promoter that is operable in cells of a particular organ or tissue, such as the cardiac tissue.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “polynucleotide cassette” refers to the portion of a vector genome between the inverted terminal repeats (ITRs). A polynucleotide cassette may comprises polynucleotide sequences encoding any genetic element whose delivery to a target cell is desired, including but not limited to a coding sequence for a gene, a promoter, or a repair template for gene editing. Unless otherwise specified, the expression cassette of an AAV vector includes only the polynucleotide between (and not including) the ITRs.

An “expression vector” is a vector comprising a coding sequence which encodes a gene product of interest used to effect the expression of the gene product in target cells. An expression vector comprises control elements operatively linked to the coding sequence to facilitate expression of the gene product.

The term “expression cassette” refers to a polynucleotide cassette comprising a coding sequence which encodes a gene product of interest used to effect the expression of the gene product in target cells. Unless otherwise specified, the expression cassette of an AAV vector includes only the polynucleotides between (and not including) the ITRs.

The term “gene delivery” or “gene transfer” as used herein refers to methods or systems for reliably inserting foreign nucleic acid sequences, e.g., DNA, into host cells. Such methods can result in transient expression of non-integrated transferred DNA, extrachromosomal replication and expression of transferred replicons (e.g., episomes), or integration of transferred genetic material into the genomic DNA of host cells.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid is an rAAV that includes a nucleic acid not normally included in a naturally-occurring AAV.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a vector.

A cell is said to be “stably” altered, transduced, genetically modified, or transformed with a polynucleotide sequence if the sequence is available to perform its function during extended culture of the cell in vitro. Generally, such a cell is “heritably” altered (genetically modified) in that a genetic alteration is introduced which is also inheritable by progeny of the altered cell.

The term “transfection” is as used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells.

The term “transduction” is as used herein refers to the transfer of an exogenous nucleic acid into a cell by a recombinant virion, in contrast to “infection” by a wild-type virion. When infection is used with respect to a recombinant virion, the terms “transduction” and “infectious” are synonymous, and therefore “infectivity” and “transduction efficiency” are equivalent and can be determined using similar methods.

The phrase “assessed in a primate” refers to testing by methods described in the Examples or variations upon them. Assessment may be done using a population of rAAV virions having a common capsid protein screen or pooled testing by re-screening.

Unless otherwise specified, all medical terminology is given the ordinary meaning of the term used by medical professional as, for example, in Harrison's Principles of Internal Medicine, 15ed., which is incorporated by reference in its entirety for all purposes, in particular the chapters on cardiac or cardiovascular diseases, disorders, conditions, and dysfunctions.

“Treatment,” “treating,” and “treat” are defined as acting upon a disease, disorder, or condition with an agent to reduce or ameliorate harmful or any other undesired effects of the disease, disorder, or condition and/or its symptoms.

“Administration,” “administering” and the like, when used in connection with a composition of the invention refer both to direct administration (administration to a subject by a medical professional or by self-administration by the subject) and/or to indirect administration (prescribing a composition to a patient). Typically, an effective amount is administered, which amount can be determined by one of skill in the art. Any method of administration may be used. Administration to a subject can be achieved by, for example, intravenous, intra-arterial, intramuscular, intravascular, or intramyocardial delivery.

As used herein the term “effective amount” and the like in reference to an amount of a composition refers to an amount that is sufficient to induce a desired physiologic outcome (e.g., reprogramming of a cell or treatment of a disease). An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions (e.g., rAAV virions) for any particular subject depends upon a variety of factors including the activity of the specific agent employed, the age, body weight, general health, sex, and diet of the subject, the time of administration, the rate of excretion, the composition combination, severity of the particular disease being treated and form of administration.

The terms “individual,” “subject,” and “patient” are used interchangeably herein, and refer to a mammal, including, but not limited to, human and non-human primates (e.g., simians); mammalian sport animals (e.g., horses); mammalian farm animals (e.g., sheep, goats, etc.); mammalian pets (e.g., dogs, cats, etc.); and rodents (e.g., mice, rats, etc.).

The terms “cardiac pathology” or “cardiac dysfunction” are used interchangeably and refer to any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathies), diseases such as angina pectoris, myocardial ischemia and/or infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart.

As used herein, the term “cardiomyopathy” refers to any disease or dysfunction that affects myocardium directly. The etiology of the disease or disorder may be, for example, inflammatory, metabolic, toxic, infiltrative, fibroplastic, hematological, genetic, or unknown in origin. Two fundamental forms are recognized (1) a primary type, consisting of heart muscle disease of unknown cause; and (2) a secondary type, consisting of myocardial disease of known cause or associated with a disease involving other organ systems. “Specific cardiomyopathy” refers to heart diseases associated with certain systemic or cardiac disorders; examples include hypertensive and metabolic cardiomyopathy. The cardiomyopathies include dilated cardiomyopathy (DCM), a disorder in which left and/or right ventricular systolic pump function is impaired, leading to progressive cardiac enlargement; hypertrophic cardiomyopathy, characterized by left ventricular hypertrophy without obvious causes such as hypertension or aortic stenosis; and restrictive cardiomyopathy, characterized by abnormal diastolic function and excessively rigid ventricular walls that impede ventricular filling. Cardiomyopathies also include left ventricular non-compaction, arrhythmogenic right ventricular cardiomyopathy, and arrhythmogenic right ventricular dysplasia.

“Heart failure” refers to the pathological state in which an abnormality of cardiac function is responsible for failure of the heart to pump blood at a rate commensurate with the requirements of the metabolizing tissues and/or allows the heart to do so only from an abnormally elevated diastolic volume. Heart failure includes systolic and diastolic failure. Patient with heart failure are classified into those with low cardiac output (typically secondary to ischemic heart disease, hypertension, dilated cardiomyopathy, and/or valvular or pericardial disease) and those with elevated cardiac output (typically due to hyperthyroidism, anemia, pregnancy, arteriovenous fistulas, beriberi, and Paget's disease). Heart failure includes heart failure with reduced ejection fraction (HFrEF) and heart failure with preserved ejection fraction (HFpEF).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “purified” as used herein refers to material that has been isolated under conditions that reduce or eliminate the presence of unrelated materials, i.e. impurities, including native materials from which the material is obtained. For example, purified rAAV vector DNA is preferably substantially free of cell or culture components, including tissue culture components, contaminants, and the like.

The terms “regenerate,” “regeneration” and the like as used herein in the context of injured cardiac tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises generation of cardiomyocytes.

The term “therapeutic gene” as used herein refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in a cell or mammal.

As used herein, the term “functional cardiomyocyte” refers to a differentiated cardiomyocyte that is able to send or receive electrical signals. In some embodiments, a cardiomyocyte is said to be a functional cardiomyocyte if it exhibits electrophysiological properties such as action potentials and/or Ca²⁺ transients.

As used herein, a “differentiated non-cardiac cell” can refer to a cell that is not able to differentiate into all cell types of an adult organism (i.e., is not a pluripotent cell), and which is of a cellular lineage other than a cardiac lineage (e.g., a neuronal lineage or a connective tissue lineage). Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

As used herein, the term “totipotent” means the ability of a cell to form all cell lineages of an organism. For example, in mammals, only the zygote and the first cleavage stage blastomeres are totipotent.

As used herein, the term “pluripotent” means the ability of a cell to form all lineages of the body or soma. For example, embryonic stem cells are a type of pluripotent stem cells that are able to form cells from each of the three germs layers, the ectoderm, the mesoderm, and the endoderm. Pluripotent cells can be recognized by their expression of markers such as Nanog and Rex1.

As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “reprogramming” or “transdifferentiation” refers to the generation of a cell of a certain lineage (e.g., a cardiac cell) from a different type of cell (e.g., a fibroblast cell) without an intermediate process of de-differentiating the cell into a cell exhibiting pluripotent stem cell characteristics.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules, e.g., proteins like myosin heavy chain, myosin light chain, cardiac α-actinin. The term “cardiomyocyte” as used herein is an umbrella term comprising any cardiomyocyte subpopulation or cardiomyocyte subtype, e.g., atrial, ventricular and pacemaker cardiomyocytes.

The term “cardiomyocyte-like cells” is intended to mean cells sharing features with cardiomyocytes, but which may not share all features. For example, a cardiomyocyte-like cell may differ from a cardiomyocyte in expression of certain cardiac genes.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g., a pluripotent state, a quiescent state, etc.), to mature cells—in some instances, specifically, to promote the differentiation of progenitor cells into cells of a particular lineage (e.g., a cardiomyocyte).

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample.

The term “induced cardiomyocyte” or the abbreviation “iCM” refers to a non-cardiomyocyte (and its progeny) that has been transformed into a cardiomyocyte (and/or cardiomyocyte-like cell). The methods of the present disclosure can be used in conjunction with any methods now known or later discovered for generating induced cardiomyocytes, for example, to enhance other techniques.

The term “induced pluripotent stem cell-derived cardiomyocytes” as used herein refers to human induced pluripotent stem cells that have been differentiated into cardiomyocyte-like cells. Exemplary methods for prepared iPS-CM cells are provided by Karakikes et al. Circ Res. 2015 Jun. 19; 117 (1): 80-88.

The terms “human cardiac fibroblast” and “mouse cardiac fibroblast” as used herein refer to primary cell isolated from the ventricles of the adult heart of a human or mouse, respectively, and maintain in culture ex vivo.

The term “non-cardiomyocyte” as used herein refers to any cell or population of cells in a cell preparation not fulfilling the criteria of a “cardiomyocyte” as defined and used herein. Non-limiting examples of non-cardiomyocytes include somatic cells, cardiac fibroblasts, non-cardiac fibroblasts, cardiac progenitor cells, and stem cells.

As used herein “reprogramming” includes transdifferentiation, dedifferentiation and the like.

As used herein, the term “reprogramming efficiency” refers to the number of cells in a sample that are successfully reprogrammed to cardiomyocytes relative to the total number of cells in the sample.

The term “reprogramming factor” as used herein includes a factor that is introduced for expression in a cell to assist in the reprogramming of the cell from one cell type into another. For example, a reprogramming factor may include a transcription factor that, in combination with other transcription factors and/or small molecules, is capable of reprogramming a cardiac fibroblast into an induced cardiomyocyte. Unless otherwise clear from context, a reprogramming factor refers to a polypeptide that can be encoded by an AAV-delivered polynucleotide. Reprogramming factors may also include small molecules.

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, deletions, additions, fusions and truncations in the polypeptide encoded by the reference sequence. Generally, differences are limited so that the sequences of the reference polypeptide and the variant are closely similar overall and, in many regions, identical.

As used herein, the term “progenitor cell” refers to a cell that is committed to differentiate into a specific type of cell or to form a specific type of tissue. A progenitor cell, like a stem cell, can further differentiate into one or more kinds of cells, but is more mature than a stem cell such that it has a more limited/restricted differentiation capacity.

The term “genetic modification” refers to a permanent or transient genetic change induced in a cell following introduction of new nucleic acid (i.e., nucleic acid exogenous to the cell). Genetic change can be accomplished by incorporation of the new nucleic acid into the genome of the cardiac cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism. In some embodiments, the compositions for inducing cardiomycocyte phenotype can be used on a population of cells to induce reprogramming. In other embodiments, the compositions induce a cardiomycocyte phenotype.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency. See, for example, Takahashi et al. (2007) Cell 131 (5): 861-872, Kim et al. (2011) Proc. Natl. Acad. Sci. 108 (19): 7838-7843, Sell (2013) Stem Cells Handbook.

The term “transduction efficiency” refers to the percentage of cells transduced with at least one AAV genome. For example, if 1×10⁶ cells are exposed to a virus and 0.5×10⁶ cells are determined to contain at least one copy of the AAV genome, then the transduction efficiency is 50%. An illustrative method for determining transduction efficiency is flow cytometry. For example, the percentage of GFP+ cells is a measure of transduction efficiency when the AAV genome comprises a polynucleotide encoding green fluorescence protein (GFP).

The term “selectivity” refers to the ratio of transduction efficiency for one cell type over another, or over all other cells types.

The term “infectivity” refers to the ability of an AAV virion to infect a cell, in particularly an in vivo cell. Infectivity therefore is a function of, at least, biodistribution and neutralizing antibody escape.

Unless stated otherwise, the abbreviations used throughout the specification have the following meanings: AAV, adeno-associated virus, rAAV, recombinant adeno-associated virus; AHCF, adult human cardiac fibroblast; APCF, adult pig cardiac fibroblast, a-MHC-GFP; alpha-myosin heavy chain green fluorescence protein; CF, cardiac fibroblast; cm, centimeter; CO, cardiac output; EF, ejection fraction; FACS, fluorescence activated cell sorting; GFP, green fluorescence protein; GMT, Gata4, Mef2c and Tbx5; GMTc, Gata4, Mef2c, Tbx5, TGF-βi, WNTi; GO, gene ontology; hCF, human cardiac fibroblast; iCM, induced cardiomyocyte; kg, killigram; μg, microgram; μl, microliter; mg, milligram; ml, milliliter; MI, myocardial infarction; msec, millisecond; min, minute; MyAMT, Myocardin, Ascll, Mef2c and Tbx5; MyA, Myocardin and Ascl1; MyMT, Myocardin, Mef2c and Tbx5; MyMTc, Myocardin, Mef2c, Tbx5, TGF-βi, WNTi; MRI, magnetic resonance imaging; PBS, phosphate buffered saline; PBST, phosphate buffered saline, triton; PFA, paraformaldehyde; qPCR, quantitative polymerase chain reaction; qRT-PCR, quantitative reverse transcriptase polymerase chain reaction; RNA, ribonucleic acid; RNA-seq, RNA sequencing; RT-PCR, reverse transcriptase polymerase chain reaction; sec, second; SV, stroke volume; TGF-β, transforming growth factor beta; TGF-βi, transforming growth factor beta inhibitor; WNT, wingless-Int; WNTi, wingless-Int inhibitor; YFP, yellow fluorescence protein; 4F, Gata4, Mef2c, TBX5, and Myocardin; 4Fc, Gata4, Mef2c, TBX5, and Myocardin+TGF-βi and WNTi; 7F, Gata4, Mef2c, and Tbx5, Essrg, Myocardin, Zfpm2, and Mesp1; 7Fc, Gata4, Mef2c, and Tbx5, Essrg, Myocardin, Zfpm2, and Mesp1+TGF-β and WNTi.

The amino acid abbreviations used herein are abbreviations commonly known and used in the art, and as follows:
Alanine-Ala-A
Arginine-Arg-R
Asparagine-Asn-N
Aspartic acid-Asp-D
Cysteine-Cys-C
Glutamic acid-Glu-E
Glutamine-Gln-Q
Glycine-Gly-G
Histidine-His-H
Isoleucine-Ile-I
Leucine-Leu-L
Lysine-Lys-K
Methionine-Met-M
Phenylalanine-Phe-F
Proline-Pro-P
Serine-Ser-S
Threonine-Thr-T
Tryptophan-Trp-W
Tyrosine-Tyr-Y
Valine-Val-V

Reference to amino acid substitutions are in the format commonly used in the art. E.g., reference to “N452K” substitution, indicates that at position number 452 of the reference sequence, the wild type amino acid in front of the number (here “N”) has been substituted with the amino acid following the number (here “K”).

The term “conservative amino-acid substitutions” refers to substitutions of amino acid residues that share similar sidechain physical properties with the residues being substituted. Conservative substitutions include polar for polar residues, non-polar for non-polar residues, hydrophobic for hydrophobic residues, small for small residues, and large for large residues. Conservative substitutions further comprise substitutions within the following groups: {S, T}, {A, G)}, {F, Y}, {R, H, K, N, E}, {S, T, N, Q}, {C, U, G, P, A}, and {A, V, I, L, M, F, Y, W}.

### Sequences

### Sequences of Certain Recombinant AAV Capsid Proteins:

>ZC377 (SEQ ID NO: 488):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIK
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC378 (SEQ ID NO: 489):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
ASGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC379 (SEQ ID NO: 490):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GTGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC380 (SEQ ID NO: 491):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGLNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC381 (SEQ ID NO: 492):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIA
NDNKLIQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC382 (SEQ ID NO: 493):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIV
NDNKVIQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC383 (SEQ ID NO: 494):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNHQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC384 (SEQ ID NO: 495):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIA
NDNKVIQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQANYGQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC385 (SEQ ID NO: 496):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHTSFQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC386 (SEQ ID NO: 497):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHCSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC387 (SEQ ID NO: 498):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHVDSLRIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC388 (SEQ ID NO: 499):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNRQTAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC389 (SEQ ID NO: 500):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHTGTSIIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC390 (SEQ ID NO: 501):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHLSNFNSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC391 (SEQ ID NO: 502):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHCTLNSIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC392 (SEQ ID NO: 503):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVADVQQKPGSQIQTQWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC393 (SEQ ID NO: 504):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNMNRVNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC394 (SEQ ID NO: 505):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNNVISGAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC395 (SEQ ID NO: 506):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSNSVQSAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC396 (SEQ ID NO: 507):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQSPIAQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC397 (SEQ ID NO: 508):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHLSKVFDAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC398 (SEQ ID NO: 509):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQSAITQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC399 (SEQ ID NO: 510):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSTFQGAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC400 (SEQ ID NO: 511):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNSIQAQAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC401 (SEQ ID NO: 512):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHMMTTARAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC402 (SEQ ID NO: 513):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQGAYAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC403 (SEQ ID NO: 514):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVALNKQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC404 (SEQ ID NO: 515):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHENTVSIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC405 (SEQ ID NO: 516):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHVSSFTSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC406 (SEQ ID NO: 517):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHPSIHQGAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC407 (SEQ ID NO: 518):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSTTNFRAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC408 (SEQ ID NO: 519):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQHYSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC409 (SEQ ID NO: 520):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNKQTAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC410 (SEQ ID NO: 521):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSIFNSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC411 (SEQ ID NO: 522):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHAGNYNNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC412 (SEQ ID NO: 523):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVAEVQQSSMSQAQTDWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC413 (SEQ ID NO: 524):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVAANVQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC414 (SEQ ID NO: 525):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNYQQAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC415 (SEQ ID NO: 526):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQSVQGAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC416 (SEQ ID NO: 527):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHGSILTHAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC417 (SEQ ID NO: 528):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQLFSKNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC418 (SEQ ID NO: 529):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVAANMQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC419 (SEQ ID NO: 530):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNQQIAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC420 (SEQ ID NO: 531):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNTYHQSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC421 (SEQ ID NO: 532):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHCDPLHIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC422 (SEQ ID NO: 533):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHTSVISIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC423 (SEQ ID NO: 534):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQLASAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC424 (SEQ ID NO: 535):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQVTSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC425 (SEQ ID NO: 536):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHHSRVEIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC426 (SEQ ID NO: 537):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHTSFTWTAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC427 (SEQ ID NO: 538):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQSAPTQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC428 (SEQ ID NO: 539):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNSTYLGAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC429 (SEQ ID NO: 540):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQIAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC430 (SEQ ID NO: 541):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQAISAQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC431 (SEQ ID NO: 542):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHLSVVYNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC432 (SEQ ID NO: 543):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHMHQSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC433 (SEQ ID NO: 544):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHETSRLNAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC434 (SEQ ID NO: 545):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVAFNWQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC435 (SEQ ID NO: 546):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNTVMLGAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC436 (SEQ ID NO: 547):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHESSMLNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC437 (SEQ ID NO: 548):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHASITSSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC438 (SEQ ID NO: 549):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVARNEQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC439 (SEQ ID NO: 550):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHANLYQMAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC440 (SEQ ID NO: 551):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQFATAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC441 (SEQ ID NO: 552):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNFNHQSAQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC442 (SEQ ID NO: 553):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHMSHQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC443 (SEQ ID NO: 554):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQWMSAQAQAQTGWVQNQGILPGMVWQDRDVYLQG
PIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLT
RNL
>ZC444 (SEQ ID NO: 555):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQSGQQAQAQTGWVQNQGILPGMVWQDRDVYLQGPI
WAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC445 (SEQ ID NO: 556):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC446 (SEQ ID NO: 557):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHTTKTMFAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC447 (SEQ ID NO: 558):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSIIYSAQTGWVQNQGILPGMVWQDRDVYLQGPIWAK
IPHTDGNFHPSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTRYLTRNL
>ZC448 (SEQ ID NO: 559):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHMLLKSNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC449 (SEQ ID NO: 560):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHESMQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC450 (SEQ ID NO: 561):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQMLSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC451 (SEQ ID NO: 562):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSGRDSYAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC452 (SEQ ID NO: 563):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHINVISGAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC453 (SEQ ID NO: 564):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHVSNQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC454 (SEQ ID NO: 565)
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNTKLAIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC455 (SEQ ID NO: 566):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSSYNNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC456 (SEQ ID NO: 567):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNATHQSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
>ZC457 (SEQ ID NO: 568):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHLRDNISAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC458 (SEQ ID NO: 569):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSSFSVGAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC459 (SEQ ID NO: 570):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHVNRNLSAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC460 (SEQ ID NO: 571):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHHNPSINAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC461 (SEQ ID NO: 572):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQDARAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC462 (SEQ ID NO: 573):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNDQRAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC463 (SEQ ID NO: 574):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNVQTAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC464 (SEQ ID NO: 575):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVAPNRQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC465 (SEQ ID NO: 576):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNRQIAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC466 (SEQ ID NO: 577):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHEDNIRRAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC467 (SEQ ID NO: 578):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNRNGLLAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC468 (SEQ ID NO: 579):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHESTSVRAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC469 (SEQ ID NO: 580):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHNIRTEMAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC470 (SEQ ID NO: 581):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQTLFNSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC471 (SEQ ID NO: 582):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHHSWQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC472 (SEQ ID NO: 583):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHSTKSLIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC473 (SEQ ID NO: 584):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHQKLLVNAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC474 (SEQ ID NO: 585):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHLSVSSIAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC475 (SEQ ID NO: 586):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHVSNLYGAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC476 (SEQ ID NO: 587):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNRQMAQAQAQTGWVQNQGILPGMVWQDRDVYLQGPIW
AKIPHTDGNFHPSPLMGGFGMKHPPPQILIKNTPVPADPPTAFNKDKLNSFITQYSTGQ
VSVEIEWELQKENSKRWNPEIQYTSNYYKSNNVEFAVNTEGVYSEPRPIGTRYLTRNL
>ZC477 (SEQ ID NO: 588):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHEDIIRSAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL
>ZC478 (SEQ ID NO: 589):
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNDGSQAVGRSSF
YCLEYFPSQMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFAWPGASS
WALNGRNSLMNPGPAMASHKEGEDRFFPLSGSLIFGKQGTGRDNVDADKVMITNEE
EIKTTNPVATESYGQVATNHCSTSIRAQTGWVQNQGILPGMVWQDRDVYLQGPIWA
KIPHTDGNFHPSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIGTRYLTRNL

### Additional Sequences

| | | SEQ |
|---|---|---|
| | | ID |
| Description | Sequence | NO |
| Recombinant | MAADGYLPDWLEDNLSEGIREWWALKPGAPQPKANQQHQD | 705 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC373 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHENTVSIAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 706 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC374 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQTLFNSAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 707 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC375 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHNSTYLGAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| Recombinant | MAADGYLPDWLEDNLSEGIREWWALKPGAPQPKANQQHQD | 708 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC376 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHGSILTHAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| AAV capsid | MAADGYLPDWLEDNLSEGIREWWALKPGAPQPKANQQHQD | 709 |
| protein ACE5 | NARGLVLPGYKYLGPGNGLDKGEPVNAADAAALEHDKAYD | |
| | QQLKAGDNPYLKYNHADAEFQERLKEDTSFGGNLGRAVFQA | |
| | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIIGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQANYGQAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| AAV capsid | MAADGYLPDWLEDNLSEGIREWWALKPGAPQPKANQQHQD | 710 |
| protein | NARGLVLPGYKYLGPGNGLDKGEPVNAADAAALEHDKAYD | |
| ACE10 | QQLKAGDNPYLKYNHADAEFQERLKEDTSFGGNLGRAVFQA | |
| | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTINGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHEDNIRSAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | X₁-X₂-X₃-X₄- | 711 |
| AAV capsid | wherein X₁ is S, T or N; X₂ is T, L, or I; X₃ is V, F, Y, or L; and | |
| protein partial | X₄ is A, N, L or T | |
| VR-VIII | | |
| Recombinant | NTVS | 712 |
| AAV capsid | | |
| protein partial | | |
| ZC373 VR- | | |
| VIII | | |
| Recombinant | TLFN | 713 |
| AAV capsid | | |
| protein partial | | |
| ZC374 VR- | | |
| VIII | | |
| Recombinant | STYL | 714 |
| AAV capsid | | |
| protein partial | | |
| ZC375 VR- | | |
| VIII | | |
| Recombinant | SILT | 715 |
| AAV capsid | | |
| protein partial | | |
| ZC376 VR- | | |
| VIII | | |
| Recombinant | MTTA | 716 |
| AAV capsid | | |
| protein partial | | |
| ZC531 VR- | | |
| VIII | | |
| Recombinant | STSI | 717 |
| AAV capsid | | |
| protein partial | | |
| ZC533 VR- | | |
| VIII | | |
| Recombinant | -X₁-X₂-X₃-X₄-X₅-X₆ | 718 |
| AAV capsid | wherein X₁ is Q, E, N, G, M, or C; X₂ is S, N, T, or M; X₃ is A, T, L, | |
| protein partial | I, or S; X₄ is Q, V, F, Y, L, or I; X₅ is A, S, N, L, T, or I; and X₆ | |
| VR-VIII | is I, S, Q, G, H, or R | |
| Recombinant | ENTVSI | 719 |
| AAV capsid | | |
| protein partial | | |
| ZC373 VR- | | |
| VIII | | |
| Recombinant | QTLFNS | 720 |
| AAV capsid | | |
| protein partial | | |
| ZC374 VR- | | |
| VIII | | |
| Recombinant | NSTYLG | 721 |
| AAV capsid | | |
| protein partial | | |
| ZC375 VR- | | |
| VIII | | |
| Recombinant | GSILTH | 722 |
| AAV capsid | | |
| protein partial | | |
| ZC376 VR- | | |
| VIII | | |
| Recombinant | MMTTAR | 723 |
| AAV capsid | | |
| protein partial | | |
| ZC531 VR- | | |
| VIII | | |
| Recombinant | CSTSIR | 724 |
| AAV capsid | | |
| protein partial | | |
| ZC532 VR- | | |
| VIII | | |
| Recombinant | EDNIRS | 725 |
| AAV capsid | | |
| protein partial | | |
| ZC536 VR- | | |
| VIII | | |
| Recombinant | ATNHEDNIRSAQTG | 726 |
| AAV capsid | | |
| protein | | |
| ZC536 VR- | | |
| VIII | | |
| Recombinant | KGSGQNQQT | 727 |
| AAV capsid | | |
| protein VR- | | |
| IV | | |
| Recombinant | ATNH-X₁-X₂-X₃-X₄-X₅-X₆-AQTG | 728 |
| AAV capsid | X₁ is Q, E, N, G, M, C, V, or T; X₂ is S, N, T, M, G, or D; X₃ is A, T, | |
| protein partial | L, I, K, S, N or V; X₄ is Q, V, F, Y, L, T, S, I, R, or Q; X₅ is A, S, | |
| VR-VIII | N, L, T, I, or R, and X₆ is Q, I, S, G, H or R | |
| Recombinant | -X₁-X₂-X₃-X₄-X₅-X₆-X₇-X₈-X₉- | 729 |
| AAV capsid | wherein X₁ is K, G, S or V; X₂ is Y, Q or I; X₃ is H, W, V, or I; X₄ is | |
| protein VR- | Kor N; X₅ is S, G or I; X₆ is G or R; X₇ is A, P or V; X₈ is A or R; | |
| IV | and/or X₉ is Q or D | |
| Recombinant | -X₁-X₂-X₃-X₄-X₅-X₆-X₇-X₈-X₉- | 730 |
| AAV capsid | wherein X₁ is K and X₂-X₉ are any amino acid | |
| protein VR- | | |
| IV | | |
| Recombinant | -X₁-X₂-X₃-X₄- | 731 |
| AAV capsid | wherein X₁ is S, N, T, M, G, or D; X₂ is A, T, L, I, K, S, N or V; X₃ | |
| protein partial | is Q, V, F, Y, L, T, S, I, R, or Q; and X₄ is A, S, N, L, T, I, or R | |
| VR-VIII | | |
| Recombinant | -X₁-X₂-X₃-X₄- | 732 |
| AAV capsid | wherein X₁ is S, N, T, M, G, or D; X₂ is T, L, I, K, S, N or V; X₃ is | |
| protein partial | V, F, Y, L, T, S, I, R, or Q; and X₄ is A, S, N, L, T, I, or R | |
| VR-VIII | | |
| Recombinant | -X₁-X₂-X₃-X₄- | 733 |
| AAV capsid | wherein X₁ is S, N, M, or T; X₂ is A, T, L, or I; X₃ is Q, V, F, Y, T, | |
| protein partial | S, or L; and X₄ is A, S, N, L, I, or T (SEQ ID NO: 733) | |
| VR-VIII | | |
| Recombinant | -X₁-X₂-X₃-X₄- | 734 |
| AAV capsid | wherein X₁ is S, N, M, or T; X₂ is T, L, or I; X₃ is V, F, Y, T, S, or | |
| protein partial | L; and X₄ is A, S, N, L, I, or T | |
| VR-VIII | | |
| Recombinant | -X₁-X₂-X₃-X₄-X₅-X₆ | 735 |
| AAV capsid | wherein X₁ is Q, E, N, G, M, C, V, or T; X₂ is S, N, T, M, G, or D; | |
| protein partial | X₃ is A, T, L, I, K, S, N or V; X₄ is V, F, Y, L, T, S, I, R, or Q; X₅ | |
| VR-VIII | is A, S, N, L, T, I, or R, and X₆ is Q, I, S, G, H or R | |
| Recombinant | -X₁-X₂-X₃-X₄-X₅-X₆ | 736 |
| AAV capsid | wherein X₁ is Q, E, N, G, M, C, V, or T; X₂ is S, N, T, M, G, or D; | |
| protein partial | X₃ is T, L, I, K, S, N or V; X₄ is V, F, Y, L, T, S, I, R, or Q; X₅ is | |
| VR-VIII | A, S, N, L, T, I, or R, and X₆ is I, S, G, H or R | |
| Recombinant | -X₁-X₂-X₃-X₄-X₅-X₆ | 737 |
| AAV capsid | wherein X₁ is Q, E, N, M, C, or G; X₂ is S, N, M, or T; X₃ is A, T, L, | |
| protein partial | or I; X₄ is Q, V, F, Y, T, S, or L; X₅ is A, S, N, L, I, or T; and X₆ | |
| VR-VIII | is I, S, G, R, or H | |
| Recombinant | -X₁-X₂-X₃-X₄-X₅-X₆ | 738 |
| AAV capsid | wherein X₁ is E, N, M, C, or G; X₂ is S, N, M, or T; X₃ is T, L, or I; | |
| protein partial | X₄ is V, F, Y, T, S, or L; X₅ is A, S, N, L, I, or T; and X₆ is I, S, | |
| VR-VIII | G, R, or H | |
| Recombinant | ATNH-X₁-X₂-X₃-X₄-X₅-X₆-AQTG | 739 |
| AAV capsid | wherein X₁ is Q, E, N, G, M, or C; X₂ is S, N, T, or M; X₃ is A, T, L, | |
| protein VR- | I, or S; X₄ is Q, V, F, Y, L, or I; X₅ is A, S, N, L, T, or I; and X₆ | |
| VIII | is I, S, Q, G, H, or R | |
| Recombinant | ATNH-(X)ₙ-AQTG | 740 |
| AAV capsid | wherein n is 4-8, and X represents any of the 20 standard amino acids | |
| protein VR- | | |
| VIII | | |
| Partial protein | GAYA | 741 |
| of AAV | | |
| capsid | | |
| Partial protein | TKLA | 742 |
| of AAV | | |
| capsid | | |
| Partial protein | SSFT | 743 |
| of AAV | | |
| capsid | | |
| Partial protein | DNIR | 744 |
| of AAV | | |
| capsid | | |
| Partial protein | NVIS | 745 |
| of AAV | | |
| capsid | | |
| Partial protein | GTSI | 746 |
| of AAV | | |
| capsid | | |
| Partial protein | DARA | 747 |
| of AAV | | |
| capsid | | |
| Partial protein | SAQA | 748 |
| of AAV | | |
| capsid | | |
| Partial protein | QGAYAQ | 749 |
| of AAV | | |
| capsid | | |
| Partial protein | NTKLAI | 750 |
| of AAV | | |
| capsid | | |
| Partial protein | VSSFTS | 751 |
| of AAV | | |
| capsid | | |
| Partial protein | NNVISG | 752 |
| of AAV | | |
| capsid | | |
| Partial protein | TGTSII | 753 |
| of AAV | | |
| capsid | | |
| Partial protein | QANYGQ | 754 |
| of AAV | | |
| capsid | | |
| Partial protein | QDARAQ | 755 |
| of AAV | | |
| capsid | | |
| Partial protein | QSAQAQ | 756 |
| of AAV | | |
| capsid | | |
| Partial protein | KYHKSGAAQ | 757 |
| of AAV | | |
| capsid | | |
| Partial protein | KQVNGRPRD | 758 |
| of AAV | | |
| capsid | | |
| Partial protein | QHYSAQAQ | 759 |
| of AAV | | |
| capsid | | |
| Recombinant | -X₁-X₂-X₃-X₄- | 760 |
| AAV capsid | wherein X₁ is S, M, D, N, G, A,T, R, or I; X₂ is T, N, V, A, L, I, S, | |
| protein partial | R, or P; X₃ is Y, T, S, I, V, F, L, R, N, D, G, or Q; and X₄ is L, A, | |
| VR-VIII | I, R, S, G, N, T, V, Q, F, E, or Y | |
| Recombinant | -X₁-X₂-X₃-X₄- | 761 |
| AAV capsid | wherein X₁ is S, M, D, N, G, or A; X₂ is T, N, V, or A; X₃ is Y, T, S, | |
| protein partial | I, or V; and X₄ is L, A, I, R, S, or G | |
| VR-VIII | | |
| Recombinant | -X₁-X₂-X₃-X₄-X₅-X₆- | 762 |
| AAV capsid | wherein X₁ is N, M, C, E, G, S, V, A, T, H, L, or Q; X₂ is M, D, N, | |
| protein partial | G, A, T, R, I, or S; X₃ is T, N, V, L, I, S, R, P, or A; X₄ is Y, T, | |
| VR-VIII | S, I, V, F, L, R, N, D, G, or Q; X₅ is L, I, R, S, G, N, T, V, Q, F, | |
| | E, Y, or A, and X₆ is G, R, S, I, H, N, Y, L, M, or Q | |
| Recombinant | -X₁-X₂-X₃-X₄-X₅-X₆- | 763 |
| AAV capsid | wherein X₁ is N, M, C, E, G, S, V, A, T, H, or L; X₂ is M, D, N, G, | |
| protein partial | A, T, R, or I; X₃ is T, N, V, L, I, S, R, or P; X₄ is Y, T, S, I, V, | |
| VR-VIII | F, L, R, N, D, or G; X₅ is L, I, R, S, G, N, T, V, Q, F, E, or Y, and | |
| | X₆ is G, R, S, I, H, N, Y, L, or M | |
| Recombinant | -X₁-X₂-X₃-X₄-X₅-X₆- | 764 |
| AAV capsid | wherein X₁ is E, N, G, M, C, V, or T; X₂ is N, T, M, G, or D; X₃ is T, | |
| protein partial | L, I, K, S, N or V; X₄ is V, F, Y, L, T, S, I, R; X₅ is S, N, L, T, I, | |
| VR-VIII | or R, and X₆ is I, S, G, H or R | |
| Recombinant | -X₁-X₂-X₃-X₄-X₅-X₆- | 765 |
| AAV capsid | wherein X₁ is E, N, M, C, or Q; X₂ is A, M, G, D, N, or S; X₃ is T, N, | |
| protein partial | V, or A; X₄ is V, Y, T, S, I, or Q; X₅ is S, G, L, I, R, or A; and X₆ | |
| VR-VIII | is I, S, G, R, or Q | |
| Recombinant | -X₁-X₂-X₃-X₄-X₅-X₆- | 766 |
| AAV capsid | wherein X₁ is E, N, M, or C; X₂ is A, M, G, D, or N; X₃ is T, N, or V; | |
| protein partial | X₄ is V, Y, T, S, or I; X₅ is S, G, L, I, or R; and X₆ is I, S, G, or | |
| VR-VIII | R | |
| Recombinant | MAADGYLPDWLEDNLSEGIREWWALKPGAPQPKANQQHQD | 767 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC531 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHMMTTARAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| Recombinant | MAADGYLPDWLEDNLSEGIREWWALKPGAPQPKANQQHQD | 768 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC532 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHCSTSIRAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 769 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC533 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQGAYAQAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| Recombinant | MAADGYLPDWLEDNLSEGIREWWALKPGAPQPKANQQHQD | 770 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC534 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHNTKLAIAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 771 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC535 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHVSSFTSAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 772 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC536 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHEDNIRSAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 773 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC537 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQSAQAQAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| Recombinant | MAADGYLPDWLEDNLSEGIREWWALKPGAPQPKANQQHQD | 774 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC538 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHNNVISGAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 775 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC539 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHTGTSIIAQTGWVQNQGILPGMVWQDRDVYLQGPIWAKIPH | |
| | TDGNFHPSPLMGGFGMKHPPPQILIKNTPVPADPPTAFNKDKL | |
| | NSFITQYSTGQVSVEIEWELQKENSKRWNPEIQYTSNYYKSNN | |
| | VEFAVNTEGVYSEPRPIGTRYLTRNL | |
| Recombinant | MAADGYLPDWLEDNLSEGIREWWALKPGAPQPKANQQHQD | 776 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC540 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQWMSAQAQAQTGWVQNQGILPGMVWQDRDVYLQGPIW | |
| | AKIPHTDGNFHPSPLMGGFGMKHPPPQILIKNTPVPADPPTAF | |
| | NKDKLNSFITQYSTGQVSVEIEWELQKENSKRWNPEIQYTSN | |
| | YYKSNNVEFAVNTEGVYSEPRPIGTRYLTRNL | |
| Recombinant | MAADGYLPDWLEDNLSEGIREWWALKPGAPQPKANQQHQD | 777 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC541 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | 778 |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQDARAQAQTGWVQNQGILPGMVWQDRDVYLQGPIWAKI | |
| | PHTDGNFHPSPLMGGFGMKHPPPQILIKNTPVPADPPTAFNKD | |
| | KLNSFITQYSTGQVSVEIEWELQKENSKRWNPEIQYTSNYYKS | |
| | NNVEFAVNTEGVYSEPRPIGTRYLTRNL | |
| Recombinant | MAADGYLPDWLEDNLSEGIREWWALKPGAPQPKANQQHQD | |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC542 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHQHYSAQAQAQTGWVQNQGILPGMVWQDRDVYLQGPIW | |
| | AKIPHTDGNFHPSPLMGGFGMKHPPPQILIKNTPVPADPPTAF | |
| | NKDKLNSFITQYSTGQVSVEIEWELQKENSKRWNPEIQYTSN | |
| | YYKSNNVEFAVNTEGVYSEPRPIGTRYLTRNL | |
| Recombinant | MAADGYLPDWLEDNLSEGIREWWALKPGAPQPKANQQHQD | 779 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC369 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHNIRTEMAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | MAADGYLPDWLEDNLSEGIREWWALKPGAPQPKANQQHQD | 780 |
| AAV capsid | NARGLVLPGYKYLGPGNGLDKGEPVNAADAAALEHDKAYD | |
| protein | QQLKAGDNPYLKYNHADAEFQERLKEDTSFGGNLGRAVFQA | |
| ZC370 | KKRLLEPLGLVEEAAKTAPGKKRPVEQSPQEPDSSAGIGKSGA | |
| | QPAKKRLNFGQTGDTESVPDPQPIGEPPAAPSGVGSLTMASG | |
| | GGAPVADNNEGADGVGSSSGNWHCDSQWLGDRVITTSTRT | |
| | WALPTYNNHLYKQISNSTSGGSSNDNAYFGYSTPWGYFDFNR | |
| | FHCHFSPRDWQRLINNNWGFRPKRLNFKLFNIQVKEVTDNNG | |
| | VKTIANNLTSTVQVFTDSDYQLPYVLGSAHEGCLPPFPADVF | |
| | MIPQYGYLTLNDGSQAVGRSSFYCLEYFPSQMLRTGNNFQFS | |
| | YEFENVPFHSSYAHSQSLDRLMNPLIDQYLYYLSKTIKGSGQN | |
| | QQTLKFSVAGPSNMAVQGRNYIPGPSYRQQRVSTTVTQNNNS | |
| | EFAWPGASSWALNGRNSLMNPGPAMASHKEGEDRFFPLSGS | |
| | LIFGKQGTGRDNVDADKVMITNEEEIKTTNPVATESYGQVAT | |
| | NHSTTNFRAQTGWVQNQGILPGMVWQDRDVYLQGPIWAKIP | |
| | HTDGNFHPSPLMGGFGMKHPPPQILIKNTPVPADPPTAFNKDK | |
| | LNSFITQYSTGQVSVEIEWELQKENSKRWNPEIQYTSNYYKSN | |
| | NVEFAVNTEGVYSEPRPIGTRYLTRNL | |
| Recombinant | -X₁-X₂-X₃-X₄-X₅-X₆-X₇- | 781 |
| AAV capsid | wherein X₁ is R or H; X₂ is N, M, C, E, G, S, V, A, T, H, L, or Q; | |
| protein partial | X₃ is M, D, N, G, A, T, R, I, or S; X₄ is T, N, V, L, I, S, R, P, or | |
| VR-VIII | A; X₅ is Y, T, S, I, V, F, L, R, N, D, G, or Q; X₆ is L, I, R, S, G, | |
| | N, T, V, Q, F, E, Y, or A, and X₇ is G, R, S, I, H, N, Y, L, M, or Q | |
| | | SEQ |
| | | ID |
| Description | Sequence | NO |
| Human | MAEKAGSTFSHLLVPILLLIGWIVGCIIMIYVVFS | 826 |
| DWORF | | |
| Protein | | |
| Human | ATGGCTGAAAAAGCGGGGTCTACATTTTCACACCTTCTGGTTCC | 827 |
| DWORF | TATTCTTCTCCTGATTGGCTGGATTGTGGGCTGCATCATAATGA | |
| DNA | TTTATGTTGTCTTCTCTTAG | |
| Human | ATGGCCGAGAAGGCCGGATCTACCTTCAGCCACCTGCTGGTCC | 828 |
| DWORF | CTATTCTGCTGCTGATCGGCTGGATCGTGGGCTGCATCATCATG | |
| DNA | ATCTACGTGGTGTTCAGCTGA | |
| codon | | |
| optimized | | |
| Human | ATGAGTGGGGGCCGCTTCGACTTTGATGATGGAGGGGCGT | 782 |
| JPH2 DNA | ACTGCGGGGGCTGGGAGGGGGGAAAGGCCCATGGGCATG | |
| (the N- | GACTGTGCACAGGCCCCAAGGGCCAGGGCGAATACTCTGG | |
| terminal | CTCCTGGAACTTTGGCTTTGAGGTGGCAGGTGTCTACACCT | |
| part of the | GGCCCAGCGGAAACACCTTTGAGGGATACTGGAGCCAGGG | |
| sequence | CAAACGGCATGGGCTGGGCATAGAGACCAAGGGGCGCTGG | |
| that, in | CTCTACAAGGGCGAGTGGACACATGGCTTCAAGGGACGCT | |
| some | ACGGAATCCGGCAGAGCTCAAGCAGCGGTGCCAAGTATGA | |
| instances, | GGGCACCTGGAACAATGGCCTGCAAGACGGCTATGGCACC | |
| can be used | GAGACCTATGCTGATGGAGGGACGTACCAAGGCCAGTTCA | |
| on its own, | CCAACGGCATGCGCCATGGCTACGGAGTACGCCAGAGCGT | |
| as an | GCCCTACGGGATGGCCGTGGTGGTGCGCTCGCCGCTGCGC | |
| alternative | ACGTCGCTGTCGTCCCTGCGCAGCGAGCACAGCAACGGCA | |
| to the full- | CGGTGGCCCCGGACTCTCCCGCCTCGCCGGCCTCCGACGG | |
| length | CCCCGCGCTGCCCTCGCCCGCCATCCCGCGTGGCGGCTTC | |
| JPH2, is | GCGCTCAGCCTCCTGGCCAATGCCGAGGCGGCCGCGCGGG | |
| shown in | CGCCCAAGGGCGGCGGCCTCTTCCAGCGGGGCGCGCTGCT | |
| bold) | GGGCAAGCTGCGGCGCGCAGAGTCGCGCACGTCCGTGGGT | |
| | AGCCAGCGCAGCCGTGTCAGCTTCCTTAAGAGCGACCTCAG | |
| | CTCGGGCGCCAGCGACGCCGCGTCCACCGCCAGCCTGGGA | |
| | GAGGCCGCCGAGGGCGCCGACGAGGCCGCACCCTTCGAGG | |
| | CCGATATCGACGCCACCACCACCGAGACCTACATGGGCGA | |
| | GTGGAAGAACGACAAACGCTCGGGCTTCGGCGTGAGCGAA | |
| | CGCTCCAGTGGCCTCCGCTACGAGGGCGAGTGGCTGGACA | |
| | ACCTGCGCCACGGCTATGGCTGCACCACGCTGCCCGACGG | |
| | CCACCGCGAGGAGGGCAAGTACCGCCACAACGTGCTGGTC | |
| | AAGGACACCAAGCGCCGCATGCTGCAGCTCAAGAGCAACA | |
| | AGGTCCGCCAGAAAGTGGAGCACAGTGTGGAGGGTGCCCA | |
| | GCGCGCCGCTGCTATCGCGCGCCAGAAGGCCGAGATTGCC | |
| | GCCTCCAGGACAAGCCACGCCAAGGCCAAAGCTGAGGCAG | |
| | CGGAACAGGCCGCCCTGGCTGCCAACCAGGAGTCCAACAT | |
| | TGCTCGCACTTTGGCCAGGGAGCTGGCTCCGGACTTCTACC | |
| | AGCCAGGTCCGGAATATCAGAAGCGCCGGCTGCTGCAGGA | |
| | GATCCTGGAGAACTCGGAGAGCCTGCTGGAGCCCCCCGAC | |
| | CGGGGCGCCGGCGCAGCGGGCCTCCCACAGCCGCCCCGCG | |
| | AGAGCCCGCAGCTGCACGAGCGTGAGACCCCTCGGCCCGA | |
| | GGGTGGCTCCCCGTCACCGGCCGGGACGCCCCCGCAGCCC | |
| | AAGCGGCCCAGGCCCGGGGTGTCCAAGGACGGCCTGCTGA | |
| | GCCCAGGCGCCTGGAACGGCGAGCCCAGCGGTGAGGGCAG | |
| | CCGGTCAGTCACTCCGTCCGAGGGCGCGGGCCGCCGCAGC | |
| | CCCGCGCGTCCAGCCACCGAGCGCATGGCCATCGAGGCTC | |
| | TGCAGGCACCGCCTGCGCCGTCGCGGGAGCCGGAGGTGGC | |
| | GCTTTACCAGGGCTACCACAGCTATGCTGTGCGCACCACGC | |
| | CGCCCGAGCCCCCACCCTTTGAGGACCAGCCCGAGCCCGAGGT | |
| | CTCCGGGTCCGAGTCCGCGCCCTCGTCCCCGGCCACCGCCCCGC | |
| | TGCAGGCCCCCACGCTCCGAGGCCCCGAGCCTGCACGCGAGAC | |
| | CCCCGCCAAGCTGGAGCCCAAGCCCATCATCCCCAAAGCCGAG | |
| | CCCAGGGCCAAGGCCCGCAAGACTGAGGCTCGAGGGCTGACC | |
| | AAGGCGGGGGCCAAGAAGAAGGCGCGGAAGGAGGCCGCACTG | |
| | GCGGCAGAGGCGGAGGTGGAGGTGGAAGAGGTCCCCAACACC | |
| | ATCCTCATCTGCATGGTGATCCTGCTGAACATCGGCCTGGCCAT | |
| | CCTCTTTGTTCACCTCCTGACCTGA | |
| Human | MSGGRFDFDDGGAYCGGWEGGKAHGHGLCTGPKGQGEYSG | 783 |
| JPH2 | SWNFGFEVAGVYTWPSGNTFEGYWSQGKRHGLGIETKGRWL | |
| protein | YKGEWTHGFKGRYGIRQSSSSGAKYEGTWNNGLQDGYGTET | |
| (the N- | YADGGTYQGQFTNGMRHGYGVRQSVPYGMAVVVRSPLRTSL | |
| terminal | SSLRSEHSNGTVAPDSPASPASDGPALPSPAIPRGGFALSLLANA | |
| part of the | EAAARAPKGGGLFQRGALLGKLRRAESRTSVGSQRSRVSFLK | |
| sequence | SDLSSGASDAASTASLGEAAEGADEAAPFEADIDATTTETYMG | |
| that, in | EWKNDKRSGFGVSERSSGLRYEGEWLDNLRHGYGCTTLPDG | |
| some | HREEGKYRHNVLVKDTKRRMLQLKSNKVRQKVEHSVEGAQR | |
| instances, | AAAIARQKAEIAASRTSHAKAKAEAAEQAALAANQESNIARTL | |
| can be used | ARELAPDFYQPGPEYQKRRLLQEILENSESLLEPPDRGAGAAG | |
| on its own, | LPQPPRESPQLHERETPRPEGGSPSPAGTPPQPKRPRPGVSKDG | |
| as an | LLSPGAWNGEPSGEGSRSVTPSEGAGRRSPARPATERMAIEAL | |
| alternative | QAPPAPSREPEVALYQGYHSYAVRTTPPEPPPFEDQPEPEVSGSE | |
| to the full- | SAPSSPATAPLQAPTLRGPEPARETPAKLEPKPIIPKAEPRAKARKT | |
| length | EARGLTKAGAKKKARKEAALAAEAEVEVEEVPNTILICMVILLNI | |
| JPH2, is | GLAILFVHLLT | |
| shown in | | |
| bold) | | |
| Human | MSAATHSPMMQVASGNGDRDPLPPGWEIKIDPQTGWPFFVDHNS | 784 |
| BAG3 | RTTTWNDPRVPSEGPKETPSSANGPSREGSRLPPAREGHPVYPQLR | |
| protein | PGYIPIPVLHEGAENRQVHPFHVYPQPGMQRFRTEAAAAAPQRSQ | |
| | SPLRGMPETTQPDKQCGQVAAAAAAQPPASHGPERSQSPAASDCS | |
| | SSSSSASLPSSGRSSLGSHQLPRGYISIPVIHEQNVTRPAAQPSFHQA | |
| | QKTHYPAQQGEYQTHQPVYHKIQGDDWEPRPLRAASPFRSSVQG | |
| | ASSREGSPARSSTPLHSPSPIRVHTVVDRPQQPMTHRETAPVSQPE | |
| | NKPESKPGPVGPELPPGHIPIQVIRKEVDSKPVSQKPPPPSEKVEVK | |
| | VPPAPVPCPPPSPGPSAVPSSPKSVATEERAAPSTAPAEATPPKPGE | |
| | AEAPPKHPGVLKVEAILEKVQGLEQAVDNFEGKKTDKKYLMIEE | |
| | YLTKELLALDSVDPEGRADVRQARRDGVRKVQTILEKLEQKAIDV | |
| | PGQVQVYELQPSNLEADQPLQAIMEMGAVAADKGKKNAGNAED | |
| | PHTETQQPEATAAATSNPSSMTDTPGNPAAP | |
| Human | ATGAGCGCCGCCACCCACTCGCCCATGATGCAGGTGGCGTCCG | 785 |
| BAG3 | GCAACGGTGACCGCGACCCTTTGCCCCCCGGATGGGAGATCAA | |
| DNA | GATCGACCCGCAGACCGGCTGGCCCTTCTTCGTGGACCACAAC | |
| | AGCCGCACCACTACGTGGAACGACCCGCGCGTGCCCTCTGAGG | |
| | GCCCCAAGGAGACTCCATCCTCTGCCAATGGCCCTTCCCGGGA | |
| | GGGCTCTAGGCTGCCGCCTGCTAGGGAAGGCCACCCTGTGTAC | |
| | CCCCAGCTCCGACCAGGCTACATTCCCATTCCTGTGCTCCATGA | |
| | AGGCGCTGAGAACCGGCAGGTGCACCCTTTCCATGTCTATCCC | |
| | CAGCCTGGGATGCAGCGATTCCGAACTGAGGCGGCAGCAGCGG | |
| | CTCCTCAGAGGTCCCAGTCACCTCTGCGGGGCATGCCAGAAAC | |
| | CACTCAGCCAGATAAACAGTGTGGACAGGTGGCAGCGGCGGC | |
| | GGCAGCCCAGCCCCCAGCCTCCCACGGACCTGAGCGGTCCCAG | |
| | TCTCCAGCTGCCTCTGACTGCTCATCCTCATCCTCCTCGGCCAG | |
| | CCTGCCTTCCTCCGGCAGGAGCAGCCTGGGCAGTCACCAGCTC | |
| | CCGCGGGGGTACATCTCCATTCCGGTGATACACGAGCAGAACG | |
| | TTACCCGGCCAGCAGCCCAGCCCTCCTTCCACCAAGCCCAGAA | |
| | GACGCACTACCCAGCGCAGCAGGGGGAGTACCAGACCCACCA | |
| | GCCTGTGTACCACAAGATCCAGGGGGATGACTGGGAGCCCCGG | |
| | CCCCTGCGGGCGGCATCCCCGTTCAGGTCATCTGTCCAGGGTGC | |
| | ATCGAGCCGGGAGGGCTCACCAGCCAGGAGCAGCACGCCACTC | |
| | CACTCCCCCTCGCCCATCCGTGTGCACACCGTGGTCGACAGGCC | |
| | TCAGCAGCCCATGACCCATCGAGAAACTGCACCTGTTTCCCAG | |
| | CCTGAAAACAAACCAGAAAGTAAGCCAGGCCCAGTTGGACCA | |
| | GAACTCCCTCCTGGACACATCCCAATTCAAGTGATCCGCAAAG | |
| | AGGTGGATTCTAAACCTGTTTCCCAGAAGCCCCCACCTCCCTCT | |
| | GAGAAGGTAGAGGTGAAAGTTCCCCCTGCTCCAGTTCCTTGTC | |
| | CTCCTCCCAGCCCTGGCCCTTCTGCTGTCCCCTCTTCCCCCAAG | |
| | AGTGTGGCTACAGAAGAGAGGGCAGCCCCCAGCACTGCCCCTG | |
| | CAGAAGCTACACCTCCAAAACCAGGAGAAGCCGAGGCTCCCCC | |
| | AAAACATCCAGGAGTGCTGAAAGTGGAAGCCATCCTGGAGAA | |
| | GGTACAGGGGCTGGAGCAGGCTGTAGACAACTTTGAAGGCAA | |
| | GAAGACTGACAAAAAGTACCTGATGATCGAAGAGTATTTGACC | |
| | AAAGAGCTGCTGGCCCTGGATTCAGTGGACCCCGAGGGACGAG | |
| | CCGATGTGCGTCAGGCCAGGAGAGACGGTGTCAGGAAGGTTCA | |
| | GACCATCTTGGAAAAACTTGAACAGAAAGCCATTGATGTCCCA | |
| | GGTCAAGTCCAGGTCTATGAACTCCAGCCCAGCAACCTTGAAG | |
| | CAGATCAGCCACTGCAGGCAATCATGGAGATGGGTGCCGTGGC | |
| | AGCAGACAAGGGCAAGAAAAATGCTGGAAATGCAGAAGATCC | |
| | CCACACAGAAACCCAGCAGCCAGAAGCCACAGCAGCAGCGAC | |
| | TTCAAACCCCAGCAGCATGACAGACACCCCTGGTAACCCAGCA | |
| | GCACCGTAG | |
| Human | MDIAIHHPWIRRPFFPFHSPSRLFDQFFGEHLLESDLFPTSTSLSPFY | 786 |
| CRYAB | LRPPSFLRAPSWFDTGLSEMRLEKDRFSVNLDVKHFSPEELKVKV | |
| protein | LGDVIEVHGKHEERQDEHGFISREFHRKYRIPADVDPLTITSSLSSD | |
| | GVLTVNGPRKQVSGPERTIPITREEKPAVTAAPKK | |
| Human | ATGGACATCGCCATCCACCACCCCTGGATCCGCCGCCCCTTCTT | 787 |
| CRYAB | TCCTTTCCACTCCCCCAGCCGCCTCTTTGACCAGTTCTTCGGAG | |
| DNA | AGCACCTGTTGGAGTCTGATCTTTTCCCGACGTCTACTTCCCTG | |
| | AGTCCCTTCTACCTTCGGCCACCCTCCTTCCTGCGGGCACCCAG | |
| | CTGGTTTGACACTGGACTCTCAGAGATGCGCCTGGAGAAGGAC | |
| | AGGTTCTCTGTCAACCTGGATGTGAAGCACTTCTCCCCAGAGG | |
| | AACTCAAAGTTAAGGTGTTGGGAGATGTGATTGAGGTGCATGG | |
| | AAAACATGAAGAGCGCCAGGATGAACATGGTTTCATCTCCAGG | |
| | GAGTTCCACAGGAAATACCGGATCCCAGCTGATGTAGACCCTC | |
| | TCACCATTACTTCATCCCTGTCATCTGATGGGGTCCTCACTGTG | |
| | AATGGACCAAGGAAACAGGTCTCTGGCCCTGAGCGCACCATTC | |
| | CCATCACCCGTGAAGAGAAGCCTGCTGTCACCGCAGCCCCCAA | |
| | GAAATAG | |
| Human | METPSQRRATRSGAQASSTPLSPTRITRLQEKEDLQELNDRLAVYI | 788 |
| LMNA | DRVRSLETENAGLRLRITESEEVVSREVSGIKAAYEAELGDARKTL | |
| LaminA | DSVAKERARLQLELSKVREEFKELKARNTKKEGDLIAAQARLKDL | |
| protein | EALLNSKEAALSTALSEKRTLEGELHDLRGQVAKLEAALGEAKK | |
| | QLQDEMLRRVDAENRLQTMKEELDFQKNIYSEELRETKRRHETRL | |
| | VEIDNGKQREFESRLADALQELRAQHEDQVEQYKKELEKTYSAK | |
| | LDNARQSAERNSNLVGAAHEELQQSRIRIDSLSAQLSQLQKQLAA | |
| | KEAKLRDLEDSLARERDTSRRLLAEKEREMAEMRARMQQQLDEY | |
| | QELLDIKLALDMEIHAYRKLLEGEEERLRLSPSPTSQRSRGRASSHS | |
| | SQTQGGGSVTKKRKLESTESRSSFSQHARTSGRVAVEEVDEEGKF | |
| | VRLRNKSNEDQSMGNWQIKRQNGDDPLLTYRFPPKFTLKAGQVV | |
| | TIWAAGAGATHSPPTDLVWKAQNTWGCGNSLRTALINSTGEEVA | |
| | MRKLVRSVTVVEDDEDEDGDDLLHHHHGSHCSSSGDPAEYNLRS | |
| | RTVLCGTCGQPADKASASGSGAQVGGPISSGSSASSVTVTRSYRSV | |
| | GGSGGGSFGDNLVTRSYLLGNSSPRTQSPQNCSIM | |
| Human | ATGGAGACCCCGTCCCAGCGGCGCGCCACCCGCAGCGGGGCGC | 789 |
| LMNA | AGGCCAGCTCCACTCCGCTGTCGCCCACCCGCATCACCCGGCT | |
| LaminA | GCAGGAGAAGGAGGACCTGCAGGAGCTCAATGATCGCTTGGC | |
| DNA | GGTCTACATCGACCGTGTGCGCTCGCTGGAAACGGAGAACGCA | |
| | GGGCTGCGCCTTCGCATCACCGAGTCTGAAGAGGTGGTCAGCC | |
| | GCGAGGTGTCCGGCATCAAGGCCGCCTACGAGGCCGAGCTCGG | |
| | GGATGCCCGCAAGACCCTTGACTCAGTAGCCAAGGAGCGCGCC | |
| | CGCCTGCAGCTGGAGCTGAGCAAAGTGCGTGAGGAGTTTAAGG | |
| | AGCTGAAAGCGCGCAATACCAAGAAGGAGGGTGACCTGATAG | |
| | CTGCTCAGGCTCGGCTGAAGGACCTGGAGGCTCTGCTGAACTC | |
| | CAAGGAGGCCGCACTGAGCACTGCTCTCAGTGAGAAGCGCACG | |
| | CTGGAGGGCGAGCTGCATGATCTGCGGGGCCAGGTGGCCAAGC | |
| | TTGAGGCAGCCCTAGGTGAGGCCAAGAAGCAACTTCAGGATGA | |
| | GATGCTGCGGCGGGTGGATGCTGAGAACAGGCTGCAGACCATG | |
| | AAGGAGGAACTGGACTTCCAGAAGAACATCTACAGTGAGGAG | |
| | CTGCGTGAGACCAAGCGCCGTCATGAGACCCGACTGGTGGAGA | |
| | TTGACAATGGGAAGCAGCGTGAGTTTGAGAGCCGGCTGGCGGA | |
| | TGCGCTGCAGGAACTGCGGGCCCAGCATGAGGACCAGGTGGA | |
| | GCAGTATAAGAAGGAGCTGGAGAAGACTTATTCTGCCAAGCTG | |
| | GACAATGCCAGGCAGTCTGCTGAGAGGAACAGCAACCTGGTGG | |
| | GGGCTGCCCACGAGGAGCTGCAGCAGTCGCGCATCCGCATCGA | |
| | CAGCCTCTCTGCCCAGCTCAGCCAGCTCCAGAAGCAGCTGGCA | |
| | GCCAAGGAGGCGAAGCTTCGAGACCTGGAGGACTCACTGGCCC | |
| | GTGAGCGGGACACCAGCCGGCGGCTGCTGGCGGAAAAGGAGC | |
| | GGGAGATGGCCGAGATGCGGGCAAGGATGCAGCAGCAGCTGG | |
| | ACGAGTACCAGGAGCTTCTGGACATCAAGCTGGCCCTGGACAT | |
| | GGAGATCCACGCCTACCGCAAGCTCTTGGAGGGCGAGGAGGA | |
| | GAGGCTACGCCTGTCCCCCAGCCCTACCTCGCAGCGCAGCCGT | |
| | GGCCGTGCTTCCTCTCACTCATCCCAGACACAGGGTGGGGGCA | |
| | GCGTCACCAAAAAGCGCAAACTGGAGTCCACTGAGAGCCGCA | |
| | GCAGCTTCTCACAGCACGCACGCACTAGCGGGCGCGTGGCCGT | |
| | GGAGGAGGTGGATGAGGAGGGCAAGTTTGTCCGGCTGCGCAA | |
| | CAAGTCCAATGAGGACCAGTCCATGGGCAATTGGCAGATCAAG | |
| | CGCCAGAATGGAGATGATCCCTTGCTGACTTACCGGTTCC | |
| | CACCAAAGTTCACCCTGAAGGCTGGGCAGGTGGTGACGATCTG | |
| | GGCTGCAGGAGCTGGGGCCACCCACAGCCCCCCTACCGACCTG | |
| | GTGTGGAAGGCACAGAACACCTGGGGCTGCGGGAACAGCCTG | |
| | CGTACGGCTCTCATCAACTCCACTGGGGAAGAAGTGGCCATGC | |
| | GCAAGCTGGTGCGCTCAGTGACTGTGGTTGAGGACGACGAGGA | |
| | TGAGGATGGAGATGACCTGCTCCATCACCACCACGGCTCCCAC | |
| | TGCAGCAGCTCGGGGGACCCCGCTGAGTACAACCTGCGCTCGC | |
| | GCACCGTGCTGTGCGGGACCTGCGGGCAGCCTGCCGACAAGGC | |
| | ATCTGCCAGCGGCTCAGGAGCCCAGGTGGGCGGACCCATCTCC | |
| | TCTGGCTCTTCTGCCTCCAGTGTCACGGTCACTCGCAGCTACCG | |
| | CAGTGTGGGGGGCAGTGGGGGTGGCAGCTTCGGGGACAATCTG | |
| | GTCACCCGCTCCTACCTCCTGGGCAACTCCAGCCCCCGAACCCA | |
| | GAGCCCCCAGAACTGCAGCATCATGTAA | |
| Human | METPSQRRATRSGAQASSTPLSPTRITRLQEKEDLQELNDRLAVYI | 790 |
| LMNA | DRVRSLETENAGLRLRITESEEVVSREVSGIKAAYEAELGDARKTL | |
| LaminC | DSVAKERARLQLELSKVREEFKELKARNTKKEGDLIAAQARLKDL | |
| protein | EALLNSKEAALSTALSEKRTLEGELHDLRGQVAKLEAALGEAKK | |
| | QLQDEMLRRVDAENRLQTMKEELDFQKNIYSEELRETKRRHETRL | |
| | VEIDNGKQREFESRLADALQELRAQHEDQVEQYKKELEKTYSAK | |
| | LDNARQSAERNSNLVGAAHEELQQSRIRIDSLSAQLSQLQKQLAA | |
| | KEAKLRDLEDSLARERDTSRRLLAEKEREMAEMRARMQQQLDEY | |
| | QELLDIKLALDMEIHAYRKLLEGEEERLRLSPSPTSQRSRGRASSHS | |
| | SQTQGGGSVTKKRKLESTESRSSFSQHARTSGRVAVEEVDEEGKF | |
| | VRLRNKSNEDQSMGNWQIKRQNGDDPLLTYRFPPKFTLKAGQVV | |
| | TIWAAGAGATHSPPTDLVWKAQNTWGCGNSLRTALINSTGEEVA | |
| | MRKLVRSVTVVEDDEDEDGDDLLHHHHVSGSRR | |
| Human | ATGGAGACCCCGTCCCAGCGGCGCGCCACCCGCAGCGGGGCGC | 791 |
| LMNA | AGGCCAGCTCCACTCCGCTGTCGCCCACCCGCATCACCCGGCT | |
| LaminC | GCAGGAGAAGGAGGACCTGCAGGAGCTCAATGATCGCTTGGC | |
| DNA | GGTCTACATCGACCGTGTGCGCTCGCTGGAAACGGAGAACGCA | |
| | GGGCTGCGCCTTCGCATCACCGAGTCTGAAGAGGTGGTCAGCC | |
| | GCGAGGTGTCCGGCATCAAGGCCGCCTACGAGGCCGAGCTCGG | |
| | GGATGCCCGCAAGACCCTTGACTCAGTAGCCAAGGAGCGCGCC | |
| | CGCCTGCAGCTGGAGCTGAGCAAAGTGCGTGAGGAGTTTAAGG | |
| | AGCTGAAAGCGCGCAATACCAAGAAGGAGGGTGACCTGATAG | |
| | CTGCTCAGGCTCGGCTGAAGGACCTGGAGGCTCTGCTGAACTC | |
| | CAAGGAGGCCGCACTGAGCACTGCTCTCAGTGAGAAGCGCACG | |
| | CTGGAGGGCGAGCTGCATGATCTGCGGGGCCAGGTGGCCAAGC | |
| | TTGAGGCAGCCCTAGGTGAGGCCAAGAAGCAACTTCAGGATGA | |
| | GATGCTGCGGCGGGTGGATGCTGAGAACAGGCTGCAGACCATG | |
| | AAGGAGGAACTGGACTTCCAGAAGAACATCTACAGTGAGGAG | |
| | CTGCGTGAGACCAAGCGCCGTCATGAGACCCGACTGGTGGAGA | |
| | TTGACAATGGGAAGCAGCGTGAGTTTGAGAGCCGGCTGGCGGA | |
| | TGCGCTGCAGGAACTGCGGGCCCAGCATGAGGACCAGGTGGA | |
| | GCAGTATAAGAAGGAGCTGGAGAAGACTTATTCTGCCAAGCTG | |
| | GACAATGCCAGGCAGTCTGCTGAGAGGAACAGCAACCTGGTGG | |
| | GGGCTGCCCACGAGGAGCTGCAGCAGTCGCGCATCCGCATCGA | |
| | CAGCCTCTCTGCCCAGCTCAGCCAGCTCCAGAAGCAGCTGGCA | |
| | GCCAAGGAGGCGAAGCTTCGAGACCTGGAGGACTCACTGGCCC | |
| | GTGAGCGGGACACCAGCCGGCGGCTGCTGGCGGAAAAGGAGC | |
| | GGGAGATGGCCGAGATGCGGGCAAGGATGCAGCAGCAGCTGG | |
| | ACGAGTACCAGGAGCTTCTGGACATCAAGCTGGCCCTGGACAT | |
| | GGAGATCCACGCCTACCGCAAGCTCTTGGAGGGCGAGGAGGA | |
| | GAGGCTACGCCTGTCCCCCAGCCCTACCTCGCAGCGCAGCCGT | |
| | GGCCGTGCTTCCTCTCACTCATCCCAGACACAGGGTGGGGGCA | |
| | GCGTCACCAAAAAGCGCAAACTGGAGTCCACTGAGAGCCGCA | |
| | GCAGCTTCTCACAGCACGCACGCACTAGCGGGCGCGTGGCCGT | |
| | GGAGGAGGTGGATGAGGAGGGCAAGTTTGTCCGGCTGCGCAA | |
| | CAAGTCCAATGAGGACCAGTCCATGGGCAATTGGCAGATCAAG | |
| | CGCCAGAATGGAGATGATCCCTTGCTGACTTACCGGTTCC | |
| | CACCAAAGTTCACCCTGAAGGCTGGGCAGGTGGTGACGATCTG | |
| | GGCTGCAGGAGCTGGGGCCACCCACAGCCCCCCTACCGACCTG | |
| | GTGTGGAAGGCACAGAACACCTGGGGCTGCGGGAACAGCCTG | |
| | CGTACGGCTCTCATCAACTCCACTGGGGAAGAAGTGGCCATGC | |
| | GCAAGCTGGTGCGCTCAGTGACTGTGGTTGAGGACGACGAGGA | |
| | TGAGGATGGAGATGACCTGCTCCATCACCACCACGTGAGTGGT | |
| | AGCCGCCGCTGA | |
| Human | MADGSSDAAREPRPAPAPIRRRSSNYRAYATEPHAKKKSKISASR | 792 |
| TNNI3 | KLQLKTLLLQIAKQELEREAEERRGEKGRALSTRCQPLELAGLGF | |
| protein | AELQDLCRQLHARVDKVDEERYDIEAKVTKNITEIADLTQKIFDLR | |
| | GKFKRPTLRRVRISADAMMQALLGARAKESLDLRAHLKQVKKED | |
| | TEKENREVGDWRKNIDALSGMEGRKKKFES | |
| Human | ATGGCGGATGGGAGCAGCGATGCGGCTAGGGAACCTCGCCCTG | 793 |
| TNNI3 | CACCAGCCCCAATCAGACGCCGCTCCTCCAACTACCGCGCTTAT | |
| DNA | GCCACGGAGCCGCACGCCAAGAAAAAATCTAAGATCTCCGCCT | |
| | CGAGAAAATTGCAGCTGAAGACTCTGCTGCTGCAGATTGCAAA | |
| | GCAAGAGCTGGAGCGAGAGGCGGAGGAGCGGCGCGGAGAGAA | |
| | GGGGCGCGCTCTGAGCACCCGCTGCCAGCCGCTGGAGTTGGCC | |
| | GGGCTGGGCTTCGCGGAGCTGCAGGACTTGTGCCGACAGCTCC | |
| | ACGCCCGTGTGGACAAGGTGGATGAAGAGAGATACGACATAG | |
| | AGGCAAAAGTCACCAAGAACATCACGGAGATTGCAGATCTGAC | |
| | TCAGAAGATCTTTGACCTTCGAGGCAAGTTTAAGCGGCCCACC | |
| | CTGCGGAGAGTGAGGATCTCTGCAGATGCCATGATGCAGGCGC | |
| | TGCTGGGGGCCCGGGCTAAGGAGTCCCTGGACCTGCGGGCCCA | |
| | CCTCAAGCAGGTGAAGAAGGAGGACACCGAGAAGGAAAACCG | |
| | GGAGGTGGGAGACTGGCGCAAGAACATCGATGCACTGAGTGG | |
| | AATGGAGGGCCGCAAGAAAAAGTTTGAGAGCTGA | |
| Human | MVCFRLFPVPGSGLVLVCLVLGAVRSYALELNLTDSENATCLYAK | 794 |
| LAMP2a | WQMNFTVRYETTNKTYKTVTISDHGTVTYNGSICGDDQNGPKIA | |
| protein | VQFGPGFSWIANFTKAASTYSIDSVSFSYNTGDNTTFPDAEDKGIL | |
| | TVDELLAIRIPLNDLFRCNSLSTLEKNDVVQHYWDVLVQAFVQNG | |
| | TVSTNEFLCDKDKTSTVAPTIHTTVPSPTTTPTPKEKPEAGTYSVN | |
| | NGNDTCLLATMGLQLNITQDKVASVININPNTTHSTGSCRSHTALL | |
| | RLNSSTIKYLDFVFAVKNENRFYLKEVNISMYLVNGSVFSIANNNL | |
| | SYWDAPLGSSYMCNKEQTVSVSGAFQINTFDLRVQPFNVTQGKY | |
| | STAQDCSADDDNFLVPIAVGAALAGVLILVLLAYFIGLKHHHAGY | |
| | EQF | |
| Human | ATGGTGTGCTTCCGCCTCTTCCCGGTTCCGGGCTCAGGGCTCGT | 795 |
| LAMP2a | TCTGGTCTGCCTAGTCCTGGGAGCTGTGCGGTCTTATGCATTGG | |
| DNA | AACTTAATTTGACAGATTCAGAAAATGCCACTTGCCTTTATGCA | |
| | AAATGGCAGATGAATTTCACAGTACGCTATGAAACTACAAATA | |
| | AAACTTATAAAACTGTAACCATTTCAGACCATGGCACTGTGAC | |
| | ATATAATGGAAGCATTTGTGGGGATGATCAGAATGGTCCCAAA | |
| | ATAGCAGTGCAGTTCGGACCTGGCTTTTCCTGGATTGCGAATTT | |
| | TACCAAGGCAGCATCTACTTATTCAATTGACAGCGTCTCATTTT | |
| | CCTACAACACTGGTGATAACACAACATTTCCTGATGCTGAAGA | |
| | TAAAGGAATTCTTACTGTTGATGAACTTTTGGCCATCAGAATTC | |
| | CATTGAATGACCTTTTTAGATGCAATAGTTTATCAACTTTGGAA | |
| | AAGAATGATGTTGTCCAACACTACTGGGATGTTCTTGTACAAG | |
| | CTTTTGTCCAAAATGGCACAGTGAGCACAAATGAGTTCCTGTGT | |
| | GATAAAGACAAAACTTCAACAGTGGCACCCACCATACACACCA | |
| | CTGTGCCATCTCCTACTACAACACCTACTCCAAAGGAAAAACC | |
| | AGAAGCTGGAACCTATTCAGTTAATAATGGCAATGATACTTGT | |
| | CTGCTGGCTACCATGGGGCTGCAGCTGAACATCACTCAGGATA | |
| | AGGTTGCTTCAGTTATTAACATCAACCCCAATACAACTCACTCC | |
| | ACAGGCAGCTGCCGTTCTCACACTGCTCTACTTAGACTCAATAG | |
| | CAGCACCATTAAGTATCTAGACTTTGTCTTTGCTGTGAAAAATG | |
| | AAAACCGATTTTATCTGAAGGAAGTGAACATCAGCATGTATTT | |
| | GGTTAATGGCTCCGTTTTCAGCATTGCAAATAACAATCTCAGCT | |
| | ACTGGGATGCCCCCCTGGGAAGTTCTTATATGTGCAACAAAGA | |
| | GCAGACTGTTTCAGTGTCTGGAGCATTTCAGATAAATACCTTTG | |
| | ATCTAAGGGTTCAGCCTTTCAATGTGACACAAGGAAAGTATTC | |
| | TACAGCTCAAGACTGCAGTGCAGATGACGACAACTTCCTTGTG | |
| | CCCATAGCGGTGGGAGCTGCCTTGGCAGGAGTACTTATTCTAG | |
| | TGTTGCTGGCTTATTTTATTGGTCTCAAGCACCATCATGCTGGA | |
| | TATGAGCAATTTTAG | |
| Human | MVCFRLFPVPGSGLVLVCLVLGAVRSYALELNLTDSENATCLYAK | 796 |
| LAMP2b | WQMNFTVRYETTNKTYKTVTISDHGTVTYNGSICGDDQNGPKIA | |
| protein | VQFGPGFSWIANFTKAASTYSIDSVSFSYNTGDNTTFPDAEDKGIL | |
| | TVDELLAIRIPLNDLFRCNSLSTLEKNDVVQHYWDVLVQAFVQNG | |
| | TVSTNEFLCDKDKTSTVAPTIHTTVPSPTTTPTPKEKPEAGTYSVN | |
| | NGNDTCLLATMGLQLNITQDKVASVININPNTTHSTGSCRSHTALL | |
| | RLNSSTIKYLDFVFAVKNENRFYLKEVNISMYLVNGSVFSIANNNL | |
| | SYWDAPLGSSYMCNKEQTVSVSGAFQINTFDLRVQPFNVTQGKY | |
| | STAQECSLDDDTILIPIIVGAGLSGLIIVIVIAYVIGRRKSYAGYQTL | |
| Human | ATGGTGTGCTTCCGCCTCTTCCCGGTTCCGGGCTCAGGGCTCGT | 797 |
| LAMP2b | TCTGGTCTGCCTAGTCCTGGGAGCTGTGCGGTCTTATGCATTGG | |
| DNA | AACTTAATTTGACAGATTCAGAAAATGCCACTTGCCTTTATGCA | |
| | AAATGGCAGATGAATTTCACAGTACGCTATGAAACTACAAATA | |
| | AAACTTATAAAACTGTAACCATTTCAGACCATGGCACTGTGAC | |
| | ATATAATGGAAGCATTTGTGGGGATGATCAGAATGGTCCCAAA | |
| | ATAGCAGTGCAGTTCGGACCTGGCTTTTCCTGGATTGCGAATTT | |
| | TACCAAGGCAGCATCTACTTATTCAATTGACAGCGTCTCATTTT | |
| | CCTACAACACTGGTGATAACACAACATTTCCTGATGCTGAAGA | |
| | TAAAGGAATTCTTACTGTTGATGAACTTTTGGCCATCAGAATTC | |
| | CATTGAATGACCTTTTTAGATGCAATAGTTTATCAACTTTGGAA | |
| | AAGAATGATGTTGTCCAACACTACTGGGATGTTCTTGTACAAG | |
| | CTTTTGTCCAAAATGGCACAGTGAGCACAAATGAGTTCCTGTGT | |
| | GATAAAGACAAAACTTCAACAGTGGCACCCACCATACACACCA | |
| | CTGTGCCATCTCCTACTACAACACCTACTCCAAAGGAAAAACC | |
| | AGAAGCTGGAACCTATTCAGTTAATAATGGCAATGATACTTGT | |
| | CTGCTGGCTACCATGGGGCTGCAGCTGAACATCACTCAGGATA | |
| | AGGTTGCTTCAGTTATTAACATCAACCCCAATACAACTCACTCC | |
| | ACAGGCAGCTGCCGTTCTCACACTGCTCTACTTAGACTCAATAG | |
| | CAGCACCATTAAGTATCTAGACTTTGTCTTTGCTGTGAAAAATG | |
| | AAAACCGATTTTATCTGAAGGAAGTGAACATCAGCATGTATTT | |
| | GGTTAATGGCTCCGTTTTCAGCATTGCAAATAACAATCTCAGCT | |
| | ACTGGGATGCCCCCCTGGGAAGTTCTTATATGTGCAACAAAGA | |
| | GCAGACTGTTTCAGTGTCTGGAGCATTTCAGATAAATACCTTTG | |
| | ATCTAAGGGTTCAGCCTTTCAATGTGACACAAGGAAAGTATTC | |
| | TACAGCCCAAGAGTGTTCGCTGGATGATGACACCATTCTAATC | |
| | CCAATTATAGTTGGTGCTGGTCTTTCAGGCTTGATTATCGTTAT | |
| | AGTGATTGCTTACGTAATTGGCAGAAGAAAAAGTTATGCTGGA | |
| | TATCAGACTCTGTAA | |
| Human | MVCFRLFPVPGSGLVLVCLVLGAVRSYALELNLTDSENATCLYAK | 798 |
| LAMP2c | WQMNFTVRYETTNKTYKTVTISDHGTVTYNGSICGDDQNGPKIA | |
| protein | VQFGPGFSWIANFTKAASTYSIDSVSFSYNTGDNTTFPDAEDKGIL | |
| | TVDELLAIRIPLNDLFRCNSLSTLEKNDVVQHYWDVLVQAFVQNG | |
| | TVSTNEFLCDKDKTSTVAPTIHTTVPSPTTTPTPKEKPEAGTYSVN | |
| | NGNDTCLLATMGLQLNITQDKVASVININPNTTHSTGSCRSHTALL | |
| | RLNSSTIKYLDFVFAVKNENRFYLKEVNISMYLVNGSVFSIANNNL | |
| | SYWDAPLGSSYMCNKEQTVSVSGAFQINTFDLRVQPFNVTQGKY | |
| | STAEECSADSDLNFLIPVAVGVALGFLIIVVFISYMIGRRKSRTGYQ | |
| | SV | |
| Human | ATGGTGTGCTTCCGCCTCTTCCCGGTTCCGGGCTCAGGGCTCGT | 799 |
| LAMP2c | TCTGGTCTGCCTAGTCCTGGGAGCTGTGCGGTCTTATGCATTGG | |
| DNA | AACTTAATTTGACAGATTCAGAAAATGCCACTTGCCTTTATGCA | |
| | AAATGGCAGATGAATTTCACAGTACGCTATGAAACTACAAATA | |
| | AAACTTATAAAACTGTAACCATTTCAGACCATGGCACTGTGAC | |
| | ATATAATGGAAGCATTTGTGGGGATGATCAGAATGGTCCCAAA | |
| | ATAGCAGTGCAGTTCGGACCTGGCTTTTCCTGGATTGCGAATTT | |
| | TACCAAGGCAGCATCTACTTATTCAATTGACAGCGTCTCATTTT | |
| | CCTACAACACTGGTGATAACACAACATTTCCTGATGCTGAAGA | |
| | TAAAGGAATTCTTACTGTTGATGAACTTTTGGCCATCAGAATTC | |
| | CATTGAATGACCTTTTTAGATGCAATAGTTTATCAACTTTGGAA | |
| | AAGAATGATGTTGTCCAACACTACTGGGATGTTCTTGTACAAG | |
| | CTTTTGTCCAAAATGGCACAGTGAGCACAAATGAGTTCCTGTGT | |
| | GATAAAGACAAAACTTCAACAGTGGCACCCACCATACACACCA | |
| | CTGTGCCATCTCCTACTACAACACCTACTCCAAAGGAAAAACC | |
| | AGAAGCTGGAACCTATTCAGTTAATAATGGCAATGATACTTGT | |
| | CTGCTGGCTACCATGGGGCTGCAGCTGAACATCACTCAGGATA | |
| | AGGTTGCTTCAGTTATTAACATCAACCCCAATACAACTCACTCC | |
| | ACAGGCAGCTGCCGTTCTCACACTGCTCTACTTAGACTCAATAG | |
| | CAGCACCATTAAGTATCTAGACTTTGTCTTTGCTGTGAAAAATG | |
| | AAAACCGATTTTATCTGAAGGAAGTGAACATCAGCATGTATTT | |
| | GGTTAATGGCTCCGTTTTCAGCATTGCAAATAACAATCTCAGCT | |
| | ACTGGGATGCCCCCCTGGGAAGTTCTTATATGTGCAACAAAGA | |
| | GCAGACTGTTTCAGTGTCTGGAGCATTTCAGATAAATACCTTTG | |
| | ATCTAAGGGTTCAGCCTTTCAATGTGACACAAGGAAAGTATTC | |
| | TACAGCTGAAGAATGTTCTGCTGACTCTGACCTCAACTTTCTTA | |
| | TTCCTGTTGCAGTGGGTGTGGCCTTGGGCTTCCTTATAATTGTT | |
| | GTCTTTATCTCTTATATGATTGGAAGAAGGAAAAGTCGTACTGG | |
| | TTATCAGTCTGTGTAA | |
| Human | MSCNGGSHPRINTLGRMIRAESGPDLRYEVTSGGGGTSRMYYSRR | 800 |
| DSP_DPI | GVITDQNSDGYCQTGTMSRHQNQNTIQELLQNCSDCLMRAELIVQ | |
| protein | PELKYGDGIQLTRSRELDECFAQANDQMEILDSLIREMRQMGQPC | |
| | DAYQKRLLQLQEQMRALYKAISVPRVRRASSKGGGGYTCQSGSG | |
| | WDEFTKHVTSECLGWMRQQRAEMDMVAWGVDLASVEQHINSH | |
| | RGIHNSIGDYRWQLDKIKADLREKSAIYQLEEEYENLLKASFERM | |
| | DHLRQLQNIIQATSREIMWINDCEEEELLYDWSDKNTNIAQKQEA | |
| | FSIRMSQLEVKEKELNKLKQESDQLVLNQHPASDKIEAYMDTLQT | |
| | QWSWILQITKCIDVHLKENAAYFQFFEEAQSTEAYLKGLQDSIRK | |
| | KYPCDKNMPLQHLLEQIKELEKEREKILEYKRQVQNLVNKSKKIV | |
| | QLKPRNPDYRSNKPIILRALCDYKQDQKIVHKGDECILKDNNERSK | |
| | WYVTGPGGVDMLVPSVGLIIPPPNPLAVDLSCKIEQYYEAILALW | |
| | NQLYINMKSLVSWHYCMIDIEKIRAMTIAKLKTMRQEDYMKTIAD | |
| | LELHYQEFIRNSQGSEMFGDDDKRKIQSQFTDAQKHYQTLVIQLP | |
| | GYPQHQTVTTTEITHHGTCQDVNHNKVIETNRENDKQETWMLME | |
| | LQKIRRQIEHCEGRMTLKNLPLADQGSSHHITVKINELKSVQNDSQ | |
| | AIAEVLNQLKDMLANFRGSEKYCYLQNEVFGLFQKLENINGVTD | |
| | GYLNSLCTVRALLQAILQTEDMLKVYEARLTEEETVCLDLDKVEA | |
| | YRCGLKKIKNDLNLKKSLLATMKTELQKAQQIHSQTSQQYPLYDL | |
| | DLGKFGEKVTQLTDRWQRIDKQIDFRLWDLEKQIKQLRNYRDNY | |
| | QAFCKWLYDAKRRQDSLESMKFGDSNTVMRFLNEQKNLHSEISG | |
| | KRDKSEEVQKIAELCANSIKDYELQLASYTSGLETLLNIPIKRTMIQ | |
| | SPSGVILQEAADVHARYIELLTRSGDYYRFLSEMLKSLEDLKLKNT | |
| | KIEVLEEELRLARDANSENCNKNKFLDQNLQKYQAECSQFKAKL | |
| | ASLEELKRQAELDGKSAKQNLDKCYGQIKELNEKITRLTYEIEDEK | |
| | RRRKSVEDRFDQQKNDYDQLQKARQCEKENLGWQKLESEKAIKE | |
| | KEYEIERLRVLLQEEGTRKREYENELAKVRNHYNEEMSNLRNKY | |
| | ETEINITKTTIKEISMQKEDDSKNLRNQLDRLSRENRDLKDEIVRLN | |
| | DSILQATEQRRRAEENALQQKACGSEIMQKKQHLEIELKQVMQQR | |
| | SEDNARHKQSLEEAAKTIQDKNKEIERLKAEFQEEAKRRWEYENE | |
| | LSKVRNNYDEEIISLKNQFETEINITKTTIHQLTMQKEEDTSGYRAQ | |
| | IDNLTRENRSLSEEIKRLKNTLTQTTENLRRVEEDIQQQKATGSEVS | |
| | QRKQQLEVELRQVTQMRTEESVRYKQSLDDAAKTIQDKNKEIERL | |
| | KQLIDKETNDRKCLEDENARLQRVQYDLQKANSSATETINKLKVQ | |
| | EQELTRLRIDYERVSQERTVKDQDITRFQNSLKELQLQKQKVEEEL | |
| | NRLKRTASEDSCKRKKLEEELEGMRRSLKEQAIKITNLTQQLEQAS | |
| | IVKKRSEDDLRQQRDVLDGHLREKQRTQEELRRLSSEVEALRRQL | |
| | LQEQESVKQAHLRNEHFQKAIEDKSRSLNESKIEIERLQSLTENLTK | |
| | EHLMLEEELRNLRLEYDDLRRGRSEADSDKNATILELRSQLQISNN | |
| | RTLELQGLINDLQRERENLRQEIEKFQKQALEASNRIQESKNQCTQ | |
| | VVQERESLLVKIKVLEQDKARLQRLEDELNRAKSTLEAETRVKQR | |
| | LECEKQQIQNDLNQWKTQYSRKEEAIRKIESEREKSEREKNSLRSE | |
| | IERLQAEIKRIEERCRRKLEDSTRETQSQLETERSRYQREIDKLRQR | |
| | PYGSHRETQTECEWTVDTSKLVFDGLRKKVTAMQLYECQLIDKT | |
| | TLDKLLKGKKSVEEVASEIQPFLRGAGSIAGASASPKEKYSLVEAK | |
| | RKKLISPESTVMLLEAQAATGGIIDPHRNEKLTVDSAIARDLIDFDD | |
| | RQQIYAAEKAITGFDDPFSGKTVSVSEAIKKNLIDRETGMRLLEAQ | |
| | IASGGVVDPVNSVFLPKDVALARGLIDRDLYRSLNDPRDSQKNFV | |
| | DPVTKKKVSYVQLKERCRIEPHTGLLLLSVQKRSMSFQGIRQPVT | |
| | VTELVDSGILRPSTVNELESGQISYDEVGERIKDFLQGSSCIAGIYN | |
| | ETTKQKLGIYEAMKIGLVRPGTALELLEAQAATGFIVDPVSNLRLP | |
| | VEEAYKRGLVGIEFKEKLLSAERAVTGYNDPETGNIISLFQAMNKE | |
| | LIEKGHGIRLLEAQIATGGIIDPKESHRLPVDIAYKRGYFNEELSEIL | |
| | SDPSDDTKGFFDPNTEENLTYLQLKERCIKDEETGLCLLPLKEKKK | |
| | QVQTSQKNTLRKRRVVIVDPETNKEMSVQEAYKKGLIDYETFKEL | |
| | CEQECEWEEITITGSDGSTRVVLVDRKTGSQYDIQDAIDKGLVDR | |
| | KFFDQYRSGSLSLTQFADMISLKNGVGTSSSMGSGVSDDVESSSRH | |
| | ESVSKISTISSVRNLTIRSSSFSDTLEESSPIAAIFDTENLEKISITEGIE | |
| | RGIVDSITGQRLLEAQACTGGIIHPTTGQKLSLQDAVSQGVIDQDM | |
| | ATRLKPAQKAFIGFEGVKGKKKMSAAEAVKEKWLPYEAGQRFLE | |
| | FQYLTGGLVDPEVHGRISTEEAIRKGFIDGRAAQRLQDTSSYAKIL | |
| | TCPKTKLKISYKDAINRSMVEDITGLRLLEAASVSSKGLPSPYNMS | |
| | SAPGSRSGSRSGSRSGSRSGSRSGSRRGSFDATGNSSYSYSYSFSSS | |
| | SIGH | |
| Human | ATGAGCTGCAACGGAGGCTCCCACCCGCGGATCAACACTCTGG | 801 |
| DSP_DPI | GCCGCATGATCCGCGCCGAGTCTGGCCCGGACCTGCGCTACGA | |
| DNA | GGTGACCAGCGGCGGCGGGGGCACCAGCAGGATGTACTATTCT | |
| | CGGCGCGGCGTGATCACCGACCAGAACTCGGACGGCTACTGTC | |
| | AAACCGGCACGATGTCCAGGCACCAGAACCAGAACACCATCCA | |
| | GGAGCTGCTGCAGAACTGCTCCGACTGCTTGATGCGAGCAGAG | |
| | CTCATCGTGCAGCCTGAATTGAAGTATGGAGATGGAATACAAC | |
| | TGACTCGGAGTCGAGAATTGGATGAGTGTTTTGCCCAGGCCAA | |
| | TGACCAAATGGAAATCCTCGACAGCTTGATCAGAGAGATGCGG | |
| | CAGATGGGCCAGCCCTGTGATGCTTACCAGAAAAGGCTTCTTC | |
| | AGCTCCAAGAGCAAATGCGAGCCCTTTATAAAGCCATCAGTGT | |
| | CCCTCGAGTCCGCAGGGCCAGCTCCAAGGGTGGTGGAGGCTAC | |
| | ACTTGTCAGAGTGGCTCTGGCTGGGATGAGTTCACCAAACATG | |
| | TCACCAGTGAATGTTTGGGGTGGATGAGGCAGCAAAGGGCGGA | |
| | GATGGACATGGTGGCCTGGGGTGTGGACCTGGCCTCAGTGGAG | |
| | CAGCACATTAACAGCCACCGGGGCATCCACAACTCCATCGGCG | |
| | ACTATCGCTGGCAGCTGGACAAAATCAAAGCCGACCTGCGCGA | |
| | GAAATCTGCGATCTACCAGTTGGAGGAGGAGTATGAAAACCTG | |
| | CTGAAAGCGTCCTTTGAGAGGATGGATCACCTGCGACAGCTGC | |
| | AGAACATCATTCAGGCCACGTCCAGGGAGATCATGTGGATCAA | |
| | TGACTGCGAGGAGGAGGAGCTGCTGTACGACTGGAGCGACAA | |
| | GAACACCAACATCGCTCAGAAACAGGAGGCCTTCTCCATACGC | |
| | ATGAGTCAACTGGAAGTTAAAGAAAAAGAGCTCAATAAGCTG | |
| | AAACAAGAAAGTGACCAACTTGTCCTCAATCAGCATCCAGCTT | |
| | CAGACAAAATTGAGGCCTATATGGACACTCTGCAGACGCAGTG | |
| | GAGTTGGATTCTTCAGATCACCAAGTGCATTGATGTTCATCTGA | |
| | AAGAAAATGCTGCCTACTTTCAGTTTTTTGAAGAGGCGCAGTCT | |
| | ACTGAAGCATACCTGAAGGGGCTCCAGGACTCCATCAGGAAGA | |
| | AGTACCCCTGCGACAAGAACATGCCCCTGCAGCACCTGCTGGA | |
| | ACAGATCAAGGAGCTGGAGAAAGAACGAGAGAAAATCCTTGA | |
| | ATACAAGCGTCAGGTGCAGAACTTGGTAAACAAGTCTAAGAAG | |
| | ATTGTACAGCTGAAGCCTCGTAACCCAGACTACAGAAGCAATA | |
| | AACCCATTATTCTCAGAGCTCTCTGTGACTACAAACAAGATCA | |
| | GAAAATCGTGCATAAGGGGGATGAGTGTATCCTGAAGGACAAC | |
| | AACGAGCGCAGCAAGTGGTACGTGACGGGCCCGGGAGGCGTT | |
| | GACATGCTTGTTCCCTCTGTGGGGCTGATCATCCCTCCTCCGAA | |
| | CCCACTGGCCGTGGACCTCTCTTGCAAGATTGAGCAGTACTAC | |
| | GAAGCCATCTTGGCTCTGTGGAACCAGCTCTACATCAACATGA | |
| | AGAGCCTGGTGTCCTGGCACTACTGCATGATTGACATAGAGAA | |
| | GATCAGGGCCATGACAATCGCCAAGCTGAAAACAATGCGGCA | |
| | GGAAGATTACATGAAGACGATAGCCGACCTTGAGTTACATTAC | |
| | CAAGAGTTCATCAGAAATAGCCAAGGCTCAGAGATGTTTGGAG | |
| | ATGATGACAAGCGGAAAATACAGTCTCAGTTCACCGATGCCCA | |
| | GAAGCATTACCAGACCCTGGTCATTCAGCTCCCTGGCTATCCCC | |
| | AGCACCAGACAGTGACCACAACTGAAATCACTCATCATGGAAC | |
| | CTGCCAAGATGTCAACCATAATAAAGTAATTGAAACCAACAGA | |
| | GAAAATGACAAGCAAGAAACATGGATGCTGATGGAGCTGCAG | |
| | AAGATTCGCAGGCAGATAGAGCACTGCGAGGGCAGGATGACT | |
| | CTCAAAAACCTCCCTCTAGCAGACCAGGGATCTTCTCACCACAT | |
| | CACAGTGAAAATTAACGAGCTTAAGAGTGTGCAGAATGATTCA | |
| | CAAGCAATTGCTGAGGTTCTCAACCAGCTTAAAGATATGCTTG | |
| | CCAACTTCAGAGGTTCTGAAAAGTACTGCTATTTACAGAATGA | |
| | AGTATTTGGACTATTTCAGAAACTGGAAAATATCAATGGTGTT | |
| | ACAGATGGCTACTTAAATAGCTTATGCACAGTAAGGGCACTGC | |
| | TCCAGGCTATTCTCCAAACAGAAGACATGTTAAAGGTTTATGA | |
| | AGCCAGGCTCACTGAGGAGGAAACTGTCTGCCTGGACCTGGAT | |
| | AAAGTGGAAGCTTACCGCTGTGGACTGAAGAAAATAAAAAAT | |
| | GACTTGAACTTGAAGAAGTCGTTGTTGGCCACTATGAAGACAG | |
| | AACTACAGAAAGCCCAGCAGATCCACTCTCAGACTTCACAGCA | |
| | GTATCCACTTTATGATCTGGACTTGGGCAAGTTCGGTGAAAAA | |
| | GTCACACAGCTGACAGACCGCTGGCAAAGGATAGATAAACAG | |
| | ATCGACTTTAGGTTATGGGACCTGGAGAAACAAATCAAGCAAT | |
| | TGAGGAATTATCGTGATAACTATCAGGCTTTCTGCAAGTGGCTC | |
| | TATGATGCTAAACGCCGCCAGGATTCCTTAGAATCCATGAAAT | |
| | TTGGAGATTCCAACACAGTCATGCGGTTTTTGAATGAGCAGAA | |
| | GAACTTGCACAGTGAAATATCTGGCAAACGAGACAAATCAGAG | |
| | GAAGTACAAAAAATTGCTGAACTTTGCGCCAATTCAATTAAGG | |
| | ATTATGAGCTCCAGCTGGCCTCATACACCTCAGGACTGGAAAC | |
| | TCTGCTGAACATACCTATCAAGAGGACCATGATTCAGTCCCCTT | |
| | CTGGGGTGATTCTGCAAGAGGCTGCAGATGTTCATGCTCGGTA | |
| | CATTGAACTACTTACAAGATCTGGAGACTATTACAGGTTCTTAA | |
| | GTGAGATGCTGAAGAGTTTGGAAGATCTGAAGCTGAAAAATAC | |
| | CAAGATCGAAGTTTTGGAAGAGGAGCTCAGACTGGCCCGAGAT | |
| | GCCAACTCGGAAAACTGTAATAAGAACAAATTCCTGGATCAGA | |
| | ACCTGCAGAAATACCAGGCAGAGTGTTCCCAGTTCAAAGCGAA | |
| | GCTTGCGAGCCTGGAGGAGCTGAAGAGACAGGCTGAGCTGGAT | |
| | GGGAAGTCGGCTAAGCAAAATCTAGACAAGTGCTACGGCCAA | |
| | ATAAAAGAACTCAATGAGAAGATCACCCGACTGACTTATGAGA | |
| | TTGAAGATGAAAAGAGAAGAAGAAAATCTGTGGAAGACAGAT | |
| | TTGACCAACAGAAGAATGACTATGACCAACTGCAGAAAGCAA | |
| | GGCAATGTGAAAAGGAGAACCTTGGTTGGCAGAAATTAGAGTC | |
| | TGAGAAAGCCATCAAGGAGAAGGAGTACGAGATTGAAAGGTT | |
| | GAGGGTTCTACTGCAGGAAGAAGGCACCCGGAAGAGAGAATA | |
| | TGAAAATGAGCTGGCAAAGGTAAGAAACCACTATAATGAGGA | |
| | GATGAGTAATTTAAGGAACAAGTATGAAACAGAGATTAACATT | |
| | ACGAAGACCACCATCAAGGAGATATCCATGCAAAAAGAGGAT | |
| | GATTCCAAAAATCTTAGAAACCAGCTTGATAGACTTTCAAGGG | |
| | AAAATCGAGATCTGAAGGATGAAATTGTCAGGCTCAATGACAG | |
| | CATCTTGCAGGCCACTGAGCAGCGAAGGCGAGCTGAAGAAAA | |
| | CGCCCTTCAGCAAAAGGCCTGTGGCTCTGAGATAATGCAGAAG | |
| | AAGCAGCATCTGGAGATAGAACTGAAGCAGGTCATGCAGCAG | |
| | CGCTCTGAGGACAATGCCCGGCACAAGCAGTCCCTGGAGGAGG | |
| | CTGCCAAGACCATTCAGGACAAAAATAAGGAGATCGAGAGAC | |
| | TCAAAGCTGAGTTTCAGGAGGAGGCCAAGCGCCGCTGGGAATA | |
| | TGAAAATGAACTGAGTAAGGTAAGAAACAATTATGATGAGGA | |
| | GATCATTAGCTTAAAAAATCAGTTTGAGACCGAGATCAACATC | |
| | ACCAAGACCACCATCCACCAGCTCACCATGCAGAAGGAAGAG | |
| | GATACCAGTGGCTACCGGGCTCAGATAGACAATCTCACCCGAG | |
| | AAAACAGGAGCTTATCTGAAGAAATAAAGAGGCTGAAGAACA | |
| | CTCTAACCCAGACCACAGAGAATCTCAGGAGGGTGGAAGAAG | |
| | ACATCCAACAGCAAAAGGCCACTGGCTCTGAGGTGTCTCAGAG | |
| | GAAACAGCAGCTGGAGGTTGAGCTGAGACAAGTCACTCAGATG | |
| | CGAACAGAGGAGAGCGTAAGATATAAGCAATCTCTTGATGATG | |
| | CTGCCAAAACCATCCAGGATAAAAACAAGGAGATAGAAAGGT | |
| | TAAAACAACTGATCGACAAAGAAACAAATGACCGGAAATGCC | |
| | TGGAAGATGAAAACGCGAGATTACAAAGGGTCCAGTATGACCT | |
| | GCAGAAAGCAAACAGTAGTGCGACGGAGACAATAAACAAACT | |
| | GAAGGTTCAGGAGCAAGAACTGACACGCCTGAGGATCGACTAT | |
| | GAAAGGGTTTCCCAGGAGAGGACTGTGAAGGACCAGGATATC | |
| | ACGCGGTTCCAGAACTCTCTGAAAGAGCTGCAGCTGCAGAAGC | |
| | AGAAGGTGGAAGAGGAGCTGAATCGGCTGAAGAGGACCGCGT | |
| | CAGAAGACTCCTGCAAGAGGAAGAAGCTGGAGGAAGAGCTGG | |
| | AAGGCATGAGGAGGTCGCTGAAGGAGCAAGCCATCAAAATCA | |
| | CCAACCTGACCCAGCAGCTGGAGCAGGCATCCATTGTTAAGAA | |
| | GAGGAGTGAGGATGACCTCCGGCAGCAGAGGGACGTGCTGGA | |
| | TGGCCACCTGAGGGAAAAGCAGAGGACCCAGGAAGAGCTGAG | |
| | GAGGCTCTCTTCTGAGGTCGAGGCCCTGAGGCGGCAGTTACTC | |
| | CAGGAACAGGAAAGTGTCAAACAAGCTCACTTGAGGAATGAG | |
| | CATTTCCAGAAGGCGATAGAAGATAAAAGCAGAAGCTTAAATG | |
| | AAAGCAAAATAGAAATTGAGAGGCTGCAGTCTCTCACAGAGA | |
| | ACCTGACCAAGGAGCACTTGATGTTAGAAGAAGAACTGCGGAA | |
| | CCTGAGGCTGGAGTACGATGACCTGAGGAGAGGACGAAGCGA | |
| | AGCGGACAGTGATAAAAATGCAACCATCTTGGAACTAAGGAGC | |
| | CAGCTGCAGATCAGCAACAACCGGACCCTGGAACTGCAGGGGC | |
| | TGATTAATGATTTACAGAGAGAGAGGGAAAATTTGAGACAGGA | |
| | AATTGAGAAATTCCAAAAGCAGGCTTTAGAGGCATCTAATAGG | |
| | ATTCAGGAATCAAAGAATCAGTGTACTCAGGTGGTACAGGAAA | |
| | GAGAGAGCCTTCTGGTGAAAATCAAAGTCCTGGAGCAAGACAA | |
| | GGCAAGGCTGCAGAGGCTGGAGGATGAGCTGAATCGTGCAAA | |
| | ATCAACTCTAGAGGCAGAAACCAGGGTGAAACAGCGCCTGGA | |
| | GTGTGAGAAACAGCAAATTCAGAATGACCTGAATCAGTGGAAG | |
| | ACTCAATATTCCCGCAAGGAGGAGGCTATTAGGAAGATAGAAT | |
| | CGGAAAGAGAAAAGAGTGAGAGAGAGAAGAACAGTCTTAGGA | |
| | GTGAGATCGAAAGACTCCAAGCAGAGATCAAGAGAATTGAAG | |
| | AGAGGTGCAGGCGTAAGCTGGAGGATTCTACCAGGGAGACAC | |
| | AGTCACAGTTAGAAACAGAACGCTCCCGATATCAGAGGGAGAT | |
| | TGATAAACTCAGACAGCGCCCATATGGGTCCCATCGAGAGACC | |
| | CAGACTGAGTGTGAGTGGACCGTTGACACCTCCAAGCTGGTGT | |
| | TTGATGGGCTGAGGAAGAAGGTGACAGCAATGCAGCTCTATGA | |
| | GTGTCAGCTGATCGACAAAACAACCTTGGACAAACTATTGAAG | |
| | GGGAAGAAGTCAGTGGAAGAAGTTGCTTCTGAAATCCAGCCAT | |
| | TCCTTCGGGGTGCAGGATCTATCGCTGGAGCATCTGCTTCTCCT | |
| | AAGGAAAAATACTCTTTGGTAGAGGCCAAGAGAAAGAAATTA | |
| | ATCAGCCCAGAATCCACAGTCATGCTTCTGGAGGCCCAGGCAG | |
| | CTACAGGTGGTATAATTGATCCCCATCGGAATGAGAAGCTGAC | |
| | TGTCGACAGTGCCATAGCTCGGGACCTCATTGACTTCGATGACC | |
| | GTCAGCAGATATATGCAGCAGAAAAAGCTATCACTGGTTTTGA | |
| | TGATCCATTTTCAGGCAAGACAGTATCTGTTTCAGAAGCCATCA | |
| | AGAAAAATTTGATTGATAGAGAAACCGGAATGCGCCTGCTGGA | |
| | AGCCCAGATTGCTTCAGGGGGTGTAGTAGACCCTGTGAACAGT | |
| | GTCTTTTTGCCAAAAGATGTCGCCTTGGCCCGGGGGCTGATTGA | |
| | TAGAGATTTGTATCGATCCCTGAATGATCCCCGAGATAGTCAG | |
| | AAAAACTTTGTGGATCCAGTCACCAAAAAGAAGGTCAGTTACG | |
| | TGCAGCTGAAGGAACGGTGCAGAATCGAACCACATACTGGTCT | |
| | GCTCTTGCTTTCAGTACAGAAGAGAAGCATGTCCTTCCAAGGA | |
| | ATCAGACAACCTGTGACCGTCACTGAGCTAGTAGATTCTGGTA | |
| | TATTGAGACCGTCCACTGTCAATGAACTGGAATCTGGTCAGATT | |
| | TCTTATGACGAGGTTGGTGAGAGAATTAAGGACTTCCTCCAGG | |
| | GTTCAAGCTGCATAGCAGGCATATACAATGAGACCACAAAACA | |
| | GAAGCTTGGCATTTATGAGGCCATGAAAATTGGCTTAGTCCGA | |
| | CCTGGTACTGCTCTGGAGTTGCTGGAAGCCCAAGCAGCTACTG | |
| | GCTTTATAGTGGATCCTGTTAGCAACTTGAGGTTACCAGTGGAG | |
| | GAAGCCTACAAGAGAGGTCTGGTGGGCATTGAGTTCAAAGAGA | |
| | AGCTCCTGTCTGCAGAACGAGCTGTCACTGGGTATAATGATCCT | |
| | GAAACAGGAAACATCATCTCTTTGTTCCAAGCCATGAATAAGG | |
| | AACTCATCGAAAAGGGCCACGGTATTCGCTTATTAGAAGCACA | |
| | GATCGCAACCGGGGGGATCATTGACCCAAAGGAGAGCCATCGT | |
| | TTACCAGTTGACATAGCATATAAGAGGGGCTATTTCAATGAGG | |
| | AACTCAGTGAGATTCTCTCAGATCCAAGTGATGATACCAAAGG | |
| | ATTTTTTGACCCCAACACTGAAGAAAATCTTACCTATCTGCAAC | |
| | TAAAAGAAAGATGCATTAAGGATGAGGAAACAGGGCTCTGTCT | |
| | TCTGCCTCTGAAAGAAAAGAAGAAACAGGTGCAGACATCACA | |
| | AAAGAATACCCTCAGGAAGCGTAGAGTGGTCATAGTTGACCCA | |
| | GAAACCAATAAAGAAATGTCTGTTCAGGAGGCCTACAAGAAG | |
| | GGCCTAATTGATTATGAAACCTTCAAAGAACTGTGTGAGCAGG | |
| | AATGTGAATGGGAAGAAATAACCATCACGGGATCAGATGGCTC | |
| | CACCAGGGTGGTCCTGGTAGATAGAAAGACAGGCAGTCAGTAT | |
| | GATATTCAAGATGCTATTGACAAGGGCCTTGTTGACAGGAAGT | |
| | TCTTTGATCAGTACCGATCCGGCAGCCTCAGCCTCACTCAATTT | |
| | GCTGACATGATCTCCTTGAAAAATGGTGTCGGCACCAGCAGCA | |
| | GCATGGGCAGTGGTGTCAGCGATGATGTTTTTAGCAGCTCCCG | |
| | ACATGAATCAGTAAGTAAGATTTCCACCATATCCAGCGTCAGG | |
| | AATTTAACCATAAGGAGCAGCTCTTTTTCAGACACCCTGGAAG | |
| | AATCGAGCCCCATTGCAGCCATCTTTGACACAGAAAACCTGGA | |
| | GAAAATCTCCATTACAGAAGGTATAGAGCGGGGCATCGTTGAC | |
| | AGCATCACGGGTCAGAGGCTTCTGGAGGCTCAGGCCTGCACAG | |
| | GTGGCATCATCCACCCAACCACGGGCCAGAAGCTGTCACTTCA | |
| | GGACGCAGTCTCCCAGGGTGTGATTGACCAAGACATGGCCACC | |
| | AGGCTGAAGCCTGCTCAGAAAGCCTTCATAGGCTTCGAGGGTG | |
| | TGAAGGGAAAGAAGAAGATGTCAGCAGCAGAGGCAGTGAAAG | |
| | AAAAATGGCTCCCGTATGAGGCTGGCCAGCGCTTCCTGGAGTT | |
| | CCAGTACCTCACGGGAGGTCTTGTTGACCCGGAAGTGCATGGG | |
| | AGGATAAGCACCGAAGAAGCCATCCGGAAGGGGTTCATAGAT | |
| | GGCCGCGCCGCACAGAGGCTGCAAGACACCAGCAGCTATGCCA | |
| | AAATCCTGACCTGCCCCAAAACCAAATTAAAAATATCCTATAA | |
| | GGATGCCATAAATCGCTCCATGGTAGAAGATATCACTGGGCTG | |
| | CGCCTTCTGGAAGCCGCCTCCGTGTCGTCCAAGGGCTTACCCAG | |
| | CCCTTACAACATGTCTTCGGCTCCGGGGTCCCGCTCCGGCTCCC | |
| | GCTCGGGATCTCGCTCCGGATCTCGCTCCGGGTCCCGCAGTGG | |
| | GTCCCGGAGAGGAAGCTTTGACGCCACAGGGAATTCTTCCTAC | |
| | TCTTATTCCTACTCATTTAGCAGTAGTTCTATTGGGCACTAG | |
| Human | MSCNGGSHPRINTLGRMIRAESGPDLRYEVTSGGGGTSRMYYSRR | 802 |
| DSP DPII | GVITDQNSDGYCQTGTMSRHQNQNTIQELLQNCSDCLMRAELIVQ | |
| isoform | PELKYGDGIQLTRSRELDECFAQANDQMEILDSLIREMRQMGQPC | |
| protein | DAYQKRLLQLQEQMRALYKAISVPRVRRASSKGGGGYTCQSGSG | |
| | WDEFTKHVTSECLGWMRQQRAEMDMVAWGVDLASVEQHINSH | |
| | RGIHNSIGDYRWQLDKIKADLREKSAIYQLEEEYENLLKASFERM | |
| | DHLRQLQNIIQATSREIMWINDCEEEELLYDWSDKNTNIAQKQEA | |
| | FSIRMSQLEVKEKELNKLKQESDQLVLNQHPASDKIEAYMDTLQT | |
| | QWSWILQITKCIDVHLKENAAYFQFFEEAQSTEAYLKGLQDSIRK | |
| | KYPCDKNMPLQHLLEQIKELEKEREKILEYKRQVQNLVNKSKKIV | |
| | QLKPRNPDYRSNKPIILRALCDYKQDQKIVHKGDECILKDNNERSK | |
| | WYVTGPGGVDMLVPSVGLIIPPPNPLAVDLSCKIEQYYEAILALW | |
| | NQLYINMKSLVSWHYCMIDIEKIRAMTIAKLKTMRQEDYMKTIAD | |
| | LELHYQEFIRNSQGSEMFGDDDKRKIQSQFTDAQKHYQTLVIQLP | |
| | GYPQHQTVTTTEITHHGTCQDVNHNKVIETNRENDKQETWMLME | |
| | LQKIRRQIEHCEGRMTLKNLPLADQGSSHHITVKINELKSVQNDSQ | |
| | AIAEVLNQLKDMLANFRGSEKYCYLQNEVFGLFQKLENINGVTD | |
| | GYLNSLCTVRALLQAILQTEDMLKVYEARLTEEETVCLDLDKVEA | |
| | YRCGLKKIKNDLNLKKSLLATMKTELQKAQQIHSQTSQQYPLYDL | |
| | DLGKFGEKVTQLTDRWQRIDKQIDFRLWDLEKQIKQLRNYRDNY | |
| | QAFCKWLYDAKRRQDSLESMKFGDSNTVMRFLNEQKNLHSEISG | |
| | KRDKSEEVQKIAELCANSIKDYELQLASYTSGLETLLNIPIKRTMIQ | |
| | SPSGVILQEAADVHARYIELLTRSGDYYRFLSEMLKSLEDLKLKNT | |
| | KIEVLEEELRLARDANSENCNKNKFLDQNLQKYQAECSQFKAKL | |
| | ASLEELKRQAELDGKSAKQNLDKCYGQIKELNEKITRLTYEIEDEK | |
| | RRRKSVEDRFDQQKNDYDQLQKARQCEKENLGWQKLESEKAIKE | |
| | KEYEIERLRVLLQEEGTRKREYENELAKASNRIQESKNQCTQVVQ | |
| | ERESLLVKIKVLEQDKARLQRLEDELNRAKSTLEAETRVKQRLEC | |
| | EKQQIQNDLNQWKTQYSRKEEAIRKIESEREKSEREKNSLRSEIER | |
| | LQAEIKRIEERCRRKLEDSTRETQSQLETERSRYQREIDKLRQRPYG | |
| | SHRETQTECEWTVDTSKLVFDGLRKKVTAMQLYECQLIDKTTLD | |
| | KLLKGKKSVEEVASEIQPFLRGAGSIAGASASPKEKYSLVEAKRKK | |
| | LISPESTVMLLEAQAATGGIIDPHRNEKLTVDSAIARDLIDFDDRQQ | |
| | IYAAEKAITGFDDPFSGKTVSVSEAIKKNLIDRETGMRLLEAQIASG | |
| | GVVDPVNSVFLPKDVALARGLIDRDLYRSLNDPRDSQKNFVDPVT | |
| | KKKVSYVQLKERCRIEPHTGLLLLSVQKRSMSFQGIRQPVTVTELV | |
| | DSGILRPSTVNELESGQISYDEVGERIKDFLQGSSCIAGIYNETTKQ | |
| | KLGIYEAMKIGLVRPGTALELLEAQAATGFIVDPVSNLRLPVEEAY | |
| | KRGLVGIEFKEKLLSAERAVTGYNDPETGNIISLFQAMNKELIEKG | |
| | HGIRLLEAQIATGGIIDPKESHRLPVDIAYKRGYFNEELSEILSDPSD | |
| | DTKGFFDPNTEENLTYLQLKERCIKDEETGLCLLPLKEKKKQVQTS | |
| | QKNTLRKRRVVIVDPETNKEMSVQEAYKKGLIDYETFKELCEQEC | |
| | EWEEITITGSDGSTRVVLVDRKTGSQYDIQDAIDKGLVDRKFFDQ | |
| | YRSGSLSLTQFADMISLKNGVGTSSSMGSGVSDDVFSSSRHESVSK | |
| | ISTISSVRNLTIRSSSFSDTLEESSPIAAIFDTENLEKISITEGIERGIVD | |
| | SITGQRLLEAQACTGGIIHPTTGQKLSLQDAVSQGVIDQDMATRLK | |
| | PAQKAFIGFEGVKGKKKMSAAEAVKEKWLPYEAGQRFLEFQYLT | |
| | GGLVDPEVHGRISTEEAIRKGFIDGRAAQRLQDTSSYAKILTCPKT | |
| | KLKISYKDAINRSMVEDITGLRLLEAASVSSKGLPSPYNMSSAPGS | |
| | RSGSRSGSRSGSRSGSRSGSRRGSFDATGNSSYSYSYSFSSSSIGH | |
| Human | ATGAGCTGCAACGGAGGCTCCCACCCGCGGATCAACACTCTGG | 803 |
| DSP DPII | GCCGCATGATCCGCGCCGAGTCTGGCCCGGACCTGCGCTACGA | |
| isoform | GGTGACCAGCGGCGGCGGGGGCACCAGCAGGATGTACTATTCT | |
| DNA | CGGCGCGGCGTGATCACCGACCAGAACTCGGACGGCTACTGTC | |
| | AAACCGGCACGATGTCCAGGCACCAGAACCAGAACACCATCCA | |
| | GGAGCTGCTGCAGAACTGCTCCGACTGCTTGATGCGAGCAGAG | |
| | CTCATCGTGCAGCCTGAATTGAAGTATGGAGATGGAATACAAC | |
| | TGACTCGGAGTCGAGAATTGGATGAGTGTTTTGCCCAGGCCAA | |
| | TGACCAAATGGAAATCCTCGACAGCTTGATCAGAGAGATGCGG | |
| | CAGATGGGCCAGCCCTGTGATGCTTACCAGAAAAGGCTTCTTC | |
| | AGCTCCAAGAGCAAATGCGAGCCCTTTATAAAGCCATCAGTGT | |
| | CCCTCGAGTCCGCAGGGCCAGCTCCAAGGGTGGTGGAGGCTAC | |
| | ACTTGTCAGAGTGGCTCTGGCTGGGATGAGTTCACCAAACATG | |
| | TCACCAGTGAATGTTTGGGGTGGATGAGGCAGCAAAGGGCGGA | |
| | GATGGACATGGTGGCCTGGGGTGTGGACCTGGCCTCAGTGGAG | |
| | CAGCACATTAACAGCCACCGGGGCATCCACAACTCCATCGGCG | |
| | ACTATCGCTGGCAGCTGGACAAAATCAAAGCCGACCTGCGCGA | |
| | GAAATCTGCGATCTACCAGTTGGAGGAGGAGTATGAAAACCTG | |
| | CTGAAAGCGTCCTTTGAGAGGATGGATCACCTGCGACAGCTGC | |
| | AGAACATCATTCAGGCCACGTCCAGGGAGATCATGTGGATCAA | |
| | TGACTGCGAGGAGGAGGAGCTGCTGTACGACTGGAGCGACAA | |
| | GAACACCAACATCGCTCAGAAACAGGAGGCCTTCTCCATACGC | |
| | ATGAGTCAACTGGAAGTTAAAGAAAAAGAGCTCAATAAGCTG | |
| | AAACAAGAAAGTGACCAACTTGTCCTCAATCAGCATCCAGCTT | |
| | CAGACAAAATTGAGGCCTATATGGACACTCTGCAGACGCAGTG | |
| | GAGTTGGATTCTTCAGATCACCAAGTGCATTGATGTTCATCTGA | |
| | AAGAAAATGCTGCCTACTTTCAGTTTTTTGAAGAGGCGCAGTCT | |
| | ACTGAAGCATACCTGAAGGGGCTCCAGGACTCCATCAGGAAGA | |
| | AGTACCCCTGCGACAAGAACATGCCCCTGCAGCACCTGCTGGA | |
| | ACAGATCAAGGAGCTGGAGAAAGAACGAGAGAAAATCCTTGA | |
| | ATACAAGCGTCAGGTGCAGAACTTGGTAAACAAGTCTAAGAAG | |
| | ATTGTACAGCTGAAGCCTCGTAACCCAGACTACAGAAGCAATA | |
| | AACCCATTATTCTCAGAGCTCTCTGTGACTACAAACAAGATCA | |
| | GAAAATCGTGCATAAGGGGGATGAGTGTATCCTGAAGGACAAC | |
| | AACGAGCGCAGCAAGTGGTACGTGACGGGCCCGGGAGGCGTT | |
| | GACATGCTTGTTCCCTCTGTGGGGCTGATCATCCCTCCTCCGAA | |
| | CCCACTGGCCGTGGACCTCTCTTGCAAGATTGAGCAGTACTAC | |
| | GAAGCCATCTTGGCTCTGTGGAACCAGCTCTACATCAACATGA | |
| | AGAGCCTGGTGTCCTGGCACTACTGCATGATTGACATAGAGAA | |
| | GATCAGGGCCATGACAATCGCCAAGCTGAAAACAATGCGGCA | |
| | GGAAGATTACATGAAGACGATAGCCGACCTTGAGTTACATTAC | |
| | CAAGAGTTCATCAGAAATAGCCAAGGCTCAGAGATGTTTGGAG | |
| | ATGATGACAAGCGGAAAATACAGTCTCAGTTCACCGATGCCCA | |
| | GAAGCATTACCAGACCCTGGTCATTCAGCTCCCTGGCTATCCCC | |
| | AGCACCAGACAGTGACCACAACTGAAATCACTCATCATGGAAC | |
| | CTGCCAAGATGTCAACCATAATAAAGTAATTGAAACCAACAGA | |
| | GAAAATGACAAGCAAGAAACATGGATGCTGATGGAGCTGCAG | |
| | AAGATTCGCAGGCAGATAGAGCACTGCGAGGGCAGGATGACT | |
| | CTCAAAAACCTCCCTCTAGCAGACCAGGGATCTTCTCACCACAT | |
| | CACAGTGAAAATTAACGAGCTTAAGAGTGTGCAGAATGATTCA | |
| | CAAGCAATTGCTGAGGTTCTCAACCAGCTTAAAGATATGCTTG | |
| | CCAACTTCAGAGGTTCTGAAAAGTACTGCTATTTACAGAATGA | |
| | AGTATTTGGACTATTTCAGAAACTGGAAAATATCAATGGTGTT | |
| | ACAGATGGCTACTTAAATAGCTTATGCACAGTAAGGGCACTGC | |
| | TCCAGGCTATTCTCCAAACAGAAGACATGTTAAAGGTTTATGA | |
| | AGCCAGGCTCACTGAGGAGGAAACTGTCTGCCTGGACCTGGAT | |
| | AAAGTGGAAGCTTACCGCTGTGGACTGAAGAAAATAAAAAAT | |
| | GACTTGAACTTGAAGAAGTCGTTGTTGGCCACTATGAAGACAG | |
| | AACTACAGAAAGCCCAGCAGATCCACTCTCAGACTTCACAGCA | |
| | GTATCCACTTTATGATCTGGACTTGGGCAAGTTCGGTGAAAAA | |
| | GTCACACAGCTGACAGACCGCTGGCAAAGGATAGATAAACAG | |
| | ATCGACTTTAGGTTATGGGACCTGGAGAAACAAATCAAGCAAT | |
| | TGAGGAATTATCGTGATAACTATCAGGCTTTCTGCAAGTGGCTC | |
| | TATGATGCTAAACGCCGCCAGGATTCCTTAGAATCCATGAAAT | |
| | TTGGAGATTCCAACACAGTCATGCGGTTTTTGAATGAGCAGAA | |
| | GAACTTGCACAGTGAAATATCTGGCAAACGAGACAAATCAGAG | |
| | GAAGTACAAAAAATTGCTGAACTTTGCGCCAATTCAATTAAGG | |
| | ATTATGAGCTCCAGCTGGCCTCATACACCTCAGGACTGGAAAC | |
| | TCTGCTGAACATACCTATCAAGAGGACCATGATTCAGTCCCCTT | |
| | CTGGGGTGATTCTGCAAGAGGCTGCAGATGTTCATGCTCGGTA | |
| | CATTGAACTACTTACAAGATCTGGAGACTATTACAGGTTCTTAA | |
| | GTGAGATGCTGAAGAGTTTGGAAGATCTGAAGCTGAAAAATAC | |
| | CAAGATCGAAGTTTTGGAAGAGGAGCTCAGACTGGCCCGAGAT | |
| | GCCAACTCGGAAAACTGTAATAAGAACAAATTCCTGGATCAGA | |
| | ACCTGCAGAAATACCAGGCAGAGTGTTCCCAGTTCAAAGCGAA | |
| | GCTTGCGAGCCTGGAGGAGCTGAAGAGACAGGCTGAGCTGGAT | |
| | GGGAAGTCGGCTAAGCAAAATCTAGACAAGTGCTACGGCCAA | |
| | ATAAAAGAACTCAATGAGAAGATCACCCGACTGACTTATGAGA | |
| | TTGAAGATGAAAAGAGAAGAAGAAAATCTGTGGAAGACAGAT | |
| | TTGACCAACAGAAGAATGACTATGACCAACTGCAGAAAGCAA | |
| | GGCAATGTGAAAAGGAGAACCTTGGTTGGCAGAAATTAGAGTC | |
| | TGAGAAAGCCATCAAGGAGAAGGAGTACGAGATTGAAAGGTT | |
| | GAGGGTTCTACTGCAGGAAGAAGGCACCCGGAAGAGAGAATA | |
| | TGAAAATGAGCTGGCAAAGGCATCTAATAGGATTCAGGAATCA | |
| | AAGAATCAGTGTACTCAGGTGGTACAGGAAAGAGAGAGCCTTC | |
| | TGGTGAAAATCAAAGTCCTGGAGCAAGACAAGGCAAGGCTGC | |
| | AGAGGCTGGAGGATGAGCTGAATCGTGCAAAATCAACTCTAGA | |
| | GGCAGAAACCAGGGTGAAACAGCGCCTGGAGTGTGAGAAACA | |
| | GCAAATTCAGAATGACCTGAATCAGTGGAAGACTCAATATTCC | |
| | CGCAAGGAGGAGGCTATTAGGAAGATAGAATCGGAAAGAGAA | |
| | AAGAGTGAGAGAGAGAAGAACAGTCTTAGGAGTGAGATCGAA | |
| | AGACTCCAAGCAGAGATCAAGAGAATTGAAGAGAGGTGCAGG | |
| | CGTAAGCTGGAGGATTCTACCAGGGAGACACAGTCACAGTTAG | |
| | AAACAGAACGCTCCCGATATCAGAGGGAGATTGATAAACTCAG | |
| | ACAGCGCCCATATGGGTCCCATCGAGAGACCCAGACTGAGTGT | |
| | GAGTGGACCGTTGACACCTCCAAGCTGGTGTTTGATGGGCTGA | |
| | GGAAGAAGGTGACAGCAATGCAGCTCTATGAGTGTCAGCTGAT | |
| | CGACAAAACAACCTTGGACAAACTATTGAAGGGGAAGAAGTC | |
| | AGTGGAAGAAGTTGCTTCTGAAATCCAGCCATTCCTTCGGGGT | |
| | GCAGGATCTATCGCTGGAGCATCTGCTTCTCCTAAGGAAAAAT | |
| | ACTCTTTGGTAGAGGCCAAGAGAAAGAAATTAATCAGCCCAGA | |
| | ATCCACAGTCATGCTTCTGGAGGCCCAGGCAGCTACAGGTGGT | |
| | ATAATTGATCCCCATCGGAATGAGAAGCTGACTGTCGACAGTG | |
| | CCATAGCTCGGGACCTCATTGACTTCGATGACCGTCAGCAGAT | |
| | ATATGCAGCAGAAAAAGCTATCACTGGTTTTGATGATCCATTTT | |
| | CAGGCAAGACAGTATCTGTTTCAGAAGCCATCAAGAAAAATTT | |
| | GATTGATAGAGAAACCGGAATGCGCCTGCTGGAAGCCCAGATT | |
| | GCTTCAGGGGGTGTAGTAGACCCTGTGAACAGTGTCTTTTTGCC | |
| | AAAAGATGTCGCCTTGGCCCGGGGGCTGATTGATAGAGATTTG | |
| | TATCGATCCCTGAATGATCCCCGAGATAGTCAGAAAAACTTTG | |
| | TGGATCCAGTCACCAAAAAGAAGGTCAGTTACGTGCAGCTGAA | |
| | GGAACGGTGCAGAATCGAACCACATACTGGTCTGCTCTTGCTTT | |
| | CAGTACAGAAGAGAAGCATGTCCTTCCAAGGAATCAGACAACC | |
| | TGTGACCGTCACTGAGCTAGTAGATTCTGGTATATTGAGACCGT | |
| | CCACTGTCAATGAACTGGAATCTGGTCAGATTTCTTATGACGAG | |
| | GTTGGTGAGAGAATTAAGGACTTCCTCCAGGGTTCAAGCTGCA | |
| | TAGCAGGCATATACAATGAGACCACAAAACAGAAGCTTGGCAT | |
| | TTATGAGGCCATGAAAATTGGCTTAGTCCGACCTGGTACTGCTC | |
| | TGGAGTTGCTGGAAGCCCAAGCAGCTACTGGCTTTATAGTGGA | |
| | TCCTGTTAGCAACTTGAGGTTACCAGTGGAGGAAGCCTACAAG | |
| | AGAGGTCTGGTGGGCATTGAGTTCAAAGAGAAGCTCCTGTCTG | |
| | CAGAACGAGCTGTCACTGGGTATAATGATCCTGAAACAGGAAA | |
| | CATCATCTCTTTGTTCCAAGCCATGAATAAGGAACTCATCGAAA | |
| | AGGGCCACGGTATTCGCTTATTAGAAGCACAGATCGCAACCGG | |
| | GGGGATCATTGACCCAAAGGAGAGCCATCGTTTACCAGTTGAC | |
| | ATAGCATATAAGAGGGGCTATTTCAATGAGGAACTCAGTGAGA | |
| | TTCTCTCAGATCCAAGTGATGATACCAAAGGATTTTTTGACCCC | |
| | AACACTGAAGAAAATCTTACCTATCTGCAACTAAAAGAAAGAT | |
| | GCATTAAGGATGAGGAAACAGGGCTCTGTCTTCTGCCTCTGAA | |
| | AGAAAAGAAGAAACAGGTGCAGACATCACAAAAGAATACCCT | |
| | CAGGAAGCGTAGAGTGGTCATAGTTGACCCAGAAACCAATAAA | |
| | GAAATGTCTGTTCAGGAGGCCTACAAGAAGGGCCTAATTGATT | |
| | ATGAAACCTTCAAAGAACTGTGTGAGCAGGAATGTGAATGGGA | |
| | AGAAATAACCATCACGGGATCAGATGGCTCCACCAGGGTGGTC | |
| | CTGGTAGATAGAAAGACAGGCAGTCAGTATGATATTCAAGATG | |
| | CTATTGACAAGGGCCTTGTTGACAGGAAGTTCTTTGATCAGTAC | |
| | CGATCCGGCAGCCTCAGCCTCACTCAATTTGCTGACATGATCTC | |
| | CTTGAAAAATGGTGTCGGCACCAGCAGCAGCATGGGCAGTGGT | |
| | GTCAGCGATGATGTTTTTAGCAGCTCCCGACATGAATCAGTAA | |
| | GTAAGATTTCCACCATATCCAGCGTCAGGAATTTAACCATAAG | |
| | GAGCAGCTCTTTTTCAGACACCCTGGAAGAATCGAGCCCCATT | |
| | GCAGCCATCTTTGACACAGAAAACCTGGAGAAAATCTCCATTA | |
| | CAGAAGGTATAGAGCGGGGCATCGTTGACAGCATCACGGGTCA | |
| | GAGGCTTCTGGAGGCTCAGGCCTGCACAGGTGGCATCATCCAC | |
| | CCAACCACGGGCCAGAAGCTGTCACTTCAGGACGCAGTCTCCC | |
| | AGGGTGTGATTGACCAAGACATGGCCACCAGGCTGAAGCCTGC | |
| | TCAGAAAGCCTTCATAGGCTTCGAGGGTGTGAAGGGAAAGAAG | |
| | AAGATGTCAGCAGCAGAGGCAGTGAAAGAAAAATGGCTCCCG | |
| | TATGAGGCTGGCCAGCGCTTCCTGGAGTTCCAGTACCTCACGG | |
| | GAGGTCTTGTTGACCCGGAAGTGCATGGGAGGATAAGCACCGA | |
| | AGAAGCCATCCGGAAGGGGTTCATAGATGGCCGCGCCGCACAG | |
| | AGGCTGCAAGACACCAGCAGCTATGCCAAAATCCTGACCTGCC | |
| | CCAAAACCAAATTAAAAATATCCTATAAGGATGCCATAAATCG | |
| | CTCCATGGTAGAAGATATCACTGGGCTGCGCCTTCTGGAAGCC | |
| | GCCTCCGTGTCGTCCAAGGGCTTACCCAGCCCTTACAACATGTC | |
| | TTCGGCTCCGGGGTCCCGCTCCGGCTCCCGCTCGGGATCTCGCT | |
| | CCGGATCTCGCTCCGGGTCCCGCAGTGGGTCCCGGAGAGGAAG | |
| | CTTTGACGCCACAGGGAATTCTTCCTACTCTTATTCCTACTCATT | |
| | TAGCAGTAGTTCTATTGGGCACTAG | |
| Human | MARSPGRAYALLLLLICFNVGSGLHLQVLSTRNENKLLPKHPHLV | 804 |
| DSG2 | RQKRAWITAPVALREGEDLSKKNPIAKIHSDLAEERGLKITYKYTG | |
| protein | KGITEPPFGIFVFNKDTGELNVTSILDREETPFFLLTGYALDARGNN | |
| | VEKPLELRIKVLDINDNEPVFTQDVFVGSVEELSAAHTLVMKINAT | |
| | DADEPNTLNSKISYRIVSLEPAYPPVFYLNKDTGEIYTTSVTLDREE | |
| | HSSYTLTVEARDGNGEVTDKPVKQAQVQIRILDVNDNIPVVENKV | |
| | LEGMVEENQVNVEVTRIKVFDADEIGSDNWLANFTFASGNEGGY | |
| | FHIETDAQTNEGIVTLIKEVDYEEMKNLDFSVIVANKAAFHKSIRS | |
| | KYKPTPIPIKVKVKNVKEGIHFKSSVISIYVSESMDRSSKGQIIGNFQ | |
| | AFDEDTGLPAHARYVKLEDRDNWISVDSVTSEIKLAKLPDFESRY | |
| | VQNGTYTVKIVAISEDYPRKTITGTVLINVEDINDNCPTLIEPVQTIC | |
| | HDAEYVNVTAEDLDGHPNSGPFSFSVIDKPPGMAEKWKIARQEST | |
| | SVLLQQSEKKLGRSEIQFLISDNQGFSCPEKQVLTLTVCECLHGSG | |
| | CREAQHDSYVGLGPAAIALMILAFLLLLLVPLLLLMCHCGKGAKG | |
| | FTPIPGTIEMLHPWNNEGAPPEDKVVPSFLPVDQGGSLVGRNGVG | |
| | GMAKEATMKGSSSASIVKGQHEMSEMDGRWEEHRSLLSGRATQF | |
| | TGATGAIMTTETTKTARATGASRDMAGAQAAAVALNEEFLRNYF | |
| | TDKAASYTEEDENHTAKDCLLVYSQEETESLNASIGCCSFIEGELD | |
| | DRFLDDLGLKFKTLAEVCLGQKIDINKEIEQRQKPATETSMNTASH | |
| | SLCEQTMVNSENTYSSGSSFPVPKSLQEANAEKVTQEIVTERSVSS | |
| | RQAQKVATPLPDPMASRNVIATETSYVTGSTMPPTTVILGPSQPQS | |
| | LIVTERVYAPASTLVDQPYANEGTVVVTERVIQPHGGGSNPLEGT | |
| | QHLQDVPYVMVRERESFLAPSSGVQPTLAMPNIAVGQNVTVTER | |
| | VLAPASTLQSSYQIPTENSMTARNTTVSGAGVPGPLPDFGLEESGH | |
| | SNSTITTSSTRVTKHSTVQHSYS | |
| Human | ATGGCGCGGAGCCCGGGACGCGCGTACGCCCTGCTGCTTCTCC | 805 |
| DSG2 | TGATCTGCTTTAACGTTGGAAGTGGACTTCACTTACAGGTCTTA | |
| DNA | AGCACAAGAAATGAAAATAAGCTGCTTCCTAAACATCCTCATT | |
| | TAGTGCGGCAAAAGCGCGCCTGGATCACCGCCCCCGTGGCTCT | |
| | TCGGGAGGGAGAGGATCTGTCCAAGAAGAATCCAATTGCCAAG | |
| | ATACATTCTGATCTTGCAGAAGAAAGAGGACTCAAAATTACTT | |
| | ACAAATACACTGGAAAAGGGATTACAGAGCCACCTTTTGGTAT | |
| | ATTTGTCTTTAACAAAGATACTGGAGAACTGAATGTTACCAGC | |
| | ATTCTTGATCGAGAAGAAACACCATTTTTTCTGCTAACAGGTTA | |
| | CGCTTTGGATGCAAGAGGAAACAATGTAGAGAAACCCTTAGAG | |
| | CTACGCATTAAGGTTCTTGATATCAATGACAACGAACCAGTGTT | |
| | CACACAGGATGTCTTTGTTGGGTCTGTTGAAGAGTTGAGTGCA | |
| | GCACATACTCTTGTGATGAAAATCAATGCAACAGATGCAGATG | |
| | AGCCCAATACCCTGAATTCGAAAATTTCCTATAGAATCGTATCT | |
| | CTGGAGCCTGCTTATCCTCCAGTGTTCTACCTAAATAAAGATAC | |
| | AGGAGAGATTTATACAACCAGTGTTACCTTGGACAGAGAGGAA | |
| | CACAGCAGCTACACTTTGACAGTAGAAGCAAGAGATGGCAATG | |
| | GAGAAGTTACAGACAAACCTGTAAAACAAGCTCAAGTTCAGAT | |
| | TCGTATTTTGGATGTCAATGACAATATACCTGTAGTAGAAAATA | |
| | AAGTGCTTGAAGGGATGGTTGAAGAAAATCAAGTCAACGTAGA | |
| | AGTTACGCGCATAAAAGTGTTCGATGCAGATGAAATAGGTTCT | |
| | GATAATTGGCTGGCAAATTTTACATTTGCATCAGGAAATGAAG | |
| | GAGGTTATTTCCACATAGAAACAGATGCTCAAACTAACGAAGG | |
| | AATTGTGACCCTTATTAAGGAAGTAGATTATGAAGAAATGAAG | |
| | AATCTTGACTTCAGTGTTATTGTCGCTAATAAAGCAGCTTTTCA | |
| | CAAGTCGATTAGGAGTAAATACAAGCCTACACCCATTCCCATC | |
| | AAGGTCAAAGTGAAAAATGTGAAAGAAGGCATTCATTTTAAAA | |
| | GCAGCGTCATCTCAATTTATGTTAGCGAGAGCATGGATAGATC | |
| | AAGCAAAGGCCAAATAATTGGAAATTTTCAAGCTTTTGATGAG | |
| | GACACTGGACTACCAGCCCATGCAAGATATGTAAAATTAGAAG | |
| | ATAGAGATAATTGGATCTCTGTGGATTCTGTCACATCTGAAATT | |
| | AAACTTGCAAAACTTCCTGATTTTGAATCTAGATATGTTCAAAA | |
| | TGGCACATACACTGTAAAGATTGTGGCCATATCAGAAGATTAT | |
| | CCTAGAAAAACCATCACTGGCACAGTCCTTATCAATGTTGAAG | |
| | ACATCAACGACAACTGTCCCACACTGATAGAGCCTGTGCAGAC | |
| | AATCTGTCACGATGCAGAGTATGTGAATGTTACTGCAGAGGAC | |
| | CTGGATGGACACCCAAACAGTGGCCCTTTCAGTTTCTCCGTCAT | |
| | TGACAAACCACCTGGCATGGCAGAAAAATGGAAAATAGCACG | |
| | CCAAGAAAGTACCAGTGTGCTGCTGCAACAAAGTGAGAAAAA | |
| | GCTTGGGAGAAGTGAAATTCAGTTCCTGATTTCAGACAATCAG | |
| | GGTTTTAGTTGTCCTGAAAAGCAGGTCCTTACACTCACAGTTTG | |
| | TGAGTGTCTGCATGGCAGCGGCTGCAGGGAAGCACAGCATGAC | |
| | TCCTATGTGGGCCTGGGACCCGCAGCAATTGCGCTCATGATTTT | |
| | GGCCTTTCTGCTCCTGCTATTGGTACCACTTTTACTGCTGA | |
| | TGTGCCATTGCGGAAAGGGCGCCAAAGGCTTTACCCCCATACC | |
| | TGGCACCATAGAGATGCTGCATCCTTGGAATAATGAAGGAGCA | |
| | CCACCTGAAGACAAGGTGGTGCCATCATTTCTGCCAGTGGATC | |
| | AAGGGGGCAGTCTAGTAGGAAGAAATGGAGTAGGAGGTATGG | |
| | CCAAGGAAGCCACGATGAAAGGAAGTAGCTCTGCTTCCATTGT | |
| | CAAAGGGCAACATGAGATGTCCGAGATGGATGGAAGGTGGGA | |
| | AGAACACAGAAGCCTGCTTTCTGGTAGAGCTACCCAGTTTACA | |
| | GGGGCCACAGGCGCTATCATGACCACTGAAACCACGAAGACCG | |
| | CAAGGGCCACAGGGGCTTCCAGAGACATGGCCGGAGCTCAGG | |
| | CAGCTGCTGTTGCACTGAACGAAGAATTCTTAAGAAATTATTTC | |
| | ACTGATAAAGCGGCCTCTTACACTGAGGAAGATGAAAATCACA | |
| | CAGCCAAAGATTGCCTTCTGGTTTATTCTCAGGAAGAAACTGA | |
| | ATCGCTGAATGCTTCTATTGGTTGTTGCAGTTTTATTGAAGGAG | |
| | AGCTAGATGACCGCTTCTTAGATGATTTGGGACTTAAATTCAAG | |
| | ACACTAGCTGAAGTTTGCCTGGGTCAAAAAATAGATATAAATA | |
| | AGGAAATTGAGCAGAGACAAAAACCTGCCACAGAAACAAGTA | |
| | TGAACACAGCTTCACATTCACTCTGTGAGCAAACTATGGTTAAT | |
| | TCAGAGAATACCTACTCCTCTGGCAGTAGCTTCCCAGTTCCAAA | |
| | ATCTTTGCAAGAAGCCAATGCAGAGAAAGTAACTCAGGAAATA | |
| | GTCACTGAAAGATCTGTGTCTTCTAGGCAGGCGCAAAAGGTAG | |
| | CTACACCTCTTCCTGACCCAATGGCTTCTAGAAATGTGATAGCA | |
| | ACAGAAACTTCCTATGTCACAGGGTCCACTATGCCACCAACCA | |
| | CTGTGATCCTGGGTCCTAGCCAGCCACAGAGCCTTATTGTGACA | |
| | GAGAGGGTGTATGCTCCAGCTTCTACCTTGGTAGATCAGCCTTA | |
| | TGCTAATGAAGGTACAGTTGTGGTCACTGAAAGAGTAATACAG | |
| | CCTCATGGGGGTGGATCGAATCCTCTGGAAGGCACTCAGCATC | |
| | TTCAAGATGTACCTTACGTCATGGTGAGGGAAAGAGAGAGCTT | |
| | CCTTGCCCCCAGCTCAGGTGTGCAGCCTACTCTGGCCATGCCTA | |
| | ATATAGCAGTAGGACAGAATGTGACAGTGACAGAAAGAGTTCT | |
| | AGCACCTGCTTCCACTCTGCAATCCAGTTACCAGATTCCCACTG | |
| | AAAATTCTATGACGGCTAGGAACACCACGGTGTCTGGAGCTGG | |
| | AGTCCCTGGCC | |
| | CTCTGCCAGATTTTGGTTTAGAGGAATCTGGTCATTCTAATTCT | |
| | ACCATAACCACATCTTCCACCAGAGTTACCAAGCATAGCACTG | |
| | TACAGCATTCTTACTCCTAA | |
| Human | MEVMNLMEQPIKVTEWQQTYTYDSGIHSGANTCVPSVSSKGIME | 806 |
| JUP | EDEACGRQYTLKKTTTYTQGVPPSQGDLEYQMSTTARAKRVREA | |
| protein | MCPGVSGEDSSLLLATQVEGQATNLQRLAEPSQLLKSAIVHLINY | |
| | QDDAELATRALPELTKLLNDEDPVVVTKAAMIVNQLSKKEASRR | |
| | ALMGSPQLVAAVVRTMQNTSDLDTARCTTSILHNLSHHREGLLAI | |
| | FKSGGIPALVRMLSSPVESVLFYAITTLHNLLLYQEGAKMAVRLA | |
| | DGLQKMVPLLNKNNPKFLAITTDCLQLLAYGNQESKLIILANGGP | |
| | QALVQIMRNYSYEKLLWTTSRVLKVLSVCPSNKPAIVEAGGMQA | |
| | LGKHLTSNSPRLVQNCLWTLRNLSDVATKQEGLESVLKILVNQLS | |
| | VDDVNVLTCATGTLSNLTCNNSKNKTLVTQNSGVEALIHAILRAG | |
| | DKDDITEPAVCALRHLTSRHPEAEMAQNSVRLNYGIPAIVKLLNQ | |
| | PNQWPLVKATIGLIRNLALCPANHAPLQEAAVIPRLVQLLVKAHQ | |
| | DAQRHVAAGTQQPYTDGVRMEEIVEGCTGALHILARDPMNRMEI | |
| | FRLNTIPLFVQLLYSSVENIQRVAAGVLCELAQDKEAADAIDAEGA | |
| | SAPLMELLHSRNEGTATYAAAVLFRISEDKNPDYRKRVSVELTNS | |
| | LFKHDPAAWEAAQSMIPINEPYGDDMDATYRPMYSSDVPLDPLE | |
| | MHMDMDGDYPIDTYSDGLRPPYPTADHMLA | |
| Human | ATGGAGGTGATGAACCTGATGGAGCAGCCTATCAAGGTGACTG | 807 |
| JUP DNA | AGTGGCAGCAGACATACACCTACGACTCGGGTATCCACTCGGG | |
| | CGCCAACACCTGCGTGCCCTCCGTCAGCAGCAAGGGCATCATG | |
| | GAGGAGGATGAGGCCTGCGGGCGCCAGTACACGCTCAAGAAA | |
| | ACCACCACTTACACCCAGGGGGTGCCCCCCAGCCAAGGTGATC | |
| | TGGAGTACCAGATGTCCACAACAGCCAGGGCCAAACGGGTGCG | |
| | GGAGGCCATGTGCCCTGGTGTGTCAGGCGAGGACAGCTCGCTT | |
| | CTGCTGGCCACCCAGGTGGAGGGGCAGGCCACCAACCTGCAGC | |
| | GACTGGCCGAGCCGTCCCAGCTGCTCAAGTCGGCCATTGTGCA | |
| | TCTCATCAACTACCAGGACGATGCCGAGCTGGCCACTCGCGCC | |
| | CTGCCCGAGCTCACCAAACTGCTCAACGACGAGGACCCGGTGG | |
| | TGGTGACCAAGGCGGCCATGATTGTGAACCAGCTGTCGAAGAA | |
| | GGAGGCGTCGCGGCGGGCCCTGATGGGCTCGCCCCAGCTGGTG | |
| | GCCGCTGTCGTGCGTACCATGCAGAATACCAGCGACCTGGACA | |
| | CAGCCCGCTGCACCACCAGCATCCTGCACAACCTCTCCCACCA | |
| | CCGGGAGGGGCTGCTCGCCATCTTCAAGTCGGGTGGCATCCCT | |
| | GCTCTGGTCCGCATGCTCAGCTCCCCTGTGGAGTCGGTCCTGTT | |
| | CTATGCCATCACCACGCTGCACAACCTGCTCCTGTACCAGGAG | |
| | GGCGCCAAGATGGCCGTGCGCCTGGCCGACGGGCTGCAAAAG | |
| | ATGGTGCCCCTGCTCAACAAGAACAACCCCAAGTTCCTGGCCA | |
| | TCACCACCGACTGCCTGCAGCTCCTGGCCTACGGCAACCAGGA | |
| | GAGCAAGCTGATCATCCTGGCCAATGGTGGGCCCCAGGCCCTC | |
| | GTGCAGATCATGCGTAACTACAGTTATGAAAAGCTGCTCTGGA | |
| | CCACCAGTCGTGTGCTCAAGGTGCTATCCGTGTGTCCCAGCAAT | |
| | AAGCCTGCCATTGTGGAGGCTGGTGGGATGCAGGCCCTGGGCA | |
| | AGCACCTGACCAGCAACAGCCCCCGCCTGGTGCAGAACTGCCT | |
| | GTGGACCCTGCGCAACCTCTCAGATGTGGCCACCAAGCAGGAG | |
| | GGCCTGGAGAGTGTGCTGAAGATTCTGGTGAATCAGCTGAGTG | |
| | TGGATGACGTCAACGTCCTCACCTGTGCCACGGGCACACTCTCC | |
| | AACCTGACATGCAACAACAGCAAGAACAAGACGCTGGTGACA | |
| | CAGAACAGCGGTGTGGAGGCTCTCATCCATGCCATCCTGCGTG | |
| | CTGGTGACAAGGACGACATCACGGAGCCTGCCGTCTGCGCTCT | |
| | GCGCCACCTCACTAGCCGCCACCCTGAGGCCGAGATGGCCCAG | |
| | AACTCTGTGCGTCTCAACTATGGCATCCCAGCCATCGTGAAGCT | |
| | GCTCAACCAGCCCAACCAGTGGCCACTGGTCAAGGCAACCATC | |
| | GGCTTGATCAGGAATCTGGCCCTGTGCCCAGCCAACCATGCCC | |
| | CGCTGCAGGAGGCAGCGGTCATCCCCCGCCTCGTCCAACTGCT | |
| | GGTGAAGGCCCACCAGGATGCCCAGCGCCACGTAGCTGCAGGC | |
| | ACACAGCAGCCCTACACGGATGGTGTGAGGATGGAGGAGATTG | |
| | TGGAGGGCTGCACCGGAGCACTGCACATCCTCGCCCGGGACCC | |
| | CATGAACCGCATGGAGATCTTCCGGCTCAACACCATTCCCCTGT | |
| | TTGTGCAGCTCCTGTACTCGTCGGTGGAGAACATCCAGCGCGT | |
| | GGCTGCCGGGGTGCTGTGTGAGCTGGCCCAGGACAAGGAGGCG | |
| | GCCGACGCCATTGATGCAGAGGGGGCCTCGGCCCCACTCATGG | |
| | AGTTGCTGCACTCCCGCAACGAGGGCACTGCCACCTACGCTGC | |
| | TGCCGTCCTGTTCCGCATCTCCGAGGACAAGAACCCAGACTAC | |
| | CGGAAGCGCGTGTCCGTGGAGCTCACCAACTCCCTCTTCAAGC | |
| | ATGACCCGGCTGCCTGGGAGGCTGCCCAGAGCATGATTCCCAT | |
| | CAATGAGCCCTATGGAGATGACATGGATGCCACCTACCGCCCC | |
| | ATGTACTCCAGCGATGTGCCCCTTGACCCGCTGGAGATGCACA | |
| | TGGACATGGATGGAGACTACCCCATCGACACCTACAGCGACGG | |
| | CCTCAGGCCCCCGTACCCCACTGCAGACCACATGCTGGCCTAG | |
| Human | MSGGRFDFDDGGAYCGGWEGGKAHGHGLCTGPKGQGEYSGSW | 808 |
| JPH2 N- | NFGFEVAGVYTWPSGNTFEGYWSQGKRHGLGIETKGRWLYKGE | |
| terminal | WTHGFKGRYGIRQSSSSGAKYEGTWNNGLQDGYGTETYADGGT | |
| fragment | YQGQFTNGMRHGYGVRQSVPYGMAVVVRSPLRTSLSSLRSEHSN | |
| protein | GTVAPDSPASPASDGPALPSPAIPRGGFALSLLANAEAAARAPKGG | |
| | GLFQRGALLGKLRRAESRTSVGSQRSRVSFLKSDLSSGASDAAST | |
| | ASLGEAAEGADEAAPFEADIDATTTETYMGEWKNDKRSGFGVSE | |
| | RSSGLRYEGEWLDNLRHGYGCTTLPDGHREEGKYRHNVLVKDTK | |
| | RRMLQLKSNKVRQKVEHSVEGAQRAAAIARQKAEIAASRTSHAK | |
| | AKAEAAEQAALAANQESNIARTLARELAPDFYQPGPEYQKRRLLQ | |
| | EILENSESLLEPPDRGAGAAGLPQPPRESPQLHERETPRPEGGSPSP | |
| | AGTPPQPKRPRPGVSKDGLLSPGAWNGEPSGEGSRSVTPSEGAGR | |
| | RSPARPATERMAIEALQAPPAPSREPEVALYQGYHSYAVR | |
| Human | ATGAGTGGGGGCCGCTTCGACTTTGATGATGGAGGGGCGTACT | 809 |
| JPH2 N- | GCGGGGGCTGGGAGGGGGGAAAGGCCCATGGGCATGGACTGT | |
| terminal | GCACAGGCCCCAAGGGCCAGGGCGAATACTCTGGCTCCTGGAA | |
| fragment | CTTTGGCTTTGAGGTGGCAGGTGTCTACACCTGGCCCAGCGGA | |
| DNA | AACACCTTTGAGGGATACTGGAGCCAGGGCAAACGGCATGGGC | |
| | TGGGCATAGAGACCAAGGGGCGCTGGCTCTACAAGGGCGAGT | |
| | GGACACATGGCTTCAAGGGACGCTACGGAATCCGGCAGAGCTC | |
| | AAGCAGCGGTGCCAAGTATGAGGGCACCTGGAACAATGGCCTG | |
| | CAAGACGGCTATGGCACCGAGACCTATGCTGATGGAGGGACGT | |
| | ACCAAGGCCAGTTCACCAACGGCATGCGCCATGGCTACGGAGT | |
| | ACGCCAGAGCGTGCCCTACGGGATGGCCGTGGTGGTGCGCTCG | |
| | CCGCTGCGCACGTCGCTGTCGTCCCTGCGCAGCGAGCACAGCA | |
| | ACGGCACGGTGGCCCCGGACTCTCCCGCCTCGCCGGCCTCCGA | |
| | CGGCCCCGCGCTGCCCTCGCCCGCCATCCCGCGTGGCGGCTTCG | |
| | CGCTCAGCCTCCTGGCCAATGCCGAGGCGGCCGCGCGGGCGCC | |
| | CAAGGGCGGCGGCCTCTTCCAGCGGGGCGCGCTGCTGGGCAAG | |
| | CTGCGGCGCGCAGAGTCGCGCACGTCCGTGGGTAGCCAGCGCA | |
| | GCCGTGTCAGCTTCCTTAAGAGCGACCTCAGCTCGGGCGCCAG | |
| | CGACGCCGCGTCCACCGCCAGCCTGGGAGAGGCCGCCGAGGGC | |
| | GCCGACGAGGCCGCACCCTTCGAGGCCGATATCGACGCCACCA | |
| | CCACCGAGACCTACATGGGCGAGTGGAAGAACGACAAACGCT | |
| | CGGGCTTCGGCGTGAGCGAACGCTCCAGTGGCCTCCGCTACGA | |
| | GGGCGAGTGGCTGGACAACCTGCGCCACGGCTATGGCTGCACC | |
| | ACGCTGCCCGACGGCCACCGCGAGGAGGGCAAGTACCGCCAC | |
| | AACGTGCTGGTCAAGGACACCAAGCGCCGCATGCTGCAGCTCA | |
| | AGAGCAACAAGGTCCGCCAGAAAGTGGAGCACAGTGTGGAGG | |
| | GTGCCCAGCGCGCCGCTGCTATCGCGCGCCAGAAGGCCGAGAT | |
| | TGCCGCCTCCAGGACAAGCCACGCCAAGGCCAAAGCTGAGGCA | |
| | GCGGAACAGGCCGCCCTGGCTGCCAACCAGGAGTCCAACATTG | |
| | CTCGCACTTTGGCCAGGGAGCTGGCTCCGGACTTCTACCAGCC | |
| | AGGTCCGGAATATCAGAAGCGCCGGCTGCTGCAGGAGATCCTG | |
| | GAGAACTCGGAGAGCCTGCTGGAGCCCCCCGACCGGGGCGCCG | |
| | GCGCAGCGGGCCTCCCACAGCCGCCCCGCGAGAGCCCGCAGCT | |
| | GCACGAGCGTGAGACCCCTCGGCCCGAGGGTGGCTCCCCGTCA | |
| | CCGGCCGGGACGCCCCCGCAGCCCAAGCGGCCCAGGCCCGGG | |
| | GTGTCCAAGGACGGCCTGCTGAGCCCAGGCGCCTGGAACGGCG | |
| | AGCCCAGCGGTGAGGGCAGCCGGTCAGTCACTCCGTCCGAGGG | |
| | CGCGGGCCGCCGCAGCCCCGCGCGTCCAGCCACCGAGCGCATG | |
| | GCCATCGAGGCTCTGCAGGCACCGCCTGCGCCGTCGCGGGAGC | |
| | CGGAGGTGGCGCTTTACCAGGGCTACCACAGCTATGCTGTGCG | |
| | C | |
| Human | MEKVQYLTRSAIRRASTIEMPQQARQKLQNLFINFCLILICLLLICII | 830 |
| PLN | VMLL | |
| protein | | |
| Human | ATGGAGAAAGTCCAATACCTCACTCGCTCAGCTATAAGAAGAG | 810 |
| PLN DNA | CCTCAACCATTGAAATGCCTCAACAAGCACGTCAAAAGCTACA | |
| | GAATCTATTTATCAATTTCTGTCTCATCTTAATATGTCTCTTGCT | |
| | GATCTGTATCATCGTGATGCTTCTCTGA | |
| Human | ATGCCTGAGCCGGGGAAGAAGCCAGTCTCAGCTTTTAGCAAGA | 811 |
| MYBPC3 | AGCCACGGTCAGTGGAAGTGGCCGCAGGCAGCCCTGCCGTGTT | |
| DNA | CGAGGCCGAGACAGAGCGGGCAGGAGTGAAGGTGCGCTGGCA | |
| | GCGCGGAGGCAGTGACATCAGCGCCAGCAACAAGTACGGCCT | |
| | GGCCACAGAGGGCACACGGCATACGCTGACAGTGCGGGAAGT | |
| | GGGCCCTGCCGACCAGGGATCTTACGCAGTCATTGCTGGCTCCT | |
| | CCAAGGTCAAGTTCGACCTCAAGGTCATAGAGGCAGAGAAGGC | |
| | AGAGCCCATGCTGGCCCCTGCCCCTGCCCCTGCTGAGGCCACT | |
| | GGAGCCCCTGGAGAAGCCCCGGCCCCAGCCGCTGAGCTGGGAG | |
| | AAAGTGCCCCAAGTCCCAAAGGGTCAAGCTCAGCAGCTCTCAA | |
| | TGGTCCTACCCCTGGAGCCCCCGATGACCCCATTGGCCTCTTCG | |
| | TGATGCGGCCACAGGATGGCGAGGTGACCGTGGGTGGCAGCAT | |
| | CACCTTCTCAGCCCGCGTGGCCGGCGCCAGCCTCCTGAAGCCG | |
| | CCTGTGGTCAAGTGGTTCAAGGGCAAATGGGTGGACCTGAGCA | |
| | GCAAGGTGGGCCAGCACCTGCAGCTGCACGACAGCTACGACCG | |
| | CGCCAGCAAGGTCTATCTGTTCGAGCTGCACATCACCGATGCC | |
| | CAGCCTGCCTTCACTGGCAGCTACCGCTGTGAGGTGTCCACCA | |
| | AGGACAAATTTGACTGCTCCAACTTCAATCTCACTGTCCACGAG | |
| | GCCATGGGCACCGGAGACCTGGACCTCCTATCAGCCTTCCGCC | |
| | GCACGAGCCTGGCTGGAGGTGGTCGGCGGATCAGTGATAGCCA | |
| | TGAGGACACTGGGATTCTGGACTTCAGCTCACTGCTGAAAAAG | |
| | AGAGACAGTTTCCGGACCCCGAGGGACTCGAAGCTGGAGGCAC | |
| | CAGCAGAGGAGGACGTGTGGGAGATCCTACGGCAGGCACCCC | |
| | CATCTGAGTACGAGCGCATCGCCTTCCAGTACGGCGTCACTGA | |
| | CCTGCGCGGCATGCTAAAGAGGCTCAAGGGCATGAGGCGCGAT | |
| | GAGAAGAAGAGCACAGCCTTTCAGAAGAAGCTGGAGCCGGCC | |
| | TACCAGGTGAGCAAAGGCCACAAGATCCGGCTGACCGTGGAAC | |
| | TGGCTGACCATGACGCTGAGGTCAAATGGCTCAAGAATGGCCA | |
| | GGAGATCCAGATGAGCGGCAGCAAGTACATCTTTGAGTCCATC | |
| | GGTGCCAAGCGTACCCTGACCATCAGCCAGTGCTCATTGGCGG | |
| | ACGACGCAGCCTACCAGTGCGTGGTGGGTGGCGAGAAGTGTAG | |
| | CACGGAGCTCTTTGTGAAAGAGCCCCCTGTGCTCATCACGCGC | |
| | CCCTTGGAGGACCAGCTGGTGATGGTGGGGCAGCGGGTGGAGT | |
| | TTGAGTGTGAAGTATCGGAGGAGGGGGCGCAAGTCAAATGGCT | |
| | GAAGGACGGGGTGGAGCTGACCCGGGAGGAGACCTTCAAATA | |
| | CCGGTTCAAGAAGGACGGGCAGAGACACCACCTGATCATCAAC | |
| | GAGGCCATGCTGGAGGACGCGGGGCACTATGCACTGTGCACTA | |
| | GCGGGGGCCAGGCGCTGGCTGAGCTCATTGTGCAGGAAAAGA | |
| | AGCTGGAGGTGTACCAGAGCATCGCAGACCTGATGGTGGGCGC | |
| | AAAGGACCAGGCGGTGTTCAAATGTGAGGTCTCAGATGAGAAT | |
| | GTTCGGGGTGTGTGGCTGAAGAATGGGAAGGAGCTGGTGCCCG | |
| | ACAGCCGCATAAAGGTGTCCCACATCGGGCGGGTCCACAAACT | |
| | GACCATTGACGACGTCACACCTGCCGACGAGGCTGACTACAGC | |
| | TTTGTGCCCGAGGGCTTCGCCTGCAACCTGTCAGCCAAGCTCCA | |
| | CTTCATGGAGGTCAAGATTGACTTCGTACCCAGGCAGGAACCT | |
| | CCCAAGATCCACCTGGACTGCCCAGGCCGCATACCAGACACCA | |
| | TTGTGGTTGTAGCTGGAAATAAGCTACGTCTGGACGTCCCTATC | |
| | TCTGGGGACCCCGCTCCCACTGTGATCTGGCAGAAGGCTATCA | |
| | CGCAGGGGAATAAGGCCCCAGCCAGGCCAGCCCCAGATGCCCC | |
| | AGAGGACACAGGTGACAGCGATGAGTGGGTGTTTGACAAGAA | |
| | GCTGCTGTGTGAGACCGAGGGCCGGGTCCGCGTGGAGACCACC | |
| | AAGGACCGCAGCATCTTCACGGTCGAGGGGGCAGAGAAGGAA | |
| | GATGAGGGCGTCTACACGGTCACAGTGAAGAACCCTGTGGGCG | |
| | AGGACCAGGTCAACCTCACAGTCAAGGTCATCGACGTGCCAGA | |
| | CGCACCTGCGGCCCCCAAGATCAGCAACGTGGGAGAGGACTCC | |
| | TGCACAGTACAGTGGGAGCCGCCTGCCTACGATGGCGGGCAGC | |
| | CCATCCTGGGCTACATCCTGGAGCGCAAGAAGAAGAAGAGCTA | |
| | CCGGTGGATGCGGCTGAACTTCGACCTGATTCAGGAGCTGAGT | |
| | CATGAAGCGCGGCGCATGATCGAGGGCGTGGTGTACGAGATGC | |
| | GCGTCTACGCGGTCAACGCCATCGGCATGTCCAGGCCCAGCCC | |
| | TGCCTCCCAGCCCTTCATGCCTATCGGTCCCCCCAGCGAACCCA | |
| | CCCACCTGGCAGTAGAGGACGTCTCTGACACCACGGTCTCCCT | |
| | CAAGTGGCGGCCCCCAGAGCGCGTGGGAGCAGGAGGCCTGGA | |
| | TGGCTACAGCGTGGAGTACTGCCCAGAGGGCTGCTCAGAGTGG | |
| | GTGGCTGCCCTGCAGGGGCTGACAGAGCACACATCGATACTGG | |
| | TGAAGGACCTGCCCACGGGGGCCCGGCTGCTTTTCCGAGTGCG | |
| | GGCACACAATATGGCAGGGCCTGGAGCCCCTGTTACCACCACG | |
| | GAGCCGGTGACAGTGCAGGAGATCCTGCAACGGCCACGGCTTC | |
| | AGCTGCCCAGGCACCTGCGCCAGACCATTCAGAAGAAGGTCGG | |
| | GGAGCCTGTGAACCTTCTCATCCCTTTCCAGGGCAAGCCCCGGC | |
| | CTCAGGTGACCTGGACCAAAGAGGGGCAGCCCCTGGCAGGCG | |
| | AGGAGGTGAGCATCCGCAACAGCCCCACAGACACCATCCTGTT | |
| | CATCCGGGCCGCTCGCCGCGTGCATTCAGGCACTTACCAGGTG | |
| | ACGGTGCGCATTGAGAACATGGAGGACAAGGCCACGCTGGTGC | |
| | TGCAGGTTGTTGACAAGCCAAGTCCTCCCCAGGATCTCCGGGT | |
| | GACTGACGCCTGGGGTCTTAATGTGGCTCTGGAGTGGAAGCCA | |
| | CCCCAGGATGTCGGCAACACGGAACTCTGGGGGTACACAGTGC | |
| | AGAAAGCCGACAAGAAGACCATGGAGTGGTTCACCGTCTTGGA | |
| | GCATTACCGCCGCACCCACTGCGTGGTGCCAGAGCTCATCATT | |
| | GGCAATGGCTACTACTTCCGCGTCTTCAGCCAGAATATGGTTGG | |
| | CTTTAGTGACAGAGCGGCCACCACCAAGGAGCCCGTCTTTATC | |
| | CCCAGACCAGGCATCACCTATGAGCCACCCAACTATAAGGCCC | |
| | TGGACTTCTCCGAGGCCCCAAGCTTCACCCAGCCCCTGGTGAA | |
| | CCGCTCGGTCATCGCGGGCTACACTGCTATGCTCTGCTGTGCTG | |
| | TCCGGGGTAGCCCCAAGCCCAAGATTTCCTGGTTCAAGAATGG | |
| | CCTGGACCTGGGAGAAGACGCCCGCTTCCGCATGTTCAGCAAG | |
| | CAGGGAGTGTTGACTCTGGAGATTAGAAAGCCCTGCCCCTTTG | |
| | ACGGGGGCATCTATGTCTGCAGGGCCACCAACTTACAGGGCGA | |
| | GGCACGGTGTGAGTGCCGCCTGGAGGTGCGAGTGCCTCAGTAA | |
| MYBPC3- | ATGCCTGAGCCGGGGAAGAAGCCAGTCTCAGCTTTTAGCAAGA | 812 |
| delC3 | AGCCACGGTCAGTGGAAGTGGCCGCAGGCAGCCCTGCCGTGTT | |
| DNA | CGAGGCCGAGACAGAGCGGGCAGGAGTGAAGGTGCGCTGGCA | |
| | GCGCGGAGGCAGTGACATCAGCGCCAGCAACAAGTACGGCCT | |
| | GGCCACAGAGGGCACACGGCATACGCTGACAGTGCGGGAAGT | |
| | GGGCCCTGCCGACCAGGGATCTTACGCAGTCATTGCTGGCTCCT | |
| | CCAAGGTCAAGTTCGACCTCAAGGTCATAGAGGCAGAGAAGGC | |
| | AGAGCCCATGCTGGCCCCTGCCCCTGCCCCTGCTGAGGCCACT | |
| | GGAGCCCCTGGAGAAGCCCCGGCCCCAGCCGCTGAGCTGGGAG | |
| | AAAGTGCCCCAAGTCCCAAAGGGTCAAGCTCAGCAGCTCTCAA | |
| | TGGTCCTACCCCTGGAGCCCCCGATGACCCCATTGGCCTCTTCG | |
| | TGATGCGGCCACAGGATGGCGAGGTGACCGTGGGTGGCAGCAT | |
| | CACCTTCTCAGCCCGCGTGGCCGGCGCCAGCCTCCTGAAGCCG | |
| | CCTGTGGTCAAGTGGTTCAAGGGCAAATGGGTGGACCTGAGCA | |
| | GCAAGGTGGGCCAGCACCTGCAGCTGCACGACAGCTACGACCG | |
| | CGCCAGCAAGGTCTATCTGTTCGAGCTGCACATCACCGATGCC | |
| | CAGCCTGCCTTCACTGGCAGCTACCGCTGTGAGGTGTCCACCA | |
| | AGGACAAATTTGACTGCTCCAACTTCAATCTCACTGTCCACGAG | |
| | GCCATGGGCACCGGAGACCTGGACCTCCTATCAGCCTTCCGCC | |
| | GCACGAGCCTGGCTGGAGGTGGTCGGCGGATCAGTGATAGCCA | |
| | TGAGGACACTGGGATTCTGGACTTCAGCTCACTGCTGAAAAAG | |
| | AGAGACAGTTTCCGGACCCCGAGGGACTCGAAGCTGGAGGCAC | |
| | CAGCAGAGGAGGACGTGTGGGAGATCCTACGGCAGGCACCCC | |
| | CATCTGAGTACGAGCGCATCGCCTTCCAGTACGGCGTCACTGA | |
| | CCTGCGCGGCATGCTAAAGAGGCTCAAGGGCATGAGGCGCGAT | |
| | GAGAAGAAGAGCACAGCCTTTCAGAAGAAGCTGGAGCCGGCC | |
| | TACCAGGTGAGCAAAGGCCACAAGATCCGGCTGACCGTGGAAC | |
| | TGGCTGACCATGACGCTGAGGTCAAATGGCTCAAGAATGGCCA | |
| | GGAGATCCAGATGAGCGGCAGCAAGTACATCTTTGAGTCCATC | |
| | GGTGCCAAGCGTACCCTGACCATCAGCCAGTGCTCATTGGCGG | |
| | ACGACGCAGCCTACCAGTGCGTGGTGGGTGGCGAGAAGTGTAG | |
| | CACGGAGCTCTTTGTGAAAGAGCCCCCTGTGTACCAGAGCATC | |
| | GCAGACCTGATGGTGGGCGCAAAGGACCAGGCGGTGTTCAAAT | |
| | GTGAGGTCTCAGATGAGAATGTTCGGGGTGTGTGGCTGAAGAA | |
| | TGGGAAGGAGCTGGTGCCCGACAGCCGCATAAAGGTGTCCCAC | |
| | ATCGGGCGGGTCCACAAACTGACCATTGACGACGTCACACCTG | |
| | CCGACGAGGCTGACTACAGCTTTGTGCCCGAGGGCTTCGCCTG | |
| | CAACCTGTCAGCCAAGCTCCACTTCATGGAGGTCAAGATTGAC | |
| | TTCGTACCCAGGCAGGAACCTCCCAAGATCCACCTGGACTGCC | |
| | CAGGCCGCATACCAGACACCATTGTGGTTGTAGCTGGAAATAA | |
| | GCTACGTCTGGACGTCCCTATCTCTGGGGACCCCGCTCCCACTG | |
| | TGATCTGGCAGAAGGCTATCACGCAGGGGAATAAGGCCCCAGC | |
| | CAGGCCAGCCCCAGATGCCCCAGAGGACACAGGTGACAGCGA | |
| | TGAGTGGGTGTTTGACAAGAAGCTGCTGTGTGAGACCGAGGGC | |
| | CGGGTCCGCGTGGAGACCACCAAGGACCGCAGCATCTTCACGG | |
| | TCGAGGGGGCAGAGAAGGAAGATGAGGGCGTCTACACGGTCA | |
| | CAGTGAAGAACCCTGTGGGCGAGGACCAGGTCAACCTCACAGT | |
| | CAAGGTCATCGACGTGCCAGACGCACCTGCGGCCCCCAAGATC | |
| | AGCAACGTGGGAGAGGACTCCTGCACAGTACAGTGGGAGCCG | |
| | CCTGCCTACGATGGCGGGCAGCCCATCCTGGGCTACATCCTGG | |
| | AGCGCAAGAAGAAGAAGAGCTACCGGTGGATGCGGCTGAACT | |
| | TCGACCTGATTCAGGAGCTGAGTCATGAAGCGCGGCGCATGAT | |
| | CGAGGGCGTGGTGTACGAGATGCGCGTCTACGCGGTCAACGCC | |
| | ATCGGCATGTCCAGGCCCAGCCCTGCCTCCCAGCCCTTCATGCC | |
| | TATCGGTCCCCCCAGCGAACCCACCCACCTGGCAGTAGAGGAC | |
| | GTCTCTGACACCACGGTCTCCCTCAAGTGGCGGCCCCCAGAGC | |
| | GCGTGGGAGCAGGAGGCCTGGATGGCTACAGCGTGGAGTACTG | |
| | CCCAGAGGGCTGCTCAGAGTGGGTGGCTGCCCTGCAGGGGCTG | |
| | ACAGAGCACACATCGATACTGGTGAAGGACCTGCCCACGGGGG | |
| | CCCGGCTGCTTTTCCGAGTGCGGGCACACAATATGGCAGGGCC | |
| | TGGAGCCCCTGTTACCACCACGGAGCCGGTGACAGTGCAGGAG | |
| | ATCCTGCAACGGCCACGGCTTCAGCTGCCCAGGCACCTGCGCC | |
| | AGACCATTCAGAAGAAGGTCGGGGAGCCTGTGAACCTTCTCAT | |
| | CCCTTTCCAGGGCAAGCCCCGGCCTCAGGTGACCTGGACCAAA | |
| | GAGGGGCAGCCCCTGGCAGGCGAGGAGGTGAGCATCCGCAAC | |
| | AGCCCCACAGACACCATCCTGTTCATCCGGGCCGCTCGCCGCG | |
| | TGCATTCAGGCACTTACCAGGTGACGGTGCGCATTGAGAACAT | |
| | GGAGGACAAGGCCACGCTGGTGCTGCAGGTTGTTGACAAGCCA | |
| | AGTCCTCCCCAGGATCTCCGGGTGACTGACGCCTGGGGTCTTA | |
| | ATGTGGCTCTGGAGTGGAAGCCACCCCAGGATGTCGGCAACAC | |
| | GGAACTCTGGGGGTACACAGTGCAGAAAGCCGACAAGAAGAC | |
| | CATGGAGTGGTTCACCGTCTTGGAGCATTACCGCCGCACCCACT | |
| | GCGTGGTGCCAGAGCTCATCATTGGCAATGGCTACTACTTCCGC | |
| | GTCTTCAGCCAGAATATGGTTGGCTTTAGTGACAGAGCGGCCA | |
| | CCACCAAGGAGCCCGTCTTTATCCCCAGACCAGGCATCACCTA | |
| | TGAGCCACCCAACTATAAGGCCCTGGACTTCTCCGAGGCCCCA | |
| | AGCTTCACCCAGCCCCTGGTGAACCGCTCGGTCATCGCGGGCT | |
| | ACACTGCTATGCTCTGCTGTGCTGTCCGGGGTAGCCCCAAGCCC | |
| | AAGATTTCCTGGTTCAAGAATGGCCTGGACCTGGGAGAAGACG | |
| | CCCGCTTCCGCATGTTCAGCAAGCAGGGAGTGTTGACTCTGGA | |
| | GATTAGAAAGCCCTGCCCCTTTGACGGGGGCATCTATGTCTGC | |
| | AGGGCCACCAACTTACAGGGCGAGGCACGGTGTGAGTGCCGCC | |
| | TGGAGGTGCGAGTGCCTCAGTAA | |
| MYBPC3- | ATGCCTGAGCCGGGGAAGAAGCCAGTCTCAGCTTTTAGCAAGA | 813 |
| delC4 | AGCCACGGTCAGTGGAAGTGGCCGCAGGCAGCCCTGCCGTGTT | |
| DNA | CGAGGCCGAGACAGAGCGGGCAGGAGTGAAGGTGCGCTGGCA | |
| | GCGCGGAGGCAGTGACATCAGCGCCAGCAACAAGTACGGCCT | |
| | GGCCACAGAGGGCACACGGCATACGCTGACAGTGCGGGAAGT | |
| | GGGCCCTGCCGACCAGGGATCTTACGCAGTCATTGCTGGCTCCT | |
| | CCAAGGTCAAGTTCGACCTCAAGGTCATAGAGGCAGAGAAGGC | |
| | AGAGCCCATGCTGGCCCCTGCCCCTGCCCCTGCTGAGGCCACT | |
| | GGAGCCCCTGGAGAAGCCCCGGCCCCAGCCGCTGAGCTGGGAG | |
| | AAAGTGCCCCAAGTCCCAAAGGGTCAAGCTCAGCAGCTCTCAA | |
| | TGGTCCTACCCCTGGAGCCCCCGATGACCCCATTGGCCTCTTCG | |
| | TGATGCGGCCACAGGATGGCGAGGTGACCGTGGGTGGCAGCAT | |
| | CACCTTCTCAGCCCGCGTGGCCGGCGCCAGCCTCCTGAAGCCG | |
| | CCTGTGGTCAAGTGGTTCAAGGGCAAATGGGTGGACCTGAGCA | |
| | GCAAGGTGGGCCAGCACCTGCAGCTGCACGACAGCTACGACCG | |
| | CGCCAGCAAGGTCTATCTGTTCGAGCTGCACATCACCGATGCC | |
| | CAGCCTGCCTTCACTGGCAGCTACCGCTGTGAGGTGTCCACCA | |
| | AGGACAAATTTGACTGCTCCAACTTCAATCTCACTGTCCACGAG | |
| | GCCATGGGCACCGGAGACCTGGACCTCCTATCAGCCTTCCGCC | |
| | GCACGAGCCTGGCTGGAGGTGGTCGGCGGATCAGTGATAGCCA | |
| | TGAGGACACTGGGATTCTGGACTTCAGCTCACTGCTGAAAAAG | |
| | AGAGACAGTTTCCGGACCCCGAGGGACTCGAAGCTGGAGGCAC | |
| | CAGCAGAGGAGGACGTGTGGGAGATCCTACGGCAGGCACCCC | |
| | CATCTGAGTACGAGCGCATCGCCTTCCAGTACGGCGTCACTGA | |
| | CCTGCGCGGCATGCTAAAGAGGCTCAAGGGCATGAGGCGCGAT | |
| | GAGAAGAAGAGCACAGCCTTTCAGAAGAAGCTGGAGCCGGCC | |
| | TACCAGGTGAGCAAAGGCCACAAGATCCGGCTGACCGTGGAAC | |
| | TGGCTGACCATGACGCTGAGGTCAAATGGCTCAAGAATGGCCA | |
| | GGAGATCCAGATGAGCGGCAGCAAGTACATCTTTGAGTCCATC | |
| | GGTGCCAAGCGTACCCTGACCATCAGCCAGTGCTCATTGGCGG | |
| | ACGACGCAGCCTACCAGTGCGTGGTGGGTGGCGAGAAGTGTAG | |
| | CACGGAGCTCTTTGTGAAAGAGCCCCCTGTGCTCATCACGCGC | |
| | CCCTTGGAGGACCAGCTGGTGATGGTGGGGCAGCGGGTGGAGT | |
| | TTGAGTGTGAAGTATCGGAGGAGGGGGCGCAAGTCAAATGGCT | |
| | GAAGGACGGGGTGGAGCTGACCCGGGAGGAGACCTTCAAATA | |
| | CCGGTTCAAGAAGGACGGGCAGAGACACCACCTGATCATCAAC | |
| | GAGGCCATGCTGGAGGACGCGGGGCACTATGCACTGTGCACTA | |
| | GCGGGGGCCAGGCGCTGGCTGAGCTCATTGTGCAGGAAAAGA | |
| | AGCTGGAGCCTCCCAAGATCCACCTGGACTGCCCAGGCCGCAT | |
| | ACCAGACACCATTGTGGTTGTAGCTGGAAATAAGCTACGTCTG | |
| | GACGTCCCTATCTCTGGGGACCCCGCTCCCACTGTGATCTGGCA | |
| | GAAGGCTATCACGCAGGGGAATAAGGCCCCAGCCAGGCCAGC | |
| | CCCAGATGCCCCAGAGGACACAGGTGACAGCGATGAGTGGGT | |
| | GTTTGACAAGAAGCTGCTGTGTGAGACCGAGGGCCGGGTCCGC | |
| | GTGGAGACCACCAAGGACCGCAGCATCTTCACGGTCGAGGGGG | |
| | CAGAGAAGGAAGATGAGGGCGTCTACACGGTCACAGTGAAGA | |
| | ACCCTGTGGGCGAGGACCAGGTCAACCTCACAGTCAAGGTCAT | |
| | CGACGTGCCAGACGCACCTGCGGCCCCCAAGATCAGCAACGTG | |
| | GGAGAGGACTCCTGCACAGTACAGTGGGAGCCGCCTGCCTACG | |
| | ATGGCGGGCAGCCCATCCTGGGCTACATCCTGGAGCGCAAGAA | |
| | GAAGAAGAGCTACCGGTGGATGCGGCTGAACTTCGACCTGATT | |
| | CAGGAGCTGAGTCATGAAGCGCGGCGCATGATCGAGGGCGTG | |
| | GTGTACGAGATGCGCGTCTACGCGGTCAACGCCATCGGCATGT | |
| | CCAGGCCCAGCCCTGCCTCCCAGCCCTTCATGCCTATCGGTCCC | |
| | CCCAGCGAACCCACCCACCTGGCAGTAGAGGACGTCTCTGACA | |
| | CCACGGTCTCCCTCAAGTGGCGGCCCCCAGAGCGCGTGGGAGC | |
| | AGGAGGCCTGGATGGCTACAGCGTGGAGTACTGCCCAGAGGGC | |
| | TGCTCAGAGTGGGTGGCTGCCCTGCAGGGGCTGACAGAGCACA | |
| | CATCGATACTGGTGAAGGACCTGCCCACGGGGGCCCGGCTGCT | |
| | TTTCCGAGTGCGGGCACACAATATGGCAGGGCCTGGAGCCCCT | |
| | GTTACCACCACGGAGCCGGTGACAGTGCAGGAGATCCTGCAAC | |
| | GGCCACGGCTTCAGCTGCCCAGGCACCTGCGCCAGACCATTCA | |
| | GAAGAAGGTCGGGGAGCCTGTGAACCTTCTCATCCCTTTCCAG | |
| | GGCAAGCCCCGGCCTCAGGTGACCTGGACCAAAGAGGGGCAG | |
| | CCCCTGGCAGGCGAGGAGGTGAGCATCCGCAACAGCCCCACAG | |
| | ACACCATCCTGTTCATCCGGGCCGCTCGCCGCGTGCATTCAGGC | |
| | ACTTACCAGGTGACGGTGCGCATTGAGAACATGGAGGACAAGG | |
| | CCACGCTGGTGCTGCAGGTTGTTGACAAGCCAAGTCCTCCCCA | |
| | GGATCTCCGGGTGACTGACGCCTGGGGTCTTAATGTGGCTCTG | |
| | GAGTGGAAGCCACCCCAGGATGTCGGCAACACGGAACTCTGGG | |
| | GGTACACAGTGCAGAAAGCCGACAAGAAGACCATGGAGTGGT | |
| | TCACCGTCTTGGAGCATTACCGCCGCACCCACTGCGTGGTGCCA | |
| | GAGCTCATCATTGGCAATGGCTACTACTTCCGCGTCTTCAGCCA | |
| | GAATATGGTTGGCTTTAGTGACAGAGCGGCCACCACCAAGGAG | |
| | CCCGTCTTTATCCCCAGACCAGGCATCACCTATGAGCCACCCAA | |
| | CTATAAGGCCCTGGACTTCTCCGAGGCCCCAAGCTTCACCCAG | |
| | CCCCTGGTGAACCGCTCGGTCATCGCGGGCTACACTGCTATGCT | |
| | CTGCTGTGCTGTCCGGGGTAGCCCCAAGCCCAAGATTTCCTGGT | |
| | TCAAGAATGGCCTGGACCTGGGAGAAGACGCCCGCTTCCGCAT | |
| | GTTCAGCAAGCAGGGAGTGTTGACTCTGGAGATTAGAAAGCCC | |
| | TGCCCCTTTGACGGGGGCATCTATGTCTGCAGGGCCACCAACTT | |
| | ACAGGGCGAGGCACGGTGTGAGTGCCGCCTGGAGGTGCGAGT | |
| | GCCTCAGTAA | |
| MYBPC3- | ATGCCTGAGCCGGGGAAGAAGCCAGTCTCAGCTTTTAGCAAGA | 814 |
| delC4b | AGCCACGGTCAGTGGAAGTGGCCGCAGGCAGCCCTGCCGTGTT | |
| DNA | CGAGGCCGAGACAGAGCGGGCAGGAGTGAAGGTGCGCTGGCA | |
| | GCGCGGAGGCAGTGACATCAGCGCCAGCAACAAGTACGGCCT | |
| | GGCCACAGAGGGCACACGGCATACGCTGACAGTGCGGGAAGT | |
| | GGGCCCTGCCGACCAGGGATCTTACGCAGTCATTGCTGGCTCCT | |
| | CCAAGGTCAAGTTCGACCTCAAGGTCATAGAGGCAGAGAAGGC | |
| | AGAGCCCATGCTGGCCCCTGCCCCTGCCCCTGCTGAGGCCACT | |
| | GGAGCCCCTGGAGAAGCCCCGGCCCCAGCCGCTGAGCTGGGAG | |
| | AAAGTGCCCCAAGTCCCAAAGGGTCAAGCTCAGCAGCTCTCAA | |
| | TGGTCCTACCCCTGGAGCCCCCGATGACCCCATTGGCCTCTTCG | |
| | TGATGCGGCCACAGGATGGCGAGGTGACCGTGGGTGGCAGCAT | |
| | CACCTTCTCAGCCCGCGTGGCCGGCGCCAGCCTCCTGAAGCCG | |
| | CCTGTGGTCAAGTGGTTCAAGGGCAAATGGGTGGACCTGAGCA | |
| | GCAAGGTGGGCCAGCACCTGCAGCTGCACGACAGCTACGACCG | |
| | CGCCAGCAAGGTCTATCTGTTCGAGCTGCACATCACCGATGCC | |
| | CAGCCTGCCTTCACTGGCAGCTACCGCTGTGAGGTGTCCACCA | |
| | AGGACAAATTTGACTGCTCCAACTTCAATCTCACTGTCCACGAG | |
| | GCCATGGGCACCGGAGACCTGGACCTCCTATCAGCCTTCCGCC | |
| | GCACGAGCCTGGCTGGAGGTGGTCGGCGGATCAGTGATAGCCA | |
| | TGAGGACACTGGGATTCTGGACTTCAGCTCACTGCTGAAAAAG | |
| | AGAGACAGTTTCCGGACCCCGAGGGACTCGAAGCTGGAGGCAC | |
| | CAGCAGAGGAGGACGTGTGGGAGATCCTACGGCAGGCACCCC | |
| | CATCTGAGTACGAGCGCATCGCCTTCCAGTACGGCGTCACTGA | |
| | CCTGCGCGGCATGCTAAAGAGGCTCAAGGGCATGAGGCGCGAT | |
| | GAGAAGAAGAGCACAGCCTTTCAGAAGAAGCTGGAGCCGGCC | |
| | TACCAGGTGAGCAAAGGCCACAAGATCCGGCTGACCGTGGAAC | |
| | TGGCTGACCATGACGCTGAGGTCAAATGGCTCAAGAATGGCCA | |
| | GGAGATCCAGATGAGCGGCAGCAAGTACATCTTTGAGTCCATC | |
| | GGTGCCAAGCGTACCCTGACCATCAGCCAGTGCTCATTGGCGG | |
| | ACGACGCAGCCTACCAGTGCGTGGTGGGTGGCGAGAAGTGTAG | |
| | CACGGAGCTCTTTGTGAAAGAGCCCCCTGTGCTCATCACGCGC | |
| | CCCTTGGAGGACCAGCTGGTGATGGTGGGGCAGCGGGTGGAGT | |
| | TTGAGTGTGAAGTATCGGAGGAGGGGGCGCAAGTCAAATGGCT | |
| | GAAGGACGGGGTGGAGCTGACCCGGGAGGAGACCTTCAAATA | |
| | CCGGTTCAAGAAGGACGGGCAGAGACACCACCTGATCATCAAC | |
| | GAGGCCATGCTGGAGGACGCGGGGCACTATGCACTGTGCACTA | |
| | GCGGGGGCCAGGCGCTGGCTGAGCTCATTGTGCAGGAAAAGA | |
| | AGCTGGAGCCCAGGCAGGAACCTCCCAAGATCCACCTGGACTG | |
| | CCCAGGCCGCATACCAGACACCATTGTGGTTGTAGCTGGAAAT | |
| | AAGCTACGTCTGGACGTCCCTATCTCTGGGGACCCCGCTCCCAC | |
| | TGTGATCTGGCAGAAGGCTATCACGCAGGGGAATAAGGCCCCA | |
| | GCCAGGCCAGCCCCAGATGCCCCAGAGGACACAGGTGACAGC | |
| | GATGAGTGGGTGTTTGACAAGAAGCTGCTGTGTGAGACCGAGG | |
| | GCCGGGTCCGCGTGGAGACCACCAAGGACCGCAGCATCTTCAC | |
| | GGTCGAGGGGGCAGAGAAGGAAGATGAGGGCGTCTACACGGT | |
| | CACAGTGAAGAACCCTGTGGGCGAGGACCAGGTCAACCTCACA | |
| | GTCAAGGTCATCGACGTGCCAGACGCACCTGCGGCCCCCAAGA | |
| | TCAGCAACGTGGGAGAGGACTCCTGCACAGTACAGTGGGAGCC | |
| | GCCTGCCTACGATGGCGGGCAGCCCATCCTGGGCTACATCCTG | |
| | GAGCGCAAGAAGAAGAAGAGCTACCGGTGGATGCGGCTGAAC | |
| | TTCGACCTGATTCAGGAGCTGAGTCATGAAGCGCGGCGCATGA | |
| | TCGAGGGCGTGGTGTACGAGATGCGCGTCTACGCGGTCAACGC | |
| | CATCGGCATGTCCAGGCCCAGCCCTGCCTCCCAGCCCTTCATGC | |
| | CTATCGGTCCCCCCAGCGAACCCACCCACCTGGCAGTAGAGGA | |
| | CGTCTCTGACACCACGGTCTCCCTCAAGTGGCGGCCCCCAGAG | |
| | CGCGTGGGAGCAGGAGGCCTGGATGGCTACAGCGTGGAGTACT | |
| | GCCCAGAGGGCTGCTCAGAGTGGGTGGCTGCCCTGCAGGGGCT | |
| | GACAGAGCACACATCGATACTGGTGAAGGACCTGCCCACGGGG | |
| | GCCCGGCTGCTTTTCCGAGTGCGGGCACACAATATGGCAGGGC | |
| | CTGGAGCCCCTGTTACCACCACGGAGCCGGTGACAGTGCAGGA | |
| | GATCCTGCAACGGCCACGGCTTCAGCTGCCCAGGCACCTGCGC | |
| | CAGACCATTCAGAAGAAGGTCGGGGAGCCTGTGAACCTTCTCA | |
| | TCCCTTTCCAGGGCAAGCCCCGGCCTCAGGTGACCTGGACCAA | |
| | AGAGGGGCAGCCCCTGGCAGGCGAGGAGGTGAGCATCCGCAA | |
| | CAGCCCCACAGACACCATCCTGTTCATCCGGGCCGCTCGCCGC | |
| | GTGCATTCAGGCACTTACCAGGTGACGGTGCGCATTGAGAACA | |
| | TGGAGGACAAGGCCACGCTGGTGCTGCAGGTTGTTGACAAGCC | |
| | AAGTCCTCCCCAGGATCTCCGGGTGACTGACGCCTGGGGTCTT | |
| | AATGTGGCTCTGGAGTGGAAGCCACCCCAGGATGTCGGCAACA | |
| | CGGAACTCTGGGGGTACACAGTGCAGAAAGCCGACAAGAAGA | |
| | CCATGGAGTGGTTCACCGTCTTGGAGCATTACCGCCGCACCCA | |
| | CTGCGTGGTGCCAGAGCTCATCATTGGCAATGGCTACTACTTCC | |
| | GCGTCTTCAGCCAGAATATGGTTGGCTTTAGTGACAGAGCGGC | |
| | CACCACCAAGGAGCCCGTCTTTATCCCCAGACCAGGCATCACC | |
| | TATGAGCCACCCAACTATAAGGCCCTGGACTTCTCCGAGGCCC | |
| | CAAGCTTCACCCAGCCCCTGGTGAACCGCTCGGTCATCGCGGG | |
| | CTACACTGCTATGCTCTGCTGTGCTGTCCGGGGTAGCCCCAAGC | |
| | CCAAGATTTCCTGGTTCAAGAATGGCCTGGACCTGGGAGAAGA | |
| | CGCCCGCTTCCGCATGTTCAGCAAGCAGGGAGTGTTGACTCTG | |
| | GAGATTAGAAAGCCCTGCCCCTTTGACGGGGGCATCTATGTCT | |
| | GCAGGGCCACCAACTTACAGGGCGAGGCACGGTGTGAGTGCCG | |
| | CCTGGAGGTGCGAGTGCCTCAGTAA | |
| Human | MPEPGKKPVSAFSKKPRSVEVAAGSPAVFEAETERAGVKVRWQR | 815 |
| MYBPC3 | GGSDISASNKYGLATEGTRHTLTVREVGPADQGSYAVIAGSSKVK | |
| protein | FDLKVIEAEKAEPMLAPAPAPAEATGAPGEAPAPAAELGESAPSPK | |
| | GSSSAALNGPTPGAPDDPIGLFVMRPQDGEVTVGGSITFSARVAGA | |
| | SLLKPPVVKWFKGKWVDLSSKVGQHLQLHDSYDRASKVYLFELH | |
| | ITDAQPAFTGSYRCEVSTKDKFDCSNFNLTVHEAMGTGDLDLLSA | |
| | FRRTSLAGGGRRISDSHEDTGILDFSSLLKKRDSFRTPRDSKLEAPA | |
| | EEDVWEILRQAPPSEYERIAFQYGVTDLRGMLKRLKGMRRDEKK | |
| | STAFQKKLEPAYQVSKGHKIRLTVELADHDAEVKWLKNGQEIQM | |
| | SGSKYIFESIGAKRTLTISQCSLADDAAYQCVVGGEKCSTELFVKE | |
| | PPVLITRPLEDQLVMVGQRVEFECEVSEEGAQVKWLKDGVELTRE | |
| | ETFKYRFKKDGQRHHLIINEAMLEDAGHYALCTSGGQALAELIVQ | |
| | EKKLEVYQSIADLMVGAKDQAVFKCEVSDENVRGVWLKNGKEL | |
| | VPDSRIKVSHIGRVHKLTIDDVTPADEADYSFVPEGFACNLSAKLH | |
| | FMEVKIDFVPRQEPPKIHLDCPGRIPDTIVVVAGNKLRLDVPISGDP | |
| | APTVIWQKAITQGNKAPARPAPDAPEDTGDSDEWVFDKKLLCETE | |
| | GRVRVETTKDRSIFTVEGAEKEDEGVYTVTVKNPVGEDQVNLTV | |
| | KVIDVPDAPAAPKISNVGEDSCTVQWEPPAYDGGQPILGYILERKK | |
| | KKSYRWMRLNFDLIQELSHEARRMIEGVVYEMRVYAVNAIGMSR | |
| | PSPASQPFMPIGPPSEPTHLAVEDVSDTTVSLKWRPPERVGAGGLD | |
| | GYSVEYCPEGCSEWVAALQGLTEHTSILVKDLPTGARLLFRVRAH | |
| | NMAGPGAPVTTTEPVTVQEILQRPRLQLPRHLRQTIQKKVGEPVN | |
| | LLIPFQGKPRPQVTWTKEGQPLAGEEVSIRNSPTDTILFIRAARRVH | |
| | SGTYQVTVRIENMEDKATLVLQVVDKPSPPQDLRVTDAWGLNVA | |
| | LEWKPPQDVGNTELWGYTVQKADKKTMEWFTVLEHYRRTHCV | |
| | VPELIIGNGYYFRVFSQNMVGFSDRAATTKEPVFIPRPGITYEPPNY | |
| | KALDFSEAPSFTQPLVNRSVIAGYTAMLCCAVRGSPKPKISWFKN | |
| | GLDLGEDARFRMFSKQGVLTLEIRKPCPFDGGIYVCRATNLQGEA | |
| | RCECRLEVRVPQ | |
| MYBPC3- | MPEPGKKPVSAFSKKPRSVEVAAGSPAVFEAETERAGVKVRWQR | 816 |
| delC3 | GGSDISASNKYGLATEGTRHTLTVREVGPADQGSYAVIAGSSKVK | |
| protein | FDLKVIEAEKAEPMLAPAPAPAEATGAPGEAPAPAAELGESAPSPK | |
| | GSSSAALNGPTPGAPDDPIGLFVMRPQDGEVTVGGSITESARVAGA | |
| | SLLKPPVVKWFKGKWVDLSSKVGQHLQLHDSYDRASKVYLFELH | |
| | ITDAQPAFTGSYRCEVSTKDKFDCSNFNLTVHEAMGTGDLDLLSA | |
| | FRRTSLAGGGRRISDSHEDTGILDESSLLKKRDSFRTPRDSKLEAPA | |
| | EEDVWEILRQAPPSEYERIAFQYGVTDLRGMLKRLKGMRRDEKK | |
| | STAFQKKLEPAYQVSKGHKIRLTVELADHDAEVKWLKNGQEIQM | |
| | SGSKYIFESIGAKRTLTISQCSLADDAAYQCVVGGEKCSTELFVKE | |
| | PPVYQSIADLMVGAKDQAVFKCEVSDENVRGVWLKNGKELVPDS | |
| | RIKVSHIGRVHKLTIDDVTPADEADYSFVPEGFACNLSAKLHFMEV | |
| | KIDFVPRQEPPKIHLDCPGRIPDTIVVVAGNKLRLDVPISGDPAPTVI | |
| | WQKAITQGNKAPARPAPDAPEDTGDSDEWVFDKKLLCETEGRVR | |
| | VETTKDRSIFTVEGAEKEDEGVYTVTVKNPVGEDQVNLTVKVIDV | |
| | PDAPAAPKISNVGEDSCTVQWEPPAYDGGQPILGYILERKKKKSY | |
| | RWMRLNFDLIQELSHEARRMIEGVVYEMRVYAVNAIGMSRPSPA | |
| | SQPFMPIGPPSEPTHLAVEDVSDTTVSLKWRPPERVGAGGLDGYS | |
| | VEYCPEGCSEWVAALQGLTEHTSILVKDLPTGARLLFRVRAHNM | |
| | AGPGAPVTTTEPVTVQEILQRPRLQLPRHLRQTIQKKVGEPVNLLIP | |
| | FQGKPRPQVTWTKEGQPLAGEEVSIRNSPTDTILFIRAARRVHSGT | |
| | YQVTVRIENMEDKATLVLQVVDKPSPPQDLRVTDAWGLNVALE | |
| | WKPPQDVGNTELWGYTVQKADKKTMEWFTVLEHYRRTHCVVPE | |
| | LIIGNGYYFRVFSQNMVGFSDRAATTKEPVFIPRPGITYEPPNYKAL | |
| | DFSEAPSFTQPLVNRSVIAGYTAMLCCAVRGSPKPKISWFKNGLDL | |
| | GEDARFRMFSKQGVLTLEIRKPCPFDGGIYVCRATNLQGEARCEC | |
| | RLEVRVPQ | |
| MYBPC3- | MPEPGKKPVSAFSKKPRSVEVAAGSPAVFEAETERAGVKVRWQR | 817 |
| delC4 | GGSDISASNKYGLATEGTRHTLTVREVGPADQGSYAVIAGSSKVK | |
| protein | FDLKVIEAEKAEPMLAPAPAPAEATGAPGEAPAPAAELGESAPSPK | |
| | GSSSAALNGPTPGAPDDPIGLFVMRPQDGEVTVGGSITFSARVAGA | |
| | SLLKPPVVKWFKGKWVDLSSKVGQHLQLHDSYDRASKVYLFELH | |
| | ITDAQPAFTGSYRCEVSTKDKFDCSNFNLTVHEAMGTGDLDLLSA | |
| | FRRTSLAGGGRRISDSHEDTGILDFSSLLKKRDSFRTPRDSKLEAPA | |
| | EEDVWEILRQAPPSEYERIAFQYGVTDLRGMLKRLKGMRRDEKK | |
| | STAFQKKLEPAYQVSKGHKIRLTVELADHDAEVKWLKNGQEIQM | |
| | SGSKYIFESIGAKRTLTISQCSLADDAAYQCVVGGEKCSTELFVKE | |
| | PPVLITRPLEDQLVMVGQRVEFECEVSEEGAQVKWLKDGVELTRE | |
| | ETFKYRFKKDGQRHHLIINEAMLEDAGHYALCTSGGQALAELIVQ | |
| | EKKLEPPKIHLDCPGRIPDTIVVVAGNKLRLDVPISGDPAPTVIWQK | |
| | AITQGNKAPARPAPDAPEDTGDSDEWVFDKKLLCETEGRVRVETT | |
| | KDRSIFTVEGAEKEDEGVYTVTVKNPVGEDQVNLTVKVIDVPDAP | |
| | AAPKISNVGEDSCTVQWEPPAYDGGQPILGYILERKKKKSYRWM | |
| | RLNFDLIQELSHEARRMIEGVVYEMRVYAVNAIGMSRPSPASQPF | |
| | MPIGPPSEPTHLAVEDVSDTTVSLKWRPPERVGAGGLDGYSVEYC | |
| | PEGCSEWVAALQGLTEHTSILVKDLPTGARLLFRVRAHNMAGPG | |
| | APVTTTEPVTVQEILQRPRLQLPRHLRQTIQKKVGEPVNLLIPFQGK | |
| | PRPQVTWTKEGQPLAGEEVSIRNSPTDTILFIRAARRVHSGTYQVT | |
| | VRIENMEDKATLVLQVVDKPSPPQDLRVTDAWGLNVALEWKPPQ | |
| | DVGNTELWGYTVQKADKKTMEWFTVLEHYRRTHCVVPELIIGNG | |
| | YYFRVFSQNMVGFSDRAATTKEPVFIPRPGITYEPPNYKALDESEA | |
| | PSFTQPLVNRSVIAGYTAMLCCAVRGSPKPKISWFKNGLDLGEDA | |
| | RFRMFSKQGVLTLEIRKPCPFDGGIYVCRATNLQGEARCECRLEV | |
| | RVPQ | |
| MYBPC3- | MPEPGKKPVSAFSKKPRSVEVAAGSPAVFEAETERAGVKVRWQR | 818 |
| delC4b | GGSDISASNKYGLATEGTRHTLTVREVGPADQGSYAVIAGSSKVK | |
| protein | FDLKVIEAEKAEPMLAPAPAPAEATGAPGEAPAPAAELGESAPSPK | |
| | GSSSAALNGPTPGAPDDPIGLFVMRPQDGEVTVGGSITFSARVAGA | |
| | SLLKPPVVKWFKGKWVDLSSKVGQHLQLHDSYDRASKVYLFELH | |
| | ITDAQPAFTGSYRCEVSTKDKFDCSNFNLTVHEAMGTGDLDLLSA | |
| | FRRTSLAGGGRRISDSHEDTGILDESSLLKKRDSFRTPRDSKLEAPA | |
| | EEDVWEILRQAPPSEYERIAFQYGVTDLRGMLKRLKGMRRDEKK | |
| | STAFQKKLEPAYQVSKGHKIRLTVELADHDAEVKWLKNGQEIQM | |
| | SGSKYIFESIGAKRTLTISQCSLADDAAYQCVVGGEKCSTELFVKE | |
| | PPVLITRPLEDQLVMVGQRVEFECEVSEEGAQVKWLKDGVELTRE | |
| | ETFKYRFKKDGQRHHLIINEAMLEDAGHYALCTSGGQALAELIVQ | |
| | EKKLEPRQEPPKIHLDCPGRIPDTIVVVAGNKLRLDVPISGDPAPTV | |
| | IWQKAITQGNKAPARPAPDAPEDTGDSDEWVFDKKLLCETEGRV | |
| | RVETTKDRSIFTVEGAEKEDEGVYTVTVKNPVGEDQVNLTVKVID | |
| | VPDAPAAPKISNVGEDSCTVQWEPPAYDGGQPILGYILERKKKKS | |
| | YRWMRLNFDLIQELSHEARRMIEGVVYEMRVYAVNAIGMSRPSP | |
| | ASQPFMPIGPPSEPTHLAVEDVSDTTVSLKWRPPERVGAGGLDGY | |
| | SVEYCPEGCSEWVAALQGLTEHTSILVKDLPTGARLLFRVRAHNM | |
| | AGPGAPVTTTEPVTVQEILQRPRLQLPRHLRQTIQKKVGEPVNLLIP | |
| | FQGKPRPQVTWTKEGQPLAGEEVSIRNSPTDTILFIRAARRVHSGT | |
| | YQVTVRIENMEDKATLVLQVVDKPSPPQDLRVTDAWGLNVALE | |
| | WKPPQDVGNTELWGYTVQKADKKTMEWFTVLEHYRRTHCVVPE | |
| | LIIGNGYYFRVFSQNMVGFSDRAATTKEPVFIPRPGITYEPPNYKAL | |
| | DFSEAPSFTQPLVNRSVIAGYTAMLCCAVRGSPKPKISWFKNGLDL | |
| | GEDARFRMFSKQGVLTLEIRKPCPFDGGIYVCRATNLQGEARCEC | |
| | RLEVRVPQ | |
| Human | ATGGCTCCGGCCGCCTGGCTCCGCAGCGCGGCCGCGCGCGCCC | 819 |
| MMP11 | TCCTGCCCCCGATGCTGCTGCTGCTGCTCCAGCCGCCGCCGCTG | |
| DNA | CTGGCCCGGGCTCTGCCGCCGGACGCCCACCACCTCCATGCCG | |
| | AGAGGAGGGGGCCACAGCCCTGGCATGCAGCCCTGCCCAGTAG | |
| | CCCGGCACCTGCCCCTGCCACGCAGGAAGCCCCCCGGCCTGCC | |
| | AGCAGCCTCAGGCCTCCCCGCTGTGGCGTGCCCGACCCATCTG | |
| | ATGGGCTGAGTGCCCGCAACCGACAGAAGAGGTTCGTGCTTTC | |
| | TGGCGGGCGCTGGGAGAAGACGGACCTCACCTACAGGATCCTT | |
| | CGGTTCCCATGGCAGTTGGTGCAGGAGCAGGTGCGGCAGACGA | |
| | TGGCAGAGGCCCTAAAGGTATGGAGCGATGTGACGCCACTCAC | |
| | CTTTACTGAGGTGCACGAGGGCCGTGCTGACATCATGATCGAC | |
| | TTCGCCAGGTACTGGCATGGGGACGACCTGCCGTTTGATGGGC | |
| | CTGGGGGCATCCTGGCCCATGCCTTCTTCCCCAAGACTCACCGA | |
| | GAAGGGGATGTCCACTTCGACTATGATGAGACCTGGACTATCG | |
| | GGGATGACCAGGGCACAGACCTGCTGCAGGTGGCAGCCCATGA | |
| | ATTTGGCCACGTGCTGGGGCTGCAGCACACAACAGCAGCCAAG | |
| | GCCCTGATGTCCGCCTTCTACACCTTTCGCTACCCACTGAGTCT | |
| | CAGCCCAGATGACTGCAGGGGCGTTCAACACCTATATGGCCAG | |
| | CCCTGGCCCACTGTCACCTCCAGGACCCCAGCCCTGGGCCCCC | |
| | AGGCTGGGATAGACACCAATGAGATTGCACCGCTGGAGCCAGA | |
| | CGCCCCGCCAGATGCCTGTGAGGCCTCCTTTGACGCGGTCTCCA | |
| | CCATCCGAGGCGAGCTCTTTTTCTTCAAAGCGGGCTTTGTGTGG | |
| | CGCCTCCGTGGGGGCCAGCTGCAGCCCGGCTACCCAGCATTGG | |
| | CCTCTCGCCACTGGCAGGGACTGCCCAGCCCTGTGGACGCTGC | |
| | CTTCGAGGATGCCCAGGGCCACATTTGGTTCTTCCAAGGTGCTC | |
| | AGTACTGGGTGTACGACGGTGAAAAGCCAGTCCTGGGCCCCGC | |
| | ACCCCTCACCGAGCTGGGCCTGGTGAGGTTCCCGGTCCATGCT | |
| | GCCTTGGTCTGGGGTCCCGAGAAGAACAAGATCTACTTCTTCC | |
| | GAGGCAGGGACTACTGGCGTTTCCACCCCAGCACCCGGCGTGT | |
| | AGACAGTCCCGTGCCCCGCAGGGCCACTGACTGGAGAGGGGTG | |
| | CCCTCTGAGATCGACGCTGCCTTCCAGGATGCTGATGGCTATGC | |
| | CTACTTCCTGCGCGGCCGCCTCTACTGGAAGTTTGACCCTGTGA | |
| | AGGTGAAGGCTCTGGAAGGCTTCCCCCGTCTCGTGGGTCCTGA | |
| | CTTCTTTGGCTGTGCCGAGCCTGCCAACACTTTCCTC | |
| Human | ATGGGTGCTGAGGAGGAGGTGCTGGTCACACTATCAGGGGGAG | 820 |
| SYNPO2L | CCCCCTGGGGCTTCCGACTTCATGGGGGGGCCGAGCAGAGGAA | |
| A DNA | ACCGTTACAGGTGTCTAAGATTCGAAGACGGAGCCAGGCTGGC | |
| | AGAGCAGGACTCCGAGAGAGGGACCAGCTCTTGGCAATCAATG | |
| | GGGTCTCTTGCACCAACCTCTCCCATGCCAGTGCCATGAGCCTC | |
| | ATCGATGCCTCAGGAAATCAGCTTGTCCTCACTGTGCAGCGGTT | |
| | AGCAGACGAGGGTCCTGTGCAATCTCCATCTCCCCATGAGCTTC | |
| | AGGTGCTGTCACCCTTATCTCCACTAAGTCCTGAGCCCCCTGGT | |
| | GCTCCAGTTCCTCAGCCTCTTCAGCCTGGGAGCCTTCGTTCACC | |
| | TCCTGATAGTGAGGCTTACTACGGAGAGACTGACAGTGATGCT | |
| | GATGGCCCTGCCACCCAGGAGAAGCCCCGTCGACCTCGCCGCC | |
| | GAGGCCCCACAAGGCCCACCCCTCCGGGTGCCCCACCTGATGA | |
| | GGTCTACCTGTCTGACAGCCCTGCAGAGCCAGCACCTACTATCC | |
| | CTGGCCCTCCCAGCCAGGGTGACAGCGTGTGAGCTCCCCGTCTT | |
| | GGGAGGATGGGGCAGCCCTTCAGCCACCCCCAGCTGAGGCTCT | |
| | GCTGTTACCCCATGGCCCCCTCCGACCTGGTCCTCATCTCATCC | |
| | CTATGGTGGGGCCTGTTCCCCACCCAGTGGCAGAAGATCTTACT | |
| | ACCACCTACACCCAGAAGGCCAAGCAAGCCAAACTGCAACGTG | |
| | CAGAGAGCCTCCAAGAGAAGAGCATAAAAGAGGCCAAGACCA | |
| | AATGCAGGACAATTGCATCCCTGCTCACTGCAGCCCCCAACCC | |
| | CCACTCCAAAGGGGTACTTATGTTTAAGAAACGGCGGCAGAGA | |
| | GCCAAGAAGTACACCCTGGTGAGCTTCGGGGCTGCTGCTGGGA | |
| | CAGGCGCTGAGGAGGAGGACGGCGTTCCCCCCACGAGTGAGTC | |
| | CGAGCTGGACGAAGAAGCCTTCTCTGACGCCCGCAGCCTCACC | |
| | AATCAATCTGACTGGGACAGTCCCTATCTGGACATGGAGCTTG | |
| | CCAGGGCGGGCTCAAGAGCATCAGAGGGCCAGGGCTCTGGGCT | |
| | GGGAGGGCAGCTGAGTGAGGTCTCTGGGCGAGGGGTGCAGCTC | |
| | TTTGAACAGCAGCGCCAGCGCGCAGACTCCAGCACCCAGGAAC | |
| | TGGCACGGGTCGAACCAGCAGCCATGCTCAACGGGGAAGGCCT | |
| | GCAGTCACCACCTCGGGCCCAGAGTGCTCCCCCAGAGGCAGCT | |
| | GTGCTCCCACCCAGCCCCTTGCCGGCGCCTGTAGCCAGCCCCA | |
| | GACCCTTCCAACCAGGTGGTGGAGCCCCGACCCCAGCTCCAAG | |
| | CATCTTTAACCGGTCAGCCAGGCCCTTTACCCCGGGCCTACAAG | |
| | GGCAGCGGCCAACTACCACCTCGGTTATTTTCCGGCCTTTAGCC | |
| | CCCAAAAGGGCGAACGACAGCCTGGGGGGCCTCAGCCCCGCCC | |
| | CACCCCCCTTCTTGTCTTCGCAGGGGCCCACCCCTCTGCCCAGC | |
| | TTCACTTCAGGGGTTCCCAGCCACGCGCCAGTCTCTGGTTCCCC | |
| | CAGCACCCCACGCTCCTCGGGCCCTGTGACAGCCACCAGCTCC | |
| | CTGTACATCCCAGCCCCTAGTCGGCCTGTCACCCCAGGTGGAG | |
| | CTCCAGAGCCCCCCGCTCCTCCTAGCGCAGCTGCCATGACCTCC | |
| | ACCGCTTCTATCTTCCTATCTGCGCCTTTGCGACCCTCTGCGCG | |
| | CCCAGAGGCGCCTGCCCCAGGCCCAGGGGCTCCTGAGCCCCCC | |
| | AGCGCTCGCGAGCAGCGCATCTCTGTGCCAGCTGCCCGCACGG | |
| | GTATCCTGCAGGAGGCCCGGCGCCGGGGGACCCGGAAGCAGA | |
| | TGTTCCGGCCGGGAAAGGAGGAGACGAAGAACTCGCCCAACC | |
| | CCGAGCTGCTATCGCTGGTACAGAACCTGGATGAAAAGCCTCG | |
| | GGCCGGGGGTGCAGAATCTGGTCCTGAAGAAGATGCTCTGAGC | |
| | CTCGGGGCTGAAGCCTGCAACTTCATGCAGCCAGTAGGGGCCA | |
| | GGAGTTACAAGACCCTGCCTCACGTGACACCTAAGACCCCCCC | |
| | TCCAATGGCTCCCAAGACCCCGCCCCCTATGACTCCTAAGACTC | |
| | CACCCCCAGTGGCTCCTAAGCCCCCATCTCGAGGGCTCCTTGAT | |
| | GGGCTCGTGAATGGGGCAGCCTCTTCGGCTGGAATCCCTGAGC | |
| | CACCAAGGCTGCAGGGCAGGGGTGGGGAGCTGTTTGCTAAGCG | |
| | GCAGAGCCGTGCGGACAGGTATGTGGTGGAAGGTACACCTGGT | |
| | CCTGGTCTTGGCCCTCGGCCTAGAAGTCCTTCTCCTACCCCGTC | |
| | TCTGCCCCCTTCCTGGAAATATTCACCCAACATCCGTGCCCCGC | |
| | CTCCTATTGCTTACAACCCACTGCTCTCTCCCTTTTTCCCCCAGG | |
| | CGGCCCGAACTCTCCCTAAGGCCCAATCCCAGGGGCCTCGGGC | |
| | AACACCCAAGCAGGGCATCAAGGCTCTAGATTTTATGCGGCAT | |
| | CAGCCCTATCAACTTAAAACTGCCATGTTCTGTTTTGATGAGGT | |
| | TCCCCCGACTCCTGGCCCTATCGCCTCAGGGTCCCCCAAAACTG | |
| | CCCGAGTCCAGGAGATTCGCCGGTTTTCCACTCCGGCACCCCA | |
| | GCCCACTGCAGAACCCCTGGCTCCCACTGTGCTTGCCCCCCGAG | |
| | CAGCCACTACACTGGATGAGCCCATCTGGAGAACAGAACTGGC | |
| | CTCAGCCCCTGTTCCTAGCCCAGCCCCTCCTCCAGAGGCTCCCA | |
| | GGGGCCTTGGGGCTTCTCCCAGCTCCTGCGGTTTCCAGGTAGCC | |
| | AGGCCCCGATTTTCAGCCACCAGAACAGGATTGCAAGCTCATG | |
| | TGTGGAGGCCTGGGGCAGGGCACCAG | |
| Human | ATGGAGACCTTTGAGCCCATCAGCCAAGAGCCCCTCAGCCAAG | 821 |
| SYNPO2L | CCAGCTATGACAAAGCCCCAGACCCAGTTCCTGAGCTCCAAGA | |
| B DNA | CTCGTTCTATGCAGAACTGCAACGTGCAGAGAGCCTCCAAGAG | |
| | AAGAGCATAAAAGAGGCCAAGACCAAATGCAGGACAATTGCA | |
| | TCCCTGCTCACTGCAGCCCCCAACCCCCACTCCAAAGGGGTACT | |
| | TATGTTTAAGAAACGGCGGCAGAGAGCCAAGAAGTACACCCTG | |
| | GTGAGCTTCGGGGCTGCTGCTGGGACAGGCGCTGAGGAGGAGG | |
| | ACGGCGTTCCCCCCACGAGTGAGTCCGAGCTGGACGAAGAAGC | |
| | CTTCTCTGACGCCCGCAGCCTCACCAATCAATCTGACTGGGACA | |
| | GTCCCTATCTGGACATGGAGCTTGCCAGGGCGGGCTCAAGAGC | |
| | ATCAGAGGGCCAGGGCTCTGGGCTGGGAGGGCAGCTGAGTGA | |
| | GGTCTCTGGGCGAGGGGTGCAGCTCTTTGAACAGCAGCGCCAG | |
| | CGCGCAGACTCCAGCACCCAGGAACTGGCACGGGTCGAACCAG | |
| | CAGCCATGCTCAACGGGGAAGGCCTGCAGTCACCACCTCGGGC | |
| | CCAGAGTGCTCCCCCAGAGGCAGCTGTGCTCCCACCCAGCCCC | |
| | TTGCCGGCGCCTGTAGCCAGCCCCAGACCCTTCCAACCAGGTG | |
| | GTGGAGCCCCGACCCCAGCTCCAAGCATCTTTAACCGGTCAGC | |
| | CAGGCCCTTTACCCCGGGCCTACAAGGGCAGCGGCCAACTACC | |
| | ACCTCGGTTATTTTCCGGCCTTTAGCCCCCAAAAGGGCGAACG | |
| | ACAGCCTGGGGGGCCTCAGCCCCGCCCCACCCCCCTTCTTGTCT | |
| | TCGCAGGGGCCCACCCCTCTGCCCAGCTTCACTTCAGGGGTTCC | |
| | CAGCCACGCGCCAGTCTCTGGTTCCCCCAGCACCCCACGCTCCT | |
| | CGGGCCCTGTGACAGCCACCAGCTCCCTGTACATCCCAGCCCCT | |
| | AGTCGGCCTGTCACCCCAGGTGGAGCTCCAGAGCCCCCCGCTC | |
| | CTCCTAGCGCAGCTGCCATGACCTCCACCGCTTCTATCTTCCTA | |
| | TCTGCGCCTTTGCGACCCTCTGCGCGCCCAGAGGCGCCTGCCCC | |
| | AGGCCCAGGGGCTCCTGAGCCCCCCAGCGCTCGCGAGCAGCGC | |
| | ATCTCTGTGCCAGCTGCCCGCACGGGTATCCTGCAGGAGGCCC | |
| | GGCGCCGGGGGACCCGGAAGCAGATGTTCCGGCCGGGAAAGG | |
| | AGGAGACGAAGAACTCGCCCAACCCCGAGCTGCTATCGCTGGT | |
| | ACAGAACCTGGATGAAAAGCCTCGGGCCGGGGGTGCAGAATCT | |
| | GGTCCTGAAGAAGATGCTCTGAGCCTCGGGGCTGAAGCCTGCA | |
| | ACTTCATGCAGCCAGTAGGGGCCAGGAGTTACAAGACCCTGCC | |
| | TCACGTGACACCTAAGACCCCCCCTCCAATGGCTCCCAAGACC | |
| | CCGCCCCCTATGACTCCTAAGACTCCACCCCCAGTGGCTCCTAA | |
| | GCCCCCATCTCGAGGGCTCCTTGATGGGCTCGTGAATGGGGCA | |
| | GCCTCTTCGGCTGGAATCCCTGAGCCACCAAGGCTGCAGGGCA | |
| | GGGGTGGGGAGCTGTTTGCTAAGCGGCAGAGCCGTGCGGACAG | |
| | GTATGTGGTGGAAGGTACACCTGGTCCTGGTCTTGGCCCTCGGC | |
| | CTAGAAGTCCTTCTCCTACCCCGTCTCTGCCCCCTTCCTGGAAA | |
| | TATTCACCCAACATCCGTGCCCCGCCTCCTATTGCTTACAACCC | |
| | ACTGCTCTCTCCCTTTTTCCCCCAGGCGGCCCGAACTCTCCCTA | |
| | AGGCCCAATCCCAGGGGCCTCGGGCAACACCCAAGCAGGGCAT | |
| | CAAGGCTCTAGATTTTATGCGGCATCAGCCCTATCAACTTAAAA | |
| | CTGCCATGTTCTGTTTTGATGAGGTTCCCCCGACTCCTGGCCCT | |
| | ATCGCCTCAGGGTCCCCCAAAACTGCCCGAGTCCAGGAGATTC | |
| | GCCGGTTTTCCACTCCGGCACCCCAGCCCACTGCAGAACCCCTG | |
| | GCTCCCACTGTGCTTGCCCCCCGAGCAGCCACTACACTGGATG | |
| | AGCCCATCTGGAGAACAGAACTGGCCTCAGCCCCTGTTCCTAG | |
| | CCCAGCCCCTCCTCCAGAGGCTCCCAGGGGCCTTGGGGCTTCTC | |
| | CCAGCTCCTGCGGTTTCCAGGTAGCCAGGCCCCGATTTTCAGCC | |
| | ACCAGAACAGGATTGCAAGCTCATGTGTGGAGGCCTGGGGCAG | |
| | GGCACCAG | |
| Human | MAPAAWLRSAAARALLPPMLLLLLQPPPLLARALPPDAHHLHAE | 822 |
| MMP11 | RRGPQPWHAALPSSPAPAPATQEAPRPASSLRPPRCGVPDPSDGLS | |
| protein | ARNRQKRFVLSGGRWEKTDLTYRILRFPWQLVQEQVRQTMAEAL | |
| | KVWSDVTPLTFTEVHEGRADIMIDFARYWHGDDLPFDGPGGILAH | |
| | AFFPKTHREGDVHFDYDETWTIGDDQGTDLLQVAAHEFGHVLGL | |
| | QHTTAAKALMSAFYTFRYPLSLSPDDCRGVQHLYGQPWPTVTSR | |
| | TPALGPQAGIDTNEIAPLEPDAPPDACEASEDAVSTIRGELFFFKAG | |
| | FVWRLRGGQLQPGYPALASRHWQGLPSPVDAAFEDAQGHIWFFQ | |
| | GAQYWVYDGEKPVLGPAPLTELGLVRFPVHAALVWGPEKNKIYF | |
| | FRGRDYWRFHPSTRRVDSPVPRRATDWRGVPSEIDAAFQDADGY | |
| | AYFLRGRLYWKFDPVKVKALEGFPRLVGPDFFGCAEPANTFL | |
| Human | agagcgagcgccggggccgggcgcgcaggagtgaaaaggaggcggcggccgcagctgcga | 831 |
| MTSS1 | gcaacagatccggacgccgcgagctgacccgctctgctgttgggcgatttttttttaatt | |
| mRNA | gcagaaaaatttattaaattggaaaatcttgcgtttttcaatggcgctggccccgggtca | |
| | gcgggcgattttctctgcatcaagatgggctttgccgtttccgtagtgggcaccagtggt | |
| | ggcctgattgtcagtcttctcccggcatttttaaggccaggagccgagcgctgcttgtag | |
| | gcgaataccctacagagcggtttggctttttaaattactgttattattttgggcagagaa | |
| | cagtcggtctggtgcaccccgtcctcgctgcagaagaggctgcgagtccgaggtgggtct | |
| | ctcggaaggtgaaattccttctggggtgagcgagccccggccccgcgcgcagtccagcgg | |
| | ccccgcgtgtgtgccctcgccctgccggagccgggaaaatggaggctgtgattgagaagg | |
| | aatgcagcgcgctcggaggcctcttccagaccatcatcagcgacatgaaggggagctatc | |
| | cagtttgggaagatttcataaacaaagcaggaaagctgcagtcccagcttcggacaacag | |
| | tagtagcagcagctgccttcttggacgcctttcagaaagtggctgacatggccaccaaca | |
| | cacgtggtgggaccagggagattggatctgctctcaccaggatgtgcatgaggcacagaa | |
| | gcattgaagccaagctgaggcagttttcgagcgctttaattgattgtctgataaacccac | |
| | ttcaagaacagatggaagaatggaagaaagtggccaaccagctggataaagaccacgcaa | |
| | aagaatataagaaagcccgccaagagataaaaaagaagtcctcggatacgctgaaactgc | |
| | agaagaaagcaaaaaaagggagaggtgatatccagcctcagttggacagtgctctccaag | |
| | atgtcaatgataagtatctcttattggaagaaacagaaaagcaggctgtccggaaggctt | |
| | tgattgaagaacgtggccgattctgtaccttcatctctatgctgcggccagtgattgaag | |
| | aagaaatctcaatgctaggggaaataacccaccttcagaccatctcggaagatctaaaaa | |
| | gcctgaccatggaccctcacaaactgccctcctcaagtgaacaggtgattctggacttga | |
| | aaggttctgattacagctggtcgtatcagacgccaccctcttcccccagcaccaccatgt | |
| | ccagaaagtccagtgtctgcagcagcctgaacagtgtcaacagcagtgactcccggtcca | |
| | gcggctcccactcgcattcccccagctcacattaccgctaccgcagetccaacctggccc | |
| | agcaggctcctgtgaggctgtccagcgtgtcctcccatgactcaggattcatatcccagg | |
| | atgccttccagtccaagtcaccatcccccatgccgccagaggcccccaaccagttgtcta | |
| | acgggttttctcactatagtttatcaagtgagtcccacgtggggcccacgggtgcaggcc | |
| | ttttccctcattgcctgcctgcctcccgcctgctccctegggtcacctctgtccaccttc | |
| | cagactacgctcattattacaccattgggcccggcatgttcccgtcatctcagatcccta | |
| | gctggaaggactgggctaagcctgggccctatgaccagcctctggtgaacaccctgcagc | |
| | gccgcaaagagaagcgagaaccggaccccaacgggggaggacccactaccgccagcggcc | |
| | cacctgcagcagctgaggaggctcagagaccacggagcatgactgtatcggctgccacca | |
| | ggcctggtgaggagatggaggcttgtgaggagctggccctggccctgtctcggggcctgc | |
| | agctggacacccagaggagcagccgggactcgcttcagtgctccagcggctacagcaccc | |
| | agacaaccaccccctgctgctctgaggacaccatcccttcccaagtttcagattatgatt | |
| | atttctctgtaagtggtgaccaggaggcagatcagcaggagttcgacaagtcctccacca | |
| | ttccaagaaacagcgacatcagccagtcctaccgacggatgttccaagccaagcgtccag | |
| | cctcaactgctggcctccccaccaccctgggacctgctatggtcactccaggggttgcaa | |
| | ctatccgacggaccccttccaccaagccttctgtccgccggggaaccattggagctggtc | |
| | ccatccccatcaagacacccgtgatccctgtcaagaccccaaccgtcccagacctcccag | |
| | gggtgttgccagcccctccagatgggccagaagagcggggggagcacagccctgagtcgc | |
| | catctgtgggtgagggcccccaaggtgtcaccagcatgccctcctcaatgtggagcggcc | |
| | aagcttccgttaaccctccacttccaggcccgaagcccagtatccctgaggagcacagac | |
| | aggcaattccagaaagtgaagctgaagaccaggaacgggaacccccaagtgccactgtct | |
| | ccccaggccagattccagagagtgaccctgcagacctgagcccaagggatactccacaag | |
| | gagaagacatgctgaacgccatccgaaggggcgtgaaactgaagaagaccacgacaaacg | |
| | atcgctcagcccctcgcttttcttaggttcacaagaaatgcgccggtggggaatgaactg | |
| | tttcattaataaaacctaatttgtcttgatccattccactctataataaaacaaaagatt | |
| | ttgtaggcaactcggaatatagctcttttgaaagtactcgacacctttagataagaatta | |
| | aaaccaacctatgtaactgacataatcttgatcttttaatttgtaaatattgacaatttt | |
| | ctttctgcacattttaatcttagtttcccttttgatttttctgaaggtgccaaattccat | |
| | ttaacttttttacaagtctttgtaaaattttaaatgcataaagggggttggggcagggga | |
| | accacgaagtagttaattttagaaaaggatttactatacttcactcttctttttttttcc | |
| | ccacaagcttttgtagatgcattgtagtagtctagcttagaagcaaatgcaagttatttt | |
| | aatgtacaaactaaatgggtaagaggtaaaatcttcatttaaatatactatgttctggat | |
| | gaaaagagcaggagtaacaattgatgagcaatattcagagtgaagtaaatctggaaatgg | |
| | tagactgtgttgggattggggggagggccatgggaggggtacatcgtcaacatagccgat | |
| | cctgttacatttaagagtagcctcgtaggttgaatttcttctggtagcttcatggtaaat | |
| | gcatccgaataagccatactggattgcagtgtttgtttctgtagggtgtttaaggacttg | |
| | acttcctttctcccatgattcctctggactgcacacagcacccacaaccagccccatgca | |
| | tgctgctgcctctgggcagtcgtagaatctcccacttcagtttctcgttgattgtactca | |
| | cctttatggaatccaaatacatccaaaagggtaaggcagttttaaaaatgtgaaaacatt | |
| | taaaaatgataatagcagggaattcttagattatagtaaatgccttttacttaactgtgc | |
| | ccagcaggctgggtgcgttaaaaagcccaagtattttgaaaaaactcgaacagatttgac | |
| | aagggtagccagcttggagtctagcaacttgccaatgtgtttaccaatctgggggcttgt | |
| | ttttcttttcttctttcaaataaatggcagttaactggctttacagtaaacattgaagag | |
| | aggaggatttgtttattgtcactgggaatctgaccactatactgtcctttttttgtattc | |
| | tgggtaaatgttttttggaaaagatttgtcttttctaagtggaagttaaatttgttatac | |
| | tgcccatcccctaaagccaacagagatttgtagatttaaagggatcacatttgaagacaa | |
| | tagtgtttaagaaagcaagcaagtcccttagcagtcaggtcataacagggcacatttctg | |
| | accgaaccctctcaaggcagaggaggagtttggtgggtttcatacaccctgcagattcct | |
| | gttggctctaaccctcaattacctaatcttatgctttaacacataactgcattggatgtg | |
| | agagtaacgtaccgtatggtcattgttctatatattaacattgaacactgctgcgattgc | |
| | tcaaggacattttatgttacggctttaaagcaaaggcatgattattagaaactatttaag | |
| | cttttttctttgaaaaacaagctccttttacagaatataaacaacagtagtgcctgtggt | |
| | ttagcccaccaatcttgatgactaaaagtagctgatgcattgtgcatatgatgcttgaga | |
| | tggtttttgcaaaagcagaaatcgctgcaaggtaatcacaatagataaaagtggtatttt | |
| | aaacctttgaaataaatggatgtaactgtaccttggtacagcttttcacttgtttagttt | |
| | ttaaacgttagtataatctgaataaataaaatgttgccaaattcaatgtagaaagaatgt | |
| | gacaacacaccttgggtagttctgcttgtgtttttgcatattgtaaaagcagtgtcacag | |
| | ctaaaaagaaagaaatcgtttctaacagtaaattattgtgctttagttgctagtttgtac | |
| | tgagagttgacctctccctgtgcagttttttgttctaaacttgtataaataacaattgtg | |
| | taatgtgtctccctcctacattgtaacaattgcttcagcctacgttataaataaagaacc | |
| | actagattaaaaaa | |
| Human | MGAEEEVLVTLSGGAPWGFRLHGGAEQRKPLQVSKIRRRSQAGR | 823 |
| SYNPO2L | AGLRERDQLLAINGVSCTNLSHASAMSLIDASGNQLVLTVQRLAD | |
| A | EGPVQSPSPHELQVLSPLSPLSPEPPGAPVPQPLQPGSLRSPPDSEAY | |
| protein | YGETDSDADGPATQEKPRRPRRRGPTRPTPPGAPPDEVYLSDSPAE | |
| | PAPTIPGPPSQGDSRVSSPSWEDGAALQPPPAEALLLPHGPLRPGPH | |
| | LIPMVGPVPHPVAEDLTTTYTQKAKQAKLQRAESLQEKSIKEAKT | |
| | KCRTIASLLTAAPNPHSKGVLMFKKRRQRAKKYTLVSFGAAAGT | |
| | GAEEEDGVPPTSESELDEEAFSDARSLTNQSDWDSPYLDMELARA | |
| | GSRASEGQGSGLGGQLSEVSGRGVQLFEQQRQRADSSTQELARVE | |
| | PAAMLNGEGLQSPPRAQSAPPEAAVLPPSPLPAPVASPRPFQPGGG | |
| | APTPAPSIFNRSARPFTPGLQGQRPTTTSVIFRPLAPKRANDSLGGL | |
| | SPAPPPFLSSQGPTPLPSFTSGVPSHAPVSGSPSTPRSSGPVTATSSL | |
| | YIPAPSRPVTPGGAPEPPAPPSAAAMTSTASIFLSAPLRPSARPEAPA | |
| | PGPGAPEPPSAREQRISVPAARTGILQEARRRGTRKQMFRPGKEET | |
| | KNSPNPELLSLVQNLDEKPRAGGAESGPEEDALSLGAEACNFMQP | |
| | VGARSYKTLPHVTPKTPPPMAPKTPPPMTPKTPPPVAPKPPSRGLL | |
| | DGLVNGAASSAGIPEPPRLQGRGGELFAKRQSRADRYVVEGTPGP | |
| | GLGPRPRSPSPTPSLPPSWKYSPNIRAPPPIAYNPLLSPFFPQAARTL | |
| | PKAQSQGPRATPKQGIKALDFMRHQPYQLKTAMFCFDEVPPTPGP | |
| | IASGSPKTARVQEIRRFSTPAPQPTAEPLAPTVLAPRAATTLDEPIW | |
| | RTELASAPVPSPAPPPEAPRGLGASPSSCGFQVARPRESATRTGLQ | |
| | AHVWRPGAGHQ | |
| Human | METFEPISQEPLSQASYDKAPDPVPELQDSFYAELQRAESLQEKSIK | 824 |
| SYNPOP2 | EAKTKCRTIASLLTAAPNPHSKGVLMFKKRRQRAKKYTLVSFGAA | |
| B protein | AGTGAEEEDGVPPTSESELDEEAFSDARSLTNQSDWDSPYLDMEL | |
| | ARAGSRASEGQGSGLGGQLSEVSGRGVQLFEQQRQRADSSTQEL | |
| | ARVEPAAMLNGEGLQSPPRAQSAPPEAAVLPPSPLPAPVASPRPFQ | |
| | PGGGAPTPAPSIFNRSARPFTPGLQGQRPTTTSVIFRPLAPKRANDS | |
| | LGGLSPAPPPFLSSQGPTPLPSFTSGVPSHAPVSGSPSTPRSSGPVTA | |
| | TSSLYIPAPSRPVTPGGAPEPPAPPSAAAMTSTASIFLSAPLRPSARP | |
| | EAPAPGPGAPEPPSAREQRISVPAARTGILQEARRRGTRKQMFRPG | |
| | KEETKNSPNPELLSLVQNLDEKPRAGGAESGPEEDALSLGAEACN | |
| | FMQPVGARSYKTLPHVTPKTPPPMAPKTPPPMTPKTPPPVAPKPPS | |
| | RGLLDGLVNGAASSAGIPEPPRLQGRGGELFAKRQSRADRYVVEG | |
| | TPGPGLGPRPRSPSPTPSLPPSWKYSPNIRAPPPIAYNPLLSPFFPQA | |
| | ARTLPKAQSQGPRATPKQGIKALDFMRHQPYQLKTAMFCFDEVPP | |
| | TPGPIASGSPKTARVQEIRRFSTPAPQPTAEPLAPTVLAPRAATTLD | |
| | EPIWRTELASAPVPSPAPPPEAPRGLGASPSSCGFQVARPRFSATRT | |
| | GLQAHVWRPGAGHQ | |
| Human | MEAVIEKECSALGGLFQTIISDMKGSYPVWEDFINKAGKLQSQLRT | 825 |
| MTSS1 | TVVAAAAFLDAFQKVADMATNTRGGTREIGSALTRMCMRHRSIE | |
| protein | AKLRQFSSALIDCLINPLQEQMEEWKKVANQLDKDHAKEYKKAR | |
| | QEIKKKSSDTLKLQKKAKKGRGDIQPQLDSALQDVNDKYLLLEET | |
| | EKQAVRKALIEERGRFCTFISMLRPVIEEEISMLGEITHLQTISEDLK | |
| | SLTMDPHKLPSSSEQVILDLKGSDYSWSYQTPPSSPSTTMSRKSSV | |
| | CSSLNSVNSSDSRSSGSHSHSPSSHYRYRSSNLAQQAPVRLSSVSSH | |
| | DSGFISQDAFQSKSPSPMPPEAPNQLSNGFSHYSLSSESHVGPTGAG | |
| | LFPHCLPASRLLPRVTSVHLPDYAHYYTIGPGMFPSSQIPSWKDWA | |
| | KPGPYDQPLVNTLQRRKEKREPDPNGGGPTTASGPPAAAEEAQRP | |
| | RSMTVSAATRPGEEMEACEELALALSRGLQLDTQRSSRDSLQCSS | |
| | GYSTQTTTPCCSEDTIPSQVSDYDYFSVSGDQEADQQEFDKSSTIPR | |
| | NSDISQSYRRMFQAKRPASTAGLPTTLGPAMVTPGVATIRRTPSTK | |
| | PSVRRGTIGAGPIPIKTPVIPVKTPTVPDLPGVLPAPPDGPEERGEHS | |
| | PESPSVGEGPQGVTSMPSSMWSGQASVNPPLPGPKPSIPEEHRQAIP | |
| | ESEAEDQEREPPSATVSPGQIPESDPADLSPRDTPQGEDMLNAIRR | |
| | GVKLKKTTTNDRSAPRES | |
| Human | MSAATHSPMMQVASGNGDRDPLPPGWEIKIDPQTGWPFFVDHNS | 829 |
| BAG3 | RTTTWNDPRVPSEGPKETPSSANGPSREGSRLPPAREGHPVYPQLR | |
| C151R | PGYIPIPVLHEGAENRQVHPFHVYPQPGMQRFRTEAAAAAPQRSQ | |
| mutant | SPLRGMPETTQPDKQRGQVAAAAAAQPPASHGPERSQSPAASDCS | |
| protein | SSSSSASLPSSGRSSLGSHQLPRGYISIPVIHEQNVTRPAAQPSFHQA | |
| | QKTHYPAQQGEYQTHQPVYHKIQGDDWEPRPLRAASPFRSSVQG | |
| | ASSREGSPARSSTPLHSPSPIRVHTVVDRPQQPMTHRETAPVSQPE | |
| | NKPESKPGPVGPELPPGHIPIQVIRKEVDSKPVSQKPPPPSEKVEVK | |
| | VPPAPVPCPPPSPGPSAVPSSPKSVATEERAAPSTAPAEATPPKPGE | |
| | AEAPPKHPGVLKVEAILEKVQGLEQAVDNFEGKKTDKKYLMIEE | |
| | YLTKELLALDSVDPEGRADVRQARRDGVRKVQTILEKLEQKAIDV | |
| | PGQVQVYELQPSNLEADQPLQAIMEMGAVAADKGKKNAGNAED | |
| | PHTETQQPEATAAATSNPSSMTDTPGNPAAP | |
| Staphylococcus | atggccccaaagaagaagcggaaggtcggtatccacggagtcccagcagccaagcggaactacatcc | 832 |
| aureus | tgggcctggacatcggcatcaccagcgtgggctacggcatcatcgactacgagacacgggacgtgatc | |
| saCas9 | gatgccggcgtgcggctgttcaaagaggccaacgtggaaaacaacgagggcaggcggagcaagaga | |
| DNA | ggcgccagaaggctgaagcggcggaggcggcatagaatccagagagtgaagaagctgctgttcgact | |
| | acaacctgctgaccgaccacagcgagctgagcggcatcaacccctacgaggccagagtgaagggcct | |
| | gagccagaagctgagcgaggaagagttctctgccgccctgctgcacctggccaagagaagaggcgtg | |
| | cacaacgtgaacgaggtggaagaggacaccggcaacgagctgtccaccaaagagcagatcagccgg | |
| | aacagcaaggccctggaagagaaatacgtggccgaactgcagctggaacggctgaagaaagacggc | |
| | gaagtgcggggcagcatcaacagattcaagaccagcgactacgtgaaagaagccaaacagctgctga | |
| | aggtgcagaaggcctaccaccagctggaccagagcttcatcgacacctacatcgacctgctggaaacc | |
| | cggcggacctactatgagggacctggcgagggcagccccttcggctggaaggacatcaaagaatggt | |
| | acgagatgctgatgggccactgcacctacttccccgaggaactgcggagcgtgaagtacgcctacaac | |
| | gccgacctgtacaacgccctgaacgacctgaacaatctcgtgatcaccagggacgagaacgagaagct | |
| | ggaatattacgagaagttccagatcatcgagaacgtgttcaagcagaagaagaagcccaccctgaagca | |
| | gatcgccaaagaaatcctcgtgaacgaagaggatattaagggctacagagtgaccagcaccggcaagc | |
| | ccgagttcaccaacctgaaggtgtaccacgacatcaaggacattaccgcccggaaagagattattgaga | |
| | acgccgagctgctggatcagattgccaagatcctgaccatctaccagagcagcgaggacatccaggaa | |
| | gaactgaccaatctgaactccgagctgacccaggaagagatcgagcagatctctaatctgaagggctat | |
| | accggcacccacaacctgagcctgaaggccatcaacctgatcctggacgagctgtggcacaccaacga | |
| | caaccagatcgctatcttcaaccggctgaagctggtgcccaagaaggtggacctgtcccagcagaaaga | |
| | gatccccaccaccctggtggacgacttcatcctgagccccgtcgtgaagagaagcttcatccagagcatc | |
| | aaagtgatcaacgccatcatcaagaagtacggcctgcccaacgacatcattatcgagctggcccgcgag | |
| | aagaactccaaggacgcccagaaaatgatcaacgagatgcagaagcggaaccggcagaccaacgag | |
| | cggatcgaggaaatcatccggaccaccggcaaagagaacgccaagtacctgatcgagaagatcaagc | |
| | tgcacgacatgcaggaaggcaagtgcctgtacagcctggaagccatccctctggaagatctgctgaaca | |
| | accccttcaactatgaggtggaccacatcatccccagaagcgtgtccttcgacaacagcttcaacaacaa | |
| | ggtgctcgtgaagcaggaagaaaacagcaagaagggcaaccggaccccattccagtacctgagcagc | |
| | agcgacagcaagatcagctacgaaaccttcaagaagcacatcctgaatctggccaagggcaagggca | |
| | gaatcagcaagaccaagaaagagtatctgctggaagaacgggacatcaacaggttctccgtgcagaaa | |
| | gacttcatcaaccggaacctggtggataccagatacgccaccagaggcctgatgaacctgctgcggag | |
| | ctacttcagagtgaacaacctggacgtgaaagtgaagtccatcaatggcggcttcaccagctttctgcgg | |
| | cggaagtggaagtttaagaaagagcggaacaaggggtacaagcaccacgccgaggacgccctgatca | |
| | ttgccaacgccgatttcatcttcaaagagtggaagaaactggacaaggccaaaaaagtgatggaaaacc | |
| | agatgttcgaggaaaagcaggccgagagcatgcccgagatcgaaaccgagcaggagtacaaagagat | |
| | cttcatcaccccccaccagatcaagcacattaaggacttcaaggactacaagtacagccaccgggtgga | |
| | caagaagcctaatagagagctgattaacgacaccctgtactccacccggaaggacgacaagggcaaca | |
| | ccctgatcgtgaacaatctgaacggcctgtacgacaaggacaatgacaagctgaaaaagctgatcaaca | |
| | agagccccgaaaagctgctgatgtaccaccacgacccccagacctaccagaaactgaagctgattatgg | |
| | aacagtacggcgacgagaagaatcccctgtacaagtactacgaggaaaccgggaactacctgaccaag | |
| | tactccaaaaaggacaacggccccgtgatcaagaagattaagtattacggcaacaaactgaacgcccat | |
| | ctggacatcaccgacgactaccccaacagcagaaacaaggtcgtgaagctgtccctgaagccctacag | |
| | attcgacgtgtacctggacaatggcgtgtacaagttcgtgaccgtgaagaatctggatgtgatcaaaaaa | |
| | gaaaactactacgaagtgaatagcaagtgctatgaggaagctaagaagctgaagaagatcagcaaccag | |
| | gccgagtttatcgcctccttctacaacaacgatctgatcaagatcaacggcgagctgtatagagtgatcg | |
| | gcgtgaacaacgacctgctgaaccggatcgaagtgaacatgatcgacatcacctaccgcgagtacctgga | |
| | aaacatgaacgacaagaggccccccaggatcattaagacaatcgcctccaagacccagagcattaaga | |
| | agtacagcacagacattctgggcaacctgtatgaagtgaaatctaagaagcaccctcagatcatcaaaaa | |
| | gggcaaaaggccggcggccacgaaaaaggccggccaggcaaaaaagaaaaagtaa | |
| Staphylococcus | MAPKKKRKVGIHGVPAAKRNYILGLDIGITSVGYGIIDYETRDVID | 833 |
| aureus | AGVRLFKEANVENNEGRRSKRGARRLKRRRRHRIQRVKKLLFDY | |
| saCas9 | NLLTDHSELSGINPYEARVKGLSQKLSEEEFSAALLHLAKRRGVH | |
| protein | NVNEVEEDTGNELSTKEQISRNSKALEEKYVAELQLERLKKDGEV | |
| | RGSINRFKTSDYVKEAKQLLKVQKAYHQLDQSFIDTYIDLLETRRT | |
| | YYEGPGEGSPFGWKDIKEWYEMLMGHCTYFPEELRSVKYAYNA | |
| | DLYNALNDLNNLVITRDENEKLEYYEKFQIIENVFKQKKKPTLKQI | |
| | AKEILVNEEDIKGYRVTSTGKPEFTNLKVYHDIKDITARKEIIENAE | |
| | LLDQIAKILTIYQSSEDIQEELTNLNSELTQEEIEQISNLKGYTGTHN | |
| | LSLKAINLILDELWHTNDNQIAIFNRLKLVPKKVDLSQQKEIPTTLV | |
| | DDFILSPVVKRSFIQSIKVINAIIKKYGLPNDIIIELAREKNSKDAQK | |
| | MINEMQKRNRQTNERIEEIIRTTGKENAKYLIEKIKLHDMQEGKCL | |
| | YSLEAIPLEDLLNNPFNYEVDHIIPRSVSFDNSFNNKVLVKQEENSK | |
| | KGNRTPFQYLSSSDSKISYETFKKHILNLAKGKGRISKTKKEYLLEE | |
| | RDINRFSVQKDFINRNLVDTRYATRGLMNLLRSYFRVNNLDVKV | |
| | KSINGGFTSFLRRKWKFKKERNKGYKHHAEDALIIANADFIFKEW | |
| | KKLDKAKKVMENQMFEEKQAESMPEIETEQEYKEIFITPHQIKHIK | |
| | DFKDYKYSHRVDKKPNRELINDTLYSTRKDDKGNTLIVNNLNGL | |
| | YDKDNDKLKKLINKSPEKLLMYHHDPQTYQKLKLIMEQYGDEKN | |
| | PLYKYYEETGNYLTKYSKKDNGPVIKKIKYYGNKLNAHLDITDDY | |
| | PNSRNKVVKLSLKPYRFDVYLDNGVYKFVTVKNLDVIKKENYYE | |
| | VNSKCYEEAKKLKKISNQAEFIASFYNNDLIKINGELYRVIGVNND | |
| | LLNRIEVNMIDITYREYLENMNDKRPPRIIKTIASKTQSIKKYSTDIL | |
| | GNLYEVKSKKHPQIIKKGKRPAATKKAGQAKKKK | |

The following non-limiting Examples illustrate some of the experimental work involved in developing the invention.

### EXAMPLES

### Example 1: AAV Capsid Engineering Through Directed Evolution in NHP

This Examples discloses directed evolution of the AAV9 capsid to identify variants have higher heart transduction and/or high heart-to-liver trafficking ratio (“liver detargeting”). In clinical use, higher heart transduction may result in more efficient therapeutic gene delivery, and thus lead to better efficacy at the same dosage or lower dosage requirement to reach the desired efficacy. Higher heart-to-liver ratio may reduce toxicity and adverse effects related to high liver viral load, while having heart gene delivery unaffected or improved.

### Library Generation and AAV Selection

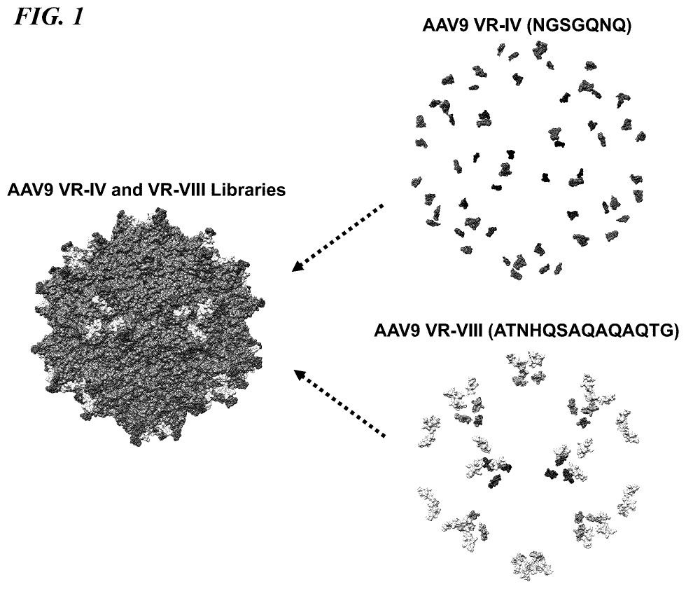

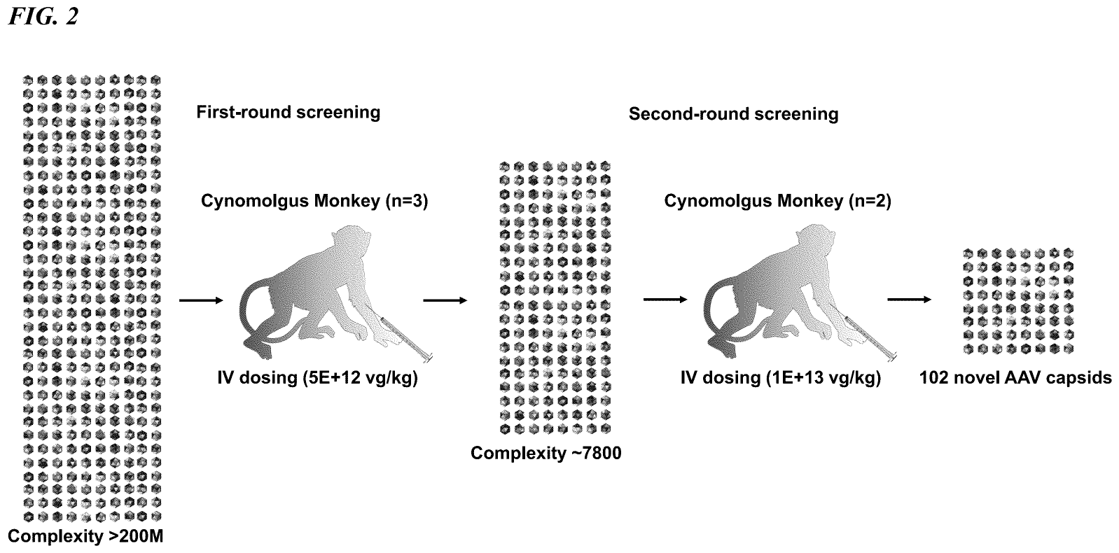

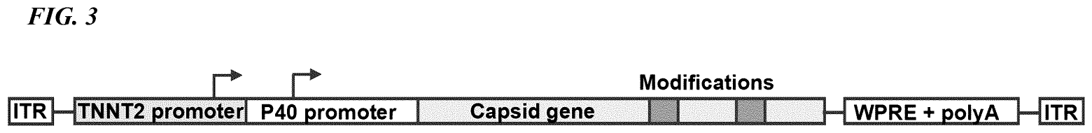

Variable regions (VR-IV and VR-VIII sites) on the AAV9 capsid are shown in FIG. 1. A library screening strategy (FIG. 2) was employed to identify novel AAV capsid variants for better heart gene delivery through systemic administration. A library of more than 200 million capsid variants was generating through synthesis of pools of oligonucleotides encoding random amino-acid residues or designed insertions and substitutions at selected positions in viral proteins (VP) of the AAV9 capsid. The pools were cloned into the capsid (cap) gene in the recombinant vector genome depicted in FIG. 3 that places the capsid (cap) gene under the control of a P40 promoter to express the capsid proteins VP1, VP2, and VP3 in in vitro, thereby generating infectious virions with the modified VP proteins. Upstream of the P40 promoter, a shortened cardiac troponin (TNNT2) promoter, described in US 2022/0031866 A1, was included to drive the expression of the capsid mRNA transcript in cardiomyocytes in vivo, which enables detection of viral mRNA from the heart. This design facilitates selection of only those AAV virions that both traffic to the desired organ (heart) and express the transgene in the cells. When injecting into a subject, the cap gene is expressed in the heart if the encapsulating AAV virion traffics to the heart, enters the cells, and deliver the transgene to the nucleus. Accordingly, the mRNA expressed in the heart should be mRNA corresponding to AAV virions having the desired tropism. Off-target trafficking can be detected by DNA. Trafficking of the AAV virion to a target causes vector genome DNA to be present in that issue.

The library was formed by pooling seven sub-libraries, listed in Table 4. In sub-library 1, positions in the VR-IV site in the capsid sequence of a variant AAV9 capsid were randomized; the variant AAV9 capsid had the artificial sequence ANYG (replacing the native SAQA sequence) in its in VR-VIII site. In sub-libraries 2 through 7, positions in the VR-VIII site of the AAV9 capsid were randomized.

TABLE 4
Variant libraries

| Sub- | |
|---|---|
| Library | |
| No. | Description |
| 1 | SAQA sequence at positions 586-589 (VR-VIII site) replaced with ANYG |
| | Positions 452-458 (VR-IV site) replaced with NNNNNNN, where N represents a |
| | random amino acid |
| 2 | Two amino acids inserted at each of positions 582-588 |
| | All 2800 possible variants |
| | (seven positions × 400 possible two amino acid insertions) |
| 3 | Positions 581-594 replaced with sequences predicted by machine learning |
| | algorithm |
| | 200 variants |
| 4 | Positions 582-584, 583-585, 584-586, 585-587, 586-588, or 587-589 replaced |
| | with NXN, where N represents a random amino acid, and X represents the amino |
| | acid in the reference sequence |
| | All 2394 possible variants |
| 5 | Single amino acid substitution at each of positions 581-594 |
| | All 266 possible variants |
| 6 | Position 585-590 replaced with six residue random sequence |
| 7 | Single amino acid substitution at position 587 and single amino acid insertion at |
| | position 589 |

As shown in FIG. 1 and FIG. 2, these initial libraries, which theoretically included more than 200 million cap gene sequences, were packaged using conventional HEK293T AAV production system, where the Adenovirus helper plasmid, the AAV2 REP plasmid, and the CAP library plasmids were transfected into HEK293T cells. Cell lysate was collected after 72 hours and AAV virions were purified by iodixanol gradient ultracentrifugation. The resulting pool of AAV virions were intravenously injected into three Cynomolgus monkeys 5E+12 viral genomes per kilogram body weight. At 4-weeks post-injection, animals were sacrificed and biopsies were taken from the heart and liver. RNA was isolated from heart samples and viral transcripts were amplified and sequenced by Next-Generation Sequencing to detect novel variants that transduced heart. DNA was isolated from liver samples and viral genome sequences were amplified and sequenced by Next-Generation Sequencing (NGS) to detect variants that infected liver. Using computational analysis of the sequencing results, approximately 7800 variants were selected for having high heart-transduction, high heart-to-liver ratio, or both.

A second-round screening was performed by re-synthesizing and cloning DNA sequences encoding the selected 7800 variants, expressing AAV virions, and injecting the resulting AAV pool into two Cynomolgus monkeys intravenously at 1E+13 viral genomes per kilogram body weight. At 4-weeks post-injection, animals were sacrificed and biopsies were taken from the heart and liver. RNA was isolated from heart samples and viral transcripts were amplified and sequenced by Next-Generation Sequencing to detect novel variants that transduced heart. DNA was isolated from liver samples and viral genome sequences were amplified and sequenced by Next-Generation Sequencing (NGS) to detect variants that infected liver. After NGS, computational analysis of second-round screen identified 102 novel AAV capsids as superior to AAV9 in heart transduction, heart-to-liver ratio, or both.

### Screening Results

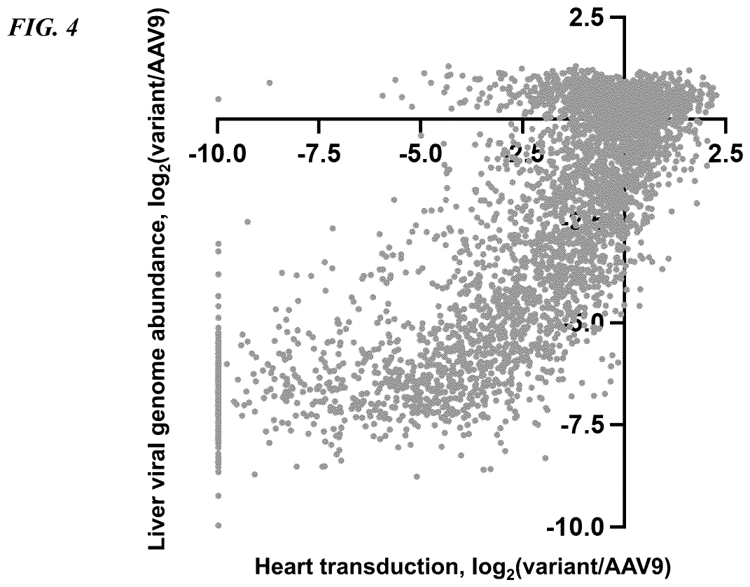

FIG. 4 plots the data from the second-round screening. All the detectable variants (including two or more synonymous codon replicates for each variant) are plotted. Heart transduction and liver infection measurements were obtained by deep sequencing viral transcripts recovered from heart biopsies or viral genome sequences recovered from liver biopsies, identifying the variant coding sequence on each Next-Generation Sequencing reads, counting the appearance of each unique variant and normalizing the read count of each unique variant to the total number of reads from that biopsy. We also normalized the frequency of each unique variant to its abundance in the input virus library. We report the frequency of each unique variant relative to the frequency of the AAV9 control present in the sample input virus library, and calculating the log 2 value of the resulting ratio. Heart mRNA abundance (a measure of infection of cardiomyocytes) is plotted against liver DNA abundance (a measure of trafficking to the liver).

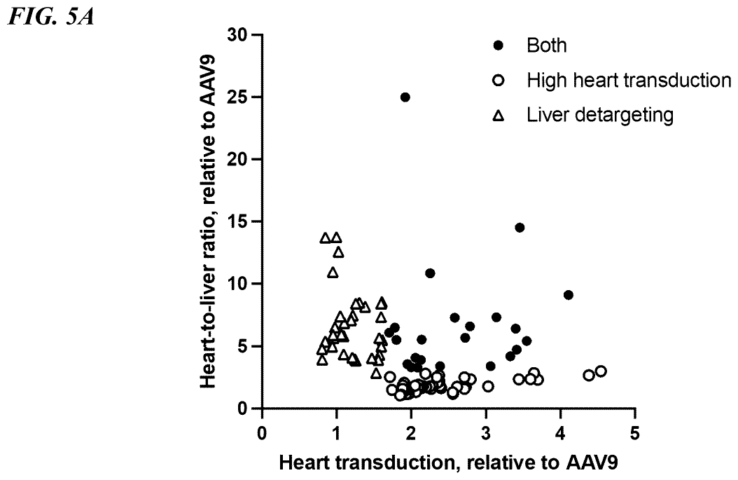

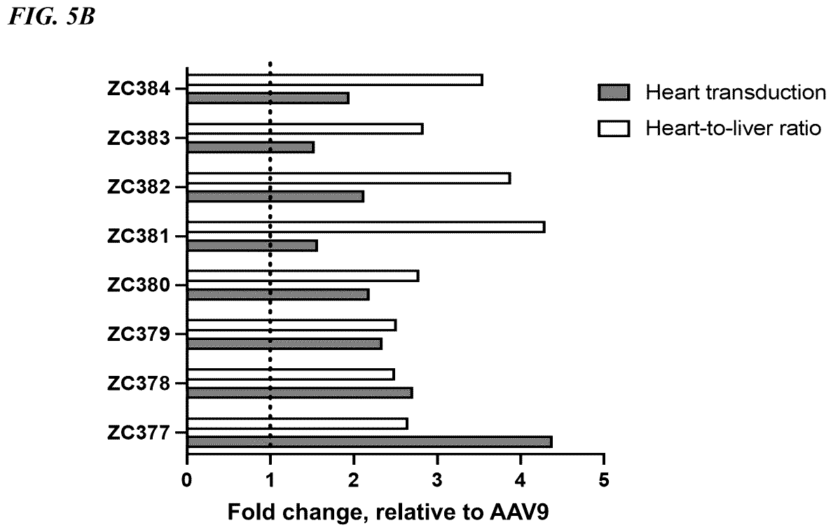

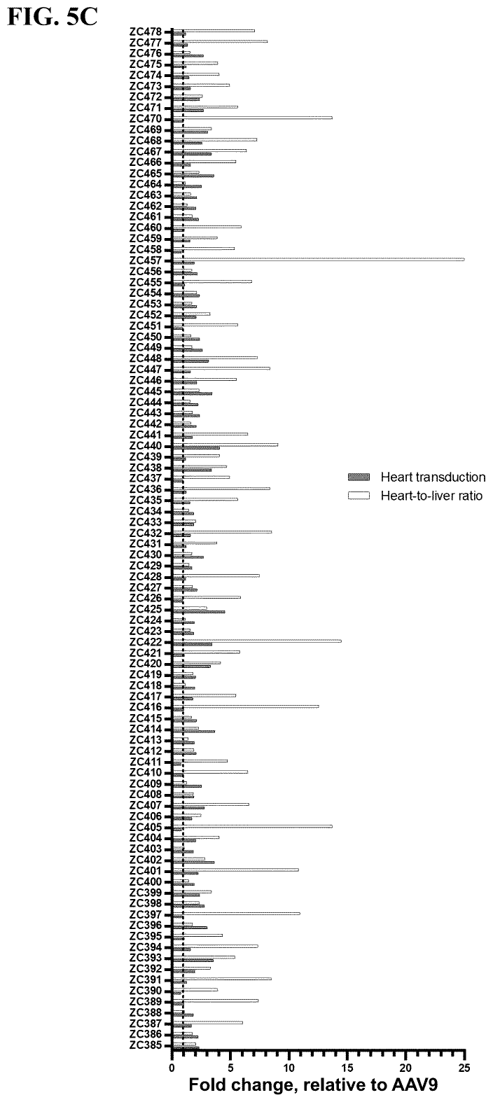

FIGS. 5A-5C plot 102 variants selected as having the desired properties (high heart transduction relative to AAV9, high heart-to-liver ratio relative to AAV9, or both).

FIG. 5A plots heart transduction measurements of the 102 selected variants on x-axis and heart-to-liver ratios on y-axis. Heart-to-liver ratio was calculated by dividing heart transduction measurement by liver infection measurement of each variant. All the values are shown as fold-change relative to AAV9. Variants with improved heart transduction are shown as open circles. Variants with improved heart-to-liver ratio (“liver detargetting”) are shown as open triangles. Variants with both improved heart transduction and improved heart-to-liver ratio are shown as filled circles.

FIG. 5B shows the subset of variants from the sub-library no. 1 in Table 4 with both randomized VR-IV (amino acids 452 to 458 of AAV9 VP1) and substituted VR-VIII (amino acids 586-589 of AAV9 VP1). The measurements are the same as those in FIG. 5A.

FIG. 5C shows novel variants with modified VR-VIII (amino acids 581 to 594 on AAV9 VP1). The measurements are the same as those in FIG. 5A.

### Re-Testing in Mouse Model

Selected AAV variants were tested in mice to confirm performance relative to AAV9.

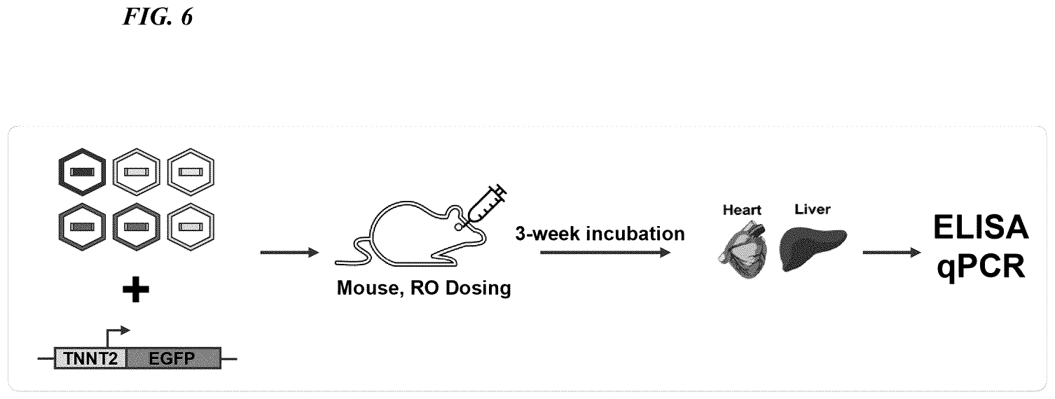

As shown in FIG. 6, five selected capsid sequence, ZC377, ZC399, ZC407, ZC425, and ZC469, and a reference AAV9 capsid sequence were used to produce AAV virions having a vector genome encoding EGFP driven by TNNT2 promoter. The resulting AAV virions were each injected into 6-week-old mice at 6E+12 viral genomes per kilogram body weight through retro-orbital administration, one capsid per animal. At three weeks, animals were sacrificed, and heart and liver were harvested. Heart transduction was measured by ELISA detecting EGFP protein in heart lysates and liver viral load was measure by qPCR targeting EGFP coding sequence in DNA recovered from liver samples.

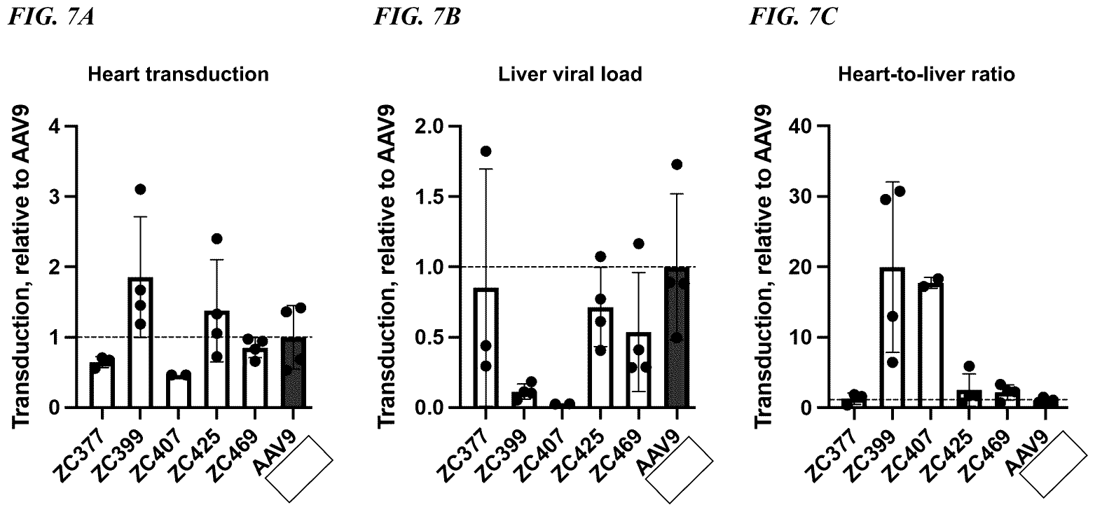

FIGS. 7A-7C show heart transduction (FIG. 7A), liver viral load (FIG. 7B), and heart-to-liver ratio (FIG. 7C) measurements of the selected variants and AAV9 reference. All the numbers are shown as fold-change relative to AAV9 control. Each dot represent data from one animal. ZC399 shows ˜1.8-fold average heart transduction compared to AAV9. ZC399 and ZC407 show lower liver viral load and ˜20-fold heart-to-liver ratio compared to AAV9.

### Analysis of Sequences of Selected Capsid Variants

The sequences of the selected variants are provided in Table 5 and Table 6.

TABLE 5
VR-IV variants

| | VP1 | | VR-IV | | VR-VIII |
|---|---|---|---|---|---|
| Identifier | SEQ | VR-IV | SEQ | VR-VIII | SEQ |
| No. | ID NO: | (452-458) | ID NO: | (581-594) | ID NO: |
| ZC377 | 488 | KGSGQNQ | 590 | ATNHQANYG | 598 |
| | | | | QAQTG | |
| ZC378 | 489 | NASGQNQ | 591 | ATNHQANYG | 598 |
| | | | | QAQTG | |
| ZC379 | 490 | NGTGQNQ | 592 | ATNHQANYG | 598 |
| | | | | QAQTG | |
| ZC380 | 491 | NGSGLNQ | 593 | ATNHQANYG | 598 |
| | | | | QAQTG | |
| ZC381 | 492 | ANDNKLI | 594 | ATNHQANYG | 598 |
| | | | | QAQTG | |
| ZC382 | 493 | VNDNKVI | 595 | ATNHQANYG | 598 |
| | | | | QAQTG | |
| ZC383 | 494 | NGSGQNH | 596 | ATNHQANYG | 598 |
| | | | | QAQTG | |
| ZC384 | 495 | ANDNKVI | 597 | ATNHQANYG | 598 |
| | | | | QAQTG | |

TABLE 6
VI-VIII variants

| | VP1 | | |
|---|---|---|---|
| Identifier | SEQ | | VR-VIII |
| No. | ID NO: | VR-VIII (581-594) | SEQ ID NO: |
| ZC385 | 496 | ATNHTSFQAQAQTG | 599 |
| ZC386 | 497 | ATNHCSAQAQAQTG | 600 |
| ZC387 | 498 | ATNHVDSLRIAQTG | 601 |
| ZC388 | 499 | ATNRQTAQAQAQTG | 602 |
| ZC389 | 500 | ATNHTGTSIIAQTG | 603 |
| ZC390 | 501 | ATNHLSNENSAQTG | 604 |
| ZC391 | 502 | ATNHCTLNSIAQTG | 605 |
| ZC392 | 503 | ADVQQKPGSQIQTQ | 606 |
| ZC393 | 504 | ATNHNMNRVNAQTG | 607 |
| ZC394 | 505 | ATNHNNVISGAQTG | 608 |
| ZC395 | 506 | ATNHSNSVQSAQTG | 609 |
| ZC396 | 507 | ATNHQSPIAQAQAQTG | 610 |
| ZC397 | 508 | ATNHLSKVEDAQTG | 611 |
| ZC398 | 509 | ATNHQSAITQAQAQTG | 612 |
| ZC399 | 510 | ATNHSSTFQGAQTG | 613 |
| ZC400 | 511 | ATNHNSIQAQAQTG | 614 |
| ZC401 | 512 | ATNHMMTTARAQTG | 615 |
| ZC402 | 513 | ATNHQGAYAQAQTG | 616 |
| ZC403 | 514 | ALNKQSAQAQAQTG | 617 |
| ZC404 | 515 | ATNHENTVSIAQTG | 618 |
| ZC405 | 516 | ATNHVSSFTSAQTG | 619 |
| ZC406 | 517 | ATNHPSIHQGAQTG | 620 |
| ZC407 | 518 | ATNHSTINFRAQTG | 621 |
| ZC408 | 519 | ATNHQHYSAQAQAQTG | 622 |
| ZC409 | 520 | ATNKQTAQAQAQTG | 623 |
| ZC410 | 521 | ATNHSSIFNSAQTG | 624 |
| ZC411 | 522 | ATNHAGNYNNAQTG | 625 |
| ZC412 | 523 | AEVQQSSMSQAQTD | 626 |
| ZC413 | 524 | AANVQSAQAQAQTG | 627 |
| ZC414 | 525 | ATNYQQAQAQAQTG | 628 |
| ZC415 | 526 | ATNHQSVQGAQAQTG | 629 |
| ZC416 | 527 | ATNHGSILTHAQTG | 630 |
| ZC417 | 528 | ATNHQLFSKNAQTG | 631 |
| ZC418 | 529 | AANMQSAQAQAQTG | 632 |
| ZC419 | 530 | ATNQQIAQAQAQTG | 633 |
| ZC420 | 531 | ATNTYHQSAQAQAQTG | 634 |
| ZC421 | 532 | ATNHCDPLHIAQTG | 635 |
| ZC422 | 533 | ATNHTSVISIAQTG | 636 |
| ZC423 | 534 | ATNHQLASAQAQTG | 637 |
| ZC424 | 535 | ATNHQVTSAQAQAQTG | 638 |
| ZC425 | 536 | ATNHHSRVEIAQTG | 639 |
| ZC426 | 537 | ATNHTSFTWTAQTG | 640 |
| ZC427 | 538 | ATNHQSAPTQAQAQTG | 641 |
| ZC428 | 539 | ATNHNSTYLGAQTG | 642 |
| ZC429 | 540 | ATNHQIAQAQAQTG | 643 |
| ZC430 | 541 | ATNHQAISAQAQAQTG | 644 |
| ZC431 | 542 | ATNHLSVVYNAQTG | 645 |
| ZC432 | 543 | ATNHMHQSAQAQAQTG | 646 |
| ZC433 | 544 | ATNHETSRLNAQTG | 647 |
| ZC434 | 545 | AFNWQSAQAQAQTG | 648 |
| ZC435 | 546 | ATNHNTVMLGAQTG | 649 |
| ZC436 | 547 | ATNHESSMLNAQTG | 650 |
| ZC437 | 548 | ATNHASITSSAQTG | 651 |
| ZC438 | 549 | ARNEQSAQAQAQTG | 652 |
| ZC439 | 550 | ATNHANLYQMAQTG | 653 |
| ZC440 | 551 | ATNHQFATAQAQTG | 654 |
| ZC441 | 552 | ATNFNHQSAQAQAQTG | 655 |
| ZC442 | 553 | ATNHMSHQAQAQTG | 656 |
| ZC443 | 554 | ATNHQWMSAQAQAQTG | 657 |
| ZC444 | 555 | ATNHQSGQQAQAQTG | 658 |
| ZC445 | 556 | ATNHSSAQAQAQTG | 659 |
| ZC446 | 557 | ATNHTTKTMFAQTG | 660 |
| ZC447 | 558 | ATNHSSIIYSAQTG | 661 |
| ZC448 | 559 | ATNHMLLKSNAQTG | 662 |
| ZC449 | 560 | ATNHESMQAQAQTG | 663 |
| ZC450 | 561 | ATNHQMLSAQAQAQTG | 664 |
| ZC451 | 562 | ATNHSGRDSYAQTG | 665 |
| ZC452 | 563 | ATNHINVISGAQTG | 666 |
| ZC453 | 564 | ATNHVSNQAQAQTG | 667 |
| ZC454 | 565 | ATNHNTKLAIAQTG | 668 |
| ZC455 | 566 | ATNHSSSYNNAQTG | 669 |
| ZC456 | 567 | ATNATHQSAQAQAQTG | 670 |
| ZC457 | 568 | ATNHLRDNISAQTG | 671 |
| ZC458 | 569 | ATNHSSFSVGAQTG | 672 |
| ZC459 | 570 | ATNHVNRNLSAQTG | 673 |
| ZC460 | 571 | ATNHHNPSINAQTG | 674 |
| ZC461 | 572 | ATNHQDARAQAQTG | 675 |
| ZC462 | 573 | ATNDQRAQAQAQTG | 676 |
| ZC463 | 574 | ATNVQTAQAQAQTG | 677 |
| ZC464 | 575 | APNRQSAQAQAQTG | 678 |
| ZC465 | 576 | ATNRQIAQAQAQTG | 679 |
| ZC466 | 577 | ATNHEDNIRRAQTG | 680 |
| ZC467 | 578 | ATNHNRNGLLAQTG | 681 |
| ZC468 | 579 | ATNHESTSVRAQTG | 682 |
| ZC469 | 580 | ATNHNIRTEMAQTG | 683 |
| ZC470 | 581 | ATNHQTLENSAQTG | 684 |
| ZC471 | 582 | ATNHHSWQAQAQTG | 685 |
| ZC472 | 583 | ATNHSTKSLIAQTG | 686 |
| ZC473 | 584 | ATNHQKLLVNAQTG | 687 |
| ZC474 | 585 | ATNHLSVSSIAQTG | 688 |
| ZC475 | 586 | ATNHVSNLYGAQTG | 689 |
| ZC476 | 587 | ATNRQMAQAQAQTG | 690 |
| ZC477 | 588 | ATNHEDIIRSAQTG | 691 |
| ZC478 | 589 | ATNHCSTSIRAQTG | 692 |

Variants having insertions in the VR-VIII site were manually aligned to show the insertions. Results are shown in Table 7.

TABLE 7

| Identifier | | | Insertion position | |
|---|---|---|---|---|
| No. | Alignment | SEQ ID NO: | (after) | Insertion |
| ZC396 | N--H--Q--SPIA--Q | 693 | 586 | PI |
| ZC398 | N--H--Q--S--AITQ | 694 | 587 | IT |
| ZC408 | N--H--QHYS--A--Q | 695 | 585 | HY |
| ZC420 | NTYH--Q--S--A--Q | 696 | 583 | TY |
| ZC424 | N--H--QVTS--A--Q | 697 | 585 | VT |
| ZC427 | N--H--Q--S--APTQ | 698 | 587 | PT |
| ZC430 | N--H--QAIS--A--Q | 699 | 585 | AI |
| ZC432 | N--HMHQ--S--A--Q | 700 | 584 | MH |
| ZC441 | NFNH--Q--S--A--Q | 701 | 583 | FN |
| ZC443 | N--H--QWMS--A--Q | 702 | 585 | WM |
| ZC450 | N--H--QMLS--A--Q | 703 | 585 | ML |
| ZC456 | NATH--Q--S--A--Q | 704 | 583 | AT |

Selected variant sequences are further tested as described below.

### Example 2: Confirmatory Re-Screening in Primates

Selected AAV variants are tested in primates to confirm performance relative to AAV9. To perform the experiment with further animals, a pool-injection-and-screen strategy analogous to the original screening protocol is used.

All the 102 novel capsids are packaged individually using transgene cassette carrying barcoded EGFP reporter driven by TNNT2 promoter. The resulting AAV virions are pooled and injected into Cynomolgus monkeys through intravenous administration. At 4-week post-injection, animals are sacrificed and biopsies taken from heart and liver, as well as other tissues. RNA is isolated from heart samples. The barcoded region on the viral transcript is amplified and sequenced by Next-Generation Sequencing. The heart transduction ability of each variant is quantified with normalized read counts of corresponding barcodes. DNA is isolated from liver samples. The barcoded region on the viral transgene is processed in the same way as the heart RNA samples. The liver tropism of each variant is quantified with normalized read counts of corresponding barcodes.

A similar study in mouse is performed in parallel.

### Example 3: Neutralizing Antibody Prevalence Study

Selected capsids are assayed against pooled human IgG samples, as well as individual human serum samples. The ability to escape from neutralization by pooled IgG and increased percentage of sero-negative human individuals indicate potential wider patient coverage than AAV9.

### Example 4: Biodistribution Study in Primates

Selected capsid and AAV9 control are tested in a biodistribution study in Cynomolgus monkeys. Each animal receives one test article and each test article is tested on three animals. The transduction and viral genome distribution is examined in various organs and tissues.

### Example 5: Novel AAV Capsids with Improved Transduction Properties

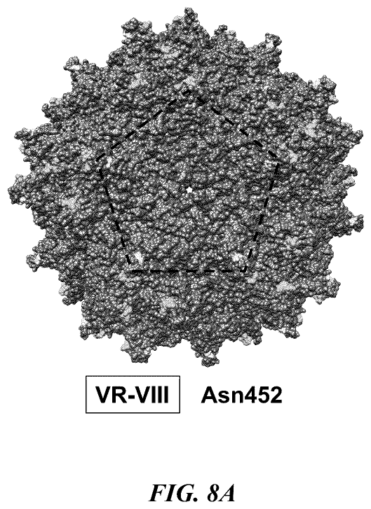

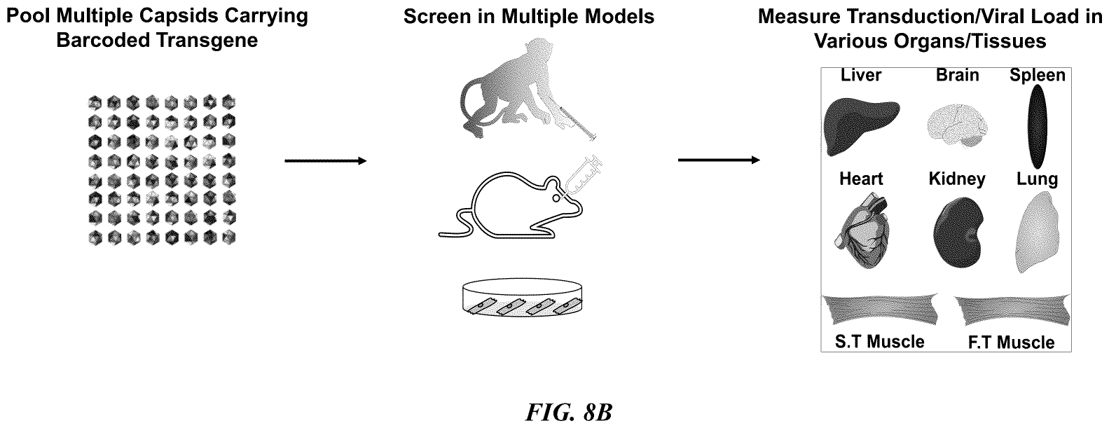

The purpose of this study was to identify novel AAV capsid variants that have superior properties, such as improved transduction. Capsids with modified VR-VIII (amino acids 585 to 590, AAV9 VP1 numbering) were further modified at amino acid 452 (AAV9 VP1 numbering, Asparagine/Asn/N452) (FIG. 8A; Mutations in Tables 8 and 9). The resulting capsid variants were packaged individually using barcoded transgene cassettes, pooled together, and tested in non-human primates (cynomolgus monkey/Macaca fascicularis/Cyno), CD-1 mice, and human iPSC-derived cardiomyocytes (iPSC-CMs). The doses provided herein represent the total amount of virus in the pool. Cynomolgus monkeys and pigs were administered virus at 1E+13 vg/kg via intravenous bolus administration and tissue was collected 4-weeks after injection. Two groups of mice were administered virus, three mice at 1E+13 vg/kg and three mice at 5E+13 vg/kg via retro-orbital administration and tissue was collected 18-days post injection. For the iPSC-CMs, two populations of cells were administered virus at different doses, 1.6E+4 vg/cell and 1.6E+5 vg/cell and samples were collected 4 days later. The transduction/viral load levels of capsids variants in different organs (such as heart, liver, brain, skeletal muscle) and iPSC-CMs were measured by quantifying the barcodes by next-generation sequencing (NGS) (FIG. 8B). The average of the two dose level groups for the mice and iPSCs is shown.

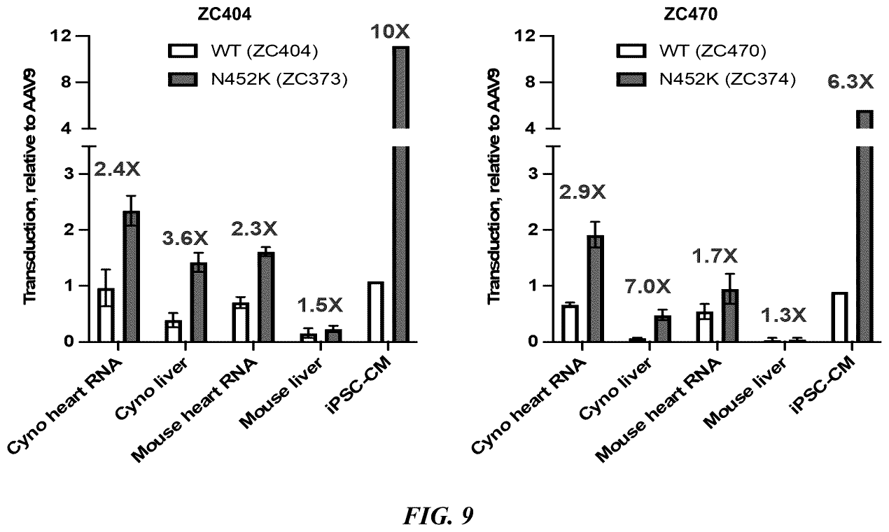

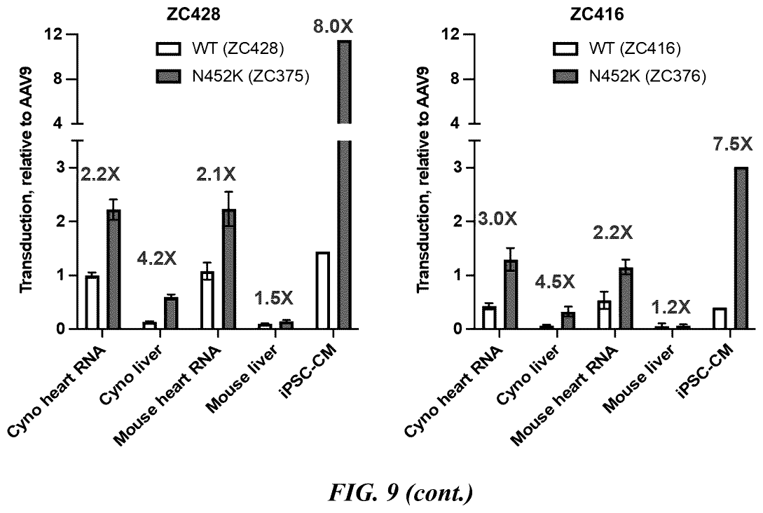

Experiments with some of the identified mutated capsids from the screen are shown in FIG. 9. In particular, ZC404 (SEQ ID NO: 618), ZC470 (SEQ ID NO: 684), ZC428 (SEQ ID NO: 642), are ZC416 (SEQ ID NO: 630) are variants with VR-VIII modifications. Their transduction and viral load levels in Cyno heart, Cyno liver, mouse heart, mouse liver, and human iPSC-CMs are shown as white bars in FIG. 9. Introduction of the N452K mutation into these capsids (Tables 8 and 9) resulted in four combinatory capsids, ZC373 (SEQ ID NO: 705), ZC374 (SEQ ID NO: 706), ZC375 (SEQ ID NO: 707), and ZC376 (SEQ ID NO: 708), and their transduction/viral load levels in Cyno heart, Cyno liver, mouse heart, mouse liver, and human iPSC-CMs was measured, with the results shown in FIG. 9 as dark/shaded bars. All these combinatory capsids showed improved transduction/viral load (particularly in the heart) compared to the original VR-VIII variants, showing that N452K can enhance the transduction of AAV9-based capsids regardless of what modifications have been made in other regions of the capsid.

A capsid (ZC537) with N452K mutation as the only modification was also generated. In addition, capsids ZC531, ZC532, ZC533, ZC534, ZC535, ZC536, ZC538, ZC539, ZC540, ZC541, ZC542 with N452K mutation in addition to other mutations in VR-VIII were generated.

TABLE 8
Novel VP1 Capsids

| Capsid | | VR-VIII | VR-VIII | VP1 Capsid |
|---|---|---|---|---|
| Identifier No. | Position 452 | Alignment (581-594) | SEQ ID NO: | SEQ ID NO: |
| ZC373 | K | ATNHENTVSIAQTG | 618 | 705 |
| ZC374 | K | ATNHQTLFNSAQTG | 684 | 706 |
| ZC375 | K | ATNHNSTYLGAQTG | 642 | 707 |
| ZC376 | K | ATNHGSILTHAQTG | 630 | 708 |
| ZC404 | N | ATNHENTVSIAQTG | 618 | 515 |
| ZC470 | N | ATNHQTLFNSAQTG | 684 | 581 |
| ZC428 | N | ATNHNSTYLGAQTG | 642 | 539 |
| ZC416 | N | ATNHGSILTHAQTG | 630 | 527 |
| ZC531 | K | ATNHMMTTARAQTG | 615 | 767 |
| ZC532 | K | ATNHCSTSIRAQTG | 692 | 768 |
| ZC533 | K | ATNHQGAYAQAQTG | 616 | 769 |
| ZC534 | K | ATNHNTKLAIAQTG | 668 | 770 |
| ZC535 | K | ATNHVSSFTSAQTG | 619 | 771 |
| ZC536 | K | ATNHEDNIRSAQTG | 726 | 772 |
| ZC537 | K | ATNHQSAQAQAQTG | 5 | 773 |
| ZC538 | K | ATNHNNVISGAQTG | 608 | 774 |
| ZC539 | K | ATNHTGTSIIAQTG | 603 | 775 |
| ZC540 | K | ATNHQWMSAQAQAQTG | 657 | 776 |
| ZC541 | K | ATNHQDARAQAQTG | 675 | 777 |
| ZC542 | K | ATNHQHYSAQAQAQTG | 622 | 778 |

TABLE 9
VR-VIII and N452 Substitutions in Certain Novel VP1 Capsids*

| Capsid | | | | | | | |
|---|---|---|---|---|---|---|---|
| Identifier | Position | Position | Position | Position | Position | Position | Position |
| No. | 585 | 586 | 587 | 588 | 589 | 590 | 452 |
| ZC373 | Q585E | S586N | A587T | Q588V | A589S | Q590I | N452K |
| ZC374 | | S586T | A587L | Q588F | A589N | Q590S | N452K |
| ZC375 | Q585N | | A587T | Q588Y | A589L | Q590G | N452K |
| ZC376 | Q585G | | A587I | Q588L | A589T | Q590H | N452K |
| ZC404 | Q585E | S586N | A587T | Q588V | A589S | Q590I | |
| ZC470 | | S586T | A587L | Q588F | A589N | Q590S | |
| ZC428 | Q585N | | A587T | Q588Y | A589L | Q590G | |
| ZC416 | Q585G | | A587I | Q588L | A589T | Q590H | |
| ZC531 | Q585M | S586M | A587T | Q588T | | Q590R | N452K |
| ZC532 | Q585C | | A587T | Q588S | A589I | Q590R | N452K |
| ZC533 | | S586G | | Q588Y | | | N452K |
| ZC534 | Q585N | S586T | A587K | Q588L | | Q590I | N452K |
| ZC535 | Q585V | | A587S | Q588F | A589T | Q590S | N452K |
| ZC536 | Q585E | S586D | A587N | Q588I | A589R | Q590S | N452K |
| ZC537 | | | | | | | N452K |
| ZC538 | Q585N | S586N | A587V | Q588I | A589S | Q590G | N452K |
| ZC539 | Q585T | S586G | A587T | Q588S | A589I | Q590I | N452K |
| ZC541 | | S586D | | Q588R | | | N452K |
| ZC369 | Q585N | S586I | A587R | Q588T | A589E | Q590M | N452K |
| ZC370 | Q585S | S586T | A587T | Q588N | A589F | Q590R | N452K |
| AAV9 | | | | | | | |

*No entry in the table = no substitution
Note:
all of the capsids in Table 9 have (i) ATNH at positions 581, 582, 583 and 584, respectively, and (ii) AQTG at positions 591, 592, 593 and 594, respectively.

Accordingly, the identified capsids comprise the indicated amino acids at indicated positions of VR-VIII (where the only or last amino acid corresponds to the unmodified AAV9 amino acid):

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, | N, | T, | V, | S, | I, | A | Q | T | G |
| | | | | N, | T, | L, | F, | N, | S, | | | | |
| | | | | G, | M, | I, | Y, | L, | G, | | | | |
| | | | | M, | G, | K, | L, | T, | H, | | | | |
| | | | | C, | D, | S, | T, | I, | R, | | | | |
| | | | | V, | S | N, | S, | R, | Q | | | | |
| | | | | T, | | V, | I, | A | | | | | |
| | | | | Q | | A | R, | | | | | | |
| | | | | | | | Q | | | | | | |

TABLE 11
VR-VIII Insertions in Certain Novel VP1 Capsids

| Capsid | | | | |
|---|---|---|---|---|
| Identifier | | VR-VIII | VR-VIII | |
| No. | Position 452 | Alignment (581-594) | SEQ ID NO: | Comments |
| ZC540 | K | ATNHQWMSAQAQAQTG | 657 | Insertion of |
| | | | | WM before |
| | | | | position 586 |
| | | | | (between |
| | | | | positions 585 |
| | | | | and 586 of |
| | | | | SEQ ID NO: 1) |
| ZC542 | K | ATNHQHYSAQAQAQTG | 622 | Insertion of |
| | | | | HY before |
| | | | | position 586 |
| | | | | (between |
| | | | | positions 585 |
| | | | | and 586 of |
| | | | | SEQ ID NO: 1) |
| AAV9 | N | ATNHQ SAQAQAQTG | 5 | |

### Example 6: Characterizing Novel AAV Capsids in Multiple Mammalian Models

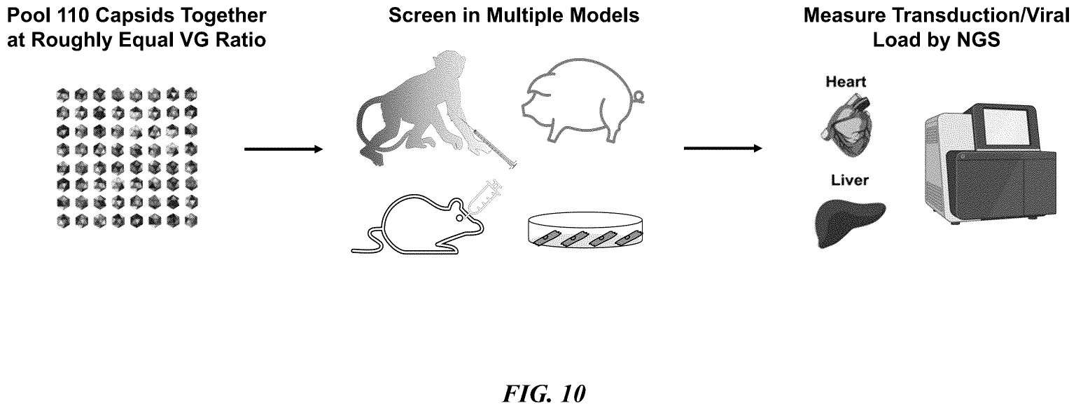

The purpose of this study was to compare the performance of the novel AAV capsids described above in multiple models including non-human primate, mouse, pig, and in vitro human iPSC-CMs. The novel AAV capsids and control capsids were packaged individually and barcoded transgene cassettes were used to enable pooled next-generation sequencing based capsid transduction assays (FIG. 10). The viruses were pooled together targeting equal viral genome ratio and the pool was tested in vivo in Cynomolgus Monkey, CD-1 mice, and pig, as well as in vitro on human iPSC-derived cardiomyocytes. Animals and cells were administered virus, and tissue was collected, as described in Example 5. To measure transduction efficiency and/or viral load, heart tissue, liver tissue, and iPSC-CMs were collected, followed by RNA and DNA extraction. The barcoded region was amplified from RNA and DNA samples and sequenced by next-generation sequencing. The RNA or DNA raw read count of each barcode was normalized to the total read number in the sequencing run and to the abundance in the initial virus pool. The average measurement of multiple barcodes belonging to the same capsid was calculated to determine the transduction efficiency or viral load of the capsid.

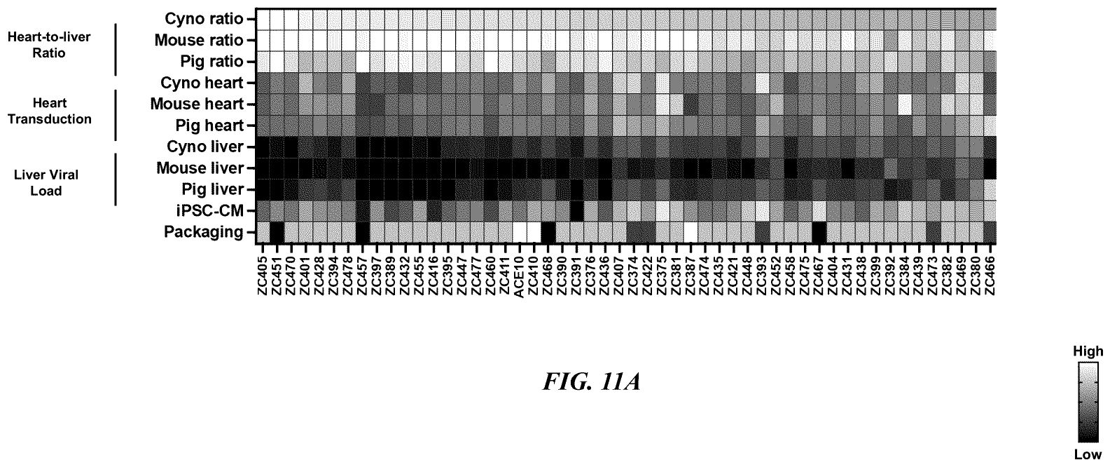

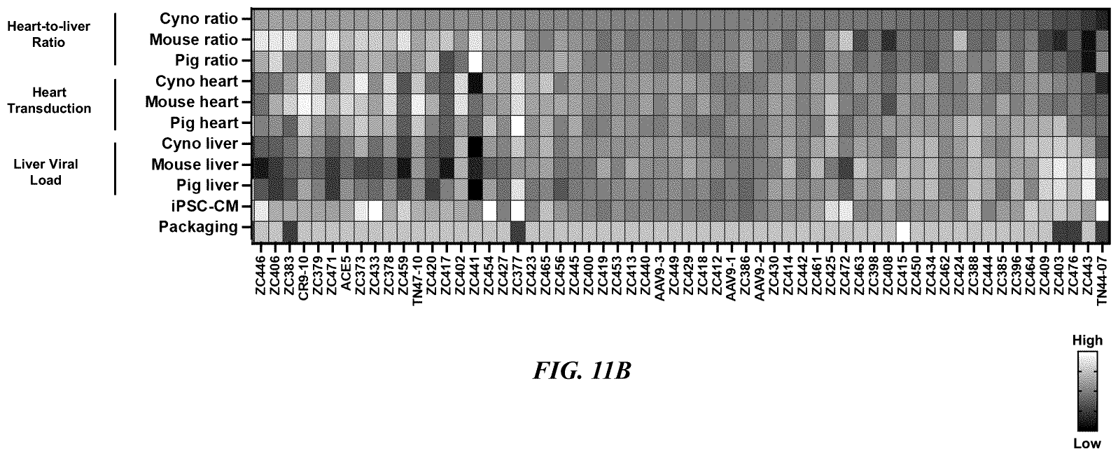

Heart transduction was measured with RNA signal in the heart tissue. Liver viral load was measured with DNA signal in the liver tissue. Heart-to-liver ratio was determined by dividing heart transduction by liver viral load. Transduction efficiency on iPSC-CMs was determined by RNA signal. Packaging scores were determined by virus yield in HEK293T production system. The average measurements of 4 animals, 3 animals, 6 animals, or 2 multiplicities of infection were shown for Cynomolgus monkey, mouse, pig, and iPSC-CMs, respectively (FIGS. 11A and 11B). FIGS. 11A and 11B represent the whole dataset heatmap. Each column on the heatmap represents one capsid and each row represents one sample type. White color means higher value and dark color means lower value, with the median grayscale representative of wildtype AAV9 control. The capsids are ranked from left to right by their heart-to-liver ratios in Cynomolgus monkey. AAV9-1, AAV9-2, and AAV9-3 are all wildtype AAV9 capsids that served as control replicates. CR9-10, TN47-10 and TN44-07 are as disclosed in WO 2021/216456 A2 (by reference to the same capsid name), the disclosures of which are specifically incorporated by reference herein. The sequences of other capsids are disclosed herein.

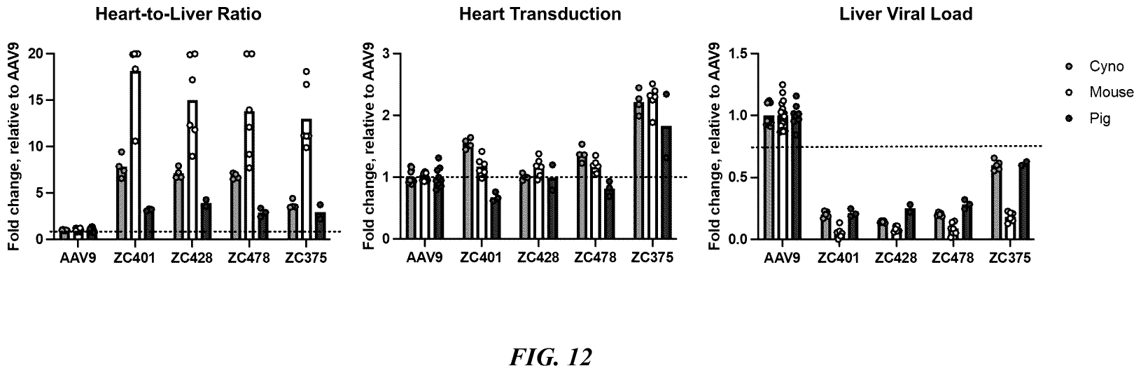

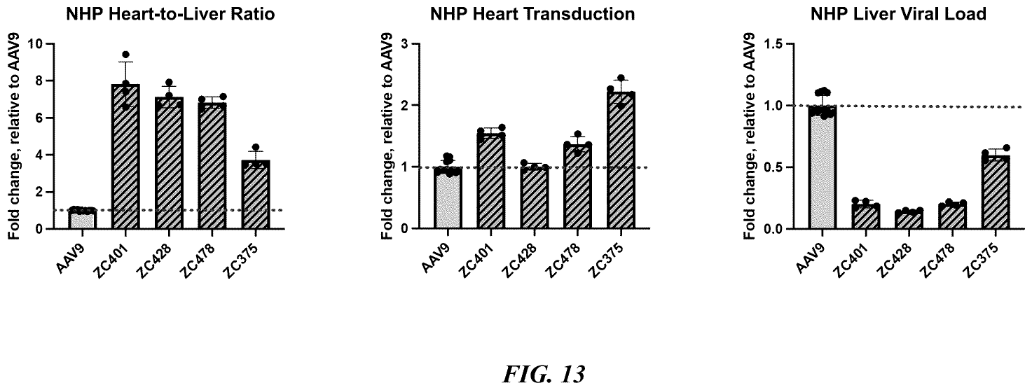

Exemplary novel capsids were selected from the screen in FIGS. 11A and 11B (Table 10) to evaluate transduction efficiency across different species. The heart-to-liver ratio, heart transduction, and liver viral load measurements of four novel capsids and AAV9 control in Cynomolgus monkey (light gray bars), mouse (white bars), and pig (dark bars) were evaluated relative to the performance of wildtype AAV9 control (FIG. 12). Animals were administered virus, and tissue was collected, as described above. These ratios are shown specifically for non-human primates (NHP) in FIG. 13 (Cynomolgus monkey). The novel capsids showed improved heart-to-liver ratio in all three species, demonstrating species consistency. Furthermore, in NHP, the novel capsids showed improved heart-to-liver ratio, at least comparable heart transduction, and less liver viral load, relative to AAV9.

TABLE 10
Capsids Used in the Study

| | VP1 Capsid |
|---|---|
| Identifier No. | SEQ ID NO |
| ZC373 | 705 |
| ZC374 | 706 |
| ZC375 | 707 |
| ZC376 | 708 |
| ACE5 | 709 |
| ACE10 | 710 |
| AAV9-1/2/3 | 1 |
| CR9-10 | 404 |
| TN44-07 | 457 |
| TN47-10 | 458 |
| ZC377 | 488 |
| ZC378 | 489 |
| ZC379 | 490 |
| ZC380 | 491 |
| ZC381 | 492 |
| ZC382 | 493 |
| ZC383 | 494 |
| ZC384 | 495 |
| ZC385 | 496 |
| ZC386 | 497 |
| ZC387 | 498 |
| ZC388 | 499 |
| ZC389 | 500 |
| ZC390 | 501 |
| ZC391 | 502 |
| ZC392 | 503 |
| ZC393 | 504 |
| ZC394 | 505 |
| ZC395 | 506 |
| ZC396 | 507 |
| ZC397 | 508 |
| ZC398 | 509 |
| ZC399 | 510 |
| ZC400 | 511 |
| ZC401 | 512 |
| ZC402 | 513 |
| ZC403 | 514 |
| ZC404 | 515 |
| ZC405 | 516 |
| ZC406 | 517 |
| ZC407 | 518 |
| ZC408 | 519 |
| ZC409 | 520 |
| ZC410 | 521 |
| ZC411 | 522 |
| ZC412 | 523 |
| ZC413 | 524 |
| ZC414 | 525 |
| ZC415 | 526 |
| ZC416 | 527 |
| ZC417 | 528 |
| ZC418 | 529 |
| ZC419 | 530 |
| ZC420 | 531 |
| ZC421 | 532 |
| ZC422 | 533 |
| ZC423 | 534 |
| ZC424 | 535 |
| ZC425 | 536 |
| ZC427 | 538 |
| ZC428 | 539 |
| ZC429 | 540 |
| ZC431 | 542 |
| ZC432 | 543 |
| ZC433 | 544 |
| ZC434 | 545 |
| ZC435 | 546 |
| ZC436 | 547 |
| ZC438 | 549 |
| ZC439 | 550 |
| ZC440 | 551 |
| ZC441 | 552 |
| ZC442 | 553 |
| ZC443 | 554 |
| ZC444 | 555 |
| ZC445 | 556 |
| ZC446 | 557 |
| ZC447 | 558 |
| ZC448 | 559 |
| ZC449 | 560 |
| ZC450 | 561 |
| ZC451 | 562 |
| ZC452 | 563 |
| ZC453 | 564 |
| ZC454 | 565 |
| ZC455 | 566 |
| ZC456 | 567 |
| ZC457 | 568 |
| ZC458 | 569 |
| ZC459 | 570 |
| ZC460 | 571 |
| ZC461 | 572 |
| ZC462 | 573 |
| ZC463 | 574 |
| ZC464 | 575 |
| ZC465 | 576 |
| ZC466 | 577 |
| ZC467 | 578 |
| ZC468 | 579 |
| ZC469 | 580 |
| ZC470 | 581 |
| ZC471 | 582 |
| ZC472 | 583 |
| ZC473 | 584 |
| ZC474 | 585 |
| ZC475 | 586 |
| ZC476 | 587 |
| ZC477 | 588 |
| ZC478 | 589 |

### Example 7: Top Novel Capsids Show Superior Performance when Administered Individually

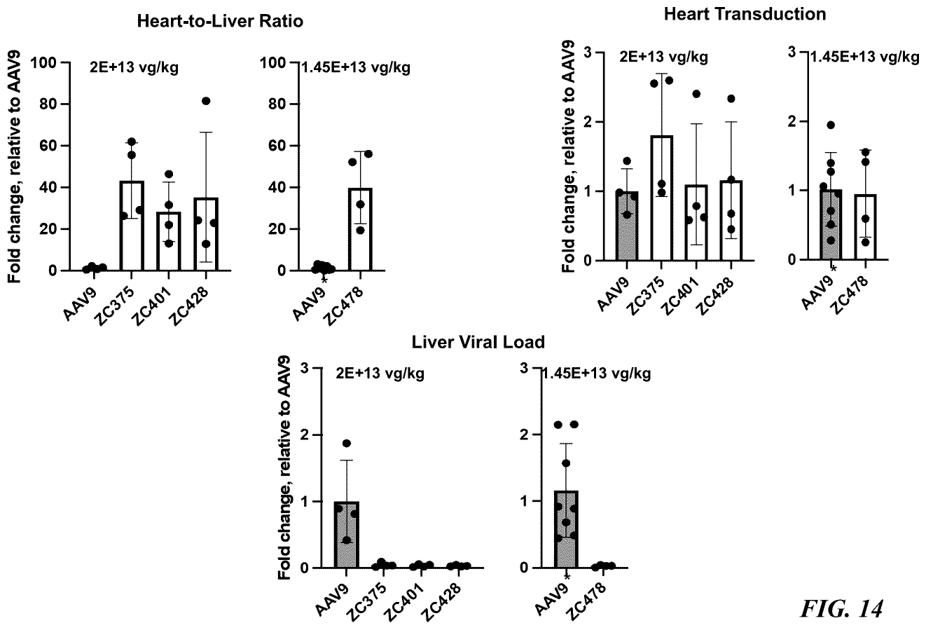

To study whether the pooled capsid comparison results can predict performance in individual animal injections (one test article per animal), the top four novel capsids and AAV9 were tested in CD-1 mice using retro-orbital injection. The viruses were administered at 2E+13 vg/kg for ZC375, ZC401, and ZC428, and 1.45E+13 vg/kg for ZC478. Dosage matched AAV9 controls were included. Animals were sacrificed at day 18 post injection. Heart transduction was measured by RT-qPCR based quantification of transgene mRNA expression in the heart. Liver viral load was measured by qPCR-based quantification of transgene DNA copies in the liver. Heart-to-liver ratio was determined by dividing heart transduction by liver viral load. All four novel capsids showed improved heart-to-liver ratio in this individual test, consistent with pooled test results (FIG. 14).

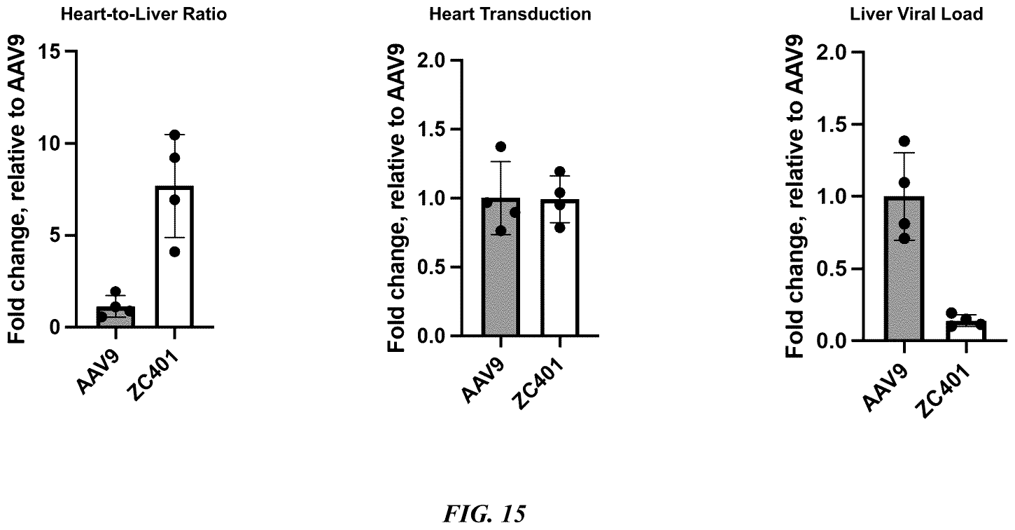

To test whether the superior performance of the novel capsids was CD-1 mouse strain specific, ZC401 and AAV9 were evaluated in a second mouse strain, C57BL/6NCrl from Charles River Laboratories. The viruses were administered at 2E+13 vg/kg by retro-orbital injection. Animals were sacrificed at day 18 post injection. Transduction was measured as described above. Novel capsid ZC401 demonstrated improved hear-to-liver ratio, consistent with CD-1 strain results (FIG. 15).

| | VR-VIII | VR-VIII | Capsid |
|---|---|---|---|
| Identifier | Alignment | SEQ | SEQ |
| No. | (581-594) | ID NO: | ID NO: |
| ZC375 | ATNHNSTYLGAQTG | 642 | 707 |
| ZC401 | ATNHMMTTARAQTG | 615 | 512 |
| ZC428 | ATNHNSTYLGAQTG | 642 | 539 |
| ZC478 | ATNHCSTSIRAQTG | 692 | 589 |

### Example 8: Novel Capsid ZC401 Achieved Increased Heart Transduction without Liver Overload

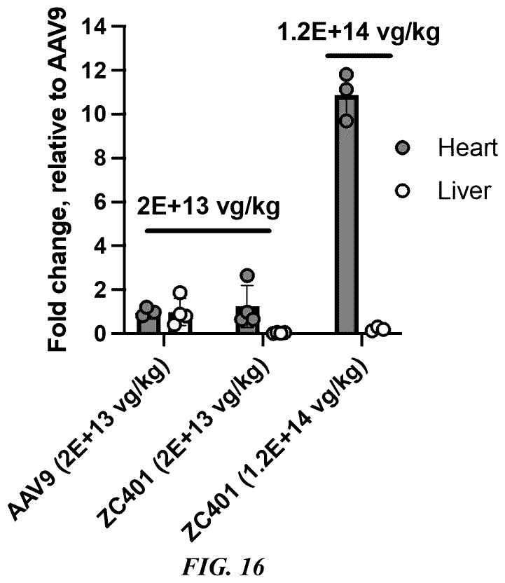

While Novel capsids with improved heart-to-liver ratio can reduce liver burden without compromising heart transduction, they can also enable higher safe dosage at which heart transduction is improved and liver viral load is still lower compared to AAV9 at regular dosage. To test the latter application, a proof-of-concept study was performed comparing ZC401 and AAV9 in CD-1 mice. The viruses were administered at 2E+13 vg/kg (AAV9 and ZC401) or 1.2E+14 vg/kg (ZC401) by retro-orbital injection. Animals were sacrificed at day 18 post injection. Heart transduction was measured by RT-qPCR based quantification of transgene mRNA expression in the heart. Liver viral load was measured by qPCR-based quantification of transgene DNA copies in the liver. Heart-to-liver ratio was determined by dividing heart transduction by liver viral load. Measured values are fold change relative to AAV9. Novel capsid ZC401 at 1.2E+14 vg/kg dose showed 8× heart transduction level compared to AAV9 at 2E+13 vg/kg, while having just 21% of its liver viral load (FIG. 16).

The data described herein characterized 102 capsids in NHPs, mice, pigs and human iPSC-derived cardiomyocytes (hiPSC-CMs) and identified multiple novel AAV capsids with superior properties including improved heart-to-liver ratio, improved cardiomyocyte transduction, and excellent consistency between different species. Together, these novel AAV capsids allow for more efficacious and safer gene therapies for cardiac disorders.

### Example 9: Characterizing AAV9 Capsids with N452K Substitution in Multiple Mammalian Models

To test the compatibility of N452K substitution with AAV9-based capsid variants and characterize how N452K affects transduction efficiency, 14 additional N452K-containing variants were generated and tested by comparing them to parental wildtype AAV9 or AAV9-based VR-VIII substitution variants. The purpose of this study was to compare the performance of AAV capsids with N452K substitution (some of which were described in Example 5 above, and further described in FIG. 17) with parental AAV9 capsids (including wild-type AAV9 and AAV9 capsids with VR-VIII substitutions) in multiple models including non-human primate, mouse, and in vitro human iPSC-CMs.

The capsids were administered and tested in vivo in Cynomolgus Monkey and C57BL/6NCrl mice, as well as in vitro on human iPSC-derived cardiomyocytes as described in Examples 5 and 6 except the following dosing was used: cynomolgus monkeys were dosed at 1.6E+13 vg/kg; mice were dosed at 3E+13 vg/kg, and iPSC-CMs were dosed at 10E+4 vg/cell and 10E+5 vg/cell.

Transduction in heart or liver was measured as described in Example 6. The average measurements of 2 animals, 4 animals, or 2 multiplicities of infection are shown for Cynomolgus monkey, mouse, and iPSC-CMs, respectively. Routes of administration, type of tissue collected and tissue collection timing was the same as in Examples 5 and 6.

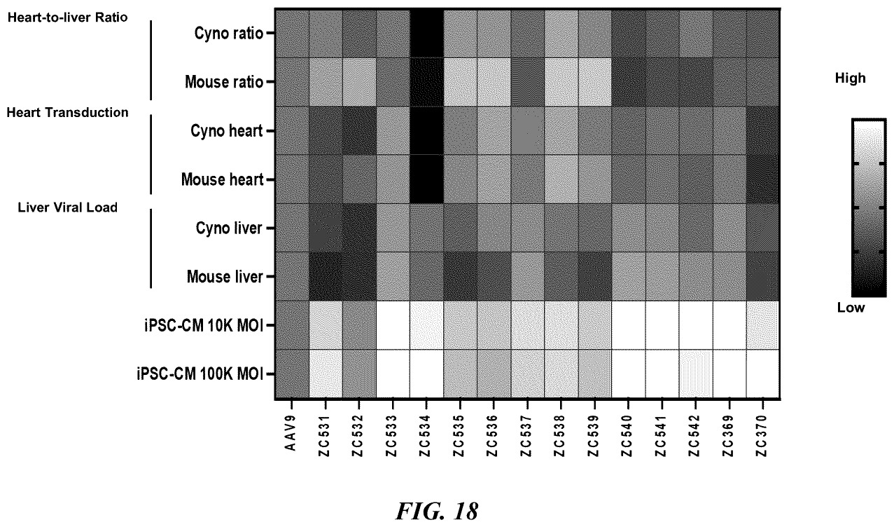

FIG. 18 represents heatmap data showing efficiency of transduction in various tissue samples. Each column on the heatmap represents one capsid and each row represents one sample type. Lighter color means higher value and darker color means lower value, with the median grayscale representative of wildtype AAV9 control. AAV9 is a wildtype capsid that served as a control. Together, this data demonstrates the viability and transduction efficiency of the new capsids in vitro and in vivo.

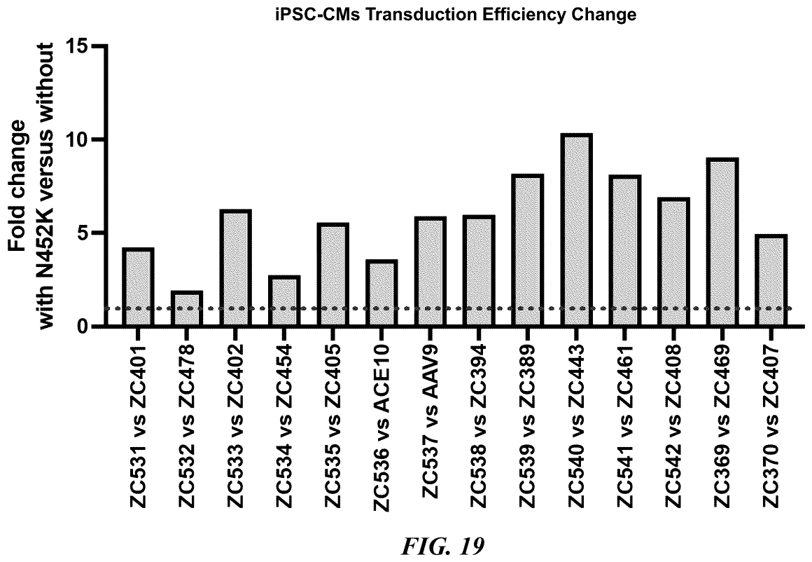

Next, transduction efficiency was evaluated in iPSC-CMs to compare transduction of controls without an N452K mutation and their counterpart capsids with an N452K mutation. FIG. 19 shows increased transduction in every capsid with an N452K mutation compared to the control showing overall improved transduction efficiency in cardiomyocytes.

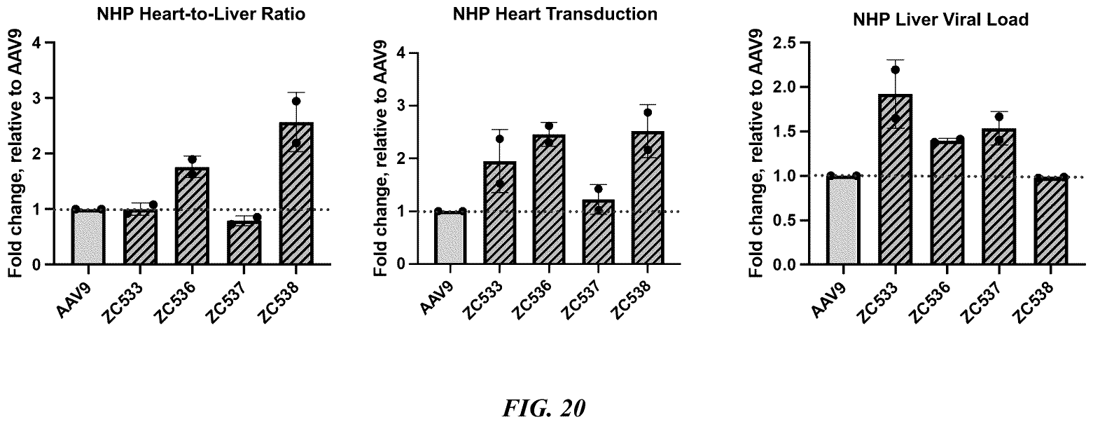

The heart-to-liver ratio, heart transduction, and liver viral load measurements of four newly generated capsids in Cynomolgus monkey were evaluated relative to the performance of wildtype AAV9 control (FIG. 20). Animals were administered virus, and tissue was collected, as described above. The ZC536 and ZC538 capsids showed improved heart-to-liver ratio and increased heart transduction was observed for each of the capsids relative to AAV9.

### Example 10: Biodistribution and Transduction of Newly Generated AAV9 Capsids in Non-Human Primates

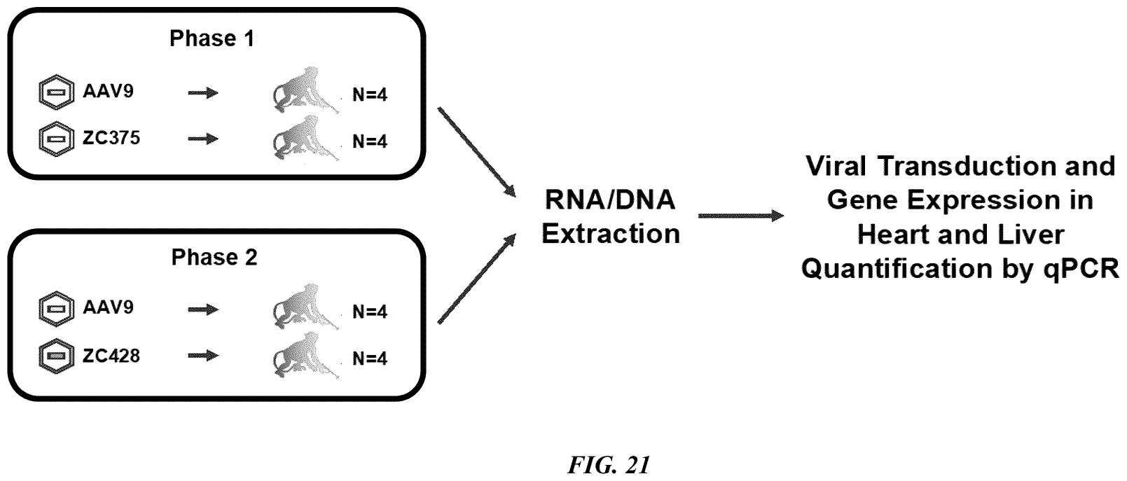

To characterize the performance of capsids described in Example 7 individually in NHPs (one test article per animal), NHPs were administered a single injection of AAV9, ZC375, or ZC428 at 6E+13 vg/kg systemically. The study was divided into two phases (according to the experimental design depicted in FIG. 21) and in each phase, one novel capsid and AAV9 control were tested with 4 Cynomolgus Monkeys per test article. Animals were sacrificed at 28-day post injection. RNA and DNA were extracted from heart and liver tissues, followed by RT-qPCR based quantification of viral transgene mRNA expression and qPCR-based quantification of viral DNA genome load.

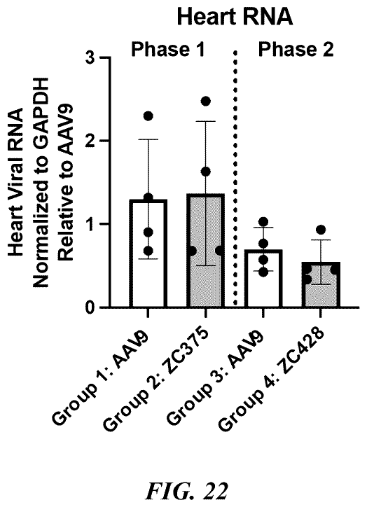

Viral transgene expression levels in the heart were measured by RT-qPCR analysis on RNA samples and normalized to the average of all AAV9 data points. Both ZC375 and ZC428 show comparable transgene expression in the heart compared to their matched AAV9 control (FIG. 22). Each measured sample represents one individual animal for which 4 heart biopsy samples were analyzed and averaged.

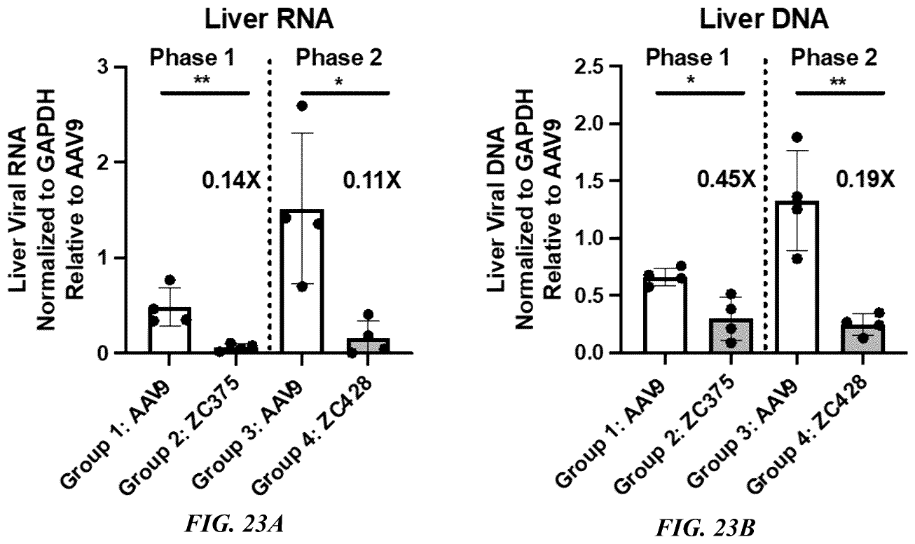

Viral transgene expression in the liver from the NHP were measured by RT-qPCR analysis on RNA samples and normalized to the average of all AAV9 data points. Viral genome load levels were measured by qPCR analysis on DNA samples and normalized to the average of all AAV9 data points. ZC375 and ZC428 show reduced transduction in the liver at both RNA and DNA levels compared to their matched AAV9 control (FIGS. 23A and 23B). Each measured sample represents one individual animal for which 2 liver biopsy samples were analyzed and averaged.

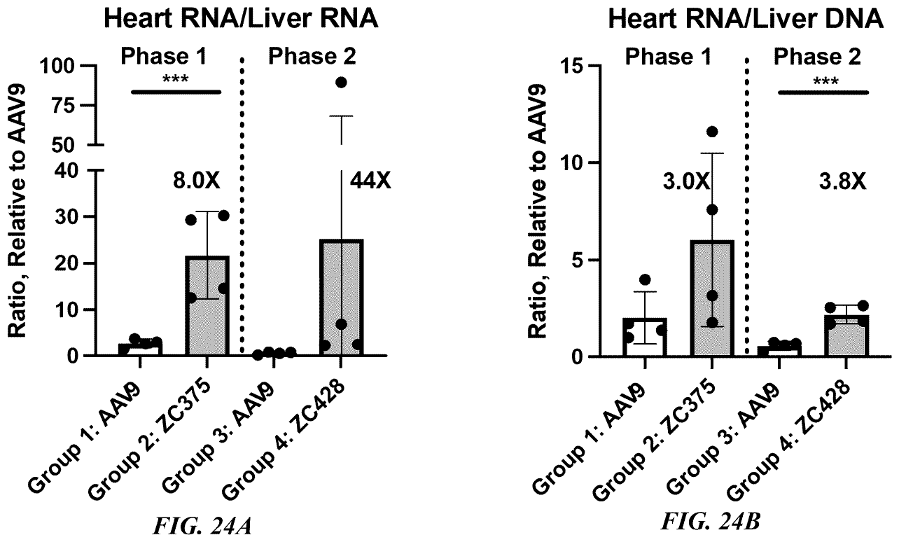

Comparison between heart transduction to liver transduction ratios from the NHP biodistribution and transduction study above was calculated. Using either heart RNA-based and liver RNA-based measurements (FIG. 24A), or heart RNA-based and liver DNA-based measurements (FIG. 24B) it was demonstrated that ZC375 and ZC428 had an improved heart-to-liver ratio compared to their matched AAV9 control. Together, this data demonstrates improved transduction efficiency and heart-to-liver ratio in NHP for both the ZC375 and ZC428 capsids compared to a wild-type AAV9.

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

What is claimed is:

1. A recombinant adeno-associated virus (rAAV) capsid protein, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion at position 584, or between positions 583 and 584, comprising one or more of an asparagine (N), a threonine (T), a tyrosine (Y), phenylalanine (F), and an alanine (A);
an amino acid insertion at position 585, or between positions 584 and 585, comprising one or more of a histidine (H) and a methionine (M);
an amino acid insertion at position 586, or between positions 585 and 586, comprising one or more of a histidine (H), a tyrosine (Y), a valine (V), a threonine (T), an alanine (A), an isoleucine (I), a tryptophan (W), a methionine (M), and a leucine (L);
an amino acid insertion at position 587, or between positions 586 and 587, comprising one or more of an isoleucine (I) and a proline (P);
an amino acid insertion at position 588, or between positions 587 and 588, comprising one or more of an isoleucine (I), a threonine (T), and a proline (P);
an amino acid insertion at position 589, or between positions 588 and 589, comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

2. The capsid protein of claim 1, wherein the capsid protein comprises one, two, three, four or more substitutions or insertions in the VR-VIII site.

3. The capsid protein of claim 2, wherein the capsid protein comprises, relative to reference SEQ ID NO:1, one, two, three, four or more substitutions or insertions at positions from 584 to 590 in the VR-VIII site, or one, two, three, four or more substitutions or insertions at positions from 585 to 590 in the VR-VIII site.

4. The capsid protein of any one of claims 1-3, wherein the capsid protein comprises, relative to reference sequence SEQ ID NO: 1:
(i) one or more amino acid substitutions selected from the group consisting of T582D, T582E, N583V, H584Q, S586K, A587P, A587S, Q588G, Q588M, A589S, A591I, G594Q, and G594D;
(ii) one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R;
(iii) one or more amino acid substitutions selected from the group consisting of Q585C, Q585S, S586I, A587V and A587G; or
(iv) one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

5. The capsid protein of any one of claims 1-4, wherein the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence.

6. The capsid protein of any one of claims 1-5, wherein the capsid protein comprises, relative to reference sequence SEQ ID NO: 1, one or more amino acid substitutions selected from the group consisting of N452K, N452A, N452V, N452I, G453A, G453N, S454T, S454D, G455N, Q456L, Q456K, N457L, N457V, Q458I, and Q458H.

7. The capsid protein of any one of claims 1-5, wherein the capsid protein comprises, relative to reference sequence SEQ ID NO: 1, at position 452 an amino acid selected from the group consisting of: K and N.

8. The capsid protein of any one of claims 1-5, wherein the capsid protein comprises, relative to reference sequence SEQ ID NO: 1, an amino acid substitution N452K.

9. The capsid protein of any one of claims 1-8, wherein the capsid protein comprises, relative to reference sequence SEQ ID NO: 1:
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q.

10. The capsid protein of any one of claims 1-5, wherein the capsid protein comprises, relative to reference sequence SEQ ID NO: 1:
at position 452 an amino acid selected from the group consisting of: K and N;
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

11. The capsid protein of any one of claims 1-8, wherein the capsid protein comprises, relative to reference sequence SEQ ID NO: 1:
at position 584 amino acid R;
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G;
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M.

12. The capsid protein of any one of claims 1-5, wherein the capsid protein comprises, relative to reference sequence SEQ ID NO: 1, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M

13. The capsid protein of any one of claims 1-8, wherein the capsid protein comprises, relative to reference sequence SEQ ID NO: 1:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q.

14. The capsid protein of any one of claims 1-5, wherein the capsid protein comprises, relative to reference sequence SEQ ID NO: 1:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

15. The capsid protein of any one of claims 1-8, wherein the capsid protein comprises, relative to reference sequence SEQ ID NO: 1:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or
at position 590 an amino acid selected from the group consisting of: I, S, G, H and R.

16. The capsid protein of any one of claims 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, G, M, C, V and T;
(iii) at position 586 an amino acid selected from the group consisting of: N, T, M, G, and D;
(iv) at position 587 an amino acid selected from the group consisting of: T, L, I, K, S, N and V;
(v) at position 588 an amino acid selected from the group consisting of: V, F, Y, L, T, S, I and R;
(vi) at position 589 an amino acid selected from the group consisting of: S, N, L, T, I and R; and
(vii) at position 590 an amino acid selected from the group consisting of: I, S, G, H and R.

17. The capsid protein of any one of claims 1-8, wherein the capsid protein comprises, relative to reference sequence SEQ ID NO: 1:
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q.

18. The capsid protein of any one of claims 1-5, wherein the capsid protein comprises, relative to reference sequence SEQ ID NO: 1:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

19. The capsid protein of any one of claims 1-8, wherein the capsid protein comprises, relative to reference sequence SEQ ID NO: 1:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or
at position 590 an amino acid selected from the group consisting of: I, S, G, and R.

20. The capsid protein of any one of claims 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N;
(iv) at position 587 an amino acid selected from the group consisting of: T, N, and V;
(v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

21. The capsid protein of any one of claims 1-20, wherein the capsid protein comprises, relative to reference sequence SEQ ID NO: 1:
at position 452 an amino acid selected from the group consisting of: K and N; and
at position 587 amino acid substitution A587T; and optionally comprises amino acid N or R at one, two or more positions selected from the group consisting of: 584, 585, 586, 588, 589, and 590.

22. The capsid protein of any one of claims 1-21, wherein the capsid protein comprises, relative to reference sequence SEQ ID NO: 1:
at position 452 an amino acid selected from the group consisting of: K and N; and
amino acid N or R at one, two or more positions selected from the group consisting of: 584, 585, 586, 588, 589, and 590.

23. The capsid protein of any one of claims 1-22, wherein the capsid protein comprises, relative to reference sequence SEQ ID NO: 1:
at position 452 an amino acid selected from the group consisting of: K and N; and
amino acid S at two or more positions selected from the group consisting of: 585, 586, 587, 588, 589 and 590.

24. The capsid protein of any one of claims 1-23, wherein the capsid protein comprises, relative to reference sequence SEQ ID NO: 1:
at position 452 an amino acid selected from the group consisting of: K and N; and
at three, four, five or six positions in the region 585-590 of the VR-VIII site, amino acids selected from the group consisting of: N, S, T, R and I.

25. The capsid protein of claim 24, wherein the capsid protein comprises, relative to reference sequence SEQ ID NO: 1:
at three, four, five or six positions in the region 585-590 of the VR-VIII site, amino acids selected from the group consisting of: N, S, T, and R.

26. The capsid protein of any one of claims 1-5, wherein the capsid protein comprises, relative to reference sequence SEQ ID NO: 1, amino acid substitutions Q585E, S586N, A587T, Q588V, A589S, Q590I, and N452K.

27. The capsid protein of any one of claims 1-5, wherein the capsid protein comprises, relative to reference sequence SEQ ID NO: 1, amino acid substitutions S586T, A587L, Q588F, A589N, Q590S, and N452K.

28. The capsid protein of any one of claims 1-5, wherein the capsid protein comprises, relative to reference sequence SEQ ID NO: 1, amino acid substitutions Q585N, A587T, Q588Y, A589L, Q590G, and N452K.

29. The capsid protein of any one of claims 1-5, wherein the capsid protein comprises, relative to reference sequence SEQ ID NO: 1, amino acid substitutions Q585G, A587I, Q588L, A589T, Q590H, and N452K.

30. The capsid protein of any one of claims 1-5, wherein the capsid protein comprises, relative to reference sequence SEQ ID NO: 1, amino acid substitutions Q585M, S586M, A587T, Q588T, and Q590R; and amino acid N at position 452.

31. The capsid protein of any one of claims 1-5, wherein the capsid protein comprises, relative to reference sequence SEQ ID NO: 1, amino acid substitutions Q585N, A587T, Q588Y, A589L, and Q590G; and amino acid N at position 452.

32. The capsid protein of any one of claims 1-5, wherein the capsid protein comprises, relative to reference sequence SEQ ID NO: 1, amino acid substitutions Q585C, A587T, Q588S, A589I, and Q590R; and amino acid N at position 452.

33. The capsid protein of any one of claims 1-5, wherein the capsid protein comprises, relative to reference sequence SEQ ID NO: 1, amino acid substitutions Q585E, S586D, A587N, Q588I, A589R, and Q590S; and amino acid N at position 452.

34. The capsid protein of any one of claims 1-5, wherein the capsid protein comprises, relative to reference sequence SEQ ID NO: 1, amino acid substitutions Q585E, S586D, A587N, Q588I, A589R, Q590S, and N452K.

35. The capsid protein of any one of claims 1-5, wherein the capsid protein comprises, relative to reference sequence SEQ ID NO: 1, amino acid substitutions Q585N, S586N, A587V, Q588I, A589S, Q590G, and N452K.

36. The capsid protein of any one of claims 1-5, wherein the capsid protein comprises, relative to reference sequence SEQ ID NO: 1, amino acid substitutions S586G and Q588Y; and amino acid N at position 452.

37. The capsid protein of any one of claims 1-5, wherein the capsid protein comprises, relative to reference sequence SEQ ID NO: 1, amino acid substitutions S586A, A587N, Q588Y, A589G, and N452K.

38. The capsid protein of any one of claims 1-37, wherein the capsid protein comprises, relative to reference sequence SEQ ID NO: 1, amino acids ATN at positions 581-583, and amino acids AQTG at positions 591-594.

39. The capsid protein of any one of claims 1-37, wherein the capsid protein comprises, relative to reference sequence SEQ ID NO:1, amino acids ATNH at positions 581-584, and amino acids AQTG at positions 591-594.

40. The capsid protein of any one of claims 1-5, wherein the capsid protein comprises, relative to reference sequence SEQ ID NO:1:
(i) amino acid sequence ATNHENTVSIAQTG at the VR-VIII positions 581-594, and amino acid K at the VR-IV position 452;
(ii) amino acid sequence ATNHQTLFNSAQTG at the VR-VIII positions 581-594, and amino acid K at the VR-IV position 452;
(iii) amino acid sequence ATNHNSTYLGAQTG at the VR-VIII positions 581-594, and amino acid K at the VR-IV position 452;
(iv) amino acid sequence ATNHGSILTHAQTG at the VR-VIII positions 581-594, and amino acid K at the VR-IV position 452;
(v) amino acid sequence ATNHMMTTARAQTG at the VR-VIII positions 581-594, and amino acid N at the VR-IV position 452;
(vi) amino acid sequence ATNHNSTYLGAQTG at the VR-VIII positions 581-594, and amino acid N at the VR-IV position 452;
(vii) amino acid sequence ATNHCSTSIRAQTG at the VR-VIII positions 581-594, and amino acid N at the VR-IV position 452;
(viii) amino acid sequence ATNHEDNIRSAQTG at the VR-VIII positions 581-594, and amino acid N at the VR-IV position 452;
(ix) amino acid sequence ATNHEDNIRSAQTG at the VR-VIII positions 581-594, and amino acid K at the VR-IV position 452;
(x) amino acid sequence ATNHNNVISGAQTG at the VR-VIII positions 581-594, and amino acid K at the VR-IV position 452;
(xi) amino acid sequence ATNHQGAYAQAQTG at the VR-VIII positions 581-594, and amino acid N at the VR-IV position 452;
(xii) amino acid sequence ATNHQANYGQAQTG at the VR-VIII positions 581-594, and amino acid K at the VR-IV position 452;
(xiii) amino acid sequence ATNHNMNRVNAQTG at the VR-VIII positions 581-594, and amino acid N at the VR-IV position 452;
(xiv) amino acid sequence ATNHNNVISGAQTG at the VR-VIII positions 581-594, and amino acid N at the VR-IV position 452;
(xv) amino acid sequence ATNHSNSVQSAQTG at the VR-VIII positions 581-594, and amino acid N at the VR-IV position 452;
(xvi) amino acid sequence ATNHSSTFQGAQTG at the VR-VIII positions 581-594, and amino acid N at the VR-IV position 452;
(xvii) amino acid sequence ATNHVSSFTSAQTG at the VR-VIII positions 581-594, and amino acid N at the VR-IV position 452;
(xviii) amino acid sequence ATNHSTTNFRAQTG at the VR-VIII positions 581-594, and amino acid N at the VR-IV position 452;
(xix) amino acid sequence ATNHSSIFNSAQTG at the VR-VIII positions 581-594, and amino acid N at the VR-IV position 452;
(xx) amino acid sequence ATNHAGNYNNAQTG at the VR-VIII positions 581-594, and amino acid N at the VR-IV position 452;
(xxi) amino acid sequence ATNHTSVISIAQTG at the VR-VIII positions 581-594, and amino acid N at the VR-IV position 452;
(xxii) amino acid sequence ATNHHSRVEIAQTG at the VR-VIII positions 581-594, and amino acid N at the VR-IV position 452;
(xxiii) amino acid sequence ATNHSSIIYSAQTG at the VR-VIII positions 581-594, and amino acid N at the VR-IV position 452;
(xxiv) amino acid sequence ATNHSGRDSYAQTG at the VR-VIII positions 581-594, and amino acid N at the VR-IV position 452;
(xxv) amino acid sequence ATNHSSSYNNAQTG at the VR-VIII positions 581-594, and amino acid N at the VR-IV position 452;
(xxvi) amino acid sequence ATNHHNPSINAQTG at the VR-VIII positions 581-594, and amino acid N at the VR-IV position 452;
(xxvii) amino acid sequence ATNHNRNGLLAQTG at the VR-VIII positions 581-594, and amino acid N at the VR-IV position 452;
(xxviii) amino acid sequence ATNHESTSVRAQTG at the VR-VIII positions 581-594, and amino acid N at the VR-IV position 452;
(xxix) amino acid sequence ATNHNIRTEMAQTG at the VR-VIII positions 581-594, and amino acid N at the VR-IV position 452;
(xxx) amino acid sequence ATNHQTLFNSAQTG at the VR-VIII positions 581-594, and amino acid N at the VR-IV position 452;
(xxxi) amino acid sequence ATNHLSVSSIAQTG at the VR-VIII positions 581-594, and amino acid N at the VR-IV position 452;
(xxxii) amino acid sequence ATNHEDIIRSAQTG at the VR-VIII positions 581-594, and amino acid N at the VR-IV position 452;
(xxxiii) amino acid sequence ATNRQTAQAQAQTG at the VR-VIII positions 581-594, and amino acid N at the VR-IV position 452; or
(xxxiv) amino acid sequence ATNRQIAQAQAQTG at the VR-VIII positions 581-594, and amino acid N at the VR-IV position 452.

41. The capsid protein of any one of claims 1-8, wherein the capsid protein comprises, relative to reference sequence SEQ ID NO: 1:
(i) an amino acid insertion at position 584 comprising one or more of an asparagine (N), a threonine (T), a tyrosine (Y), phenylalanine (F), and an alanine (A);
(ii) an amino acid insertion at position 585 comprising one or more of a histidine (H) and a methionine (M);
(iii) an amino acid insertion at position 586 comprising one or more of a histidine (H), a tyrosine (Y), a valine (V), a threonine (T), an alanine (A), an isoleucine (I), a tryptophan (W), a methionine (M), and a leucine;
(iv) an amino acid insertion at position 587 comprising one or more of an isoleucine (I) and a proline (P);
(v) an amino acid insertion at position 588 comprising one or more of an isoleucine (I), a threonine (T), and a proline (P); and/or
(vi) an amino acid insertion at position 589 comprising one or more of a glycine (G) and a glutamine (Q).

42. The capsid protein of claim 41, wherein the capsid protein comprises, relative to reference sequence SEQ ID NO: 1:
(i) an amino acid insertion at position 584 consisting of a TY, FN, or AT;
(ii) an amino acid insertion at position 585 consisting of MH;
(iii) an amino acid insertion at position 586 consisting of HY, VT, AI, WM, or ML;
(iv) an amino acid insertion at position 587 consisting of PI; and/or
(v) an amino acid insertion at position 588 consisting of IT or PT.

43. The capsid protein of any one of claims 1-42, wherein the capsid protein shares, or comprises a sequence sharing, at least 85%, at least 90%, at least 95%, at least 96%, at least 97%, at least 99%, or 100% amino acid sequence identity to an AAV9 VP3 sequence according to SEQ ID NO: 487, except for the specified modifications.

44. The capsid protein of any one of claims 1-43, wherein the capsid protein shares, or comprises a sequence sharing, at least 85%, at least 90%, at least 95%, at least 96%, at least 97%, at least 99%, or 100% amino acid sequence identity to an AAV9 VP2 sequence according to SEQ ID NO: 486, except for the specified modifications.

45. The capsid protein of any one of claims 1-44, wherein the capsid protein shares, or comprises a sequence sharing, at least 85%, at least 90%, at least 95%, at least 96%, at least 97%, at least 99%, or 100% amino acid sequence identity to an AAV9 VP1 sequence according to SEQ ID NO: 1, except for the specified modifications.

46. The capsid protein of any one of claims 1-45, wherein the capsid protein comprises, consists essentially of, or consists of an amino acid sequence sharing at least 85%, 90%, 95%, 96%, 97%, 98%, 99%, or 100% sequence identity to any sequence selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

47. The capsid protein of claim 1, wherein the capsid protein comprises, consists essentially of, or consists of a polypeptide sequence selected from the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774.

48. A recombinant adeno-associated virus (rAAV) virion, comprising a capsid protein according to any one of claims 1-47 and a vector genome comprising a polynucleotide cassette flanked by inverted terminal repeats (ITRs).

49. The rAAV virion of claim 48, wherein the rAAV virion transduces heart cells.

50. The rAAV virion of claim 48 or claim 49, wherein the rAAV virion transduces cardiomyocytes.

51. The rAAV virion of any one of claims 48-50, wherein the rAAV virion traffics to at least one organ other than the liver.

52. The rAAV virion of any one of claims 48-50, wherein the rAAV virion traffics to the heart.

53. The rAAV virion of any one of claims 48-52, wherein the rAAV virion exhibits a higher heart transduction efficiency than an rAAV virion having an AAV9 VP1 capsid protein according to SEQ ID NO: 1.

54. The rAAV virion of any one of claims 48-53, wherein the rAAV virion exhibits a higher heart-to-liver transduction ratio than an rAAV virion having an AAV9 VP1 capsid protein according to SEQ ID NO: 1, optionally at least 2, 3, 4, 5, 6, 7, 8, 9 or 10 times higher.

55. The rAAV virion of any one of claims 48-54, wherein administration of the rAAV virion to a subject leads to a lower liver viral load than administration of an rAAV virion having an AAV9 VP1 capsid protein according to SEQ ID NO: 1, optionally at least 2, 3, 4, 5, 6, 7, 8, 9 or 10 times lower.

56. The rAAV virion of any one of claims 48-55, wherein the rAAV virion exhibits a higher transduction efficiency, optionally higher heart transduction efficiency, than an rAAV virion having an AAV9 VP1 capsid protein according to SEQ ID NO: 1, assessed in a primate.

57. The rAAV virion of any one of claims 48-56, wherein the rAAV virion exhibits a higher heart-to-liver transduction ratio than an rAAV virion having an AAV9 VP1 capsid protein according to SEQ ID NO: 1, assessed in a primate, optionally at least 2, 3, 4, 5, 6, 7, 8, 9 or 10 times higher.

58. The rAAV virion of any one of claims 48-57, wherein administration of the rAAV virion to a subject leads to a lower liver viral load than administration of an rAAV virion having an AAV9 VP1 capsid protein according to SEQ ID NO: 1, assessed in a primate, optionally at least 2, 3, 4, 5, 6, 7, 8, 9 or 10 times lower.

59. The rAAV virion of any one of claims 48-58, wherein the polynucleotide cassette comprises a polynucleotide sequence encoding MYBPC3, DWORF, PKP2, KCNH2, TRPM4, DSG2, TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3, ACTN2, JPH2, PLN, ATP2A2, CACNA1C, DMD, DMPK, EPG5, EVC, EVC2, FBN1, NF1, SCN5A, SOS1, NPR1, ERBB4, VIP, MYH6, MYH7, Cas9, RBM20, MYOCD, ASCL1, GATA4, MEF2C, TBX5, miR-133, or MESP1.

60. The rAAV virion of any one of claims 48-59, wherein the polynucleotide cassette comprises a polynucleotide sequence which encodes a protein selected from the group consisting of: MYBPC3, DWORF, PKP2, LMNA, LAMP2, BAG3, CRYAB, JPH2, PLN, TTNI3, MYOCD, ASCL1, DSP, JUP, DSP, MYH6, MYH7, RBM20, and Cas9.

61. A pharmaceutical composition comprising an rAAV virion according to any one of claims 48-60 and a pharmaceutically acceptable carrier.

62. A polynucleotide encoding the capsid protein of any one of claims 1-47.

63. A method of transducing a cardiac cell, comprising contacting the cardiac cell with an rAAV virion according to any one of claims 48-60, wherein the rAAV virion transduces the cardiac cell.

64. The method of claim 63, wherein the cardiac cell is a cardiomyocyte.

65. The method of claim 63 or claim 64, wherein the rAAV virion exhibits higher transduction efficiency in the cell than an rAAV virion having an AAV9 VP1 capsid protein according to SEQ ID NO: 1.

66. A method of delivering one or more gene products to a cardiac cell, comprising contacting the cardiac cell with an rAAV virion according to any one of claims 48-60.

67. The method of claim 66, wherein the cardiac cell is a cardiomyocyte.

68. A method of treating a cardiac pathology in a subject in need thereof, comprising administering a therapeutically effective amount of an rAAV virion according to any one of claims 48-60 to the subject, wherein the rAAV virion transduces cardiac tissue.

69. A method of treating a heart disease or condition in a subject in need thereof, comprising administering a therapeutically effective amount of an rAAV virion according to any one of claims 48-60 to the subject.

70. A kit comprising a pharmaceutical composition according to claim 61 and instructions for use.


SEQUENCE LISTING (833 sequences)

SEQ ID NO: 1 - protein, 736 aa, Adeno-associated dependoparvovirus A
  source 1..736: note = Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 7 aa, Adeno-associated dependoparvovirus A
  source 1..7: note = Adeno-associated virus serotype 9
NGSGQNQ

SEQ ID NO: 3 - protein, 6 aa, Adeno-associated dependoparvovirus A
  source 1..6: note = Adeno-associated virus serotype 9
NNSEFA

SEQ ID NO: 4 - protein, 5 aa, Adeno-associated dependoparvovirus A
  source 1..5: note = Adeno-associated virus serotype 9
GRDNV

SEQ ID NO: 5 - protein, 14 aa, Adeno-associated dependoparvovirus A
  source 1..14: note = Adeno-associated virus serotype 9
ATNHQSAQAQAQTG

SEQ ID NO: 6 - protein, 9 aa, synthetic construct
  REGION 1..9: note = computer generated peptide library sequence
GYHKSGAAQ

SEQ ID NO: 7 - protein, 9 aa, synthetic construct
  REGION 1..9: note = computer generated peptide library sequence
VIIKSGAAQ

SEQ ID NO: 8 - protein, 9 aa, synthetic construct
  REGION 1..9: note = computer generated peptide library sequence
GYHKIGAAQ

SEQ ID NO: 9 - protein, 9 aa, synthetic construct
  REGION 1..9: note = computer generated peptide library sequence
GYHKSGVAQ

SEQ ID NO: 10 - protein, 9 aa, synthetic construct
  REGION 1..9: note = computer generated peptide library sequence
VYHKSGAAQ

SEQ ID NO: 11 - protein, 9 aa, synthetic construct
  REGION 1..9: note = computer generated peptide library sequence
GYHKISAAQ

SEQ ID NO: 12 - protein, 9 aa, synthetic construct
  REGION 1..9: note = computer generated peptide library sequence
TTVPSSSRY

SEQ ID NO: 13 - protein, 9 aa, synthetic construct
  REGION 1..9: note = computer generated peptide library sequence
VIIRVVRLS

SEQ ID NO: 14 - protein, 9 aa, synthetic construct
  REGION 1..9: note = computer generated peptide library sequence
TVLGQNQQT

SEQ ID NO: 15 - protein, 9 aa, synthetic construct
  REGION 1..9: note = computer generated peptide library sequence
IYHKSGAAQ

SEQ ID NO: 16 - protein, 9 aa, synthetic construct
  REGION 1..9: note = computer generated peptide library sequence
TVLDKNQQT

SEQ ID NO: 17 - protein, 9 aa, synthetic construct
  REGION 1..9: note = computer generated peptide library sequence
YSGTDVRYK

SEQ ID NO: 18 - protein, 9 aa, synthetic construct
  REGION 1..9: note = computer generated peptide library sequence
VTASGKEHR

SEQ ID NO: 19 - protein, 9 aa, synthetic construct
  REGION 1..9: note = computer generated peptide library sequence
GYRKSGAAQ

SEQ ID NO: 20 - protein, 9 aa, synthetic construct
  REGION 1..9: note = computer generated peptide library sequence
NRTVSNGSE

SEQ ID NO: 21 - protein, 9 aa, synthetic construct
  REGION 1..9: note = computer generated peptide library sequence
TVLDRINKT

SEQ ID NO: 22 - protein, 9 aa, synthetic construct
  REGION 1..9: note = computer generated peptide library sequence
TGVGHLTSA

SEQ ID NO: 23 - protein, 9 aa, synthetic construct
  REGION 1..9: note = computer generated peptide library sequence
GYHKGGAAQ

SEQ ID NO: 24 - protein, 9 aa, synthetic construct
  REGION 1..9: note = computer generated peptide library sequence
VIAKSGAAQ

SEQ ID NO: 25 - protein, 9 aa, synthetic construct
  REGION 1..9: note = computer generated peptide library sequence
GYHKSGAAH

SEQ ID NO: 26 - protein, 9 aa, synthetic construct
  REGION 1..9: note = computer generated peptide library sequence
FIIKSGAAQ

SEQ ID NO: 27 - protein, 9 aa, synthetic construct
  REGION 1..9: note = computer generated peptide library sequence
GYHKVVRLS

SEQ ID NO: 28 - protein, 9 aa, synthetic construct
  REGION 1..9: note = computer generated peptide library sequence
GATRSAVES

SEQ ID NO: 29 - protein, 9 aa, synthetic construct
  REGION 1..9: note = computer generated peptide library sequence
TVSGQNQQT

SEQ ID NO: 30 - protein, 9 aa, synthetic construct
  REGION 1..9: note = computer generated peptide library sequence
LSHKSGAAQ

SEQ ID NO: 31 - protein, 9 aa, synthetic construct
  REGION 1..9: note = computer generated peptide library sequence
SSSGQNQQT

SEQ ID NO: 32 - protein, 9 aa, synthetic construct
  REGION 1..9: note = computer generated peptide library sequence
SGSGQNQQT

SEQ ID NO: 33 - protein, 9 aa, synthetic construct
  REGION 1..9: note = computer generated peptide library sequence
SQVNGRPRD

SEQ ID NO: 34 - protein, 9 aa, synthetic construct
  REGION 1..9: note = computer generated peptide library sequence
GYHKEWCGS

SEQ ID NO: 35 - protein, 9 aa, synthetic construct
  REGION 1..9: note = computer generated peptide library sequence
VVSSKSLNS

SEQ ID NO: 36 - protein, 9 aa, synthetic construct
  REGION 1..9: note = computer generated peptide library sequence
GYHKSGAAP

SEQ ID NO: 37 - protein, 9 aa, synthetic construct
  REGION 1..9: note = computer generated peptide library sequence
DASSREKVR

SEQ ID NO: 38 - protein, 9 aa, synthetic construct
  REGION 1..9: note = computer generated peptide library sequence
SYHKSGAAQ

SEQ ID NO: 39 - protein, 9 aa, synthetic construct
  REGION 1..9: note = computer generated peptide library sequence
TANGSQKYL

SEQ ID NO: 40 - protein, 9 aa, synthetic construct
  REGION 1..9: note = computer generated peptide library sequence
VIIRVGAAQ

SEQ ID NO: 41 - protein, 9 aa, synthetic construct
  REGION 1..9: note = computer generated peptide library sequence
SSTNKISTA

SEQ ID NO: 42 - protein, 9 aa, synthetic construct
  REGION 1..9: note = computer generated peptide library sequence
TVLDRIQQT

SEQ ID NO: 43 - protein, 9 aa, synthetic construct
  REGION 1..9: note = computer generated peptide library sequence
GYHKSGAVQ

SEQ ID NO: 44 - protein, 9 aa, synthetic construct
  REGION 1..9: note = computer generated peptide library sequence
TVLDQNQQT

SEQ ID NO: 45 - protein, 9 aa, synthetic construct
  REGION 1..9: note = computer generated peptide library sequence
VNMSSPIKT

SEQ ID NO: 46 - protein, 9 aa, synthetic construct
  REGION 1..9: note = computer generated peptide library sequence
AAYNSNSAF

SEQ ID NO: 47 - protein, 9 aa, synthetic construct
  REGION 1..9: note = computer generated peptide library sequence
GYHKSGAAR

SEQ ID NO: 48 - protein, 9 aa, synthetic construct
  REGION 1..9: note = computer generated peptide library sequence
VIIRVVRLQ

SEQ ID NO: 49 - protein, 9 aa, synthetic construct
  REGION 1..9: note = computer generated peptide library sequence
RFWTQNQQT

SEQ ID NO: 50 - protein, 9 aa, synthetic construct
  REGION 1..9: note = computer generated peptide library sequence
SSPRASSAL

SEQ ID NO: 51 - protein, 9 aa, synthetic construct
  REGION 1..9: note = computer generated peptide library sequence
IIIRVVRLS

SEQ ID NO: 52 - protein, 9 aa, synthetic construct
  REGION 1..9: note = computer generated peptide library sequence
KSSNLTAMP

SEQ ID NO: 53 - protein, 9 aa, synthetic construct
  REGION 1..9: note = computer generated peptide library sequence
NLNSDRHSA

SEQ ID NO: 54 - protein, 9 aa, synthetic construct
  REGION 1..9: note = computer generated peptide library sequence
LSLKSGAAQ

SEQ ID NO: 55 - protein, 9 aa, synthetic construct
  REGION 1..9: note = computer generated peptide library sequence
TVLDRNQQT

SEQ ID NO: 56 - protein, 9 aa, synthetic construct
  REGION 1..9: note = computer generated peptide library sequence
GSERVSNSG

SEQ ID NO: 57 - protein, 9 aa, synthetic construct
  REGION 1..9: note = computer generated peptide library sequence
VIAKIGAAQ

SEQ ID NO: 58 - protein, 9 aa, synthetic construct
  REGION 1..9: note = computer generated peptide library sequence
VYHKIGAAQ

SEQ ID NO: 59 - protein, 9 aa, synthetic construct
  REGION 1..9: note = computer generated peptide library sequence
LSYKSGAAQ

SEQ ID NO: 60 - protein, 9 aa, synthetic construct
  REGION 1..9: note = computer generated peptide library sequence
STVSQPVRT

SEQ ID NO: 61 - protein, 9 aa, synthetic construct
  REGION 1..9: note = computer generated peptide library sequence
GHHKSGAAQ

SEQ ID NO: 62 - protein, 9 aa, synthetic construct
  REGION 1..9: note = computer generated peptide library sequence
YAGIDPRYH

SEQ ID NO: 63 - protein, 9 aa, synthetic construct
  REGION 1..9: note = computer generated peptide library sequence
DRSRKSMCD

SEQ ID NO: 64 - protein, 9 aa, synthetic construct
  REGION 1..9: note = computer generated peptide library sequence
VIIRSGAAQ

SEQ ID NO: 65 - protein, 9 aa, synthetic construct
  REGION 1..9: note = computer generated peptide library sequence
GYHKSGGSA

SEQ ID NO: 66 - protein, 9 aa, synthetic construct
  REGION 1..9: note = computer generated peptide library sequence
VIIKIGAAQ

SEQ ID NO: 67 - protein, 9 aa, synthetic construct
  REGION 1..9: note = computer generated peptide library sequence
GYHKVVQLS

SEQ ID NO: 68 - protein, 9 aa, synthetic construct
  REGION 1..9: note = computer generated peptide library sequence
VIIKLVAAQ

SEQ ID NO: 69 - protein, 9 aa, synthetic construct
  REGION 1..9: note = computer generated peptide library sequence
KVSSHSVCD

SEQ ID NO: 70 - protein, 9 aa, synthetic construct
  REGION 1..9: note = computer generated peptide library sequence
GYHKRVRLS

SEQ ID NO: 71 - protein, 9 aa, synthetic construct
  REGION 1..9: note = computer generated peptide library sequence
GYHKSSAAQ

SEQ ID NO: 72 - protein, 9 aa, synthetic construct
  REGION 1..9: note = computer generated peptide library sequence
GYRKIGAAQ

SEQ ID NO: 73 - protein, 9 aa, synthetic construct
  REGION 1..9: note = computer generated peptide library sequence
GYHKSGAAC

SEQ ID NO: 74 - protein, 9 aa, synthetic construct
  REGION 1..9: note = computer generated peptide library sequence
GYRQSGAAQ

SEQ ID NO: 75 - protein, 9 aa, synthetic construct
  REGION 1..9: note = computer generated peptide library sequence
VIIKLIAAQ

SEQ ID NO: 76 - protein, 9 aa, synthetic construct
  REGION 1..9: note = computer generated peptide library sequence
VIIRVVRAQ

SEQ ID NO: 77 - protein, 9 aa, synthetic construct
  REGION 1..9: note = computer generated peptide library sequence
GYHKSGAAW

SEQ ID NO: 78 - protein, 9 aa, synthetic construct
  REGION 1..9: note = computer generated peptide library sequence
GYHKSGAVS

SEQ ID NO: 79 - protein, 9 aa, synthetic construct
  REGION 1..9: note = computer generated peptide library sequence
GYHKEWCSS

SEQ ID NO: 80 - protein, 9 aa, synthetic construct
  REGION 1..9: note = computer generated peptide library sequence
SSSSNRLAD

SEQ ID NO: 81 - protein, 9 aa, synthetic construct
  REGION 1..9: note = computer generated peptide library sequence
SNNSSSAKF

SEQ ID NO: 82 - protein, 9 aa, synthetic construct
  REGION 1..9: note = computer generated peptide library sequence
VKLSSTSSS

SEQ ID NO: 83 - protein, 9 aa, synthetic construct
  REGION 1..9: note = computer generated peptide library sequence
GYHKEWCAQ

SEQ ID NO: 84 - protein, 9 aa, synthetic construct
  REGION 1..9: note = computer generated peptide library sequence
AGSGQNQQT

SEQ ID NO: 85 - protein, 9 aa, synthetic construct
  REGION 1..9: note = computer generated peptide library sequence
NPHGTATYL

SEQ ID NO: 86 - protein, 9 aa, synthetic construct
  REGION 1..9: note = computer generated peptide library sequence
NGSGQNQHT

SEQ ID NO: 87 - protein, 9 aa, synthetic construct
  REGION 1..9: note = computer generated peptide library sequence
GYHKVGAAQ

SEQ ID NO: 88 - protein, 9 aa, synthetic construct
  REGION 1..9: note = computer generated peptide library sequence
VIIRVVRLK

SEQ ID NO: 89 - protein, 9 aa, synthetic construct
  REGION 1..9: note = computer generated peptide library sequence
NSIPSTSKW

SEQ ID NO: 90 - protein, 9 aa, synthetic construct
  REGION 1..9: note = computer generated peptide library sequence
VIIRVVQLQ

SEQ ID NO: 91 - protein, 9 aa, synthetic construct
  REGION 1..9: note = computer generated peptide library sequence
SQVNGRPQD

SEQ ID NO: 92 - protein, 9 aa, synthetic construct
  REGION 1..9: note = computer generated peptide library sequence
NGSGQDQQT

SEQ ID NO: 93 - protein, 9 aa, synthetic construct
  REGION 1..9: note = computer generated peptide library sequence
GLNSSDRRL

SEQ ID NO: 94 - protein, 9 aa, synthetic construct
  REGION 1..9: note = computer generated peptide library sequence
IYHKIGAAQ

SEQ ID NO: 95 - protein, 9 aa, synthetic construct
  REGION 1..9: note = computer generated peptide library sequence
YHKSGAAQL

SEQ ID NO: 96 - protein, 9 aa, synthetic construct
  REGION 1..9: note = computer generated peptide library sequence
YSGTDVQYK

SEQ ID NO: 97 - protein, 9 aa, synthetic construct
  REGION 1..9: note = computer generated peptide library sequence
LGSGQNQQT

SEQ ID NO: 98 - protein, 9 aa, synthetic construct
  REGION 1..9: note = computer generated peptide library sequence
PVSSGADRR

SEQ ID NO: 99 - protein, 9 aa, synthetic construct
  REGION 1..9: note = computer generated peptide library sequence
EHSTKLNAC

SEQ ID NO: 100 - protein, 9 aa, synthetic construct
  REGION 1..9: note = computer generated peptide library sequence
NGSDRINKR

SEQ ID NO: 101 - protein, 9 aa, synthetic construct
  REGION 1..9: note = computer generated peptide library sequence
VIIKGGAAQ

SEQ ID NO: 102 - protein, 9 aa, synthetic construct
  REGION 1..9: note = computer generated peptide library sequence
GYHRVVRLS

SEQ ID NO: 103 - protein, 9 aa, synthetic construct
  REGION 1..9: note = computer generated peptide library sequence
VIIRVVRLL

SEQ ID NO: 104 - protein, 9 aa, synthetic construct
  REGION 1..9: note = computer generated peptide library sequence
VILKSGAAQ

SEQ ID NO: 105 - protein, 6 aa, synthetic construct
  REGION 1..6: note = computer generated peptide library sequence
LNSMLI

SEQ ID NO: 106 - protein, 6 aa, synthetic construct
  REGION 1..6: note = computer generated peptide library sequence
NGMSFT

SEQ ID NO: 107 - protein, 6 aa, synthetic construct
  REGION 1..6: note = computer generated peptide library sequence
HKTFSI

SEQ ID NO: 108 - protein, 6 aa, synthetic construct
  REGION 1..6: note = computer generated peptide library sequence
SMSNFV

SEQ ID NO: 109 - protein, 6 aa, synthetic construct
  REGION 1..6: note = computer generated peptide library sequence
ATIPPI

SEQ ID NO: 110 - protein, 6 aa, synthetic construct
  REGION 1..6: note = computer generated peptide library sequence
SSTHFD

SEQ ID NO: 111 - protein, 6 aa, synthetic construct
  REGION 1..6: note = computer generated peptide library sequence
NNQFSY

SEQ ID NO: 112 - protein, 6 aa, synthetic construct
  REGION 1..6: note = computer generated peptide library sequence
NMGHYS

SEQ ID NO: 113 - protein, 6 aa, synthetic construct
  REGION 1..6: note = computer generated peptide library sequence
SKQMFQ

SEQ ID NO: 114 - protein, 6 aa, synthetic construct
  REGION 1..6: note = computer generated peptide library sequence
WPSAGV

SEQ ID NO: 115 - protein, 6 aa, synthetic construct
  REGION 1..6: note = computer generated peptide library sequence
NGGYQC

SEQ ID NO: 116 - protein, 6 aa, synthetic construct
  REGION 1..6: note = computer generated peptide library sequence
STSPIV

SEQ ID NO: 117 - protein, 6 aa, synthetic construct
  REGION 1..6: note = computer generated peptide library sequence
SQSGLW

SEQ ID NO: 118 - protein, 6 aa, synthetic construct
  REGION 1..6: note = computer generated peptide library sequence
VNSQFS

SEQ ID NO: 119 - protein, 6 aa, synthetic construct
  REGION 1..6: note = computer generated peptide library sequence
SGIEFR

SEQ ID NO: 120 - protein, 6 aa, synthetic construct
  REGION 1..6: note = computer generated peptide library sequence
SASKFT

SEQ ID NO: 121 - protein, 6 aa, synthetic construct
  REGION 1..6: note = computer generated peptide library sequence
QLNWTS

SEQ ID NO: 122 - protein, 6 aa, synthetic construct
  REGION 1..6: note = computer generated peptide library sequence
SMGFPV

SEQ ID NO: 123 - protein, 6 aa, synthetic construct
  REGION 1..6: note = computer generated peptide library sequence
SSFMGL

SEQ ID NO: 124 - protein, 6 aa, synthetic construct
  REGION 1..6: note = computer generated peptide library sequence
GSNFHV

SEQ ID NO: 125 - protein, 6 aa, synthetic construct
  REGION 1..6: note = computer generated peptide library sequence
DMTLYA

SEQ ID NO: 126 - protein, 6 aa, synthetic construct
  REGION 1..6: note = computer generated peptide library sequence
MGCLFT

SEQ ID NO: 127 - protein, 6 aa, synthetic construct
  REGION 1..6: note = computer generated peptide library sequence
ALAFNS

SEQ ID NO: 128 - protein, 6 aa, synthetic construct
  REGION 1..6: note = computer generated peptide library sequence
SKFLFA

SEQ ID NO: 129 - protein, 6 aa, synthetic construct
  REGION 1..6: note = computer generated peptide library sequence
QDAGLL

SEQ ID NO: 130 - protein, 6 aa, synthetic construct
  REGION 1..6: note = computer generated peptide library sequence
QDASLL

SEQ ID NO: 131 - protein, 6 aa, synthetic construct
  REGION 1..6: note = computer generated peptide library sequence
RDDMFS

SEQ ID NO: 132 - protein, 6 aa, synthetic construct
  REGION 1..6: note = computer generated peptide library sequence
LSRCFQ

SEQ ID NO: 133 - protein, 6 aa, synthetic construct
  REGION 1..6: note = computer generated peptide library sequence
LSRDFQ

SEQ ID NO: 134 - protein, 6 aa, synthetic construct
  REGION 1..6: note = computer generated peptide library sequence
QGLTPV

SEQ ID NO: 135 - protein, 6 aa, synthetic construct
  REGION 1..6: note = computer generated peptide library sequence
QWDVFT

SEQ ID NO: 136 - protein, 6 aa, synthetic construct
  REGION 1..6: note = computer generated peptide library sequence
PRVSFA

SEQ ID NO: 137 - protein, 6 aa, synthetic construct
  REGION 1..6: note = computer generated peptide library sequence
QSYYNP

SEQ ID NO: 138 - protein, 6 aa, synthetic construct
  REGION 1..6: note = computer generated peptide library sequence
RASHLG

SEQ ID NO: 139 - protein, 6 aa, synthetic construct
  REGION 1..6: note = computer generated peptide library sequence
IILFVP

SEQ ID NO: 140 - protein, 6 aa, synthetic construct
  REGION 1..6: note = computer generated peptide library sequence
IISFSY

SEQ ID NO: 141 - protein, 6 aa, synthetic construct
  REGION 1..6: note = computer generated peptide library sequence
LDSMLI

SEQ ID NO: 142 - protein, 6 aa, synthetic construct
  REGION 1..6: note = computer generated peptide library sequence
NIGHYS

SEQ ID NO: 143 - protein, 6 aa, synthetic construct
  REGION 1..6: note = computer generated peptide library sequence
NRMSFT

SEQ ID NO: 144 - protein, 6 aa, synthetic construct
  REGION 1..6: note = computer generated peptide library sequence
NGMSFA

SEQ ID NO: 145 - protein, 6 aa, synthetic construct
  REGION 1..6: note = computer generated peptide library sequence
IILLLP

SEQ ID NO: 146 - protein, 6 aa, synthetic construct
  REGION 1..6: note = computer generated peptide library sequence
RMRSLL

SEQ ID NO: 147 - protein, 6 aa, synthetic construct
  REGION 1..6: note = computer generated peptide library sequence
RRRCRF

SEQ ID NO: 148 - protein, 6 aa, synthetic construct
  REGION 1..6: note = computer generated peptide library sequence
PKQMFQ

SEQ ID NO: 149 - protein, 6 aa, synthetic construct
  REGION 1..6: note = computer generated peptide library sequence
LMSNFV

SEQ ID NO: 150 - protein, 6 aa, synthetic construct
  REGION 1..6: note = computer generated peptide library sequence
GASHLG

SEQ ID NO: 151 - protein, 6 aa, synthetic construct
  REGION 1..6: note = computer generated peptide library sequence
CASISW

SEQ ID NO: 152 - protein, 6 aa, synthetic construct
  REGION 1..6: note = computer generated peptide library sequence
SMTTFR

SEQ ID NO: 153 - protein, 6 aa, synthetic construct
  REGION 1..6: note = computer generated peptide library sequence
AAIPPI

SEQ ID NO: 154 - protein, 6 aa, synthetic construct
  REGION 1..6: note = computer generated peptide library sequence
PGCESL

SEQ ID NO: 155 - protein, 6 aa, synthetic construct
  REGION 1..6: note = computer generated peptide library sequence
SMGFAC

SEQ ID NO: 156 - protein, 6 aa, synthetic construct
  REGION 1..6: note = computer generated peptide library sequence
FLPSLM

SEQ ID NO: 157 - protein, 6 aa, synthetic construct
  REGION 1..6: note = computer generated peptide library sequence
NGISFT

SEQ ID NO: 158 - protein, 6 aa, synthetic construct
  REGION 1..6: note = computer generated peptide library sequence
ESSRWA

SEQ ID NO: 159 - protein, 6 aa, synthetic construct
  REGION 1..6: note = computer generated peptide library sequence
QLYFVP

SEQ ID NO: 160 - protein, 6 aa, synthetic construct
  REGION 1..6: note = computer generated peptide library sequence
SSNFHV

SEQ ID NO: 161 - protein, 6 aa, synthetic construct
  REGION 1..6: note = computer generated peptide library sequence
LEFMLI

SEQ ID NO: 162 - protein, 6 aa, synthetic construct
  REGION 1..6: note = computer generated peptide library sequence
QFDSFD

SEQ ID NO: 163 - protein, 6 aa, synthetic construct
  REGION 1..6: note = computer generated peptide library sequence
SPVFAC

SEQ ID NO: 164 - protein, 6 aa, synthetic construct
  REGION 1..6: note = computer generated peptide library sequence
VRLIFD

SEQ ID NO: 165 - protein, 6 aa, synthetic construct
  REGION 1..6: note = computer generated peptide library sequence
NGMSFI

SEQ ID NO: 166 - protein, 6 aa, synthetic construct
  REGION 1..6: note = computer generated peptide library sequence
LLFPPI

SEQ ID NO: 167 - protein, 6 aa, synthetic construct
  REGION 1..6: note = computer generated peptide library sequence
GAGVTG

SEQ ID NO: 168 - protein, 6 aa, synthetic construct
  REGION 1..6: note = computer generated peptide library sequence
QWMSFT

SEQ ID NO: 169 - protein, 6 aa, synthetic construct
  REGION 1..6: note = computer generated peptide library sequence
SIGFPV

SEQ ID NO: 170 - protein, 6 aa, synthetic construct
  REGION 1..6: note = computer generated peptide library sequence
RMQSLL

SEQ ID NO: 171 - protein, 6 aa, synthetic construct
  REGION 1..6: note = computer generated peptide library sequence
TSALQV

SEQ ID NO: 172 - protein, 6 aa, synthetic construct
  REGION 1..6: note = computer generated peptide library sequence
SLTHFD

SEQ ID NO: 173 - protein, 6 aa, synthetic construct
  REGION 1..6: note = computer generated peptide library sequence
QELPFL

SEQ ID NO: 174 - protein, 6 aa, synthetic construct
  REGION 1..6: note = computer generated peptide library sequence
LYFLLP

SEQ ID NO: 175 - protein, 6 aa, synthetic construct
  REGION 1..6: note = computer generated peptide library sequence
LSFFFA

SEQ ID NO: 176 - protein, 6 aa, synthetic construct
  REGION 1..6: note = computer generated peptide library sequence
LSRIFQ

SEQ ID NO: 177 - protein, 6 aa, synthetic construct
  REGION 1..6: note = computer generated peptide library sequence
DEVILF

SEQ ID NO: 178 - protein, 6 aa, synthetic construct
  REGION 1..6: note = computer generated peptide library sequence
RAGVAG

SEQ ID NO: 179 - protein, 6 aa, synthetic construct
  REGION 1..6: note = computer generated peptide library sequence
NGMSLP

SEQ ID NO: 180 - protein, 6 aa, synthetic construct
  REGION 1..6: note = computer generated peptide library sequence
PFEDFQ

SEQ ID NO: 181 - protein, 6 aa, synthetic construct
  REGION 1..6: note = computer generated peptide library sequence
QYGSLF

SEQ ID NO: 182 - protein, 6 aa, synthetic construct
  REGION 1..6: note = computer generated peptide library sequence
NYTFVL

SEQ ID NO: 183 - protein, 6 aa, synthetic construct
  REGION 1..6: note = computer generated peptide library sequence
MSGYQC

SEQ ID NO: 184 - protein, 6 aa, synthetic construct
  REGION 1..6: note = computer generated peptide library sequence
NYAFVP

SEQ ID NO: 185 - protein, 6 aa, synthetic construct
  REGION 1..6: note = computer generated peptide library sequence
RAGVTG

SEQ ID NO: 186 - protein, 6 aa, synthetic construct
  REGION 1..6: note = computer generated peptide library sequence
WNSMLI

SEQ ID NO: 187 - protein, 6 aa, synthetic construct
  REGION 1..6: note = computer generated peptide library sequence
IRRFSI

SEQ ID NO: 188 - protein, 6 aa, synthetic construct
  REGION 1..6: note = computer generated peptide library sequence
NGMSFY

SEQ ID NO: 189 - protein, 6 aa, synthetic construct
  REGION 1..6: note = computer generated peptide library sequence
IIQFSY

SEQ ID NO: 190 - protein, 6 aa, synthetic construct
  REGION 1..6: note = computer generated peptide library sequence
NGCLFT

SEQ ID NO: 191 - protein, 6 aa, synthetic construct
  REGION 1..6: note = computer generated peptide library sequence
RDASLL

SEQ ID NO: 192 - protein, 6 aa, synthetic construct
  REGION 1..6: note = computer generated peptide library sequence
ADSMLI

SEQ ID NO: 193 - protein, 6 aa, synthetic construct
  REGION 1..6: note = computer generated peptide library sequence
VDSQFS

SEQ ID NO: 194 - protein, 6 aa, synthetic construct
  REGION 1..6: note = computer generated peptide library sequence
SIGNFV

SEQ ID NO: 195 - protein, 6 aa, synthetic construct
  REGION 1..6: note = computer generated peptide library sequence
NGMSLL

SEQ ID NO: 196 - protein, 6 aa, synthetic construct
  REGION 1..6: note = computer generated peptide library sequence
NYTFVP

SEQ ID NO: 197 - protein, 6 aa, synthetic construct
  REGION 1..6: note = computer generated peptide library sequence
IRRLVF

SEQ ID NO: 198 - protein, 6 aa, synthetic construct
  REGION 1..6: note = computer generated peptide library sequence
PMSNFV

SEQ ID NO: 199 - protein, 6 aa, synthetic construct
  REGION 1..6: note = computer generated peptide library sequence
LWVFPV

SEQ ID NO: 200 - protein, 6 aa, synthetic construct
  REGION 1..6: note = computer generated peptide library sequence
VRLHFD

SEQ ID NO: 201 - protein, 6 aa, synthetic construct
  REGION 1..6: note = computer generated peptide library sequence
SMSNLF

SEQ ID NO: 202 - protein, 6 aa, synthetic construct
  REGION 1..6: note = computer generated peptide library sequence
STSLIV

SEQ ID NO: 203 - protein, 6 aa, synthetic construct
  REGION 1..6: note = computer generated peptide library sequence
HKTFGI

SEQ ID NO: 204 - protein, 5 aa, synthetic construct
  REGION 1..5: note = computer generated peptide library sequence
RGNQV

SEQ ID NO: 205 - protein, 5 aa, synthetic construct
  REGION 1..5: note = computer generated peptide library sequence
VSLNR

SEQ ID NO: 206 - protein, 5 aa, synthetic construct
  REGION 1..5: note = computer generated peptide library sequence
CDYSV

SEQ ID NO: 207 - protein, 5 aa, synthetic construct
  REGION 1..5: note = computer generated peptide library sequence
QHGHI

SEQ ID NO: 208 - protein, 5 aa, synthetic construct
  REGION 1..5: note = computer generated peptide library sequence
LCSLV

SEQ ID NO: 209 - protein, 5 aa, synthetic construct
  REGION 1..5: note = computer generated peptide library sequence
PTIYV

SEQ ID NO: 210 - protein, 5 aa, synthetic construct
  REGION 1..5: note = computer generated peptide library sequence
DVIHI

SEQ ID NO: 211 - protein, 5 aa, synthetic construct
  REGION 1..5: note = computer generated peptide library sequence
AEFYA

SEQ ID NO: 212 - protein, 5 aa, synthetic construct
  REGION 1..5: note = computer generated peptide library sequence
NSVVC

SEQ ID NO: 213 - protein, 5 aa, synthetic construct
  REGION 1..5: note = computer generated peptide library sequence
VRSNC

SEQ ID NO: 214 - protein, 5 aa, synthetic construct
  REGION 1..5: note = computer generated peptide library sequence
LANNI

SEQ ID NO: 215 - protein, 5 aa, synthetic construct
  REGION 1..5: note = computer generated peptide library sequence
NLQFM

SEQ ID NO: 216 - protein, 5 aa, synthetic construct
  REGION 1..5: note = computer generated peptide library sequence
EFRDL

SEQ ID NO: 217 - protein, 5 aa, synthetic construct
  REGION 1..5: note = computer generated peptide library sequence
DFGSL

SEQ ID NO: 218 - protein, 5 aa, synthetic construct
  REGION 1..5: note = computer generated peptide library sequence
VTNYC

SEQ ID NO: 219 - protein, 5 aa, synthetic construct
  REGION 1..5: note = computer generated peptide library sequence
WNTNA

SEQ ID NO: 220 - protein, 5 aa, synthetic construct
  REGION 1..5: note = computer generated peptide library sequence
TESTC

SEQ ID NO: 221 - protein, 5 aa, synthetic construct
  REGION 1..5: note = computer generated peptide library sequence
SGAAV

SEQ ID NO: 222 - protein, 5 aa, synthetic construct
  REGION 1..5: note = computer generated peptide library sequence
GGCDI

SEQ ID NO: 223 - protein, 5 aa, synthetic construct
  REGION 1..5: note = computer generated peptide library sequence
SGSVV

SEQ ID NO: 224 - protein, 5 aa, synthetic construct
  REGION 1..5: note = computer generated peptide library sequence
SSNAC

SEQ ID NO: 225 - protein, 5 aa, synthetic construct
  REGION 1..5: note = computer generated peptide library sequence
YNTTV

SEQ ID NO: 226 - protein, 5 aa, synthetic construct
  REGION 1..5: note = computer generated peptide library sequence
SKCLA

SEQ ID NO: 227 - protein, 5 aa, synthetic construct
  REGION 1..5: note = computer generated peptide library sequence
SAYTV

SEQ ID NO: 228 - protein, 5 aa, synthetic construct
  REGION 1..5: note = computer generated peptide library sequence
VRDTV

SEQ ID NO: 229 - protein, 5 aa, synthetic construct
  REGION 1..5: note = computer generated peptide library sequence
WRSMV

SEQ ID NO: 230 - protein, 5 aa, synthetic construct
  REGION 1..5: note = computer generated peptide library sequence
AYHGV

SEQ ID NO: 231 - protein, 5 aa, synthetic construct
  REGION 1..5: note = computer generated peptide library sequence
GMNTI

SEQ ID NO: 232 - protein, 5 aa, synthetic construct
  REGION 1..5: note = computer generated peptide library sequence
AETSL

SEQ ID NO: 233 - protein, 5 aa, synthetic construct
  REGION 1..5: note = computer generated peptide library sequence
TLVYV

SEQ ID NO: 234 - protein, 5 aa, synthetic construct
  REGION 1..5: note = computer generated peptide library sequence
NHDWI

SEQ ID NO: 235 - protein, 5 aa, synthetic construct
  REGION 1..5: note = computer generated peptide library sequence
TVGIV

SEQ ID NO: 236 - protein, 5 aa, synthetic construct
  REGION 1..5: note = computer generated peptide library sequence
SLPTV

SEQ ID NO: 237 - protein, 5 aa, synthetic construct
  REGION 1..5: note = computer generated peptide library sequence
TGILC

SEQ ID NO: 238 - protein, 5 aa, synthetic construct
  REGION 1..5: note = computer generated peptide library sequence
TDTYI

SEQ ID NO: 239 - protein, 5 aa, synthetic construct
  REGION 1..5: note = computer generated peptide library sequence
LPVTY

SEQ ID NO: 240 - protein, 5 aa, synthetic construct
  REGION 1..5: note = computer generated peptide library sequence
GDVYI

SEQ ID NO: 241 - protein, 5 aa, synthetic construct
  REGION 1..5: note = computer generated peptide library sequence
LYGTV

SEQ ID NO: 242 - protein, 5 aa, synthetic construct
  REGION 1..5: note = computer generated peptide library sequence
GCEFI

SEQ ID NO: 243 - protein, 5 aa, synthetic construct
  REGION 1..5: note = computer generated peptide library sequence
SAGLL

SEQ ID NO: 244 - protein, 5 aa, synthetic construct
  REGION 1..5: note = computer generated peptide library sequence
IKSNI

SEQ ID NO: 245 - protein, 5 aa, synthetic construct
  REGION 1..5: note = computer generated peptide library sequence
VTTSL

SEQ ID NO: 246 - protein, 5 aa, synthetic construct
  REGION 1..5: note = computer generated peptide library sequence
AVTSV

SEQ ID NO: 247 - protein, 5 aa, synthetic construct
  REGION 1..5: note = computer generated peptide library sequence
RDIHI

SEQ ID NO: 248 - protein, 5 aa, synthetic construct
  REGION 1..5: note = computer generated peptide library sequence
SAISL

SEQ ID NO: 249 - protein, 5 aa, synthetic construct
  REGION 1..5: note = computer generated peptide library sequence
VASTC

SEQ ID NO: 250 - protein, 5 aa, synthetic construct
  REGION 1..5: note = computer generated peptide library sequence
IKGLL

SEQ ID NO: 251 - protein, 5 aa, synthetic construct
  REGION 1..5: note = computer generated peptide library sequence
GSYHT

SEQ ID NO: 252 - protein, 5 aa, synthetic construct
  REGION 1..5: note = computer generated peptide library sequence
RIGFV

SEQ ID NO: 253 - protein, 5 aa, synthetic construct
  REGION 1..5: note = computer generated peptide library sequence
NDIYI

SEQ ID NO: 254 - protein, 5 aa, synthetic construct
  REGION 1..5: note = computer generated peptide library sequence
AVSCV

SEQ ID NO: 255 - protein, 5 aa, synthetic construct
  REGION 1..5: note = computer generated peptide library sequence
QHNLL

SEQ ID NO: 256 - protein, 5 aa, synthetic construct
  REGION 1..5: note = computer generated peptide library sequence
VSSCV

SEQ ID NO: 257 - protein, 5 aa, synthetic construct
  REGION 1..5: note = computer generated peptide library sequence
LNLDV

SEQ ID NO: 258 - protein, 5 aa, synthetic construct
  REGION 1..5: note = computer generated peptide library sequence
LGATI

SEQ ID NO: 259 - protein, 5 aa, synthetic construct
  REGION 1..5: note = computer generated peptide library sequence
PVLCV

SEQ ID NO: 260 - protein, 5 aa, synthetic construct
  REGION 1..5: note = computer generated peptide library sequence
SARHI

SEQ ID NO: 261 - protein, 5 aa, synthetic construct
  REGION 1..5: note = computer generated peptide library sequence
RATLI

SEQ ID NO: 262 - protein, 5 aa, synthetic construct
  REGION 1..5: note = computer generated peptide library sequence
PYNHA

SEQ ID NO: 263 - protein, 5 aa, synthetic construct
  REGION 1..5: note = computer generated peptide library sequence
IGDSI

SEQ ID NO: 264 - protein, 5 aa, synthetic construct
  REGION 1..5: note = computer generated peptide library sequence
SPMLC

SEQ ID NO: 265 - protein, 5 aa, synthetic construct
  REGION 1..5: note = computer generated peptide library sequence
YDSTL

SEQ ID NO: 266 - protein, 5 aa, synthetic construct
  REGION 1..5: note = computer generated peptide library sequence
ALKHV

SEQ ID NO: 267 - protein, 5 aa, synthetic construct
  REGION 1..5: note = computer generated peptide library sequence
ADLLT

SEQ ID NO: 268 - protein, 5 aa, synthetic construct
  REGION 1..5: note = computer generated peptide library sequence
NNGHL

SEQ ID NO: 269 - protein, 5 aa, synthetic construct
  REGION 1..5: note = computer generated peptide library sequence
INSEV

SEQ ID NO: 270 - protein, 5 aa, synthetic construct
  REGION 1..5: note = computer generated peptide library sequence
SNKTT

SEQ ID NO: 271 - protein, 5 aa, synthetic construct
  REGION 1..5: note = computer generated peptide library sequence
GSTGL

SEQ ID NO: 272 - protein, 5 aa, synthetic construct
  REGION 1..5: note = computer generated peptide library sequence
DSDMI

SEQ ID NO: 273 - protein, 5 aa, synthetic construct
  REGION 1..5: note = computer generated peptide library sequence
TSNFI

SEQ ID NO: 274 - protein, 5 aa, synthetic construct
  REGION 1..5: note = computer generated peptide library sequence
RNFTT

SEQ ID NO: 275 - protein, 5 aa, synthetic construct
  REGION 1..5: note = computer generated peptide library sequence
SHKYS

SEQ ID NO: 276 - protein, 5 aa, synthetic construct
  REGION 1..5: note = computer generated peptide library sequence
VSDIV

SEQ ID NO: 277 - protein, 5 aa, synthetic construct
  REGION 1..5: note = computer generated peptide library sequence
RVVQA

SEQ ID NO: 278 - protein, 5 aa, synthetic construct
  REGION 1..5: note = computer generated peptide library sequence
AACAV

SEQ ID NO: 279 - protein, 5 aa, synthetic construct
  REGION 1..5: note = computer generated peptide library sequence
RGRQI

SEQ ID NO: 280 - protein, 5 aa, synthetic construct
  REGION 1..5: note = computer generated peptide library sequence
AVANI

SEQ ID NO: 281 - protein, 5 aa, synthetic construct
  REGION 1..5: note = computer generated peptide library sequence
AGYDL

SEQ ID NO: 282 - protein, 5 aa, synthetic construct
  REGION 1..5: note = computer generated peptide library sequence
LSEAA

SEQ ID NO: 283 - protein, 5 aa, synthetic construct
  REGION 1..5: note = computer generated peptide library sequence
MSNYL

SEQ ID NO: 284 - protein, 5 aa, synthetic construct
  REGION 1..5: note = computer generated peptide library sequence
NFSDN

SEQ ID NO: 285 - protein, 5 aa, synthetic construct
  REGION 1..5: note = computer generated peptide library sequence
SCCDV

SEQ ID NO: 286 - protein, 5 aa, synthetic construct
  REGION 1..5: note = computer generated peptide library sequence
LASSV

SEQ ID NO: 287 - protein, 5 aa, synthetic construct
  REGION 1..5: note = computer generated peptide library sequence
PDHAV

SEQ ID NO: 288 - protein, 5 aa, synthetic construct
  REGION 1..5: note = computer generated peptide library sequence
KFDII

SEQ ID NO: 289 - protein, 5 aa, synthetic construct
  REGION 1..5: note = computer generated peptide library sequence
NSSSA

SEQ ID NO: 290 - protein, 5 aa, synthetic construct
  REGION 1..5: note = computer generated peptide library sequence
HTMHV

SEQ ID NO: 291 - protein, 5 aa, synthetic construct
  REGION 1..5: note = computer generated peptide library sequence
TLSYC

SEQ ID NO: 292 - protein, 5 aa, synthetic construct
  REGION 1..5: note = computer generated peptide library sequence
ADTHR

SEQ ID NO: 293 - protein, 5 aa, synthetic construct
  REGION 1..5: note = computer generated peptide library sequence
SMYSV

SEQ ID NO: 294 - protein, 5 aa, synthetic construct
  REGION 1..5: note = computer generated peptide library sequence
SVNLV

SEQ ID NO: 295 - protein, 5 aa, synthetic construct
  REGION 1..5: note = computer generated peptide library sequence
MSGHL

SEQ ID NO: 296 - protein, 5 aa, synthetic construct
  REGION 1..5: note = computer generated peptide library sequence
KISDT

SEQ ID NO: 297 - protein, 5 aa, synthetic construct
  REGION 1..5: note = computer generated peptide library sequence
TGLLA

SEQ ID NO: 298 - protein, 5 aa, synthetic construct
  REGION 1..5: note = computer generated peptide library sequence
AWTTS

SEQ ID NO: 299 - protein, 5 aa, synthetic construct
  REGION 1..5: note = computer generated peptide library sequence
GGALI

SEQ ID NO: 300 - protein, 5 aa, synthetic construct
  REGION 1..5: note = computer generated peptide library sequence
SCIEV

SEQ ID NO: 301 - protein, 5 aa, synthetic construct
  REGION 1..5: note = computer generated peptide library sequence
PPVIC

SEQ ID NO: 302 - protein, 5 aa, synthetic construct
  REGION 1..5: note = computer generated peptide library sequence
GTYNL

SEQ ID NO: 303 - protein, 4 aa, synthetic construct
  REGION 1..4: note = computer generated peptide library sequence
NVSY

SEQ ID NO: 304 - protein, 4 aa, synthetic construct
  REGION 1..4: note = computer generated peptide library sequence
SMVN

SEQ ID NO: 305 - protein, 4 aa, synthetic construct
  REGION 1..4: note = computer generated peptide library sequence
ANYG

SEQ ID NO: 306 - protein, 4 aa, synthetic construct
  REGION 1..4: note = computer generated peptide library sequence
NVGT

SEQ ID NO: 307 - protein, 4 aa, synthetic construct
  REGION 1..4: note = computer generated peptide library sequence
SAYM

SEQ ID NO: 308 - protein, 4 aa, synthetic construct
  REGION 1..4: note = computer generated peptide library sequence
EKVT

SEQ ID NO: 309 - protein, 4 aa, synthetic construct
  REGION 1..4: note = computer generated peptide library sequence
TTPG

SEQ ID NO: 310 - protein, 4 aa, synthetic construct
  REGION 1..4: note = computer generated peptide library sequence
GVYS

SEQ ID NO: 311 - protein, 4 aa, synthetic construct
  REGION 1..4: note = computer generated peptide library sequence
SYVG

SEQ ID NO: 312 - protein, 4 aa, synthetic construct
  REGION 1..4: note = computer generated peptide library sequence
LQYN

SEQ ID NO: 313 - protein, 4 aa, synthetic construct
  REGION 1..4: note = computer generated peptide library sequence
DPAK

SEQ ID NO: 314 - protein, 4 aa, synthetic construct
  REGION 1..4: note = computer generated peptide library sequence
THFS

SEQ ID NO: 315 - protein, 4 aa, synthetic construct
  REGION 1..4: note = computer generated peptide library sequence
IGGV

SEQ ID NO: 316 - protein, 4 aa, synthetic construct
  REGION 1..4: note = computer generated peptide library sequence
SSWN

SEQ ID NO: 317 - protein, 4 aa, synthetic construct
  REGION 1..4: note = computer generated peptide library sequence
SVYV

SEQ ID NO: 318 - protein, 4 aa, synthetic construct
  REGION 1..4: note = computer generated peptide library sequence
TLNG

SEQ ID NO: 319 - protein, 4 aa, synthetic construct
  REGION 1..4: note = computer generated peptide library sequence
NTSN

SEQ ID NO: 320 - protein, 4 aa, synthetic construct
  REGION 1..4: note = computer generated peptide library sequence
VQYA

SEQ ID NO: 321 - protein, 4 aa, synthetic construct
  REGION 1..4: note = computer generated peptide library sequence
DQYR

SEQ ID NO: 322 - protein, 4 aa, synthetic construct
  REGION 1..4: note = computer generated peptide library sequence
MPVS

SEQ ID NO: 323 - protein, 4 aa, synthetic construct
  REGION 1..4: note = computer generated peptide library sequence
SAQA

SEQ ID NO: 324 - protein, 4 aa, synthetic construct
  REGION 1..4: note = computer generated peptide library sequence
MTVA

SEQ ID NO: 325 - protein, 4 aa, synthetic construct
  REGION 1..4: note = computer generated peptide library sequence
TVMG

SEQ ID NO: 326 - protein, 4 aa, synthetic construct
  REGION 1..4: note = computer generated peptide library sequence
FSSI

SEQ ID NO: 327 - protein, 4 aa, synthetic construct
  REGION 1..4: note = computer generated peptide library sequence
SLRL

SEQ ID NO: 328 - protein, 4 aa, synthetic construct
  REGION 1..4: note = computer generated peptide library sequence
SAMG

SEQ ID NO: 329 - protein, 4 aa, synthetic construct
  REGION 1..4: note = computer generated peptide library sequence
YIKL

SEQ ID NO: 330 - protein, 4 aa, synthetic construct
  REGION 1..4: note = computer generated peptide library sequence
LMTM

SEQ ID NO: 331 - protein, 4 aa, synthetic construct
  REGION 1..4: note = computer generated peptide library sequence
QVHL

SEQ ID NO: 332 - protein, 4 aa, synthetic construct
  REGION 1..4: note = computer generated peptide library sequence
YNSV

SEQ ID NO: 333 - protein, 4 aa, synthetic construct
  REGION 1..4: note = computer generated peptide library sequence
CVIS

SEQ ID NO: 334 - protein, 4 aa, synthetic construct
  REGION 1..4: note = computer generated peptide library sequence
RLDG

SEQ ID NO: 335 - protein, 4 aa, synthetic construct
  REGION 1..4: note = computer generated peptide library sequence
AIMV

SEQ ID NO: 336 - protein, 4 aa, synthetic construct
  REGION 1..4: note = computer generated peptide library sequence
GTTG

SEQ ID NO: 337 - protein, 4 aa, synthetic construct
  REGION 1..4: note = computer generated peptide library sequence
ASYT

SEQ ID NO: 338 - protein, 4 aa, synthetic construct
  REGION 1..4: note = computer generated peptide library sequence
LHVG

SEQ ID NO: 339 - protein, 4 aa, synthetic construct
  REGION 1..4: note = computer generated peptide library sequence
LQFA

SEQ ID NO: 340 - protein, 4 aa, synthetic construct
  REGION 1..4: note = computer generated peptide library sequence
VRGD

SEQ ID NO: 341 - protein, 4 aa, synthetic construct
  REGION 1..4: note = computer generated peptide library sequence
NVMI

SEQ ID NO: 342 - protein, 4 aa, synthetic construct
  REGION 1..4: note = computer generated peptide library sequence
SLYG

SEQ ID NO: 343 - protein, 4 aa, synthetic construct
  REGION 1..4: note = computer generated peptide library sequence
GTVG

SEQ ID NO: 344 - protein, 4 aa, synthetic construct
  REGION 1..4: note = computer generated peptide library sequence
FNSV

SEQ ID NO: 345 - protein, 4 aa, synthetic construct
  REGION 1..4: note = computer generated peptide library sequence
TRLG

SEQ ID NO: 346 - protein, 4 aa, synthetic construct
  REGION 1..4: note = computer generated peptide library sequence
LKVL

SEQ ID NO: 347 - protein, 4 aa, synthetic construct
  REGION 1..4: note = computer generated peptide library sequence
SIRV

SEQ ID NO: 348 - protein, 4 aa, synthetic construct
  REGION 1..4: note = computer generated peptide library sequence
KIQG

SEQ ID NO: 349 - protein, 4 aa, synthetic construct
  REGION 1..4: note = computer generated peptide library sequence
QILG

SEQ ID NO: 350 - protein, 4 aa, synthetic construct
  REGION 1..4: note = computer generated peptide library sequence
QRDA

SEQ ID NO: 351 - protein, 4 aa, synthetic construct
  REGION 1..4: note = computer generated peptide library sequence
EAVR

SEQ ID NO: 352 - protein, 4 aa, synthetic construct
  REGION 1..4: note = computer generated peptide library sequence
AITV

SEQ ID NO: 353 - protein, 4 aa, synthetic construct
  REGION 1..4: note = computer generated peptide library sequence
KESI

SEQ ID NO: 354 - protein, 4 aa, synthetic construct
  REGION 1..4: note = computer generated peptide library sequence
LMVN

SEQ ID NO: 355 - protein, 4 aa, synthetic construct
  REGION 1..4: note = computer generated peptide library sequence
INLS

SEQ ID NO: 356 - protein, 4 aa, synthetic construct
  REGION 1..4: note = computer generated peptide library sequence
GQVS

SEQ ID NO: 357 - protein, 4 aa, synthetic construct
  REGION 1..4: note = computer generated peptide library sequence
TSLL

SEQ ID NO: 358 - protein, 4 aa, synthetic construct
  REGION 1..4: note = computer generated peptide library sequence
SSTL

SEQ ID NO: 359 - protein, 4 aa, synthetic construct
  REGION 1..4: note = computer generated peptide library sequence
YEKF

SEQ ID NO: 360 - protein, 4 aa, synthetic construct
  REGION 1..4: note = computer generated peptide library sequence
DGKL

SEQ ID NO: 361 - protein, 4 aa, synthetic construct
  REGION 1..4: note = computer generated peptide library sequence
QVYS

SEQ ID NO: 362 - protein, 4 aa, synthetic construct
  REGION 1..4: note = computer generated peptide library sequence
QKEG

SEQ ID NO: 363 - protein, 4 aa, synthetic construct
  REGION 1..4: note = computer generated peptide library sequence
ARDM

SEQ ID NO: 364 - protein, 4 aa, synthetic construct
  REGION 1..4: note = computer generated peptide library sequence
DNFR

SEQ ID NO: 365 - protein, 4 aa, synthetic construct
  REGION 1..4: note = computer generated peptide library sequence
SHGL

SEQ ID NO: 366 - protein, 4 aa, synthetic construct
  REGION 1..4: note = computer generated peptide library sequence
VSVN

SEQ ID NO: 367 - protein, 4 aa, synthetic construct
  REGION 1..4: note = computer generated peptide library sequence
GLKD

SEQ ID NO: 368 - protein, 4 aa, synthetic construct
  REGION 1..4: note = computer generated peptide library sequence
QPVF

SEQ ID NO: 369 - protein, 4 aa, synthetic construct
  REGION 1..4: note = computer generated peptide library sequence
VYSM

SEQ ID NO: 370 - protein, 4 aa, synthetic construct
  REGION 1..4: note = computer generated peptide library sequence
VMAQ

SEQ ID NO: 371 - protein, 4 aa, synthetic construct
  REGION 1..4: note = computer generated peptide library sequence
FVGM

SEQ ID NO: 372 - protein, 4 aa, synthetic construct
  REGION 1..4: note = computer generated peptide library sequence
WSTP

SEQ ID NO: 373 - protein, 4 aa, synthetic construct
  REGION 1..4: note = computer generated peptide library sequence
SYPV

SEQ ID NO: 374 - protein, 4 aa, synthetic construct
  REGION 1..4: note = computer generated peptide library sequence
TTYS

SEQ ID NO: 375 - protein, 4 aa, synthetic construct
  REGION 1..4: note = computer generated peptide library sequence
TVTT

SEQ ID NO: 376 - protein, 4 aa, synthetic construct
  REGION 1..4: note = computer generated peptide library sequence
KDKT

SEQ ID NO: 377 - protein, 4 aa, synthetic construct
  REGION 1..4: note = computer generated peptide library sequence
YREL

SEQ ID NO: 378 - protein, 4 aa, synthetic construct
  REGION 1..4: note = computer generated peptide library sequence
LSHF

SEQ ID NO: 379 - protein, 4 aa, synthetic construct
  REGION 1..4: note = computer generated peptide library sequence
SPGT

SEQ ID NO: 380 - protein, 4 aa, synthetic construct
  REGION 1..4: note = computer generated peptide library sequence
LMGT

SEQ ID NO: 381 - protein, 4 aa, synthetic construct
  REGION 1..4: note = computer generated peptide library sequence
AASL

SEQ ID NO: 382 - protein, 4 aa, synthetic construct
  REGION 1..4: note = computer generated peptide library sequence
FSNN

SEQ ID NO: 383 - protein, 4 aa, synthetic construct
  REGION 1..4: note = computer generated peptide library sequence
QARL

SEQ ID NO: 384 - protein, 4 aa, synthetic construct
  REGION 1..4: note = computer generated peptide library sequence
YHIA

SEQ ID NO: 385 - protein, 4 aa, synthetic construct
  REGION 1..4: note = computer generated peptide library sequence
ARQD

SEQ ID NO: 386 - protein, 4 aa, synthetic construct
  REGION 1..4: note = computer generated peptide library sequence
VAYT

SEQ ID NO: 387 - protein, 4 aa, synthetic construct
  REGION 1..4: note = computer generated peptide library sequence
TPSY

SEQ ID NO: 388 - protein, 4 aa, synthetic construct
  REGION 1..4: note = computer generated peptide library sequence
MILH

SEQ ID NO: 389 - protein, 4 aa, synthetic construct
  REGION 1..4: note = computer generated peptide library sequence
LGNV

SEQ ID NO: 390 - protein, 4 aa, synthetic construct
  REGION 1..4: note = computer generated peptide library sequence
TSIS

SEQ ID NO: 391 - protein, 4 aa, synthetic construct
  REGION 1..4: note = computer generated peptide library sequence
TMVY

SEQ ID NO: 392 - protein, 4 aa, synthetic construct
  REGION 1..4: note = computer generated peptide library sequence
LVVG

SEQ ID NO: 393 - protein, 4 aa, synthetic construct
  REGION 1..4: note = computer generated peptide library sequence
SPLY

SEQ ID NO: 394 - protein, 4 aa, synthetic construct
  REGION 1..4: note = computer generated peptide library sequence
YKSE

SEQ ID NO: 395 - protein, 4 aa, synthetic construct
  REGION 1..4: note = computer generated peptide library sequence
FTRL

SEQ ID NO: 396 - protein, 4 aa, synthetic construct
  REGION 1..4: note = computer generated peptide library sequence
VSYN

SEQ ID NO: 397 - protein, 4 aa, synthetic construct
  REGION 1..4: note = computer generated peptide library sequence
ERTP

SEQ ID NO: 398 - protein, 4 aa, synthetic construct
  REGION 1..4: note = computer generated peptide library sequence
FRSE

SEQ ID NO: 399 - protein, 4 aa, synthetic construct
  REGION 1..4: note = computer generated peptide library sequence
NYTE

SEQ ID NO: 400 - protein, 4 aa, synthetic construct
  REGION 1..4: note = computer generated peptide library sequence
QTIN

SEQ ID NO: 401 - protein, 4 aa, synthetic construct
  REGION 1..4: note = computer generated peptide library sequence
DVHR

SEQ ID NO: 402 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTISQVNGRPRDLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 403 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 404 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 405 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 406 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 407 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 408 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 409 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 410 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 411 - protein, 50 aa, synthetic construct
  REGION 1..50: note = Recombinant AAV capsid protein
MAADGYLPDWLEDNLSEGIREWWALKPGAPQPKANQQHQDNARGLVLPGY

SEQ ID NO: 412 - protein, 49 aa, synthetic construct
  REGION 1..49: note = Recombinant AAV capsid protein
MSFVDHPPDWLEEVGEGLREFLGLEAGPPKPKPNQQHQDQARGLVLPGY

SEQ ID NO: 413 - protein, 34 aa, synthetic construct
  REGION 1..34: note = Recombinant AAV capsid protein
KYLGPGNGLDKGEPVNAADAAALEHDKAYDQQLK

SEQ ID NO: 414 - protein, 34 aa, synthetic construct
  REGION 1..34: note = Recombinant AAV capsid protein
NYLGPGNGLDRGEPVNRADEVAREHDISYNEQLE

SEQ ID NO: 415 - protein, 44 aa, synthetic construct
  REGION 1..44: note = Recombinant AAV capsid protein
AGDNPYLKYNHADAEFQERLKEDTSFGGNLGRAVFQAKKRLLEP

SEQ ID NO: 416 - protein, 44 aa, synthetic construct
  REGION 1..44: note = Recombinant AAV capsid protein
AGDNPYLKYNHADAEFQEKLADDTSFGGNLGKAVFQAKKRVLEP

SEQ ID NO: 417 - protein, 84 aa, synthetic construct
  REGION 1..84: note = Recombinant AAV capsid protein
LGLVEEAAKTAPGKKRPVEQSPQEPDSSAGIGKSGAQPAKKRLNFGQTGDTESVPDPQPIGEPPAAPSGVGSLTMASGGGAPVA

SEQ ID NO: 418 - protein, 75 aa, synthetic construct
  REGION 1..75: note = Recombinant AAV capsid protein
FGLVEEGAKTAPTGKRIDDHFPKRKKARTEEDSKPSTSSDAEAGPSGSQQLQIPAQPASSLGADTMSAGGGGPLG

SEQ ID NO: 419 - protein, 524 aa, synthetic construct
  REGION 1..524: note = Recombinant AAV capsid protein
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 420 - protein, 524 aa, synthetic construct
  REGION 1..524: note = Recombinant AAV capsid protein
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 421 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 422 - protein, 725 aa, synthetic construct
  REGION 1..725: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTSNYYKSNNVEFAVNTEGVYSEPRPIGTRYLTRNL

SEQ ID NO: 423 - protein, 725 aa, synthetic construct
  REGION 1..725: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEAAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 424 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 425 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRPL

SEQ ID NO: 426 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSFAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 427 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 428 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 429 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTSNYYKSNNVEFAVNTEGVYSEPRPIGTRYLTRNL

SEQ ID NO: 430 - protein, 726 aa, synthetic construct
  REGION 1..726: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 431 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 432 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSKMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRNL

SEQ ID NO: 433 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 434 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 435 - protein, 726 aa, synthetic construct; identical to SEQ ID NO: 431
  REGION 1..726: note = Recombinant AAV capsid protein

SEQ ID NO: 436 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 437 - protein, 726 aa, synthetic construct
  REGION 1..726: note = Recombinant AAV capsid protein
MSFVDHPPDWLEEVGEGLREFLGLEAGPPKPKPNQQHQDQARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 438 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 439 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 440 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 441 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 442 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 443 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 444 - protein, 735 aa, synthetic construct; identical to SEQ ID NO: 433
  REGION 1..735: note = Recombinant AAV capsid protein

SEQ ID NO: 445 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 446 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 447 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 448 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 449 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 450 - protein, 736 aa, synthetic construct
  REGION 1..736: note = Recombinant AAV capsid protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 451 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 452 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 453 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 454 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 455 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 456 - protein, 735 aa, synthetic construct
  REGION 1..735: note = Recombinant AAV capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 457 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 458 - protein, 727 aa, synthetic construct; identical to SEQ ID NO: 438
  REGION 1..727: note = Recombinant AAV capsid protein

SEQ ID NO: 459 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 460 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 461 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 462 - protein, 727 aa, synthetic construct
  REGION 1..727: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 463 - protein, 502 aa, synthetic construct
  REGION 1..502: note = Recombinant AAV capsid protein
  VARIANT 218..226: note = Xaa is any amino acid
  VARIANT 263..268: note = Xaa is any amino acid
  VARIANT 315..319: note = Xaa is any amino acid
  VARIANT 352..355: note = Xaa is any amino acid
LGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIXXXXXXXXXLKFSVAGPSNMAVQGRNYIPGPSYRQQRVSTTVTQNXXXXXXWPGASSWALNGRNSLMNPGPAMASHKEGEDRFFPLSGSLIFGKQGTXXXXXDADKVMITNEEEIKTTNPVATESYGQVATNHQXXXXQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 464 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 465 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSMVNQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 466 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVGTQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 467 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 468 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 469 - protein, 9 aa, Adeno-associated dependoparvovirus A
  source 1..9: note = Adeno-associated virus serotype 9
NGSGQNQQT

SEQ ID NO: 470 - protein, 18 aa, Adeno-associated dependoparvovirus A
  source 1..18: note = Adeno-associated virus serotype 9
RVSTTVTQNNNSEFAWPG

SEQ ID NO: 471 - protein, 14 aa, Adeno-associated dependoparvovirus A
  source 1..14: note = Adeno-associated virus serotype 9
KQGTGRDNVDADKV

SEQ ID NO: 472 - protein, 13 aa, Adeno-associated dependoparvovirus A
  source 1..13: note = Adeno-associated virus serotype 9
ATNHQSAQAQAQT

SEQ ID NO: 473 - protein, 9 aa, synthetic construct
  REGION 1..9: note = VR-IV protein motif
  VARIANT 1: note = Xaa is Gly or Val
  VARIANT 2: note = Xaa is Tyr or Ile
  VARIANT 3: note = Xaa is His or Ile
  VARIANT 5: note = Xaa is Ser or Ile
  VARIANT 7: note = Xaa is Ala or Val
XXXKXGXAQ

SEQ ID NO: 474-481 - skipped (8 entries)

SEQ ID NO: 482 - protein, 745 aa, synthetic construct
  REGION 1..745: note = Recombinant AAV capsid protein
MAADGYLPDWLEDNLSEGIREWWALKPGSPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQSASNVNAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 483 - protein, 745 aa, synthetic construct
  REGION 1..745: note = Recombinant AAV capsid protein
MAADGYLPDWLEDNLSEGIREWWALKPGSPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAASPTTTAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 484 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRTSTTDTQNNNSEFAWPAASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 485 - protein, 736 aa, synthetic construct
  REGION 1..736: note = Recombinant AAV capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQVVSGDDTQDAGLELNIAGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 486 - protein, 599 aa, Adeno-associated dependoparvovirus A
  source 1..599: note = Adeno-associated virus serotype 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 487 - protein, 534 aa, Adeno-associated dependoparvovirus A
  source 1..534: note = Adeno-associated virus serotype 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 488 - protein, 736 aa, synthetic construct
  REGION 1..736: note = Recombinant AAV capsid protein ZC377
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 489 - protein, 736 aa, synthetic construct
  REGION 1..736: note = Recombinant AAV capsid protein ZC378
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 490 - protein, 736 aa, synthetic construct
  REGION 1..736: note = Recombinant AAV capsid protein ZC379
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 491 - protein, 736 aa, synthetic construct
  REGION 1..736: note = Recombinant AAV capsid protein ZC380
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGLNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 492 - protein, 736 aa, synthetic construct
  REGION 1..736: note = Recombinant AAV capsid protein ZC381
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIANDNKLIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 493 - protein, 736 aa, synthetic construct
  REGION 1..736: note = Recombinant AAV capsid protein ZC382
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIVNDNKVIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 494 - protein, 736 aa, synthetic construct
  REGION 1..736: note = Recombinant AAV capsid protein ZC383
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNHQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 495 - protein, 736 aa, synthetic construct
  REGION 1..736: note = Recombinant AAV capsid protein ZC384
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIANDNKVIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 496 - protein, 736 aa, synthetic construct
  REGION 1..736: note = Recombinant AAV capsid protein ZC385
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSFQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 497 - protein, 736 aa, synthetic construct
  REGION 1..736: note = Recombinant AAV capsid protein ZC386
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 498 - protein, 736 aa, synthetic construct
  REGION 1..736: note = Recombinant AAV capsid protein ZC387
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVDSLRIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 499 - protein, 736 aa, synthetic construct
  REGION 1..736: note = Recombinant AAV capsid protein ZC388
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 500 - protein, 736 aa, synthetic construct
  REGION 1..736: note = Recombinant AAV capsid protein ZC389
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTGTSIIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 501 - protein, 736 aa, synthetic construct
  REGION 1..736: note = Recombinant AAV capsid protein ZC390
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSNFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 502 - protein, 736 aa, synthetic construct
  REGION 1..736: note = Recombinant AAV capsid protein ZC391
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCTLNSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 503 - protein, 736 aa, synthetic construct
  REGION 1..736: note = Recombinant AAV capsid protein ZC392
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQKPGSQIQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 504 - protein, 736 aa, synthetic construct
  REGION 1..736: note = Recombinant AAV capsid protein ZC393
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNMNRVNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 505 - protein, 736 aa, synthetic construct
  REGION 1..736: note = Recombinant AAV capsid protein ZC394
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 506 - protein, 736 aa, synthetic construct
  REGION 1..736: note = Recombinant AAV capsid protein ZC395
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSNSVQSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 507 - protein, 738 aa, synthetic construct
  REGION 1..738: note = Recombinant AAV capsid protein ZC396
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 508 - protein, 736 aa, synthetic construct
  REGION 1..736: note = Recombinant AAV capsid protein ZC397
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSKVFDAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 509 - protein, 738 aa, synthetic construct
  REGION 1..738: note = Recombinant AAV capsid protein ZC398
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAITQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 510 - protein, 736 aa, synthetic construct
  REGION 1..736: note = Recombinant AAV capsid protein ZC399
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSTFQGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 511 - protein, 736 aa, synthetic construct
  REGION 1..736: note = Recombinant AAV capsid protein ZC400
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSIQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 512 - protein, 736 aa, synthetic construct
  REGION 1..736: note = Recombinant AAV capsid protein ZC401
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 513 - protein, 736 aa, synthetic construct
  REGION 1..736: note = Recombinant AAV capsid protein ZC402
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 514 - protein, 736 aa, synthetic construct
  REGION 1..736: note = Recombinant AAV capsid protein ZC403
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVALNKQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 515 - protein, 736 aa, synthetic construct
  REGION 1..736: note = Recombinant AAV capsid protein ZC404
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHENTVSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 516 - protein, 736 aa, synthetic construct
  REGION 1..736: note = Recombinant AAV capsid protein ZC405
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSSFTSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 517 - protein, 736 aa, synthetic construct
  REGION 1..736: note = Recombinant AAV capsid protein ZC406
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHPSIHQGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 518 - protein, 736 aa, synthetic construct
  REGION 1..736: note = Recombinant AAV capsid protein ZC407
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTTNFRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 519 - protein, 738 aa, synthetic construct
  REGION 1..738: note = Recombinant AAV capsid protein ZC408
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQHYSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 520 - protein, 736 aa, synthetic construct
  REGION 1..736: note = Recombinant AAV capsid protein ZC409
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 521 - protein, 736 aa, synthetic construct
  REGION 1..736: note = Recombinant AAV capsid protein ZC410
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 522 - protein, 736 aa, synthetic construct
  REGION 1..736: note = Recombinant AAV capsid protein ZC411
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHAGNYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 523 - protein, 736 aa, synthetic construct
  REGION 1..736: note = Recombinant AAV capsid protein ZC412
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAEVQQSSMSQAQTDWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 524 - protein, 736 aa, synthetic construct
  REGION 1..736: note = Recombinant AAV capsid protein ZC413
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAANVQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 525 - protein, 736 aa, synthetic construct
  REGION 1..736: note = Recombinant AAV capsid protein ZC414
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNYQQAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 526 - protein, 737 aa, synthetic construct
  REGION 1..737: note = Recombinant AAV capsid protein ZC415
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSVQGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 527 - protein, 736 aa, synthetic construct
  REGION 1..736: note = Recombinant AAV capsid protein ZC416
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHGSILTHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 528 - protein, 736 aa, synthetic construct
  REGION 1..736: note = Recombinant AAV capsid protein ZC417
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLFSKNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 529 - protein, 736 aa, synthetic construct
  REGION 1..736: note = Recombinant AAV capsid protein ZC418
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAANMQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 530 - protein, 736 aa, synthetic construct
  REGION 1..736: note = Recombinant AAV capsid protein ZC419
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNQQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 531 - protein, 738 aa, synthetic construct
  REGION 1..738: note = Recombinant AAV capsid protein ZC420
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNTYHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 532 - protein, 736 aa, synthetic construct
  REGION 1..736: note = Recombinant AAV capsid protein ZC421
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCDPLHIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 533 - protein, 736 aa, synthetic construct
  REGION 1..736: note = Recombinant AAV capsid protein ZC422
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSVISIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 534 - protein, 736 aa, synthetic construct
  REGION 1..736: note = Recombinant AAV capsid protein ZC423
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 535 - protein, 738 aa, synthetic construct
  REGION 1..738: note = Recombinant AAV capsid protein ZC424
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQVTSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 536 - protein, 736 aa, synthetic construct
  REGION 1..736: note = Recombinant AAV capsid protein ZC425
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHSRVEIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 537 - protein, 736 aa, synthetic construct
  REGION 1..736: note = Recombinant AAV capsid protein ZC426
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSFTWTAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 538 - protein, 738 aa, synthetic construct
  REGION 1..738: note = Recombinant AAV capsid protein ZC427
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 539 - protein, 736 aa, synthetic construct
  REGION 1..736: note = Recombinant AAV capsid protein ZC428
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 540 - protein, 736 aa, synthetic construct
  REGION 1..736: note = Recombinant AAV capsid protein ZC429
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 541 - protein, 738 aa, synthetic construct
  REGION 1..738: note = Recombinant AAV capsid protein ZC430
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQAISAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 542 - protein, 736 aa, synthetic construct
  REGION 1..736: note = Recombinant AAV capsid protein ZC431
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSVVYNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 543 - protein, 738 aa, synthetic construct
  REGION 1..738: note = Recombinant AAV capsid protein ZC432
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 544 - protein, 736 aa, synthetic construct
  REGION 1..736: note = Recombinant AAV capsid protein ZC433
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHETSRLNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 545 - protein, 736 aa, synthetic construct
  REGION 1..736: note = Recombinant AAV capsid protein ZC434
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAFNWQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 546 - protein, 736 aa, synthetic construct
  REGION 1..736: note = Recombinant AAV capsid protein ZC435
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTVMLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 547 - protein, 736 aa, synthetic construct
  REGION 1..736: note = Recombinant AAV capsid protein ZC436
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESSMLNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 548 - protein, 736 aa, synthetic construct
  REGION 1..736: note = Recombinant AAV capsid protein ZC437
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHASITSSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 549 - protein, 736 aa, synthetic construct
  REGION 1..736: note = Recombinant AAV capsid protein ZC438
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVARNEQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 550 - protein, 736 aa, synthetic construct
  REGION 1..736: note = Recombinant AAV capsid protein ZC439
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHANLYQMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 551 - protein, 736 aa, synthetic construct
  REGION 1..736: note = Recombinant AAV capsid protein ZC440
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQFATAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 552 - protein, 738 aa, synthetic construct
  REGION 1..738: note = Recombinant AAV capsid protein ZC441
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNFNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 553 - protein, 736 aa, synthetic construct
  REGION 1..736: note = Recombinant AAV capsid protein ZC442
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMSHQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 554 - protein, 738 aa, synthetic construct
  REGION 1..738: note = Recombinant AAV capsid protein ZC443
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQWMSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 555 - protein, 737 aa, synthetic construct
  REGION 1..737: note = Recombinant AAV capsid protein ZC444
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 556 - protein, 736 aa, synthetic construct
  REGION 1..736: note = Recombinant AAV capsid protein ZC445
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 557 - protein, 736 aa, synthetic construct
  REGION 1..736: note = Recombinant AAV capsid protein ZC446
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTTKTMFAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 558 - protein, 736 aa, synthetic construct
  REGION 1..736: note = Recombinant AAV capsid protein ZC447
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIIYSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 559 - protein, 736 aa, synthetic construct
  REGION 1..736: note = Recombinant AAV capsid protein ZC448
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMLLKSNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 560 - protein, 736 aa, synthetic construct
  REGION 1..736: note = Recombinant AAV capsid protein ZC449
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESMQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 561 - protein, 738 aa, synthetic construct
  REGION 1..738: note = Recombinant AAV capsid protein ZC450
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMLSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 562 - protein, 736 aa, synthetic construct
  REGION 1..736: note = Recombinant AAV capsid protein ZC451
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSGRDSYAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 563 - protein, 736 aa, synthetic construct
  REGION 1..736: note = Recombinant AAV capsid protein ZC452
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHINVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 564 - protein, 736 aa, synthetic construct
  REGION 1..736: note = Recombinant AAV capsid protein ZC453
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSNQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 565 - protein, 736 aa, synthetic construct
  REGION 1..736: note = Recombinant AAV capsid protein ZC454
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTKLAIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 566 - protein, 736 aa, synthetic construct
  REGION 1..736: note = Recombinant AAV capsid protein ZC455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSSYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 567 - protein, 738 aa, synthetic construct
  REGION 1..738: note = Recombinant AAV capsid protein ZC456
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNATHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 568 - protein, 736 aa, synthetic construct
  REGION 1..736: note = Recombinant AAV capsid protein ZC457
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLRDNISAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 569 - protein, 736 aa, synthetic construct
  REGION 1..736: note = Recombinant AAV capsid protein ZC458
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSFSVGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 570 - protein, 736 aa, synthetic construct
  REGION 1..736: note = Recombinant AAV capsid protein ZC459
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVNRNLSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 571 - protein, 736 aa, synthetic construct
  REGION 1..736: note = Recombinant AAV capsid protein ZC460
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHNPSINAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 572 - protein, 736 aa, synthetic construct
  REGION 1..736: note = Recombinant AAV capsid protein ZC461
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDARAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 573 - protein, 736 aa, synthetic construct
  REGION 1..736: note = Recombinant AAV capsid protein ZC462
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNDQRAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 574 - protein, 736 aa, synthetic construct
  REGION 1..736: note = Recombinant AAV capsid protein ZC463
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNVQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 575 - protein, 736 aa, synthetic construct
  REGION 1..736: note = Recombinant AAV capsid protein ZC464
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAPNRQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 576 - protein, 736 aa, synthetic construct
  REGION 1..736: note = Recombinant AAV capsid protein ZC465
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 577 - protein, 736 aa, synthetic construct
  REGION 1..736: note = Recombinant AAV capsid protein ZC466
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 578 - protein, 736 aa, synthetic construct
  REGION 1..736: note = Recombinant AAV capsid protein ZC467
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNRNGLLAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 579 - protein, 736 aa, synthetic construct
  REGION 1..736: note = Recombinant AAV capsid protein ZC468
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESTSVRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 580 - protein, 736 aa, synthetic construct
  REGION 1..736: note = Recombinant AAV capsid protein ZC469
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNIRTEMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 581 - protein, 736 aa, synthetic construct
  REGION 1..736: note = Recombinant AAV capsid protein ZC470
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 582 - protein, 736 aa, synthetic construct
  REGION 1..736: note = Recombinant AAV capsid protein ZC471
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHSWQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 583 - protein, 736 aa, synthetic construct
  REGION 1..736: note = Recombinant AAV capsid protein ZC472
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTKSLIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 584 - protein, 736 aa, synthetic construct
  REGION 1..736: note = Recombinant AAV capsid protein ZC473
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQKLLVNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 585 - protein, 736 aa, synthetic construct
  REGION 1..736: note = Recombinant AAV capsid protein ZC474
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSVSSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 586 - protein, 736 aa, synthetic construct
  REGION 1..736: note = Recombinant AAV capsid protein ZC475
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSNLYGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 587 - protein, 736 aa, synthetic construct
  REGION 1..736: note = Recombinant AAV capsid protein ZC476
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQMAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 588 - protein, 736 aa, synthetic construct
  REGION 1..736: note = Recombinant AAV capsid protein ZC477
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDIIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 589 - protein, 736 aa, synthetic construct
  REGION 1..736: note = Recombinant AAV capsid protein ZC478
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 590 - protein, 7 aa, synthetic construct
  REGION 1..7: note = Recombinant AAV capsid protein ZC377 VR-IV
KGSGQNQ

SEQ ID NO: 591 - protein, 7 aa, synthetic construct
  REGION 1..7: note = Recombinant AAV capsid protein ZC378 VR-IV
NASGQNQ

SEQ ID NO: 592 - protein, 7 aa, synthetic construct
  REGION 1..7: note = Recombinant AAV capsid protein ZC379 VR-IV
NGTGQNQ

SEQ ID NO: 593 - protein, 7 aa, synthetic construct
  REGION 1..7: note = Recombinant AAV capsid protein ZC380 VR-IV
NGSGLNQ

SEQ ID NO: 594 - protein, 7 aa, synthetic construct
  REGION 1..7: note = Recombinant AAV capsid protein ZC381 VR-IV
ANDNKLI

SEQ ID NO: 595 - protein, 7 aa, synthetic construct
  REGION 1..7: note = Recombinant AAV capsid protein ZC382 VR-IV
VNDNKVI

SEQ ID NO: 596 - protein, 7 aa, synthetic construct
  REGION 1..7: note = Recombinant AAV capsid protein ZC383 VR-IV
NGSGQNH

SEQ ID NO: 597 - protein, 7 aa, synthetic construct
  REGION 1..7: note = Recombinant AAV capsid protein ZC384 VR-IV
ANDNKVI

SEQ ID NO: 598 - protein, 14 aa, synthetic construct
  REGION 1..14: note = Recombinant AAV capsid protein VR-VIII
ATNHQANYGQAQTG

SEQ ID NO: 599 - protein, 14 aa, synthetic construct
  REGION 1..14: note = Recombinant AAV capsid protein ZC385 VR-VIII
ATNHTSFQAQAQTG

SEQ ID NO: 600 - protein, 14 aa, synthetic construct
  REGION 1..14: note = Recombinant AAV capsid protein ZC386 VR-VIII
ATNHCSAQAQAQTG

SEQ ID NO: 601 - protein, 14 aa, synthetic construct
  REGION 1..14: note = Recombinant AAV capsid protein ZC387 VR-VIII
ATNHVDSLRIAQTG

SEQ ID NO: 602 - protein, 14 aa, synthetic construct
  REGION 1..14: note = Recombinant AAV capsid protein ZC388 VR-VIII
ATNRQTAQAQAQTG

SEQ ID NO: 603 - protein, 14 aa, synthetic construct
  REGION 1..14: note = Recombinant AAV capsid protein ZC389 VR-VIII
ATNHTGTSIIAQTG

SEQ ID NO: 604 - protein, 14 aa, synthetic construct
  REGION 1..14: note = Recombinant AAV capsid protein ZC390 VR-VIII
ATNHLSNFNSAQTG

SEQ ID NO: 605 - protein, 14 aa, synthetic construct
  REGION 1..14: note = Recombinant AAV capsid protein ZC391 VR-VIII
ATNHCTLNSIAQTG

SEQ ID NO: 606 - protein, 14 aa, synthetic construct
  REGION 1..14: note = Recombinant AAV capsid protein ZC392 VR-VIII
ADVQQKPGSQIQTQ

SEQ ID NO: 607 - protein, 14 aa, synthetic construct
  REGION 1..14: note = Recombinant AAV capsid protein ZC393 VR-VIII
ATNHNMNRVNAQTG

SEQ ID NO: 608 - protein, 14 aa, synthetic construct
  REGION 1..14: note = Recombinant AAV capsid protein ZC394 VR-VIII
ATNHNNVISGAQTG

SEQ ID NO: 609 - protein, 14 aa, synthetic construct
  REGION 1..14: note = Recombinant AAV capsid protein ZC395 VR-VIII
ATNHSNSVQSAQTG

SEQ ID NO: 610 - protein, 16 aa, synthetic construct
  REGION 1..16: note = Recombinant AAV capsid protein ZC396 VR-VIII
ATNHQSPIAQAQAQTG

SEQ ID NO: 611 - protein, 14 aa, synthetic construct
  REGION 1..14: note = Recombinant AAV capsid protein ZC397 VR-VIII
ATNHLSKVFDAQTG

SEQ ID NO: 612 - protein, 16 aa, synthetic construct
  REGION 1..16: note = Recombinant AAV capsid protein ZC398 VR-VIII
ATNHQSAITQAQAQTG

SEQ ID NO: 613 - protein, 14 aa, synthetic construct
  REGION 1..14: note = Recombinant AAV capsid protein ZC399 VR-VIII
ATNHSSTFQGAQTG

SEQ ID NO: 614 - protein, 14 aa, synthetic construct
  REGION 1..14: note = Recombinant AAV capsid protein ZC400 VR-VIII
ATNHNSIQAQAQTG

SEQ ID NO: 615 - protein, 14 aa, synthetic construct
  REGION 1..14: note = Recombinant AAV capsid protein ZC401 VR-VIII
ATNHMMTTARAQTG

SEQ ID NO: 616 - protein, 14 aa, synthetic construct
  REGION 1..14: note = Recombinant AAV capsid protein ZC402 VR-VIII
ATNHQGAYAQAQTG

SEQ ID NO: 617 - protein, 14 aa, synthetic construct
  REGION 1..14: note = Recombinant AAV capsid protein ZC403 VR-VIII
ALNKQSAQAQAQTG

SEQ ID NO: 618 - protein, 14 aa, synthetic construct
  REGION 1..14: note = Recombinant AAV capsid protein ZC404 VR-VIII
ATNHENTVSIAQTG

SEQ ID NO: 619 - protein, 14 aa, synthetic construct
  REGION 1..14: note = Recombinant AAV capsid protein ZC405 VR-VIII
ATNHVSSFTSAQTG

SEQ ID NO: 620 - protein, 14 aa, synthetic construct
  REGION 1..14: note = Recombinant AAV capsid protein ZC406 VR-VIII
ATNHPSIHQGAQTG

SEQ ID NO: 621 - protein, 14 aa, synthetic construct
  REGION 1..14: note = Recombinant AAV capsid protein ZC407 VR-VIII
ATNHSTTNFRAQTG

SEQ ID NO: 622 - protein, 16 aa, synthetic construct
  REGION 1..16: note = Recombinant AAV capsid protein ZC408 VR-VIII
ATNHQHYSAQAQAQTG

SEQ ID NO: 623 - protein, 14 aa, synthetic construct
  REGION 1..14: note = Recombinant AAV capsid protein ZC409 VR-VIII
ATNKQTAQAQAQTG

SEQ ID NO: 624 - protein, 14 aa, synthetic construct
  REGION 1..14: note = Recombinant AAV capsid protein ZC410 VR-VIII
ATNHSSIFNSAQTG

SEQ ID NO: 625 - protein, 14 aa, synthetic construct
  REGION 1..14: note = Recombinant AAV capsid protein ZC411 VR-VIII
ATNHAGNYNNAQTG

SEQ ID NO: 626 - protein, 14 aa, synthetic construct
  REGION 1..14: note = Recombinant AAV capsid protein ZC412 VR-VIII
AEVQQSSMSQAQTD

SEQ ID NO: 627 - protein, 14 aa, synthetic construct
  REGION 1..14: note = Recombinant AAV capsid protein ZC413 VR-VIII
AANVQSAQAQAQTG

SEQ ID NO: 628 - protein, 14 aa, synthetic construct
  REGION 1..14: note = Recombinant AAV capsid protein ZC414 VR-VIII
ATNYQQAQAQAQTG

SEQ ID NO: 629 - protein, 15 aa, synthetic construct
  REGION 1..15: note = Recombinant AAV capsid protein ZC415 VR-VIII
ATNHQSVQGAQAQTG

SEQ ID NO: 630 - protein, 14 aa, synthetic construct
  REGION 1..14: note = Recombinant AAV capsid protein ZC416 VR-VIII
ATNHGSILTHAQTG

SEQ ID NO: 631 - protein, 14 aa, synthetic construct
  REGION 1..14: note = Recombinant AAV capsid protein ZC417 VR-VIII
ATNHQLFSKNAQTG

SEQ ID NO: 632 - protein, 14 aa, synthetic construct
  REGION 1..14: note = Recombinant AAV capsid protein ZC418 VR-VIII
AANMQSAQAQAQTG

SEQ ID NO: 633 - protein, 14 aa, synthetic construct
  REGION 1..14: note = Recombinant AAV capsid protein ZC419 VR-VIII
ATNQQIAQAQAQTG

SEQ ID NO: 634 - protein, 16 aa, synthetic construct
  REGION 1..16: note = Recombinant AAV capsid protein ZC420 VR-VIII
ATNTYHQSAQAQAQTG

SEQ ID NO: 635 - protein, 14 aa, synthetic construct
  REGION 1..14: note = Recombinant AAV capsid protein ZC421 VR-VIII
ATNHCDPLHIAQTG

SEQ ID NO: 636 - protein, 14 aa, synthetic construct
  REGION 1..14: note = Recombinant AAV capsid protein ZC422 VR-VIII
ATNHTSVISIAQTG

SEQ ID NO: 637 - protein, 14 aa, synthetic construct
  REGION 1..14: note = Recombinant AAV capsid protein ZC423 VR-VIII
ATNHQLASAQAQTG

SEQ ID NO: 638 - protein, 16 aa, synthetic construct
  REGION 1..16: note = Recombinant AAV capsid protein ZC424 VR-VIII
ATNHQVTSAQAQAQTG

SEQ ID NO: 639 - protein, 14 aa, synthetic construct
  REGION 1..14: note = Recombinant AAV capsid protein ZC425 VR-VIII
ATNHHSRVEIAQTG

SEQ ID NO: 640 - protein, 14 aa, synthetic construct
  REGION 1..14: note = Recombinant AAV capsid protein ZC426 VR-VIII
ATNHTSFTWTAQTG

SEQ ID NO: 641 - protein, 16 aa, synthetic construct
  REGION 1..16: note = Recombinant AAV capsid protein ZC427 VR-VIII
ATNHQSAPTQAQAQTG

SEQ ID NO: 642 - protein, 14 aa, synthetic construct
  REGION 1..14: note = Recombinant AAV capsid protein ZC428 VR-VIII
ATNHNSTYLGAQTG

SEQ ID NO: 643 - protein, 14 aa, synthetic construct
  REGION 1..14: note = Recombinant AAV capsid protein ZC429 VR-VIII
ATNHQIAQAQAQTG

SEQ ID NO: 644 - protein, 16 aa, synthetic construct
  REGION 1..16: note = Recombinant AAV capsid protein ZC430 VR-VIII
ATNHQAISAQAQAQTG

SEQ ID NO: 645 - protein, 14 aa, synthetic construct
  REGION 1..14: note = Recombinant AAV capsid protein ZC431 VR-VIII
ATNHLSVVYNAQTG

SEQ ID NO: 646 - protein, 16 aa, synthetic construct
  REGION 1..16: note = Recombinant AAV capsid protein ZC432 VR-VIII
ATNHMHQSAQAQAQTG

SEQ ID NO: 647 - protein, 14 aa, synthetic construct
  REGION 1..14: note = Recombinant AAV capsid protein ZC433 VR-VIII
ATNHETSRLNAQTG

SEQ ID NO: 648 - protein, 14 aa, synthetic construct
  REGION 1..14: note = Recombinant AAV capsid protein ZC434 VR-VIII
AFNWQSAQAQAQTG

SEQ ID NO: 649 - protein, 14 aa, synthetic construct
  REGION 1..14: note = Recombinant AAV capsid protein ZC435 VR-VIII
ATNHNTVMLGAQTG

SEQ ID NO: 650 - protein, 14 aa, synthetic construct
  REGION 1..14: note = Recombinant AAV capsid protein ZC436 VR-VIII
ATNHESSMLNAQTG

SEQ ID NO: 651 - protein, 14 aa, synthetic construct
  REGION 1..14: note = Recombinant AAV capsid protein ZC437 VR-VIII
ATNHASITSSAQTG

SEQ ID NO: 652 - protein, 14 aa, synthetic construct
  REGION 1..14: note = Recombinant AAV capsid protein ZC438 VR-VIII
ARNEQSAQAQAQTG

SEQ ID NO: 653 - protein, 14 aa, synthetic construct
  REGION 1..14: note = Recombinant AAV capsid protein ZC439 VR-VIII
ATNHANLYQMAQTG

SEQ ID NO: 654 - protein, 14 aa, synthetic construct
  REGION 1..14: note = Recombinant AAV capsid protein ZC440 VR-VIII
ATNHQFATAQAQTG

SEQ ID NO: 655 - protein, 16 aa, synthetic construct
  REGION 1..16: note = Recombinant AAV capsid protein ZC441 VR-VIII
ATNFNHQSAQAQAQTG

SEQ ID NO: 656 - protein, 14 aa, synthetic construct
  REGION 1..14: note = Recombinant AAV capsid protein ZC442 VR-VIII
ATNHMSHQAQAQTG

SEQ ID NO: 657 - protein, 16 aa, synthetic construct
  REGION 1..16: note = Recombinant AAV capsid protein ZC443 VR-VIII
ATNHQWMSAQAQAQTG

SEQ ID NO: 658 - protein, 15 aa, synthetic construct
  REGION 1..15: note = Recombinant AAV capsid protein ZC444 VR-VIII
ATNHQSGQQAQAQTG

SEQ ID NO: 659 - protein, 14 aa, synthetic construct
  REGION 1..14: note = Recombinant AAV capsid protein ZC445 VR-VIII
ATNHSSAQAQAQTG

SEQ ID NO: 660 - protein, 14 aa, synthetic construct
  REGION 1..14: note = Recombinant AAV capsid protein ZC446 VR-VIII
ATNHTTKTMFAQTG

SEQ ID NO: 661 - protein, 14 aa, synthetic construct
  REGION 1..14: note = Recombinant AAV capsid protein ZC447 VR-VIII
ATNHSSIIYSAQTG

SEQ ID NO: 662 - protein, 14 aa, synthetic construct
  REGION 1..14: note = Recombinant AAV capsid protein ZC448 VR-VIII
ATNHMLLKSNAQTG

SEQ ID NO: 663 - protein, 14 aa, synthetic construct
  REGION 1..14: note = Recombinant AAV capsid protein ZC449 VR-VIII
ATNHESMQAQAQTG

SEQ ID NO: 664 - protein, 16 aa, synthetic construct
  REGION 1..16: note = Recombinant AAV capsid protein ZC450 VR-VIII
ATNHQMLSAQAQAQTG

SEQ ID NO: 665 - protein, 14 aa, synthetic construct
  REGION 1..14: note = Recombinant AAV capsid protein ZC451 VR-VIII
ATNHSGRDSYAQTG

SEQ ID NO: 666 - protein, 14 aa, synthetic construct
  REGION 1..14: note = Recombinant AAV capsid protein ZC452 VR-VIII
ATNHINVISGAQTG

SEQ ID NO: 667 - protein, 14 aa, synthetic construct
  REGION 1..14: note = Recombinant AAV capsid protein ZC453 VR-VIII
ATNHVSNQAQAQTG

SEQ ID NO: 668 - protein, 14 aa, synthetic construct
  REGION 1..14: note = Recombinant AAV capsid protein ZC454 VR-VIII
ATNHNTKLAIAQTG

SEQ ID NO: 669 - protein, 14 aa, synthetic construct
  REGION 1..14: note = Recombinant AAV capsid protein ZC455 VR-VIII
ATNHSSSYNNAQTG

SEQ ID NO: 670 - protein, 16 aa, synthetic construct
  REGION 1..16: note = Recombinant AAV capsid protein ZC456 VR-VIII
ATNATHQSAQAQAQTG

SEQ ID NO: 671 - protein, 14 aa, synthetic construct
  REGION 1..14: note = Recombinant AAV capsid protein ZC457 VR-VIII
ATNHLRDNISAQTG

SEQ ID NO: 672 - protein, 14 aa, synthetic construct
  REGION 1..14: note = Recombinant AAV capsid protein ZC458 VR-VIII
ATNHSSFSVGAQTG

SEQ ID NO: 673 - protein, 14 aa, synthetic construct
  REGION 1..14: note = Recombinant AAV capsid protein ZC459 VR-VIII
ATNHVNRNLSAQTG

SEQ ID NO: 674 - protein, 14 aa, synthetic construct
  REGION 1..14: note = Recombinant AAV capsid protein ZC460 VR-VIII
ATNHHNPSINAQTG

SEQ ID NO: 675 - protein, 14 aa, synthetic construct
  REGION 1..14: note = Recombinant AAV capsid protein ZC461 VR-VIII
ATNHQDARAQAQTG

SEQ ID NO: 676 - protein, 14 aa, synthetic construct
  REGION 1..14: note = Recombinant AAV capsid protein ZC462 VR-VIII
ATNDQRAQAQAQTG

SEQ ID NO: 677 - protein, 14 aa, synthetic construct
  REGION 1..14: note = Recombinant AAV capsid protein ZC463 VR-VIII
ATNVQTAQAQAQTG

SEQ ID NO: 678 - protein, 14 aa, synthetic construct
  REGION 1..14: note = Recombinant AAV capsid protein ZC464 VR-VIII
APNRQSAQAQAQTG

SEQ ID NO: 679 - protein, 14 aa, synthetic construct
  REGION 1..14: note = Recombinant AAV capsid protein ZC465 VR-VIII
ATNRQIAQAQAQTG

SEQ ID NO: 680 - protein, 14 aa, synthetic construct
  REGION 1..14: note = Recombinant AAV capsid protein ZC466 VR-VIII
ATNHEDNIRRAQTG

SEQ ID NO: 681 - protein, 14 aa, synthetic construct
  REGION 1..14: note = Recombinant AAV capsid protein ZC467 VR-VIII
ATNHNRNGLLAQTG

SEQ ID NO: 682 - protein, 14 aa, synthetic construct
  REGION 1..14: note = Recombinant AAV capsid protein ZC468 VR-VIII
ATNHESTSVRAQTG

SEQ ID NO: 683 - protein, 14 aa, synthetic construct
  REGION 1..14: note = Recombinant AAV capsid protein ZC469 VR-VIII
ATNHNIRTEMAQTG

SEQ ID NO: 684 - protein, 14 aa, synthetic construct
  REGION 1..14: note = Recombinant AAV capsid protein ZC470 VR-VIII
ATNHQTLFNSAQTG

SEQ ID NO: 685 - protein, 14 aa, synthetic construct
  REGION 1..14: note = Recombinant AAV capsid protein ZC471 VR-VIII
ATNHHSWQAQAQTG

SEQ ID NO: 686 - protein, 14 aa, synthetic construct
  REGION 1..14: note = Recombinant AAV capsid protein ZC472 VR-VIII
ATNHSTKSLIAQTG

SEQ ID NO: 687 - protein, 14 aa, synthetic construct
  REGION 1..14: note = Recombinant AAV capsid protein ZC473 VR-VIII
ATNHQKLLVNAQTG

SEQ ID NO: 688 - protein, 14 aa, synthetic construct
  REGION 1..14: note = Recombinant AAV capsid protein ZC474 VR-VIII
ATNHLSVSSIAQTG

SEQ ID NO: 689 - protein, 14 aa, synthetic construct
  REGION 1..14: note = Recombinant AAV capsid protein ZC475 VR-VIII
ATNHVSNLYGAQTG

SEQ ID NO: 690 - protein, 14 aa, synthetic construct
  REGION 1..14: note = Recombinant AAV capsid protein ZC476 VR-VIII
ATNRQMAQAQAQTG

SEQ ID NO: 691 - protein, 14 aa, synthetic construct
  REGION 1..14: note = Recombinant AAV capsid protein ZC477 VR-VIII
ATNHEDIIRSAQTG

SEQ ID NO: 692 - protein, 14 aa, synthetic construct
  REGION 1..14: note = Recombinant AAV capsid protein ZC478 VR-VIII
ATNHCSTSIRAQTG

SEQ ID NO: 693 - protein, 8 aa, synthetic construct
  REGION 1..8: note = Recombinant AAV capsid protein ZC396 partial VR-VIII
NHQSPIAQ

SEQ ID NO: 694 - protein, 8 aa, synthetic construct
  REGION 1..8: note = Recombinant AAV capsid protein ZC398 partial VR-VIII
NHQSAITQ

SEQ ID NO: 695 - protein, 8 aa, synthetic construct
  REGION 1..8: note = Recombinant AAV capsid protein ZC408 partial VR-VIII
NHQHYSAQ

SEQ ID NO: 696 - protein, 8 aa, synthetic construct
  REGION 1..8: note = Recombinant AAV capsid protein ZC420 partial VR-VIII
NTYHQSAQ

SEQ ID NO: 697 - protein, 8 aa, synthetic construct
  REGION 1..8: note = Recombinant AAV capsid protein ZC424 partial VR-VIII
NHQVTSAQ

SEQ ID NO: 698 - protein, 8 aa, synthetic construct
  REGION 1..8: note = Recombinant AAV capsid protein ZC427 partial VR-VIII
NHQSAPTQ

SEQ ID NO: 699 - protein, 8 aa, synthetic construct
  REGION 1..8: note = Recombinant AAV capsid protein ZC430 partial VR-VIII
NHQAISAQ

SEQ ID NO: 700 - protein, 8 aa, synthetic construct
  REGION 1..8: note = Recombinant AAV capsid protein ZC432 partial VR-VIII
NHMHQSAQ

SEQ ID NO: 701 - protein, 8 aa, synthetic construct
  REGION 1..8: note = Recombinant AAV capsid protein ZC441 partial VR-VIII
NFNHQSAQ

SEQ ID NO: 702 - protein, 8 aa, synthetic construct
  REGION 1..8: note = Recombinant AAV capsid protein ZC443 partial VR-VIII
NHQWMSAQ

SEQ ID NO: 703 - protein, 8 aa, synthetic construct
  REGION 1..8: note = Recombinant AAV capsid protein ZC450 partial VR-VIII
NHQMLSAQ

SEQ ID NO: 704 - protein, 8 aa, synthetic construct
  REGION 1..8: note = Recombinant AAV capsid protein ZC456 partial VR-VIII
NATHQSAQ

SEQ ID NO: 705 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHENTVSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 706 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 707 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 708 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHGSILTHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 709 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIIGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 710 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 711 - skipped

SEQ ID NO: 712 - protein, 4 aa, synthetic construct
NTVS

SEQ ID NO: 713 - protein, 4 aa, synthetic construct
TLFN

SEQ ID NO: 714 - protein, 4 aa, synthetic construct
STYL

SEQ ID NO: 715 - protein, 4 aa, synthetic construct
SILT

SEQ ID NO: 716 - protein, 4 aa, synthetic construct
MTTA

SEQ ID NO: 717 - protein, 4 aa, synthetic construct
STSI

SEQ ID NO: 718 - skipped

SEQ ID NO: 719 - protein, 6 aa, synthetic construct
ENTVSI

SEQ ID NO: 720 - protein, 6 aa, synthetic construct
QTLFNS

SEQ ID NO: 721 - protein, 6 aa, synthetic construct
NSTYLG

SEQ ID NO: 722 - protein, 6 aa, synthetic construct
GSILTH

SEQ ID NO: 723 - protein, 6 aa, synthetic construct
MMTTAR

SEQ ID NO: 724 - protein, 6 aa, synthetic construct
CSTSIR

SEQ ID NO: 725 - protein, 6 aa, synthetic construct
EDNIRS

SEQ ID NO: 726 - protein, 14 aa, synthetic construct
ATNHEDNIRSAQTG

SEQ ID NO: 727 - protein, 9 aa, synthetic construct
KGSGQNQQT

SEQ ID NO: 728 - protein, 14 aa, synthetic construct
  VARIANT 5: note = Q, E, N, G, M, C, V, or T
  VARIANT 6: note = S, N, T, M, G, or D
  VARIANT 7: note = A, T, L, I, K, S, N or V
  VARIANT 8: note = Q, V, F, Y, L, T, S, I, R, or Q
  VARIANT 9: note = A, S, N, L, T, I, or R
  VARIANT 10: note = Q, I, S, G, H or R
ATNHXXXXXXAQTG

SEQ ID NO: 729-738 - skipped (10 entries)

SEQ ID NO: 739 - protein, 14 aa, synthetic construct
  VARIANT 5: note = Q, E, N, G, M, or C
  VARIANT 6: note = S, N, T, or M
  VARIANT 7: note = A, T, L, I, or S
  VARIANT 8: note = Q, V, F, Y, L, or I
  VARIANT 9: note = A, S, N, L, T, or I
  VARIANT 10: note = I, S, Q, G, H, or R
ATNHXXXXXXAQTG

SEQ ID NO: 740 - protein, 16 aa, synthetic construct
  VARIANT 5..12: note = X is any amino acid
  VARIANT 9..12: note = up to 4 amino acids may be absent
ATNHXXXXXXXXAQTG

SEQ ID NO: 741 - protein, 4 aa, synthetic construct
GAYA

SEQ ID NO: 742 - protein, 4 aa, synthetic construct
TKLA

SEQ ID NO: 743 - protein, 4 aa, synthetic construct
SSFT

SEQ ID NO: 744 - protein, 4 aa, synthetic construct
DNIR

SEQ ID NO: 745 - protein, 4 aa, synthetic construct
NVIS

SEQ ID NO: 746 - protein, 4 aa, synthetic construct
GTSI

SEQ ID NO: 747 - protein, 4 aa, synthetic construct
DARA

SEQ ID NO: 748 - protein, 4 aa, synthetic construct
SAQA

SEQ ID NO: 749 - protein, 6 aa, synthetic construct
QGAYAQ

SEQ ID NO: 750 - protein, 6 aa, synthetic construct
NTKLAI

SEQ ID NO: 751 - protein, 6 aa, synthetic construct
VSSFTS

SEQ ID NO: 752 - protein, 6 aa, synthetic construct
NNVISG

SEQ ID NO: 753 - protein, 6 aa, synthetic construct
TGTSII

SEQ ID NO: 754 - protein, 6 aa, synthetic construct
QANYGQ

SEQ ID NO: 755 - protein, 6 aa, synthetic construct
QDARAQ

SEQ ID NO: 756 - protein, 6 aa, synthetic construct
QSAQAQ

SEQ ID NO: 757 - protein, 9 aa, synthetic construct
KYHKSGAAQ

SEQ ID NO: 758 - protein, 9 aa, synthetic construct
KQVNGRPRD

SEQ ID NO: 759 - protein, 8 aa, synthetic construct
QHYSAQAQ

SEQ ID NO: 760-766 - skipped (7 entries)

SEQ ID NO: 767 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 768 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 769 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 770 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTKLAIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 771 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSSFTSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 772 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 773 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 774 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 775 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTGTSIIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 776 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQWMSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 777 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDARAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 778 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQHYSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 779 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNIRTEMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 780 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTTNFRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 781 - skipped

SEQ ID NO: 782 - DNA, 2091 bp, Homo sapiens
  source 1..2091: mol_type = genomic DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 783 - protein, 696 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVRTTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 784 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 785 - DNA, 1728 bp, Homo sapiens
  source 1..1728: mol_type = genomic DNA
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtag

SEQ ID NO: 786 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 787 - DNA, 528 bp, Homo sapiens
  source 1..528: mol_type = genomic DNA
atggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatag

SEQ ID NO: 788 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 789 - DNA, 1995 bp, Homo sapiens
  source 1..1995: mol_type = genomic DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaa

SEQ ID NO: 790 - protein, 572 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHVSGSRR

SEQ ID NO: 791 - DNA, 1719 bp, Homo sapiens
  source 1..1719: mol_type = genomic DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacgtgagtggtagccgccgctga

SEQ ID NO: 792 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 793 - DNA, 633 bp, Homo sapiens
  source 1..633: mol_type = genomic DNA
atggcggatgggagcagcgatgcggctagggaacctcgccctgcaccagccccaatcagacgccgctcctccaactaccgcgcttatgccacggagccgcacgccaagaaaaaatctaagatctccgcctcgagaaaattgcagctgaagactctgctgctgcagattgcaaagcaagagctggagcgagaggcggaggagcggcgcggagagaaggggcgcgctctgagcacccgctgccagccgctggagttggccgggctgggcttcgcggagctgcaggacttgtgccgacagctccacgcccgtgtggacaaggtggatgaagagagatacgacatagaggcaaaagtcaccaagaacatcacggagattgcagatctgactcagaagatctttgaccttcgaggcaagtttaagcggcccaccctgcggagagtgaggatctctgcagatgccatgatgcaggcgctgctgggggcccgggctaaggagtccctggacctgcgggcccacctcaagcaggtgaagaaggaggacaccgagaaggaaaaccgggaggtgggagactggcgcaagaacatcgatgcactgagtggaatggagggccgcaagaaaaagtttgagagctga

SEQ ID NO: 794 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 795 - DNA, 1233 bp, Homo sapiens
  source 1..1233: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttag

SEQ ID NO: 796 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 797 - DNA, 1233 bp, Homo sapiens
  source 1..1233: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 798 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 799 - DNA, 1236 bp, Homo sapiens
  source 1..1236: mol_type = genomic DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaa

SEQ ID NO: 800 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 801 - DNA, 8616 bp, Homo sapiens
  source 1..8616: mol_type = genomic DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggtaagaaacaattatgatgaggagatcattagcttaaaaaatcagtttgagaccgagatcaacatcaccaagaccaccatccaccagctcaccatgcagaaggaagaggataccagtggctaccgggctcagatagacaatctcacccgagaaaacaggagcttatctgaagaaataaagaggctgaagaacactctaacccagaccacagagaatctcaggagggtggaagaagacatccaacagcaaaaggccactggctctgaggtgtctcagaggaaacagcagctggaggttgagctgagacaagtcactcagatgcgaacagaggagagcgtaagatataagcaatctcttgatgatgctgccaaaaccatccaggataaaaacaaggagatagaaaggttaaaacaactgatcgacaaagaaacaaatgaccggaaatgcctggaagatgaaaacgcgagattacaaagggtccagtatgacctgcagaaagcaaacagtagtgcgacggagacaataaacaaactgaaggttcaggagcaagaactgacacgcctgaggatcgactatgaaagggtttcccaggagaggactgtgaaggaccaggatatcacgcggttccagaactctctgaaagagctgcagctgcagaagcagaaggtggaagaggagctgaatcggctgaagaggaccgcgtcagaagactcctgcaagaggaagaagctggaggaagagctggaaggcatgaggaggtcgctgaaggagcaagccatcaaaatcaccaacctgacccagcagctggagcaggcatccattgttaagaagaggagtgaggatgacctccggcagcagagggacgtgctggatggccacctgagggaaaagcagaggacccaggaagagctgaggaggctctcttctgaggtcgaggccctgaggcggcagttactccaggaacaggaaagtgtcaaacaagctcacttgaggaatgagcatttccagaaggcgatagaagataaaagcagaagcttaaatgaaagcaaaatagaaattgagaggctgcagtctctcacagagaacctgaccaaggagcacttgatgttagaagaagaactgcggaacctgaggctggagtacgatgacctgaggagaggacgaagcgaagcggacagtgataaaaatgcaaccatcttggaactaaggagccagctgcagatcagcaacaaccggaccctggaactgcaggggctgattaatgatttacagagagagagggaaaatttgagacaggaaattgagaaattccaaaagcaggctttagaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 802 - protein, 2272 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 803 - DNA, 6819 bp, Homo sapiens
  source 1..6819: mol_type = genomic DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 804 - protein, 1118 aa, Homo sapiens
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 805 - DNA, 3357 bp, Homo sapiens
  source 1..3357: mol_type = genomic DNA
atggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaacgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaaggctttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacactagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagttaccaagcatagcactgtacagcattcttactcctaa

SEQ ID NO: 806 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDMDATYRPMYSSDVPLDPLEMHMDMDGDYPIDTYSDGLRPPYPTADHMLA

SEQ ID NO: 807 - DNA, 2238 bp, Homo sapiens
  source 1..2238: mol_type = genomic DNA
atggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgatctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctag

SEQ ID NO: 808 - protein, 572 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVR

SEQ ID NO: 809 - DNA, 1716 bp, Homo sapiens
  source 1..1716: mol_type = genomic DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgc

SEQ ID NO: 810 - DNA, 159 bp, Homo sapiens
  source 1..159: mol_type = genomic DNA
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 811 - DNA, 3825 bp, Homo sapiens
  source 1..3825: mol_type = genomic DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 812 - DNA, 3546 bp, synthetic construct
  source 1..3546: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 813 - DNA, 3534 bp, synthetic construct
  source 1..3534: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggagcctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 814 - DNA, 3546 bp, synthetic construct
  source 1..3546: mol_type = other DNA
atgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggagcccaggcaggaacctcccaagatccacctggactgcccaggccgcataccagacaccattgtggttgtagctggaaataagctacgtctggacgtccctatctctggggaccccgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggaactctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtaa

SEQ ID NO: 815 - protein, 1274 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 816 - protein, 1181 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 817 - protein, 1177 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 818 - protein, 1181 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 819 - DNA, 1464 bp, Homo sapiens
  source 1..1464: mol_type = genomic DNA
atggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctc

SEQ ID NO: 820 - DNA, 2930 bp, Homo sapiens
  source 1..2930: mol_type = genomic DNA
atgggtgctgaggaggaggtgctggtcacactatcagggggagccccctggggcttccgacttcatgggggggccgagcagaggaaaccgttacaggtgtctaagattcgaagacggagccaggctggcagagcaggactccgagagagggaccagctcttggcaatcaatggggtctcttgcaccaacctctcccatgccagtgccatgagcctcatcgatgcctcaggaaatcagcttgtcctcactgtgcagcggttagcagacgagggtcctgtgcaatctccatctccccatgagcttcaggtgctgtcacccttatctccactaagtcctgagccccctggtgctccagttcctcagcctcttcagcctgggagccttcgttcacctcctgatagtgaggcttactacggagagactgacagtgatgctgatggccctgccacccaggagaagccccgtcgacctcgccgccgaggccccacaaggcccacccctccgggtgccccacctgatgaggtctacctgtctgacagccctgcagagccagcacctactatccctggccctcccagccagggtgacagcgtgtgagctccccgtcttgggaggatggggcagcccttcagccacccccagctgaggctctgctgttaccccatggccccctccgacctggtcctcatctcatccctatggtggggcctgttccccacccagtggcagaagatcttactaccacctacacccagaaggccaagcaagccaaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 821 - DNA, 2259 bp, Homo sapiens
  source 1..2259: mol_type = genomic DNA
atggagacctttgagcccatcagccaagagcccctcagccaagccagctatgacaaagccccagacccagttcctgagctccaagactcgttctatgcagaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 822 - protein, 488 aa, Homo sapiens
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 823 - protein, 977 aa, Homo sapiens
MGAEEEVLVTLSGGAPWGFRLHGGAEQRKPLQVSKIRRRSQAGRAGLRERDQLLAINGVSCTNLSHASAMSLIDASGNQLVLTVQRLADEGPVQSPSPHELQVLSPLSPLSPEPPGAPVPQPLQPGSLRSPPDSEAYYGETDSDADGPATQEKPRRPRRRGPTRPTPPGAPPDEVYLSDSPAEPAPTIPGPPSQGDSRVSSPSWEDGAALQPPPAEALLLPHGPLRPGPHLIPMVGPVPHPVAEDLTTTYTQKAKQAKLQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 824 - protein, 753 aa, Homo sapiens
METFEPISQEPLSQASYDKAPDPVPELQDSFYAELQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 825 - protein, 755 aa, Homo sapiens
MEAVIEKECSALGGLFQTIISDMKGSYPVWEDFINKAGKLQSQLRTTVVAAAAFLDAFQKVADMATNTRGGTREIGSALTRMCMRHRSIEAKLRQFSSALIDCLINPLQEQMEEWKKVANQLDKDHAKEYKKARQEIKKKSSDTLKLQKKAKKGRGDIQPQLDSALQDVNDKYLLLEETEKQAVRKALIEERGRFCTFISMLRPVIEEEISMLGEITHLQTISEDLKSLTMDPHKLPSSSEQVILDLKGSDYSWSYQTPPSSPSTTMSRKSSVCSSLNSVNSSDSRSSGSHSHSPSSHYRYRSSNLAQQAPVRLSSVSSHDSGFISQDAFQSKSPSPMPPEAPNQLSNGFSHYSLSSESHVGPTGAGLFPHCLPASRLLPRVTSVHLPDYAHYYTIGPGMFPSSQIPSWKDWAKPGPYDQPLVNTLQRRKEKREPDPNGGGPTTASGPPAAAEEAQRPRSMTVSAATRPGEEMEACEELALALSRGLQLDTQRSSRDSLQCSSGYSTQTTTPCCSEDTIPSQVSDYDYFSVSGDQEADQQEFDKSSTIPRNSDISQSYRRMFQAKRPASTAGLPTTLGPAMVTPGVATIRRTPSTKPSVRRGTIGAGPIPIKTPVIPVKTPTVPDLPGVLPAPPDGPEERGEHSPESPSVGEGPQGVTSMPSSMWSGQASVNPPLPGPKPSIPEEHRQAIPESEAEDQEREPPSATVSPGQIPESDPADLSPRDTPQGEDMLNAIRRGVKLKKTTTNDRSAPRFS

SEQ ID NO: 826 - protein, 35 aa, Homo sapiens
MAEKAGSTFSHLLVPILLLIGWIVGCIIMIYVVFS

SEQ ID NO: 827 - DNA, 108 bp, Homo sapiens
  source 1..108: mol_type = genomic DNA
atggctgaaaaagcggggtctacattttcacaccttctggttcctattcttctcctgattggctggattgtgggctgcatcataatgatttatgttgtcttctcttag

SEQ ID NO: 828 - DNA, 108 bp, synthetic construct
  source 1..108: mol_type = other DNA
atggccgagaaggccggatctaccttcagccacctgctggtccctattctgctgctgatcggctggatcgtgggctgcatcatcatgatctacgtggtgttcagctga

SEQ ID NO: 829 - protein, 575 aa, Homo sapiens [sequence omitted]

SEQ ID NO: 830 - protein, 52 aa, Homo sapiens
MEKVQYLTRSAIRRASTIEMPQQARQKLQNLFINFCLILICLLLICIIVMLL

SEQ ID NO: 831 - RNA, 4994 nt, Homo sapiens
  source 1..4994: mol_type = mRNA
agagcgagcgccggggccgggcgcgcaggagtgaaaaggaggcggcggccgcagctgcgagcaacagatccggacgccgcgagctgacccgctctgctgttgggcgatttttttttaattgcagaaaaatttattaaattggaaaatcttgcgtttttcaatggcgctggccccgggtcagcgggcgattttctctgcatcaagatgggctttgccgtttccgtagtgggcaccagtggtggcctgattgtcagtcttctcccggcatttttaaggccaggagccgagcgctgcttgtaggcgaataccctacagagcggtttggctttttaaattactgttattattttgggcagagaacagtcggtctggtgcaccccgtcctcgctgcagaagaggctgcgagtccgaggtgggtctctcggaaggtgaaattccttctggggtgagcgagccccggccccgcgcgcagtccagcggccccgcgtgtgtgccctcgccctgccggagccgggaaaatggaggctgtgattgagaaggaatgcagcgcgctcggaggcctcttccagaccatcatcagcgacatgaaggggagctatccagtttgggaagatttcataaacaaagcaggaaagctgcagtcccagcttcggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgttgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttaggttcacaagaaatgcgccggtggggaatgaactgtttcattaataaaacctaatttgtcttgatccattccactctataataaaacaaaagattttgtaggcaactcggaatatagctcttttgaaagtactcgacacctttagataagaattaaaaccaacctatgtaactgacataatcttgatcttttaatttgtaaatattgacaattttctttctgcacattttaatcttagtttcccttttgatttttctgaaggtgccaaattccatttaacttttttacaagtctttgtaaaattttaaatgcataaagggggttggggcaggggaaccacgaagtagttaattttagaaaaggatttactatacttcactcttctttttttttccccacaagcttttgtagatgcattgtagtagtctagcttagaagcaaatgcaagttattttaatgtacaaactaaatgggtaagaggtaaaatcttcatttaaatatactatgttctggatgaaaagagcaggagtaacaattgatgagcaatattcagagtgaagtaaatctggaaatggtagactgtgttgggattggggggagggccatgggaggggtacatcgtcaacatagccgatcctgttacatttaagagtagcctcgtaggttgaatttcttctggtagcttcatggtaaatgcatccgaataagccatactggattgcagtgtttgtttctgtagggtgtttaaggacttgacttcctttctcccatgattcctctggactgcacacagcacccacaaccagccccatgcatgctgctgcctctgggcagtcgtagaatctcccacttcagtttctcgttgattgtactcacctttatggaatccaaatacatccaaaagggtaaggcagttttaaaaatgtgaaaacatttaaaaatgataatagcagggaattcttagattatagtaaatgccttttacttaactgtgcccagcaggctgggtgcgttaaaaagcccaagtattttgaaaaaactcgaacagatttgacaagggtagccagcttggagtctagcaacttgccaatgtgtttaccaatctgggggcttgtttttcttttcttctttcaaataaatggcagttaactggctttacagtaaacattgaagagaggaggatttgtttattgtcactgggaatctgaccactatactgtcctttttttgtattctgggtaaatgttttttggaaaagatttgtcttttctaagtggaagttaaatttgttatactgcccatcccctaaagccaacagagatttgtagatttaaagggatcacatttgaagacaatagtgtttaagaaagcaagcaagtcccttagcagtcaggtcataacagggcacatttctgaccgaaccctctcaaggcagaggaggagtttggtgggtttcatacaccctgcagattcctgttggctctaaccctcaattacctaatcttatgctttaacacataactgcattggatgtgagagtaacgtaccgtatggtcattgttctatatattaacattgaacactgctgcgattgctcaaggacattttatgttacggctttaaagcaaaggcatgattattagaaactatttaagcttttttctttgaaaaacaagctccttttacagaatataaacaacagtagtgcctgtggtttagcccaccaatcttgatgactaaaagtagctgatgcattgtgcatatgatgcttgagatggtttttgcaaaagcagaaatcgctgcaaggtaatcacaatagataaaagtggtattttaaacctttgaaataaatggatgtaactgtaccttggtacagcttttcacttgtttagtttttaaacgttagtataatctgaataaataaaatgttgccaaattcaatgtagaaagaatgtgacaacacaccttgggtagttctgcttgtgtttttgcatattgtaaaagcagtgtcacagctaaaaagaaagaaatcgtttctaacagtaaattattgtgctttagttgctagtttgtactgagagttgacctctccctgtgcagttttttgttctaaacttgtataaataacaattgtgtaatgtgtctccctcctacattgtaacaattgcttcagcctacgttataaataaagaaccactagattaaaaaa

SEQ ID NO: 832 - DNA, 3258 bp, Staphylococcus aureus
  source 1..3258: mol_type = genomic DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 833 - protein, 1085 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK